(19) United States
(12) Patent Application Publication
(10) Pub. No.: US 2026/0224596 A1
(43) Pub. Date: Aug. 6, 2026

(54) Phosphate Prodrugs of Cannabinoids

(71) Applicant: Emory University, Atlanta, GA (US)

(72) Inventors: Dennis C. Liotta, Atlanta, GA (US); Stephen F. Traynelis, Decatur, GA (US); Luke E. Hodson, Atlanta, GA (US); Nicole Pribut, Atlanta, GA (US); Leon Jacobs, Atlanta, GA (US)

(21) Appl. No.: 19/153,315

(22) PCT Filed: Feb. 1, 2024
(86) PCT No.: PCT/US2024/014093

§ 371 Date: Aug. 1, 2025

Related U.S. Application Data
(60) Provisional application No. 63/442,686, filed on Feb. 1, 2023.

(51) Int. Cl. A61K 31/00; C07F 9/655

(52) U.S. Cl. CPC A61K 31/658; C07F 9/65522

(57) ABSTRACT

Phosphate prodrugs of cannabinoids, with enhanced physicochemical, pharmacological, and/or pharmacokinetic properties, such as enhanced aqueous solubility and enhanced oral bioavailability are disclosed herein, as are pharmaceutical compositions comprising one or more of the same. Such compounds and compositions are useful for the treatment of diseases, disorders, and/or conditions where treatment with a cannabinoid may be useful, including pain, multiple sclerosis, and epilepsy.

### CROSS-REFERENCE TO RELATED APPLICATIONS

This application claims the benefit of U.S. Provisional Application No. 63/442,686 filed Feb. 1, 2023. The entirety of this application is hereby incorporated by reference for all purposes.

### TECHNICAL FIELD

The present disclosure relates to prodrugs of cannabinoids (CBs), pharmaceutical compositions comprising one or more of the same, and uses related thereto.

### BACKGROUND

Cannabinoids refer to a heterogenous family of molecules extracted from Cannabis sativa, which have been shown to exhibit pharmacological properties by interacting with specific receptors, such as membrane receptors coupled to G proteins. The pharmacology of these compounds is rapidly expanding, and there is a growing body of pharmacological effects and therapeutic properties of CB receptor agonists. These include analgesia, muscle relaxation, immunosuppressant, anti-inflammatory effects, anti-allergic effects, anti-psychotic effects, antioxidant effects, anticancer effects, anxiolytic effects, improvement of mood, stimulation of appetite, anti-emesis, lowering of intraocular pressure, bronchodilation, neuroprotection, anti-convulsant effects, and antineoplastic effects. In this context, the clinical usefulness of cannabinoids has been well-recognized in a wide range of areas, e.g., to provide analgesia and neuroprotection, reduce inflammation, alleviate nausea and emesis, alleviate symptoms of multiple sclerosis (such as neuropathic pain, spasticity, and overactive bladder), and treat epilepsy, anxiety disorder, Tourette Syndrome, dystonia, rheumatoid arthritis, addictions (such as alcohol use disorder and opioid addiction), glaucoma, anorexia, and weight loss.

For example, pain is a common clinical problem that confronts clinicians. Millions of people in the US suffer from severe pain that, according to numerous reports, is chronically under-treated or inappropriately managed. Similarly, millions of people also suffer from nausea and/or frequent emesis. Cannabinoids are effective in alleviating pain. Moreover, cannabinoids can reduce a patient's nausea and vomiting, independent of any pain relief achieved.

A notable percentage of the US population also suffers from alcohol use disorder (“AUD”). The consumption of excessive amounts of alcohol results in a complex array of pharmacological effects that directly impact the ability to treat the condition. These effects directly impact the brain and include progressive neurodegeneration, impaired executive function, and dependence leading to withdrawal-induced negative effects. Cannabinoids have neuroprotective, anxiolytic, and anti-convulsant effects, making them effective in preventing brain damage in persons with AUD, while simultaneously decreasing the frequency of relapses.

Dystonia is a neurological movement disorder with many known causes. It is characterized by involuntary, continual muscular contractions causing twisting and repetitive movements or abnormal postures. Cannabinoids have been shown to reduce the symptoms of muscular contractions associated this disorder.

The etiological pathology of many diseases relates to the inflammatory processes caused by an individual's immune system. The inflammation may result from (1) an otherwise appropriate immune response to an outside trauma, such as brain swelling secondary to a closed head injury; (2) an overactive immune response, such as with an allergic reaction or dermatitis; or (3) an inappropriate autoimmune response, such as what causes certain forms of multiple sclerosis, inflammatory bowel disorders, and arthritis. Regardless of the underlying cause of the inflammation, it is therapeutically desirable under these circumstances to regulate the immune system and lessen the inflammatory response. Cannabinoids have been shown to regulate various steps in the immune response and generate therapeutic benefits in treating many inflammatory diseases. For example, rheumatoid arthritis affects approximately 0.5-1% of the US population. Cannabinoids have been found to be useful for treating rheumatoid arthritis and joint pain secondary to other autoimmune diseases, such as inflammatory bowel disease, multiple sclerosis, and systemic lupus erythematosus.

Notably, nabiximols (SATIVEX®) is a combination cannabinoid-based drug standardized in composition, formulation, and dose. Its principal active components are two cannabinoids: Δ⁹-tetrahydrocannabinol (Δ⁹-THC) and cannabidiol (CBD). Nabiximols is administered by oromucosal spray and has been approved for the treatment of multiple sclerosis symptoms in Europe. It has been shown that co-administration of CBD and THC may alter the pharmacological effects of the latter, potentiating some putative benefits while attenuating some of its negative effects.

Despite the promising clinical utilities in the afore-described therapeutic areas, cannabinoids often suffer from poor drug-like properties. For example, they have low aqueous solubility, generate GI tract side effects, and/or undergo substantial first-pass metabolism when absorbed from the human gut after oral administration, all of which contribute to undesirable oral bioavailability. These poor drug-like properties result in sub-optimal treatments and limited applications.

Thus, there is a need for CB drugs with enhanced physicochemical, pharmacological, and/or pharmacokinetic properties, such as enhanced aqueous solubility and enhanced oral bioavailability. For example, there is a need for improved prodrug strategies that can overcome one or more of the physicochemical, pharmacological, and/or pharmacokinetic liabilities of CBs.

### SUMMARY

Disclosed are cannabinoid prodrugs of Formula (I):

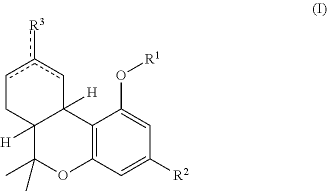

and pharmaceutically acceptable salts thereof, wherein
R¹ is chosen from

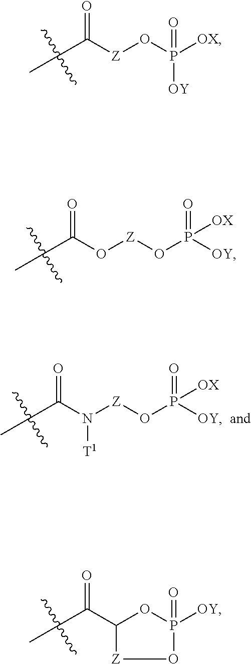

Z is chosen from divalent C₁₋₁₈ alkyl and divalent C₁₋₁₈ haloalkyl groups, wherein the divalent C₁₋₁₈ alkyl and divalent C₁₋₁₈ haloalkyl groups are optionally substituted with one or more groups independently chosen from C₆₋₁₈ aryl, C₁₋₁₃ heteroaryl, C₂₋₁₂heterocyclyl, —OC₁₋₁₈ alkyl, —SC₁₋₁₈ alkyl. —N(T²)C₁₋₁₈ alkyl, and —N(T²)AA groups, wherein T² is chosen from H and C₁₋₈ alkyl groups and AA is chosen from amino acid residues and is attached to the nitrogen via its C-terminus carbonyl group (i.e., forming an amide bond),
X and Y, which may be identical or different, are independently chosen from H, Q, C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

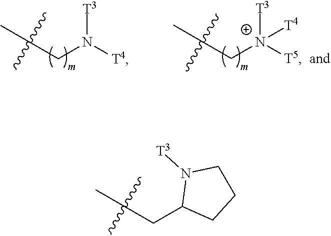

groups, wherein
   each T³, T⁴, and T⁵, which may be the same or different, are independently chosen from H and C₁₋₈ alkyl groups,
   each Q is independently chosen from pharmaceutically acceptable cations,
   each n is independently chosen from integers ranging from 1 to 12, and
   each m is independently chosen from integers ranging from 1 to 8, and
T¹ is chosen from H, C₁₋₁₈ alkyl, and C₁₋₈ haloalkyl groups;
R² is chosen from C₁₋₁₀ alkyl groups and C₁₋₁₀ haloalkyl groups; and
R³ is chosen from —CH₃, —CD₃, —CH₂OH, —CH₂OR^{a}, and ═O, wherein R^{a} is chosen from C₁₋₈ alkyl groups and C₁₋₈ haloalkyl groups.

The covalent bond between the two carbon atoms connected by each dotted line may be a double bond (i.e., when the dotted line represents a pair of shared electrons) or a single bond (i.e., when the dotted line is void). All carbon atoms, including those connected by the dotted lines, have a saturated valency. Hydrogen atoms, albeit not drawn in the chemical structure above, are present to maintain the saturated valency of the carbon atoms when appropriate.

In some embodiments, the compounds are in a non-salt form. In some embodiments, the compounds are in a salt form. In some embodiments, the compounds are in an ammonium salt form.

In some embodiments, R¹ is chosen from

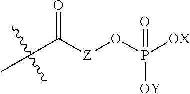

groups. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

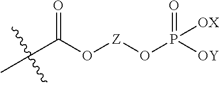

groups.

In some embodiments, R¹ is chosen from

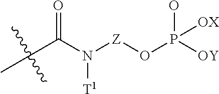

groups.

In some embodiments, R¹ is chosen from

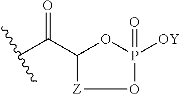

groups.

In some embodiments, Z is chosen from —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—,

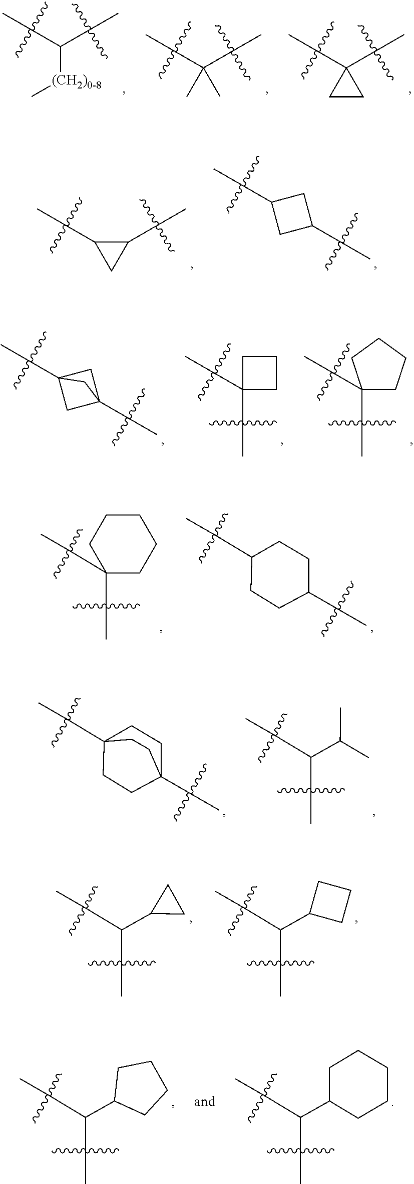

In some embodiments, Z is —CH₂—. In some embodiments, Z is —(CH₂)₂—. In some embodiments, Z is —(CH₂)₃—. In some embodiments, Z is

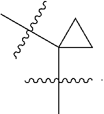 [SMILES: *C1(*)CC1]

In some embodiments, Z is

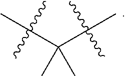 [SMILES: *C(*)(C)C]

In some embodiments, Z is

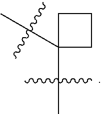 [SMILES: *C1(*)CCC1]

In some embodiments, Z is

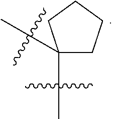 [SMILES: *C1(*)CCCC1]

In some embodiments, Z is

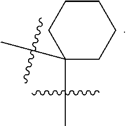 [SMILES: *C1(*)CCCCC1]

In some embodiments, Z is

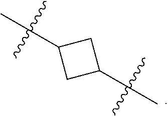 [SMILES: *C1CC(*)C1]

In some embodiments, Z is

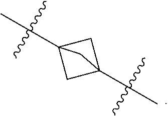 [SMILES: *C12CC(*)(C1)C2]

In some embodiments, Z is

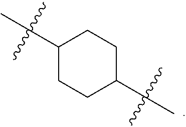 [SMILES: *C1CCC(*)CC1]

In some embodiments, Z is

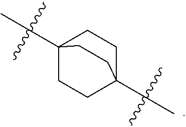 [SMILES: *C12CCC(*)(CC1)CC2]

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q.

In some embodiments, R² is chosen from C₂₋₉ alkyl groups. In some embodiments, R² is n-pentyl. In some embodiments, R² is n-propyl. In some embodiments, R² is 1,1-dimethylheptyl.

In some embodiments, R³ is chosen from —CH₃, —CD₃, —CH₂OH, and —CH₂OR^{a}. In some embodiments, R³ is —CH₃. In some embodiments, R³ is —CD₃. In some embodiments, R³ is —CH₂OH. In some embodiments, R³ is —CH₂OR^{a}. In some embodiments, R³ is ═O.

In some embodiments, R² is chosen from n-propyl, n-pentyl, and 1,1-dimethylheptyl, and R³ is chosen from —CH₃, —CD₃, —CH₂OH, and —CH₂OR^{a}. In some embodiments, R² is chosen from n-propyl, n-pentyl, and 1,1-dimethylheptyl, and R³ is ═O.

In some embodiments, the

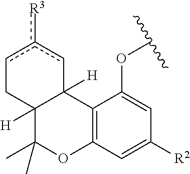

moiety of Formula (I) is chosen from

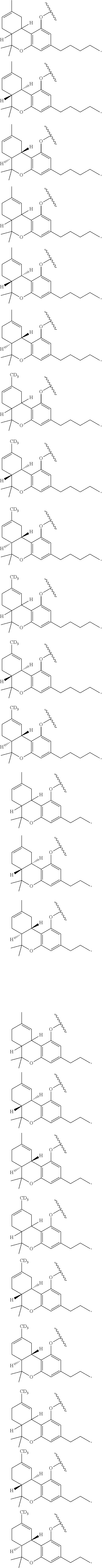

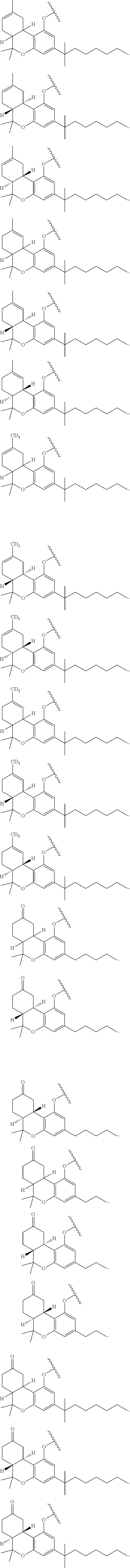

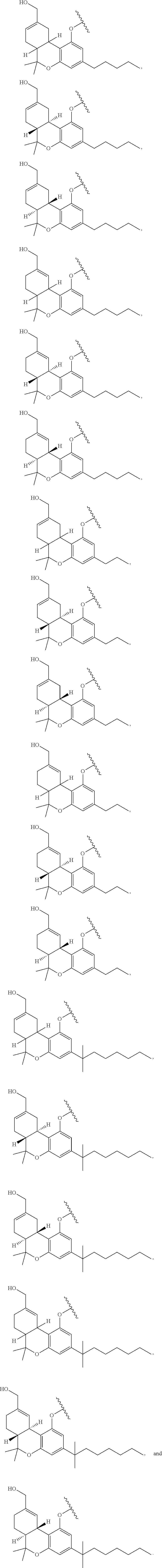

In some embodiments, pharmaceutical compositions containing at least one compound disclosed herein and a pharmaceutically acceptable carrier are presented.

In some embodiments, a method for treatment and/or prevention of at least one disease, disorder, and/or condition where treatment with a cannabinoid is useful is disclosed, the method including administering to a subject in need thereof an effective amount of at least one compound disclosed herein or a pharmaceutical composition disclosed herein.

### DETAILED DESCRIPTION

Disclosed herein are cannabinoid prodrugs and pharmaceutical compositions comprising one or more of the same. The compounds and compositions of the present disclosure may be useful for treating and/or preventing at least one disease, disorder, and/or condition that is treatable or preventable by administering a cannabinoid.

Before the present disclosure is described in greater detail, it is to be understood that this disclosure is not limited to the particular embodiments described herein, and as such, may vary in accordance with the scope of the present disclosure. Unless defined otherwise, all technical and scientific terms used herein have the same meaning as commonly understood by one of ordinary skill in the art to which this disclosure belongs.

All publications and patents cited in this specification are herein incorporated by reference as if each individual publication and patent were specifically and individually indicated to be incorporated by reference. They are incorporated by reference to disclose and describe the methods and/or materials in connection with which the publications and patents are cited.

As will be apparent to those of ordinary skill in the art upon reading this disclosure, each of the particular embodiments described and illustrated herein has discrete components and/or features that may be readily separated from or combined with one or more components and/or features of any of the other embodiments described herein, without departing from the scope or spirit of the present disclosure. Any recited method can be carried out in the order of events recited herein or in any other order that is logically possible.

Embodiments of the present disclosure will employ, unless otherwise indicated, techniques of medicine, organic chemistry, medicinal chemistry, biochemistry, molecular biology, pharmacology, neurology, and the like, which are within the skill of the art. Such techniques are explained fully in the literature, such as the publications and patents cited herein.

### I. Compounds

Disclosed are cannabinoid prodrugs. The prodrugs of the present disclosure may have at least one improved physicochemical, pharmacological, and/or pharmacokinetic property, compared to their corresponding parent compounds.

Methods of making exemplary compounds are disclosed in subsequent sections and exemplified by the Examples. The synthetic methods disclosed herein are compatible with a wide variety of functional groups and starting materials. Thus, a wide variety of compounds can be obtained from the disclosed methods.

The term “alkyl” includes saturated straight, branched, and cyclic (also identified as cycloalkyl) hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Optionally, the cyclic alkyl groups may be bridged cyclic alkyl groups, such as bicyclo[1.1.1]pentyl and bicyclo[2.2.2]octyl. Unless stated otherwise specifically in the specification, an alkyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N. O, and S. Unless stated otherwise specifically in the specification, the heterocyclyl groups may be monocyclic, bicyclic, tricyclic or tetracyclic ring systems, which may include fused, spiro, or bridged ring systems and combinations thereof, and may be partially or fully saturated; any nitrogen, carbon or sulfur atom(s) in the heterocyclyl group may be optionally oxidized; any nitrogen atom in the heterocyclyl group may be optionally quaternized. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl. 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

The term “pharmaceutically acceptable salts” includes both acid and base addition salts. Non-limiting examples of pharmaceutically acceptable acid addition salts include acetates, adipates, ascorbates, aspartates, benzoates, besylates, bicarbonates/carbonates, bisulphates/sulphates, borates, camsylates, citrates, cyclamates, edisylates, esylates, formates, fumarates, gluceptates, gluconates, glucuronates, hexafluorophosphates, hibenzates, hydrochlorides/chlorides, hydrobromides/bromides, hydroiodides/iodides, isethionates, lactates, malates, maleates, malonates, mesylates, sulphates, sulfonates, naphthylates. 2-napsylates, nicotinates, nitrates, orotates, oxalates, palmitates, pamoates, phosphates/hydrogen phosphates/dihydrogen phosphates, pyroglutamates, saccharates, salicylates, stearates, succinates, tannates, tartrates, tosylates, trifluoroacetates, and xinofoates. Non-limiting examples of pharmaceutically acceptable base addition salts include aluminium, arginine, benzathine, calcium, choline, copper, diethylamine, diolamine, glycine, iron, lithium, lysine, magnesium, manganese, meglumine, olamine, potassium, sodium, tromethamine and zinc salts. Suitable base salts also include both unsubstituted and substituted ammonium salts. Hemisalts of acids and bases may also be formed, for example, hemisulphate and hemicalcium salts. Pharmaceutically acceptable salts may, for example, be obtained using standard procedures well known in the field of pharmaceuticals. For a review on suitable salts, see Handbook of Pharmaceutical Salts: Properties, Selection and Use by Stahl and Wermuth (Wiley-VCH, 2002), incorporated herein by reference.

When the compounds of the present disclosure contain an acidic group as well as a basic group, the compounds may also form internal salts and such compounds are within the scope of the disclosure. When a compound contains a hydrogen-donating heteroatom (e.g., NH), salts are contemplated to covers isomers formed by transfer of said hydrogen atom to a basic group or atom within the molecule.

The term “substituted” includes the situation where, in any of the groups herein, at least one hydrogen atom is replaced by a non-hydrogen atom such as, for example, a halogen atom such as F, Cl, Br, and I; a carbon atom in groups such as alkyl, aryl, heterocyclyl, heteroaryl, arylalkyl, heterocyclylalkyl, and heteroarylalkyl: an oxygen atom in groups such as hydroxyl, alkoxy groups, and ester groups; a sulfur atom in groups such as thiol, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the groups above, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof, conventional resolution methods, e.g., fractional crystallization and chiral chromatography, may be used.

The present disclosure includes within its scope all possible tautomers. Furthermore, the present disclosure includes in its scope both the individual tautomers and any mixtures thereof.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell-based activity assays.

Conditions for a particular assay include temperature, buffers (including salts, cations, and media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example. “n is an integer ranging from 0 to 2” includes, independently, 0, 1, and 2.

The term “at least one” refers to one or more, such as one, two, etc. For example, the term “at least one group” refers to one or more groups, such as one group, two groups, etc.

### A. General Structure

In some embodiments, presented are cannabinoid prodrugs of Formula (I):

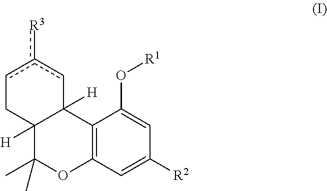

and pharmaceutically acceptable salts thereof, wherein
R¹ is chosen from

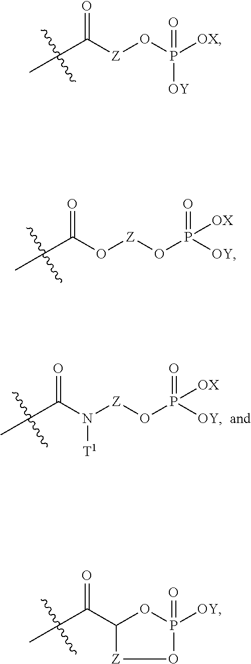

wherein
   Z is chosen from divalent C₁₋₁₈ alkyl and divalent C₁₋₁₈ haloalkyl groups, wherein the divalent C₁₋₁₈ alkyl and divalent C₁₋₁₈ haloalkyl groups are optionally substituted with one or more groups independently chosen from C₆₋₁₈ aryl, C₁₋₁₃ heteroaryl, C₂₋₁₂heterocyclyl, —OC₁₋₁₈ alkyl, —SC₁₋₁₈ alkyl, —N(T²)C₁₋₁₈ alkyl, and —N(T²)AA groups, wherein T² is chosen from H and C₁₋₈ alkyl groups and AA is chosen from amino acid residues and is attached to the nitrogen via its C-terminus carbonyl group (i.e., forming an amide bond),
   X and Y, which may be identical or different, are independently chosen from H, Q, C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

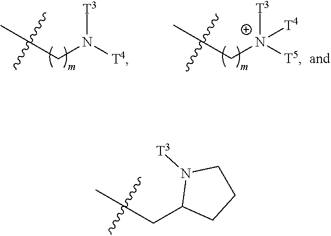

groups, wherein
   each T³, T⁴, and T⁵, which may be the same or different, are independently chosen from H and C₁₋₈ alkyl groups,
   each Q is independently chosen from pharmaceutically acceptable cations,
   each n is independently chosen from integers ranging from 1 to 12, and
   each m is independently chosen from integers ranging from 1 to 8, and
T¹ is chosen from H, C₁₋₁₈ alkyl, and C₁₋₁₈ haloalkyl groups:
R² is chosen from C₁₋₁₀ alkyl groups and C₁₋₁₀ haloalkyl groups; and
R³ is chosen from CH₃, CD₃, CH₂OH, CH₂OR^{a}, and ═O, wherein R^{a} is chosen from C₁₋₈ alkyl groups and C₁₋₈ haloalkyl groups.

The covalent bond between the two carbon atoms connected by each dotted line may be a double bond (i.e., when the dotted line represents a pair of shared electrons) or a single bond (i.e., when the dotted line is void). All carbon atoms, including those connected by the dotted lines, have a saturated valency. Hydrogen atoms, albeit not drawn in the chemical structure above, are present to maintain the saturated valency of the carbon atoms when appropriate.

In some embodiments, the compounds are in a non-salt form. In some embodiments, the compounds are in a salt form. In some embodiments, the compounds are in an ammonium salt form.

In some embodiments, Formula (I) is in the form of Formula (I)-1.

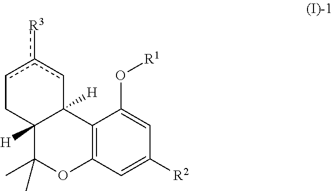

In some embodiments, Formula (I) is in the form of Formula (I)-2.

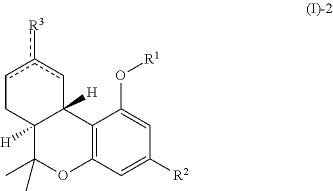

### 1. R¹

In some embodiments, R¹ is chosen from

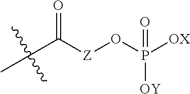

groups. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

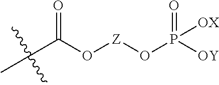

groups. In some embodiments, the carbon in Z that is next to the carbonyloxy group

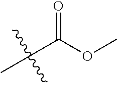 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

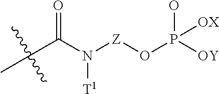

groups. In some embodiments, the carbon in Z that is next to the carbonylamino group

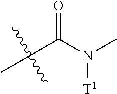

(i.e., the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, T¹ is H. In some embodiments, T¹ is methyl.

In some embodiments, R¹ is chosen from

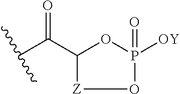

groups.

In some embodiments, Z is chosen from divalent C₁₋₁₈ alkyl groups. In some embodiments, Z is chosen from divalent C₁₋₁₂ alkyl groups. In some embodiments, Z is chosen from divalent C₁₋₈ alkyl groups. In some embodiments, Z is chosen from divalent C₁₋₆ alkyl groups. In some embodiments, Z is chosen from divalent C₁₋₄ alkyl groups. In some embodiments, Z is chosen from divalent C₁₋₃ alkyl groups. In some embodiments, Z is chosen from divalent C₁₋₂ alkyl groups. In some embodiments, Z is a divalent C₁ alkyl group, i.e., divalent methylene.

In some embodiments, Z is chosen from —CH₂—, —(CH₂)₂—, —(CH₂)₃—, —(CH₂)₄—. —(CH₂)₅—, —(CH₂)₆—, —(CH₂)₇—, —(CH₂)₈—, —(CH₂)₉—, —(CH₂)₁₀—,

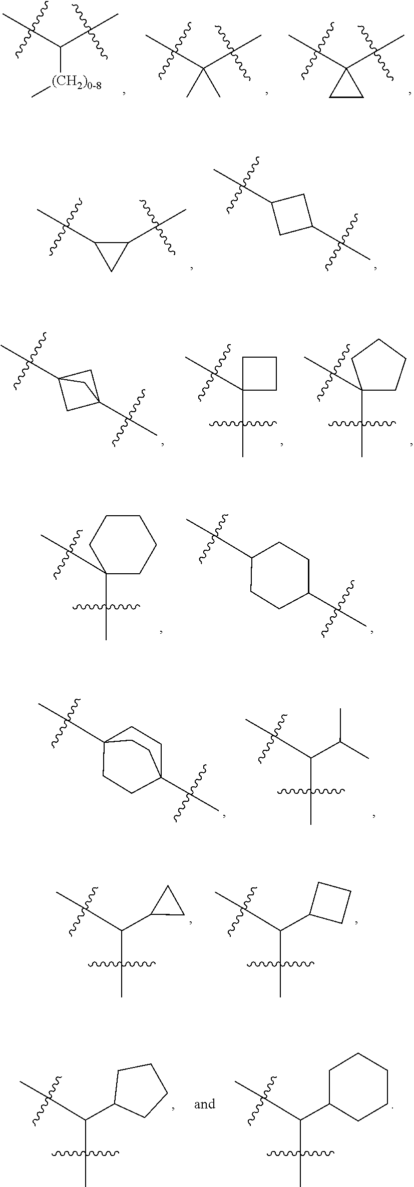

In some embodiments, Z is —CH₂—. In some embodiments, Z is —(CH₂)₂—. In some embodiments. Z is —(CH₂)₃—. In some embodiments, Z is

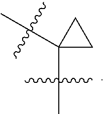 [SMILES: *C1(*)CC1]

In some embodiments, Z is

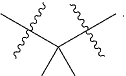 [SMILES: *C(*)(C)C]

In some embodiments Z is

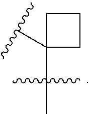 [SMILES: *C1(*)CCC1]

In some embodiments Z is

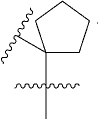 [SMILES: *C1(*)CCCC1]

In some embodiments, Z is

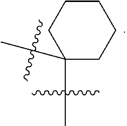 [SMILES: *C1(*)CCCCC1]

In some embodiments, Z is

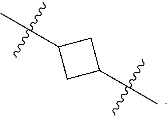 [SMILES: *C1CC(*)C1]

In some embodiments, Z is

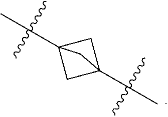 [SMILES: *C12CC(*)(C1)C2]

In some embodiments, Z is

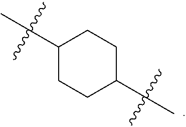 [SMILES: *C1CCC(*)CC1]

In some embodiments, Z is

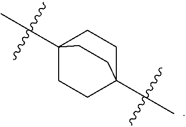 [SMILES: *C12CCC(*)(CC1)CC2]

In some embodiments, Z is chosen from divalent C₁₋₁₈ haloalkyl groups. In some embodiments, Z is chosen from divalent C₁₋₁₂ haloalkyl groups. In some embodiments, Z is chosen from divalent C₁₋₈ haloalkyl groups. In some embodiments, Z is chosen from divalent C₁₋₆ haloalkyl groups. In some embodiments, Z is chosen from divalent C₁₋₄ haloalkyl groups. In some embodiments, Z is chosen from divalent C₁₋₃ haloalkyl groups. In some embodiments, Z is chosen from divalent C₁₋₂ haloalkyl groups. In some embodiments, Z is a divalent C₁ haloalkyl group, i.e., divalent halomethylene.

In some embodiments, Z is

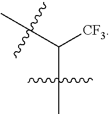 [SMILES: *C(*)C(F)(F)F]

In some embodiments, Z is

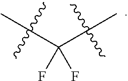 [SMILES: *C(*)(F)F]

In some embodiments, Z is chosen from divalent C₁₋₁₈ alkyl groups substituted with at least one group chosen from C₆₋₁₈ aryl groups. In some embodiments, Z is chosen from divalent C₁₋₁₆ alkyl groups substituted with at least one group chosen from C₆₋₁₈ aryl groups. In some embodiments, Z is chosen from divalent C₁₋₁₄ alkyl groups substituted with at least one group chosen from C₆₋₁₈ aryl groups. In some embodiments, Z is chosen from divalent C₁₋₁₂ alkyl groups substituted with at least one group chosen from C₆₋₁₈ aryl groups. In some embodiments, Z is chosen from divalent C₁₋₁₀ alkyl groups substituted with at least one group chosen from C₆₋₁₈ aryl groups. In some embodiments, Z is chosen from divalent C₁₋₈ alkyl groups substituted with at least one group chosen from C₆₋₁₈ aryl groups. In some embodiments, Z is chosen from divalent C₁₋₆ alkyl groups substituted with at least one group chosen from C₆₋₁₈ aryl groups. In some embodiments, Z is chosen from divalent C₁₋₄ alkyl groups substituted with at least one group chosen from C₆₋₁₈ aryl groups. In some embodiments, Z is chosen from divalent C₂ alkyl groups substituted with at least one group chosen from C₆₋₁₈ aryl groups. In some embodiments, Z is a divalent C₁ alkyl group substituted with at least one group chosen from C₆₋₁₈ aryl groups.

In some embodiments, Z is chosen from divalent C₁₋₁₈ alkyl groups substituted with at least one group chosen from C₆₋₁₀ aryl groups. In some embodiments, Z is chosen from divalent C₁₋₁₆ alkyl groups substituted with at least one group chosen from C₆₋₁₀ aryl groups. In some embodiments, Z is chosen from divalent C₁₋₁₄ alkyl groups substituted with at least one group chosen from C₆₋₁₀ aryl groups. In some embodiments, Z is chosen from divalent C₁₋₁₂ alkyl groups substituted with at least one group chosen from C₆₋₁₀ aryl groups. In some embodiments, Z is chosen from divalent C₁₋₁₀ alkyl groups substituted with at least one group chosen from C₆₋₁₀ aryl groups. In some embodiments, Z is chosen from divalent C₁₋₈ alkyl groups substituted with at least one group chosen from C₆₋₁₀ aryl groups. In some embodiments, Z is chosen from divalent C₁₋₆ alkyl groups substituted with at least one group chosen from C₆₋₁₀ aryl groups. In some embodiments, Z is chosen from divalent C₁₋₄ alkyl groups substituted with at least one group chosen from C₆₋₁₀ aryl groups. In some embodiments, Z is chosen from divalent C₂ alkyl groups substituted with at least one group chosen from C₆₋₁₀ aryl groups. In some embodiments, Z is a divalent C₁ alkyl group substituted with at least one group chosen from C₆₋₁₀ aryl groups. In some embodiments, the at least one group chosen from C₆₋₁₀ aryl groups is phenyl.

In some embodiments, Z is

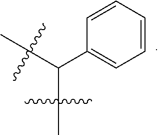 [SMILES: *C(*)c1ccccc1]

In some embodiments, Z is

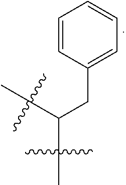 [SMILES: *C(*)Cc1ccccc1]

In some embodiments, Z is chosen from divalent C₁₋₁₈ alkyl groups substituted with at least one group chosen from C₁₋₁₃ heteroaryl groups. In some embodiments, Z is chosen from divalent C₁₋₁₆ alkyl groups substituted with at least one group chosen from C₁₋₁₃ heteroaryl groups. In some embodiments, Z is chosen from divalent C₁₋₁₄ alkyl groups substituted with at least one group chosen from C₁₋₁₃ heteroaryl groups. In some embodiments, Z is chosen from divalent C₁₋₁₂ alkyl groups substituted with at least one group chosen from C₁₋₁₃ heteroaryl groups. In some embodiments, Z is chosen from divalent C₁₋₁₀ alkyl groups substituted with at least one group chosen from C₁₋₁₃ heteroaryl groups. In some embodiments, Z is chosen from divalent C₁₋₈ alkyl groups substituted with at least one group chosen from C₁₋₁₃ heteroaryl groups. In some embodiments, Z is chosen from divalent C₁₋₆ alkyl groups substituted with at least one group chosen from C₁₋₁₃ heteroaryl groups. In some embodiments, Z is chosen from divalent C₁₋₄ alkyl groups substituted with at least one group chosen from C₁₋₁₃ heteroaryl groups. In some embodiments, Z is chosen from divalent C₂ alkyl groups substituted with at least one group chosen from C₁₋₁₃ heteroaryl groups. In some embodiments, Z is a divalent C₁ alkyl group substituted with at least one group chosen from C₁₋₁₃ heteroaryl groups.

In some embodiments, Z is chosen from divalent C₁₋₁₈ alkyl groups substituted with at least one group chosen from C₁₋₈ heteroaryl groups. In some embodiments, Z is chosen from divalent C₁₋₁₆ alkyl groups substituted with at least one group chosen from C₁₋₈ heteroaryl groups. In some embodiments, Z is chosen from divalent C₁₋₁₄ alkyl groups substituted with at least one group chosen from C₁₋₈ heteroaryl groups. In some embodiments, Z is chosen from divalent C₁₋₁₂ alkyl groups substituted with at least one group chosen from C₁₋₈ heteroaryl groups. In some embodiments, Z is chosen from divalent C₁₋₁₀ alkyl groups substituted with at least one group chosen from C₁₋₈ heteroaryl groups. In some embodiments, Z is chosen from divalent C₁₋₈ alkyl groups substituted with at least one group chosen from C₁₋₈ heteroaryl groups. In some embodiments, Z is chosen from divalent C₁₋₆ alkyl groups substituted with at least one group chosen from C₁₋₈ heteroaryl groups. In some embodiments, Z is chosen from divalent C₁₋₄ alkyl groups substituted with at least one group chosen from C₁₋₈ heteroaryl groups. In some embodiments, Z is chosen from divalent C₂ alkyl groups substituted with at least one group chosen from C₁₋₈ heteroaryl groups. In some embodiments, Z is a divalent C₁ alkyl group substituted with at least one group chosen from C₁₋₈ heteroaryl groups.

In some embodiments, Z is chosen from divalent C₁₋₁₈ alkyl groups substituted with at least one group chosen from C₁₋₅ heteroaryl groups. In some embodiments, Z is chosen from divalent C₁₋₁₆ alkyl groups substituted with at least one group chosen from C₁₋₈ heteroaryl groups. In some embodiments, Z is chosen from divalent C₁₋₁₄ alkyl groups substituted with at least one group chosen from C₁₋₅ heteroaryl groups. In some embodiments, Z is chosen from divalent C₁₋₁₂ alkyl groups substituted with at least one group chosen from C₁₋₈ heteroaryl groups. In some embodiments, Z is chosen from divalent C₁₋₁₀ alkyl groups substituted with at least one group chosen from C₁₋₅ heteroaryl groups. In some embodiments, Z is chosen from divalent C₁₋₈ alkyl groups substituted with at least one group chosen from C₁₋₅ heteroaryl groups. In some embodiments, Z is chosen from divalent C₁₋₆ alkyl groups substituted with at least one group chosen from C₁₋₅ heteroaryl groups. In some embodiments, Z is chosen from divalent C₁₋₄ alkyl groups substituted with at least one group chosen from C₁₋₅ heteroaryl groups. In some embodiments, Z is chosen from divalent C₂ alkyl groups substituted with at least one group chosen from C₁₋₅ heteroaryl groups. In some embodiments Z is a divalent C₁ alkyl group substituted with at least one group chosen from C₁₋₅ heteroaryl groups. In some embodiments, the at least one group chosen from C₁₋₅ heteroaryl groups is chosen from

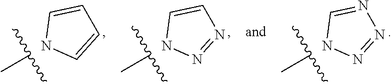

In some embodiments, Z is chosen from

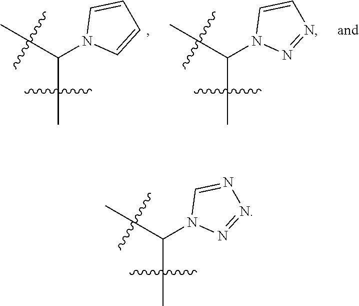 [SMILES: *C(*)n1cccc1 ; *C(*)n1ccnn1 ; *C(*)n1cnnn1]

In some embodiments, Z is chosen from divalent C₁₋₁₈ alkyl groups substituted with at least one group chosen from C₂₋₁₂ heterocyclyl groups. In some embodiments, Z is chosen from divalent C₁₋₁₆ alkyl groups substituted with at least one group chosen from C₂₋₁₂ heterocyclyl groups. In some embodiments, Z is chosen from divalent C₁₋₁₄ alkyl groups substituted with at least one group chosen from C₂₋₁₂ heterocyclyl groups. In some embodiments, Z is chosen from divalent C₁₋₁₂ alkyl groups substituted with at least one group chosen from C₂₋₁₂ heterocyclyl groups. In some embodiments, Z is chosen from divalent C₁₋₁₀ alkyl groups substituted with at least one group chosen from C₂₋₁₂ heterocyclyl groups. In some embodiments, Z is chosen from divalent C₁₋₈ alkyl groups substituted with at least one group chosen from C₂₋₁₂ heterocyclyl groups. In some embodiments, Z is chosen from divalent C₁₋₆ alkyl groups substituted with at least one group chosen from C₂₋₁₂ heterocyclyl groups. In some embodiments, Z is chosen from divalent C₁₋₄ alkyl groups substituted with at least one group chosen from C₂₋₁₂ heterocyclyl groups. In some embodiments, Z is chosen from divalent C₂ alkyl groups substituted with at least one group chosen from C₂₋₁₂ heterocyclyl groups. In some embodiments, Z is a divalent C₁ alkyl group substituted with at least one group chosen from C₂₋₁₂ heterocyclyl groups.

In some embodiments, Z is chosen from divalent C₁₋₁₈ alkyl groups substituted with at least one group chosen from C₂₋₅ heterocyclyl groups. In some embodiments, Z is chosen from divalent C₁₋₁₆ alkyl groups substituted with at least one group chosen from C₂₋₅ heterocyclyl groups. In some embodiments, Z is chosen from divalent C₁₋₁₄ alkyl groups substituted with at least one group chosen from C₂₋₅ heterocyclyl groups. In some embodiments, Z is chosen from divalent C₁₋₁₂ alkyl groups substituted with at least one group chosen from C₂₋₅ heterocyclyl groups. In some embodiments, Z is chosen from divalent C₁₋₁₀ alkyl groups substituted with at least one group chosen from C₂₋₅ heterocyclyl groups. In some embodiments, Z is chosen from divalent C₁₋₈ alkyl groups substituted with at least one group chosen from C₂₋₅ heterocyclyl groups. In some embodiments, Z is chosen from divalent C₁₋₆ alkyl groups substituted with at least one group chosen from C₂₋₅ heterocyclyl groups. In some embodiments, Z is chosen from divalent C₁₋₄ alkyl groups substituted with at least one group chosen from C₂₋₅ heterocyclyl groups. In some embodiments, Z is chosen from divalent C₂ alkyl groups substituted with at least one group chosen from C₂₋₅ heterocyclyl groups. In some embodiments, Z is a divalent C₁ alkyl group substituted with at least one group chosen from C₂₋₅ heterocyclyl groups. In some embodiments, the at least one group chosen from C₂₋₅ heterocyclyl groups is chosen from

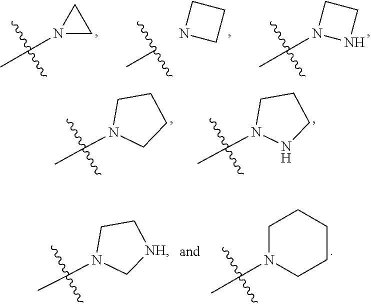

In some embodiments, Z is chosen from

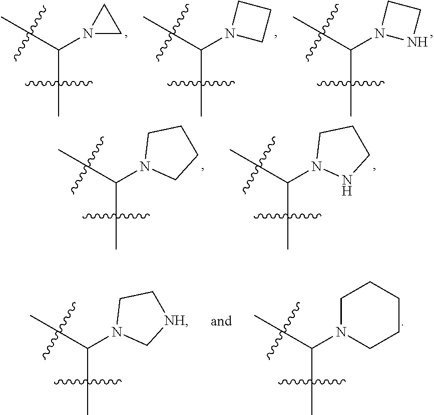

In some embodiments, Z is chosen from divalent C₁₋₁₈ alkyl groups substituted with at least one group chosen from —OC₁₋₁₈ alkyl groups. In some embodiments, Z is chosen from divalent C₁₋₁₆ alkyl groups substituted with at least one group chosen from —OC₁₋₁₈ alkyl groups. In some embodiments, Z is chosen from divalent C₁₋₁₄ alkyl groups substituted with at least one group chosen from —OC₁₋₁₈ alkyl groups. In some embodiments, Z is chosen from divalent C₁₋₁₂ alkyl groups substituted with at least one group chosen from —OC₁₋₁₈ alkyl groups. In some embodiments, Z is chosen from divalent C₁₋₁₀ alkyl groups substituted with at least one group chosen from —OC₁₋₁₈ alkyl groups. In some embodiments, Z is chosen from divalent C₁₋₈ alkyl groups substituted with at least one group chosen from —OC₁₋₁₈ alkyl groups. In some embodiments, Z is chosen from divalent C₁₋₆ alkyl groups substituted with at least one group chosen from —OC₁₋₁₈ alkyl groups. In some embodiments, Z is chosen from divalent C₁₋₄ alkyl groups substituted with at least one group chosen from —OC₁₋₁₈ alkyl groups. In some embodiments, Z is chosen from divalent C₂ alkyl groups substituted with at least one group chosen from —OC₁₋₁₈ alkyl groups. In some embodiments, Z is a divalent C₁ alkyl group substituted with at least one group chosen from —OC₁₋₁₈ alkyl groups.

In some embodiments, Z is chosen from divalent C₁₋₁₈ alkyl groups substituted with at least one group chosen from —OC₁₋₁₀ alkyl groups. In some embodiments, Z is chosen from divalent C₁₋₁₆ alkyl groups substituted with at least one group chosen from —OC₁₋₁₀ alkyl groups. In some embodiments, Z is chosen from divalent C₁₋₁₄ alkyl groups substituted with at least one group chosen from —OC₁₋₁₀ alkyl groups. In some embodiments, Z is chosen from divalent C₁₋₁₂ alkyl groups substituted with at least one group chosen from —OC₁₋₁₀ alkyl groups. In some embodiments, Z is chosen from divalent C₁₋₁₀ alkyl groups substituted with at least one group chosen from —OC₁₋₁₀ alkyl groups. In some embodiments, Z is chosen from divalent C₁₋₈ alkyl groups substituted with at least one group chosen from —OC₁₋₁₀ alkyl groups. In some embodiments, Z is chosen from divalent C₁₋₆ alkyl groups substituted with at least one group chosen from —OC₁₋₁₀ alkyl groups. In some embodiments, Z is chosen from divalent C₁₋₄ alkyl groups substituted with at least one group chosen from —OC₁₋₁₀ alkyl groups. In some embodiments, Z is chosen from divalent C₂ alkyl groups substituted with at least one group chosen from —OC₁₋₁₀ alkyl groups. In some embodiments, Z is a divalent C₁ alkyl group substituted with at least one group chosen from —OC₁₋₁₀ alkyl groups. In some embodiments, the at least one group chosen from —OC₁₋₁₀ alkyl groups is chosen from

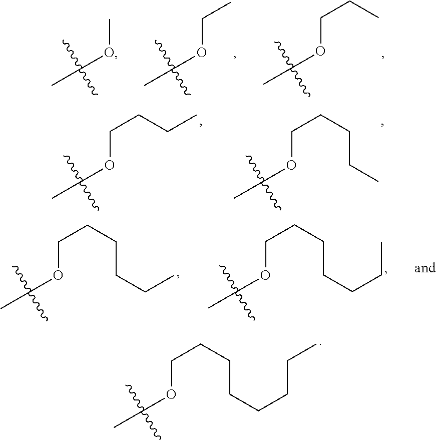

In some embodiments, Z is chosen from

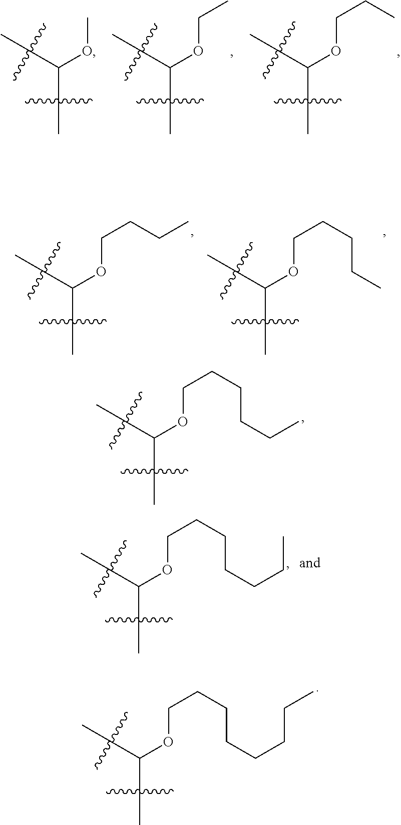

In some embodiments, Z is chosen from divalent C₁₋₁₈ alkyl groups substituted with at least one group chosen from —SC₁₋₁₈ alkyl groups. In some embodiments, Z is chosen from divalent C₁₋₁₆ alkyl groups substituted with at least one group chosen from —SC₁₋₁₈ alkyl groups. In some embodiments, Z is chosen from divalent C₁₋₁₄ alkyl groups substituted with at least one group chosen from —SC₁₋₁₈ alkyl groups. In some embodiments, Z is chosen from divalent C₁₋₁₂ alkyl groups substituted with at least one group chosen from —SC₁₋₁₈ alkyl groups. In some embodiments, Z is chosen from divalent C₁₋₁₀ alkyl groups substituted with at least one group chosen from —SC₁₋₁₈ alkyl groups. In some embodiments, Z is chosen from divalent C₁₋₈ alkyl groups substituted with at least one group chosen from —SC₁₋₁₈ alkyl groups. In some embodiments, Z is chosen from divalent C₁₋₆ alkyl groups substituted with at least one group chosen from —SC₁₋₁₈ alkyl groups. In some embodiments, Z is chosen from divalent C₁₋₄ alkyl groups substituted with at least one group chosen from —SC₁₋₁₈ alkyl groups. In some embodiments, Z is chosen from divalent C₂ alkyl groups substituted with at least one group chosen from —SC₁₋₁₈ alkyl groups. In some embodiments, Z is a divalent C₁ alkyl group substituted with at least one group chosen from —SC₁₋₁₈ alkyl groups.

In some embodiments, Z is chosen from divalent C₁₋₁₈ alkyl groups substituted with at least one group chosen from —SC₁₋₁₀ alkyl groups. In some embodiments, Z is chosen from divalent C₁₋₁₆ alkyl groups substituted with at least one group chosen from —SC₁₋₁₀ alkyl groups. In some embodiments, Z is chosen from divalent C₁₋₁₄ alkyl groups substituted with at least one group chosen from —SC₁₋₁₀ alkyl groups. In some embodiments, Z is chosen from divalent C₁₋₁₂ alkyl groups substituted with at least one group chosen from —SC₁₋₁₀ alkyl groups. In some embodiments, Z is chosen from divalent C₁₋₁₀ alkyl groups substituted with at least one group chosen from —SC₁₋₁₀ alkyl groups. In some embodiments, Z is chosen from divalent C₁₋₈ alkyl groups substituted with at least one group chosen from —SC₁₋₁₀ alkyl groups. In some embodiments, Z is chosen from divalent C₁₋₆ alkyl groups substituted with at least one group chosen from —SC₁₋₁₀ alkyl groups. In some embodiments, Z is chosen from divalent C₄ alkyl groups substituted with at least one group chosen from —SC₁₋₁₀ alkyl groups. In some embodiments, Z is chosen from divalent C₂ alkyl groups substituted with at least one group chosen from —SC₁₋₁₀ alkyl groups. In some embodiments, Z is a divalent C₁ alkyl group substituted with at least one group chosen from —SC₁₋₁₀ alkyl groups. In some embodiments, the at least one group chosen from —SC₁₋₁₀ alkyl groups is chosen from

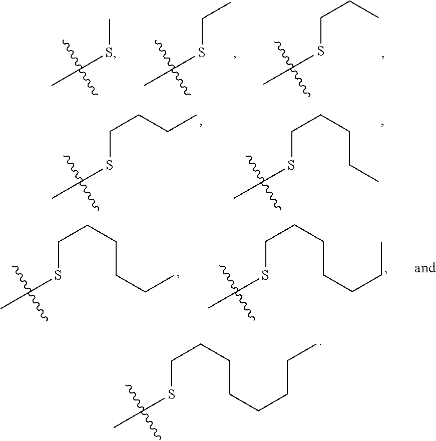

In some embodiments, Z is chosen from

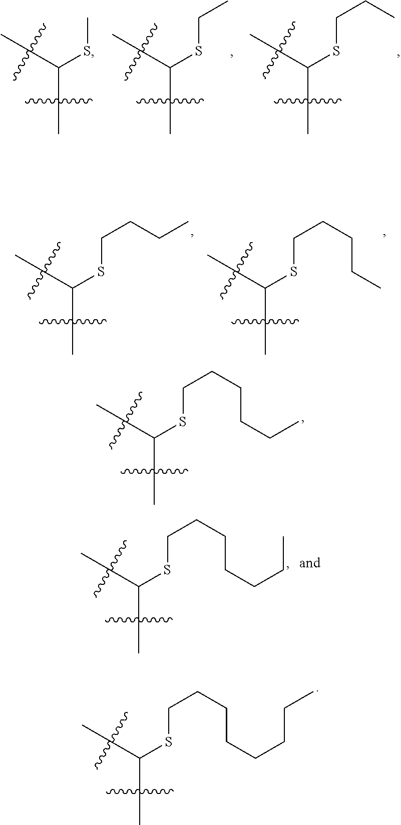

In some embodiments, Z is chosen from divalent C₁₋₁₈ alkyl groups substituted with at least one group chosen from —N(T²)C₁₋₁₈ alkyl groups. In some embodiments, Z is chosen from divalent C₁₋₁₆ alkyl groups substituted with at least one group chosen from —N(T²)C₁₋₁₈ alkyl groups. In some embodiments, Z is chosen from divalent C₁₋₁₄ alkyl groups substituted with at least one group chosen from —N(T²)C₁₋₁₈ alkyl groups. In some embodiments, Z is chosen from divalent C₁₋₁₂ alkyl groups substituted with at least one group chosen from —N(T²)C₁₋₁₈ alkyl groups. In some embodiments, Z is chosen from divalent C₁₋₁₀ alkyl groups substituted with at least one group chosen from —N(T²)C₁₋₁₈ alkyl groups. In some embodiments, Z is chosen from divalent C₁₋₈ alkyl groups substituted with at least one group chosen from —N(T²)C₁₋₁₈ alkyl groups. In some embodiments, Z is chosen from divalent C₁₋₆ alkyl groups substituted with at least one group chosen from —N(T²)C₁₋₁₈ alkyl groups. In some embodiments, Z is chosen from divalent C₁₋₄ alkyl groups substituted with at least one group chosen from —N(T²)C₁₋₁₈ alkyl groups. In some embodiments, Z is chosen from divalent C₂ alkyl groups substituted with at least one group chosen from —N(T²)C₁₋₁₈ alkyl groups. In some embodiments, Z is a divalent C₁ alkyl group substituted with at least one group chosen from —N(T²)C₁₋₁₈ alkyl groups. In some embodiments, the at least one group chosen from —N(T²)C₁₋₁₈ alkyl groups is chosen from —NHC₁₋₁₈ alkyl groups, i.e., T² is H. In some embodiments, the at least one group chosen from —N(T²)C₁₋₁₈ alkyl groups is chosen from —N(C₁₋₈ alkyl groups)C₁₋₁₈ alkyl groups, i.e., T² is C₁₋₈ alkyl groups.

In some embodiments, Z is chosen from divalent C₁₋₁₈ alkyl groups substituted with at least one group chosen from —N(T²)C₁₋₁₀ alkyl groups. In some embodiments, Z is chosen from divalent C₁₋₁₆ alkyl groups substituted with at least one group chosen from —N(T²)C₁₋₁₀ alkyl groups. In some embodiments, Z is chosen from divalent C₁₋₁₄ alkyl groups substituted with at least one group chosen from —N(T²)C₁₋₁₀ alkyl groups. In some embodiments, Z is chosen from divalent C₁₋₁₂ alkyl groups substituted with at least one group chosen from —N(T²)C₁₋₁₀ alkyl groups. In some embodiments, Z is chosen from divalent C₁₋₁₀ alkyl groups substituted with at least one group chosen from —N(T²)C₁₋₁₀ alkyl groups. In some embodiments, Z is chosen from divalent C₁₋₈ alkyl groups substituted with at least one group chosen from —N(T²)C₁₋₁₀ alkyl groups. In some embodiments, Z is chosen from divalent C₁₋₆ alkyl groups substituted with at least one group chosen from —N(T²)C₁₋₁₀ alkyl groups. In some embodiments, Z is chosen from divalent C₁₋₄ alkyl groups substituted with at least one group chosen from —N(T²)C₁₋₁₀ alkyl groups. In some embodiments, Z is chosen from divalent C₂ alkyl groups substituted with at least one group chosen from —N(T²)C₁₋₁₀ alkyl groups. In some embodiments, Z is a divalent C₁ alkyl group substituted with at least one group chosen from —N(T²)C₁₋₁₀ alkyl groups. In some embodiments, the at least one group chosen from —N(T²)C₁₋₁₀ alkyl groups is chosen from —NHC₁₋₁₀ alkyl groups. In some embodiments, the at least one group chosen from —N(T²)C₁₋₁₀ alkyl groups is chosen from —N(C₁₋₈ alkyl groups)C₁₋₁₀ alkyl groups. In some embodiments, the at least one group chosen from —N(T²)C₁₋₁₀ alkyl groups is chosen from

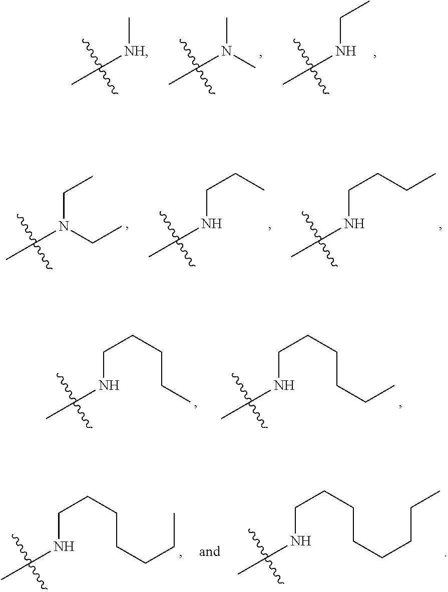

In some embodiments, Z is chosen from

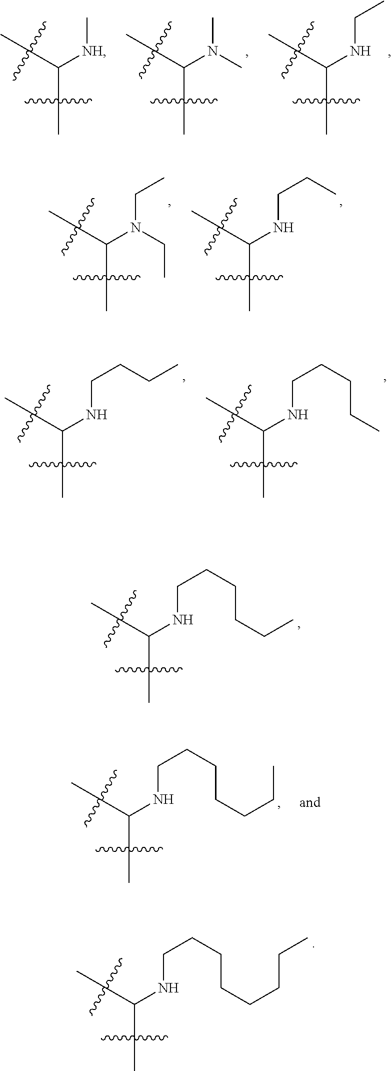

In some embodiments, Z is chosen from divalent C₁₋₁₈ alkyl groups substituted with at least one group chosen from —N(T²)AA groups. In some embodiments, Z is chosen from divalent C₁₋₁₆ alkyl groups substituted with at least one group chosen from —N(T²)AA groups. In some embodiments, Z is chosen from divalent C₁₋₁₄ alkyl groups substituted with at least one group chosen from —N(T²)AA groups. In some embodiments, Z is chosen from divalent C₁₋₁₂ alkyl groups substituted with at least one group chosen from —N(T²)AA groups. In some embodiments, Z is chosen from divalent C₁₋₁₀ alkyl groups substituted with at least one group chosen from —N(T²)AA groups. In some embodiments, Z is chosen from divalent C₁₋₈ alkyl groups substituted with at least one group chosen from —N(T²)AA groups. In some embodiments, Z is chosen from divalent C₁₋₆ alkyl groups substituted with at least one group chosen from —N(T²)AA groups. In some embodiments, Z is chosen from divalent C₁₋₄ alkyl groups substituted with at least one group chosen from —N(T²)AA groups. In some embodiments, Z is chosen from divalent C₂ alkyl groups substituted with at least one group chosen from —N(T²)AA groups. In some embodiments, Z is a divalent C₁ alkyl group substituted with at least one group chosen from —N(T²)AA groups. In some embodiments, the at least one group chosen from —N(T²)AA groups is chosen from —NHAA groups. In some embodiments, the at least one group chosen from —N(T²)AA groups is chosen from —N(C₁₋₈ alkyl groups)AA groups.

In some embodiments, Z is chosen from

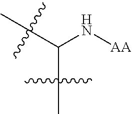

groups.

In some embodiments, AA is chosen from natural amino acid residues. In some embodiments, AA is chosen from unnatural amino acid residues. Exemplary unnatural amino acids can be found in (1) Unnatural Amino Acid: Tools for Drug Discovery, ChemFiles (by Sigma-Aldrich), Vol. 4, No. 5 and (2) Schultz et al., J. Bio., Chem, 2010, 285(15), 11039-11044. In some embodiments, AA is chosen from L-amino acid residues. In some embodiments, AA is chosen from natural L-amino acid residues.

In some embodiments, X is chosen from C₁₋₁₈ alkyl groups. In some embodiments, X is chosen from C₁₋₁₄ alkyl groups. In some embodiments, X is chosen from C₁₋₁₂ alkyl groups. In some embodiments, X is chosen from C₁₋₁₀ alkyl groups. In some embodiments, X is chosen from C₁₋₈ alkyl groups. In some embodiments, X is chosen from C₁₋₄ alkyl groups. In some embodiments, X is chosen from C₃₋₁₀ alkyl groups. In some embodiments, X is chosen from —CH₃, —(CH₂)CH₃, —(CH₂)₂CH₃, —(CH₂)₃CH₃, —(CH₂)₄CH₃, —(CH₂)₅CH₃, —(CH₂)₆CH₃, —(CH₂)₇CH₃. —(CH₂)₈CH₃, and —(CH₂)₉CH₃.

In some embodiments, X is chosen from

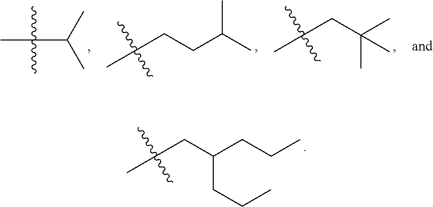

In some embodiments, X is chosen from C₁₋₁₈ haloalkyl groups. In some embodiments, X is chosen from C₁₋₁₄ haloalkyl groups. In some embodiments, X is chosen from C₁₋₁₂ haloalkyl groups. In some embodiments, X is chosen from C₁₋₁₀ haloalkyl groups. In some embodiments, X is chosen from C₁₋₈ haloalkyl groups. In some embodiments, X is chosen from C₁₋₄ haloalkyl groups. In some embodiments, X is chosen from C₃₋₁₀ haloalkyl groups.

In some embodiments, X is chosen from C₆₋₁₈ aryl and C₇₋₁₉ arylalkyl groups. In some embodiments, X is chosen from C₆₋₁₂ aryl and C₇₋₁₃ arylalkyl groups. In some embodiments, X is chosen from C₆₋₁₈ aryl groups. In some embodiments, X is chosen from C₆₋₁₂ aryl groups. In some embodiments, X is chosen from C₇₋₁₉ arylalkyl groups. In some embodiments, X is chosen from C₇₋₁₃ arylalkyl groups.

In some embodiments, X is

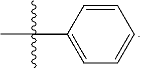 [SMILES: *c1ccccc1]

In some embodiments, X is

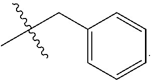 [SMILES: *Cc1ccccc1]

In some embodiments, X is chosen from C₁₋₁₃ heteroaryl and C₂₋₁₄ heteroarylalkyl groups. In some embodiments, X is chosen from C₁₋₁₃ heteroaryl groups. In some embodiments, X is chosen from C₁₋₁₀ heteroaryl groups. In some embodiments, X is chosen from C₁₋₈ heteroaryl groups. In some embodiments, X is chosen from C₁₋₆ heteroaryl groups. In some embodiments, X is chosen from C₂₋₁₄ heteroarylalkyl groups. In some embodiments, X is chosen from C₂₋₁₁ heteroarylalkyl groups. In some embodiments, X is chosen from C₂₋₉ heteroarylalkyl groups. In some embodiments, X is chosen from C₂₋₇ heteroarylalkyl groups.

In some embodiments, X is chosen from —(CH₂CH₂O)ₙCH₃ groups. In some embodiments, n is chosen from integers ranging from 1 to 10. In some embodiments, n is chosen from integers ranging from 1 to 8. In some embodiments, n is chosen from integers ranging from 1 to 6. In some embodiments, n is chosen from integers ranging from 1 to 4. In some embodiments, n is 1. In some embodiments, n is 2. In some embodiments, n is 3. In some embodiments, n is 4. In some embodiments, X is

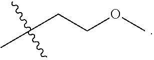 [SMILES: *CCOC]

In some embodiments, X is

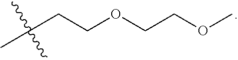 [SMILES: *CCOCCOC]

In some embodiments, X is chosen from

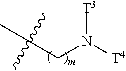

groups.

In some embodiments, X is chosen from

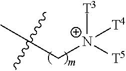

groups.

In some embodiments, X is chosen from

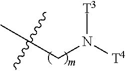

groups, wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, X is chosen from

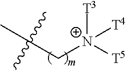

groups, wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, X is chosen from

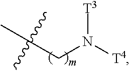

wherein T³ and T⁴, which may be the same or different, are independently chosen from H and C₁₋₅ alkyl groups. In some embodiments, T³ and T⁴ are each H. In some embodiments, T³ and T⁴, which may be the same or different, are independently chosen from C₁₋₅ alkyl groups. In some embodiments, T³ is H and T⁴ is chosen from C₁₋₅ alkyl groups.

In some embodiments, X is chosen from

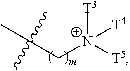

wherein T³, T⁴, and T⁵, which may be the same or different, are independently chosen from H and C₁₋₅ alkyl groups. In some embodiments, T³, T⁴, and T⁵ are each H. In some embodiments, T³, T⁴, and T⁵, which may be the same or different, are independently chosen from C₁₋₅ alkyl groups. In some embodiments. T³ is H, and T⁴ and T⁵, which may be the same or different, are independently chosen from C₁₋₅ alkyl groups. In some embodiments, T³ and T⁴ are each H. and T⁵ is chosen from C₁₋₅ alkyl groups.

In some embodiments, X is chosen from

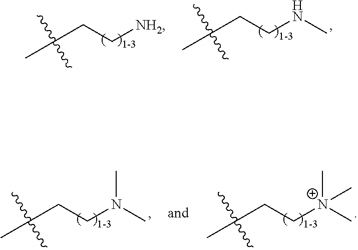

In some embodiments, X is chosen from

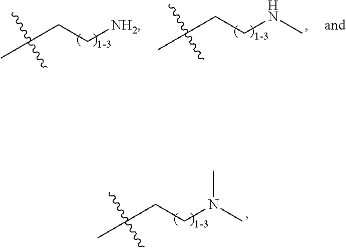

In some embodiments X is chosen from

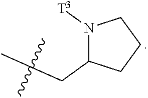

In some embodiments, X is

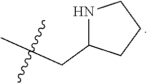 [SMILES: *CC1CCCN1]

In some embodiments, X is chosen from

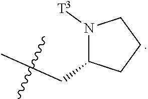

in some embodiments. X is

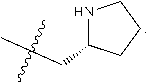 [SMILES: *C[C@H]1CCCN1]

In some embodiments, Y is chosen from C₁₋₁₈ alkyl groups. In some embodiments, Y is chosen from C₁₋₁₄ alkyl groups. In some embodiments, Y is chosen from C₁₋₁₂ alkyl groups. In some embodiments, Y is chosen from C₁₋₁₀ alkyl groups. In some embodiments, Y is chosen from C₁₋₈ alkyl groups. In some embodiments, Y is chosen from C₁₋₄ alkyl groups. In some embodiments, Y is chosen from C₃₋₁₀ alkyl groups. In some embodiments, Y is chosen from —CH₃, —(CH₂)CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, and —(CH₂)₉CH₃.

In some embodiments, Y is chosen from

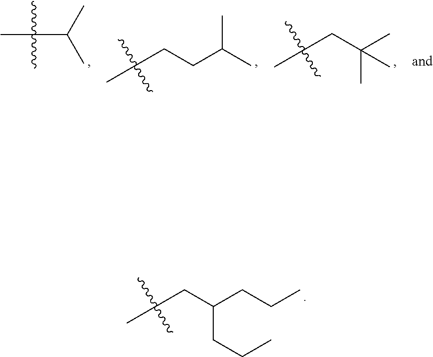

In some embodiments, Y is chosen from C₁₋₁₈ haloalkyl groups. In some embodiments, Y is chosen from C₁₋₁₄ haloalkyl groups. In some embodiments, Y is chosen from C₁₋₁₂ haloalkyl groups. In some embodiments, Y is chosen from C₁₋₁₀ haloalkyl groups. In some embodiments, Y is chosen from C₁₋₈ haloalkyl groups. In some embodiments, Y is chosen from C₁₋₄ haloalkyl groups. In some embodiments, Y is chosen from C₃₋₁₀ haloalkyl groups.

In some embodiments, Y is chosen from C₆₋₁₈ aryl and C₇₋₁₉ arylalkyl groups. In some embodiments, Y is chosen from C₆₋₁₂ aryl and C₇₋₁₃ arylalkyl groups. In some embodiments, Y is chosen from C₆₋₁₈ aryl groups. In some embodiments, Y is chosen from C₆₋₁₂ aryl groups. In some embodiments, Y is chosen from C₇₋₁₉ arylalkyl groups. In some embodiments, Y is chosen from C₇₋₁₃ arylalkyl groups.

In some embodiments, Y is

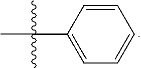 [SMILES: *c1ccccc1]

In some embodiments, Y is

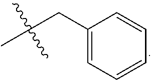 [SMILES: *Cc1ccccc1]

In some embodiments, Y is chosen from C₁₋₁₃ heteroaryl and C₂₋₁₄ heteroarylalkyl groups. In some embodiments, Y is chosen from C₁₋₁₃ heteroaryl groups. In some embodiments, Y is chosen from C₁₋₁₀ heteroaryl groups. In some embodiments, Y is chosen from C₁₋₈ heteroaryl groups. In some embodiments, Y is chosen from C₁₋₆ heteroaryl groups. In some embodiments, Y is chosen from C₂₋₁₄ heteroarylalkyl groups. In some embodiments, Y is chosen from C₂₋₁₁ heteroarylalkyl groups. In some embodiments, Y is chosen from C₂₋₉ heteroarylalkyl groups. In some embodiments, Y is chosen from C₂₋₇ heteroarylalkyl groups.

In some embodiments, Y is chosen from —(CH₂CH₂O)ₙCH₃ groups. In some embodiments, n is chosen from integers ranging from 1 to 10. In some embodiments, n is chosen from integers ranging from 1 to 8. In some embodiments, n is chosen from integers ranging from 1 to 6. In some embodiments, n is chosen from integers ranging from 1 to 4. In some embodiments, n is 1. In some embodiments, n is 2. In some embodiments, n is 3. In some embodiments, n is 4. In some embodiments, Y is

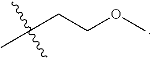 [SMILES: *CCOC]

In some embodiments, Y is

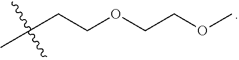 [SMILES: *CCOCCOC]

In some embodiments, Y is chosen from

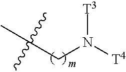

groups.

In some embodiments, Y is chosen from

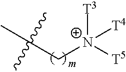

groups.

In some embodiments, Y is chosen from

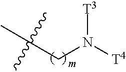

groups, wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, Y is chosen from

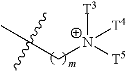

groups, wherein m is chosen from integers ranging from 1 to 6. In some embodiments, m is chosen from integers ranging from 1 to 5. In some embodiments, m is chosen from integers ranging from 1 to 4. In some embodiments, m is chosen from integers ranging from 1 to 3. In some embodiments, m is 1. In some embodiments, m is 2. In some embodiments, m is 3. In some embodiments, m is 4.

In some embodiments, Y is chosen from

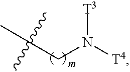

wherein T³ and T⁴, which may be the same or different, are independently chosen from H and C₁₋₅ alkyl groups. In some embodiments, T³ and T⁴ are each H. In some embodiments, T³ and T⁴, which may be the same or different, are independently chosen from C₁₋₅ alkyl groups. In some embodiments, T³ is H and T⁴ is chosen from C₁₋₅ alkyl groups.

In some embodiments, Y is chosen from

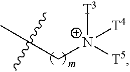

wherein T³, T⁴, and T⁵, which may be the same or different, are independently chosen from H and C₁₋₅ alkyl groups. In some embodiments, T³, T⁴, and T⁵ are each H. In some embodiments, T³. T⁴, and T⁵, which may be the same or different, are independently chosen from C₁₋₅ alkyl groups. In some embodiments, T³ is H, and T⁴ and T⁵, which may be the same or different, are independently chosen from C₁₋₅ alkyl groups. In some embodiments, T³ and T⁴ are each H, and T⁵ is chosen from C₁₋₅ alkyl groups.

In some embodiments, Y is chosen from

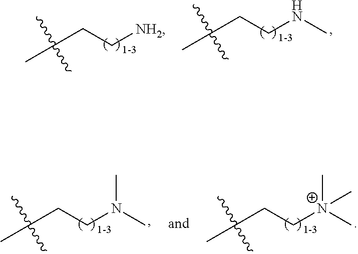

In some embodiments, Y is chosen from

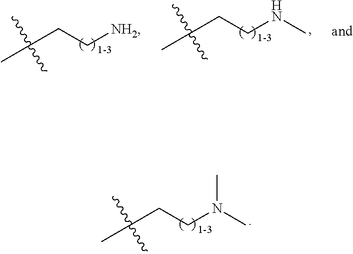

In some embodiments, Y is chosen from

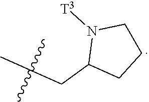

In some embodiments, Y is

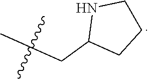 [SMILES: *CC1CCCN1]

In some embodiments, Y is chosen from

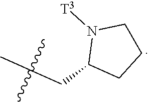

In some embodiments, Y is

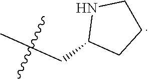 [SMILES: *C[C@H]1CCCN1]

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q.

In some embodiments, X is chosen from H and Q, and Y is chosen from C₁₋₁₈ alkyl, C₁₋₁₈ haloalkyl, C₆₋₁₈ aryl. C₁₋₁₃ heteroaryl, C₇₋₁₉ arylalkyl, C₂₋₁₄ heteroarylalkyl, —(CH₂CH₂O)ₙCH₃,

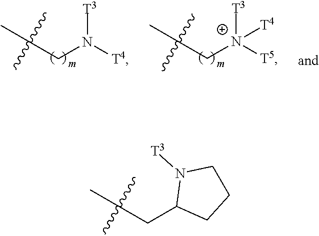

groups.

In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

Non-limiting examples of Q include cations of aluminium, arginine, benzathine, calcium, choline, copper, amines (substituted, e.g., diethylamine, and unsubstituted), ammonium (substituted and unsubstituted), diolamine, glycine, iron, lithium, lysine, magnesium, manganese, meglumine, olamine, potassium, sodium, tromethamine and zinc. In some embodiments, at least one Q is chosen from ammonium (substituted and unsubstituted) cations. In some embodiments, at least one Q is unsubstituted ammonium cation. In some embodiments, each Q is chosen from ammonium (substituted and unsubstituted) cations. In some embodiments, each Q is unsubstituted ammonium cation.

In some embodiments, T¹ is H. In some embodiments, T¹ is chosen from C₁₋₁₈ alkyl groups. In some embodiments, T¹ is chosen from C₁₋₁₂ alkyl groups. In some embodiments, T¹ is chosen from C₁₋₁₀ alkyl groups. In some embodiments, T¹ is chosen from C₁₋₈ alkyl groups. In some embodiments, T¹ is chosen from C₁₋₄ alkyl groups. In some embodiments, T¹ is chosen from Me, Et, n-Pr, i-Pr, n-Bu, s-Bu, i-Bu, t-Bu, cyclopropyl, and cyclobutyl. In some embodiments, T¹ is Me.

In some embodiments, T¹ is chosen from C₁₋₁₈ haloalkyl groups. In some embodiments, T¹ is chosen from C₁₋₁₂ haloalkyl groups. In some embodiments, T¹ is chosen from C₁₋₁₀ haloalkyl groups. In some embodiments, T¹ is chosen from C₁₋₈ haloalkyl groups. In some embodiments, T¹ is chosen from C₁₋₄ haloalkyl groups. In some embodiments, T¹ is —CF₂CF₃. In some embodiments, T¹ is —CF₃.

2. R² and R³

R² is chosen from C₁₋₁₀ alkyl groups and C₁₋₁₀ haloalkyl groups.

In some embodiments, R² is chosen from C₂₋₉ alkyl groups. In some embodiments, R² is chosen from C₂₋₈ alkyl groups. In some embodiments, R² is chosen from C₂₋₇ alkyl groups. In some embodiments, R² is chosen from C₂₋₆ alkyl groups. In some embodiments, R² is chosen from C₃ alkyl groups. In some embodiments, R² is chosen from C₄ alkyl groups. In some embodiments, R² is chosen from C₅ alkyl groups. In some embodiments, R² is chosen from C₆ alkyl groups. In some embodiments, R² is chosen from C₇ alkyl groups. In some embodiments, R² is chosen from C₈ alkyl groups. In some embodiments, R² is chosen from C₉ alkyl groups.

In some embodiments, R² is propyl. In some embodiments, R² is n-propyl. In some embodiments, R² is pentyl. In some embodiments, R² is n-pentyl. In some embodiments, R² is hexyl. In some embodiments, R² is n-hexyl. In some embodiments, R² is heptyl. In some embodiments, R² is n-heptyl. In some embodiments, R² is octyl. In some embodiments, R² is n-octyl. In some embodiments, R² is nonyl. In some embodiments, R² is n-nonyl. In some embodiments, R² is 1,1-dimethylheptyl.

In some embodiments, R² is chosen from C₂₋₉ haloalkyl groups. In some embodiments, R² is chosen from C₂₋₅ haloalkyl groups. In some embodiments, R² is chosen from C₂₋₇ haloalkyl groups. In some embodiments, R² is chosen from C₂₋₆ haloalkyl groups. In some embodiments, R² is chosen from C₃ haloalkyl groups. In some embodiments, R² is chosen from C₄ haloalkyl groups. In some embodiments, R² is chosen from C₅ haloalkyl groups. In some embodiments, R² is chosen from C₆ haloalkyl groups. In some embodiments, R² is chosen from C₇ haloalkyl groups. In some embodiments, R² is chosen from C₈ haloalkyl groups. In some embodiments, R² is chosen from C₉ haloalkyl groups.

R³ is chosen from —CH₃, —CD₃, —CH₂OH, —CH₂OR^{a}, and ═O.

In some embodiments, R³ is chosen from —CH₃, —CD₃, —CH₂OH, and —CH₂OR^{a} such that Formula (I) is in the following form.

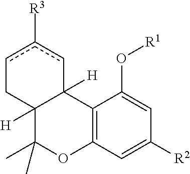

In some embodiments, R³ is chosen from —CH₃, —CD₃, —CH₂OH, and —CH₂OR^{a}, and Formula (I) is in the form of Formula (II).

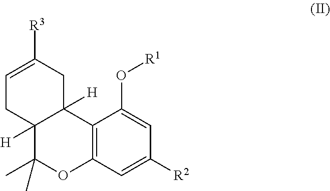

In some embodiments, Formula (II) is in the form of Formula (II)-1.

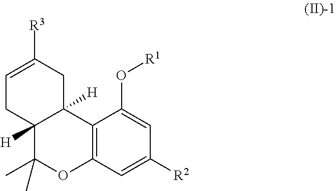

In some embodiments, Formula (II) is in the form of Formula (II)-2.

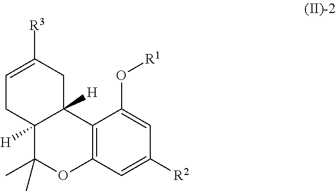

In some embodiments, R³ is chosen from —CH₃, —CD₃, —CH₂OH, and —CH₂OR^{a}, and Formula (I) is in the form of Formula (III).

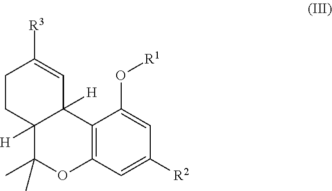

In some embodiments, Formula (III) is in the form of Formula (III)-1.

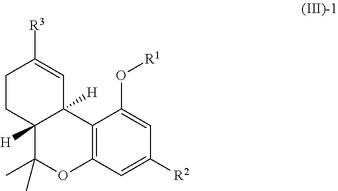

In some embodiments, Formula (III) is in the form of Formula (III)-2.

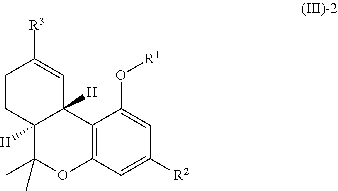

In some embodiments, R³ is —CH₃. In some embodiments, R³ is —CD₃. In some embodiments, R³ is —CH₂OH. In some embodiments, R³ is —CH₂OR^{a}.

In some embodiments, Rd is chosen from C₁₋₈ alkyl groups. In some embodiments, R^{a} is chosen from C₁₋₇ alkyl groups. In some embodiments, R^{a} is chosen from C₁₋₆ alkyl groups. In some embodiments, R^{a} is chosen from C₁₋₅ alkyl groups. In some embodiments, R^{a} is chosen from C₁₋₄ alkyl groups. In some embodiments, R^{a} is chosen from C₁₋₃ alkyl groups. In some embodiments, R^{a} is methyl. In some embodiments, R^{a} is ethyl. In some embodiments, R^{a} is propyl. In some embodiments, R^{a} is n-propyl. In some embodiments, R^{a} is isopropyl. In some embodiments, R^{a} is cyclopropyl. In some embodiments, R^{a} is butyl. In some embodiments, R^{a} is n-butyl. In some embodiments, R^{a} is isobutyl. In some embodiments, R^{a} is tert-butyl. In some embodiments, R^{a} is cyclobutyl. In some embodiments, R^{a} is pentyl. In some embodiments, R^{a} is hexyl. In some embodiments, R^{a} is heptyl. In some embodiments, R^{a} is octyl.

In some embodiments, R^{a} is chosen from C₁₋₈ haloalkyl groups. In some embodiments, R^{a} is chosen from C₁₋₇ haloalkyl groups. In some embodiments, R^{a} is chosen from C₁₋₆ haloalkyl groups. In some embodiments, R^{a} is chosen from C₁₋₅ haloalkyl groups. In some embodiments, R^{a} is chosen from C₁₋₄ haloalkyl groups. In some embodiments, R is chosen from C₁₋₃ haloalkyl groups. In some embodiments, R^{a} is —CF₂CF₃. In some embodiments, R^{a} is —CF₃.

In some embodiments, R³ is ═O such that Formula (I) is in the form of Formula (IV).

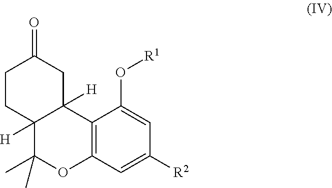

In some embodiments, Formula (IV) is in the form of Formula (IV)-1.

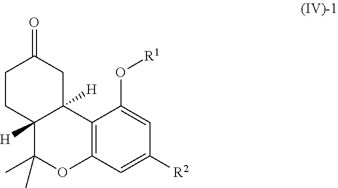

In some embodiments, Formula (IV) is in the form of Formula (IV)-2.

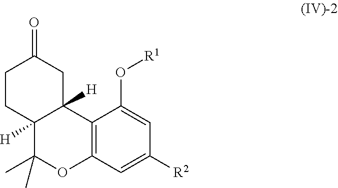

In some embodiments, R² is chosen from C₂₋₉ alkyl groups, and R³ is chosen from —CH₃, —CD₃, —CH₂OH, and —CH₂OR^{a}. In some embodiments, R² is chosen from C₂₋₉ alkyl groups, and R³ is —CH₃. In some embodiments, R² is chosen from C₂₋₉ alkyl groups, and R³ is —CD₃. In some embodiments, R² is chosen from C₂₋₉ alkyl groups, and R³ is —CH₂OH. In some embodiments, R² is chosen from C₂₋₉ alkyl groups, and R³ is —CH₂OR^{a}. In some embodiments, R² is chosen from C₂₋₉ alkyl groups, and R³ is ═O.

In some embodiments, R² is chosen from n-propyl, n-pentyl, and 1,1-dimethylheptyl, and R³ is chosen from —CH₃, —CD₃. —CH₂OH, and —CH₂OR^{a}. In some embodiments, R² is chosen from n-propyl, n-pentyl, and 1,1-dimethylheptyl, and R³ is —CH₃. In some embodiments, R² is chosen from n-propyl, n-pentyl, and 1,1-dimethylheptyl, and R³ is —CD₃. In some embodiments, R² is chosen from n-propyl, n-pentyl, and 1,1-dimethylheptyl, and R³ is —CH₂OH. In some embodiments, R² is chosen from n-propyl, n-pentyl, and 1,1-dimethylheptyl, and R³ is —CH₂OR^{a}. In some embodiments, R² is chosen from n-propyl, n-pentyl, and 1,1-dimethylheptyl, and R³ is ═O.

In some embodiments, R² is n-propyl, and R³ is chosen from —CH₃, —CD₃, —CH₂OH, and —CH₂OR^{a}. In some embodiments, R² is n-propyl, and R³ is —CH₃. In some embodiments, R² is n-propyl, and R³ is —CD₃. In some embodiments, R² is n-propyl, and R³ is —CH₂OH. In some embodiments, R² is n-propyl, and R³ is —CH₂OR^{a}. In some embodiments, R² is n-propyl, and R³ is ═O.

In some embodiments, R² is n-pentyl, and R³ is chosen from —CH₃, —CD₃, —CH₂OH, and —CH₂OR^{a}. In some embodiments, R² is n-pentyl, and R³ is —CH₃. In some embodiments, R² is n-pentyl, and R³ is —CD₃. In some embodiments, R² is n-pentyl, and PW is —CH₂OH. In some embodiments, R² is n-pentyl, and R³ is —CH₂OR^{a}. In some embodiments, R² is n-pentyl, and R³ is ═O.

In some embodiments, R² is 1,1-dimethylheptyl, and R³ is chosen from —CH₃, —CD₃, —CH₂OH, and —CH₂OR^{a}. In some embodiments, R² is 1,1-dimethylheptyl, and R³ is —CH₃. In some embodiments, R² is 1,1-dimethylheptyl, and R³ is —CD₃. In some embodiments, R² is 1,1-dimethylheptyl, and R³ is —CH₂OH. In some embodiments, R² is 1,1-dimethylheptyl, and R³ is —CH₂OR^{a}. In some embodiments, R² is 1,1-dimethylheptyl, and R³ is ═O.

### B. Exemplary Structures

### 1. Exemplary Structures

In some embodiments, the compounds have a structure of Formula (I) or a pharmaceutically acceptable salt thereof:

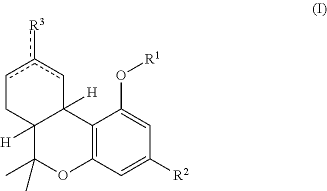

wherein the

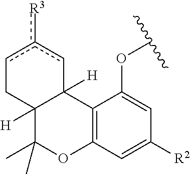

moiety is chosen from

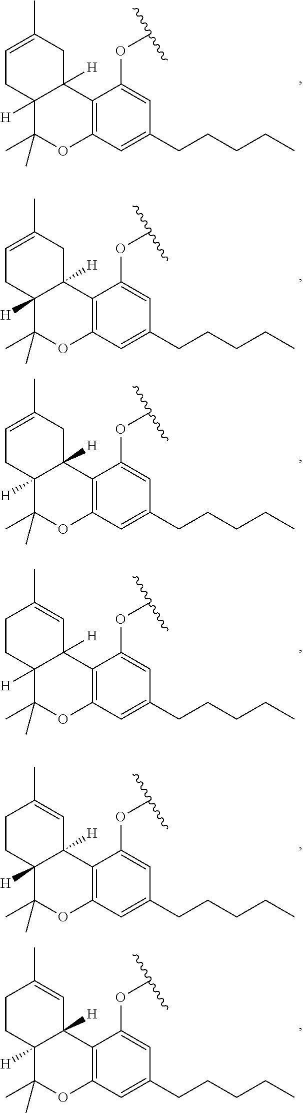

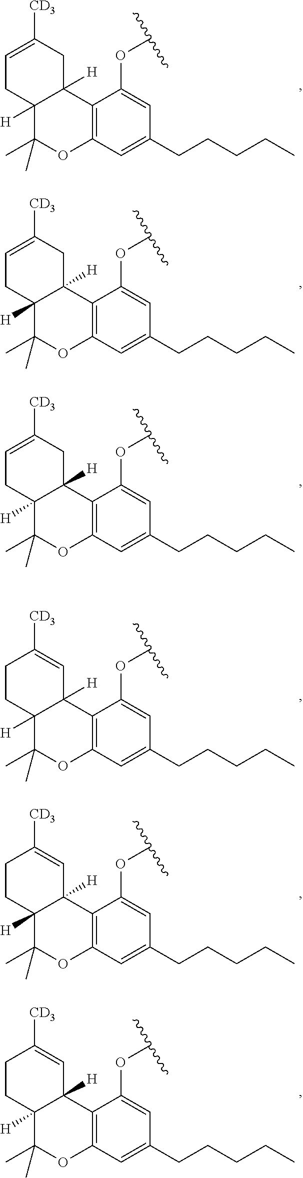

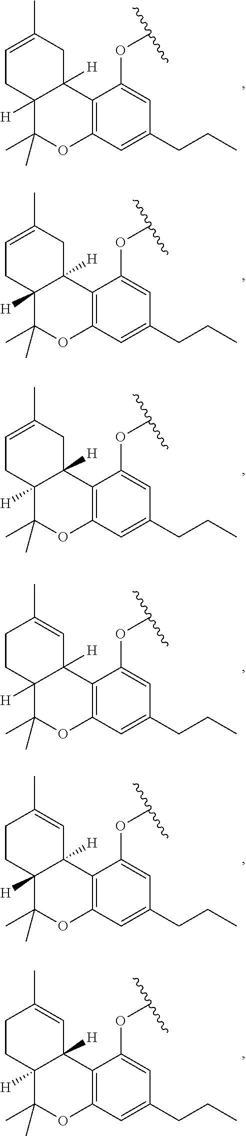

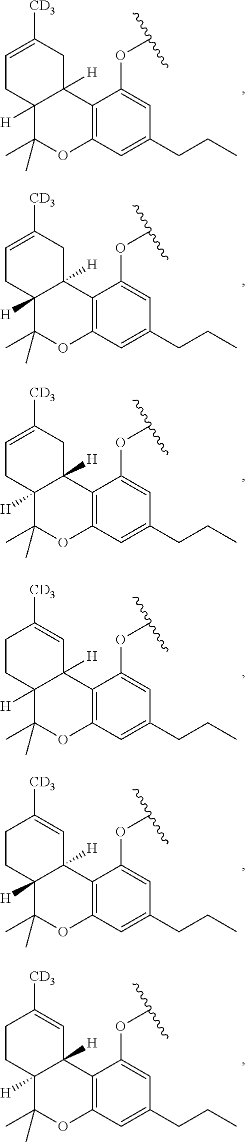

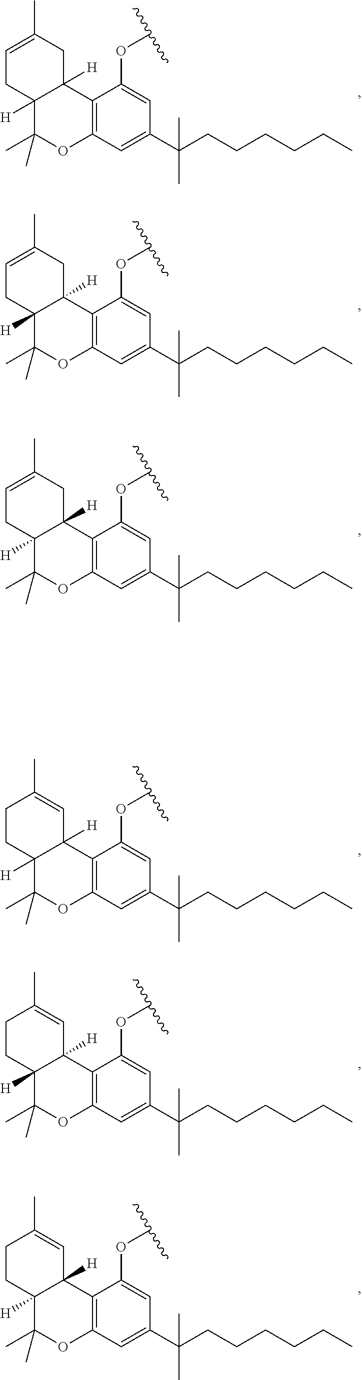

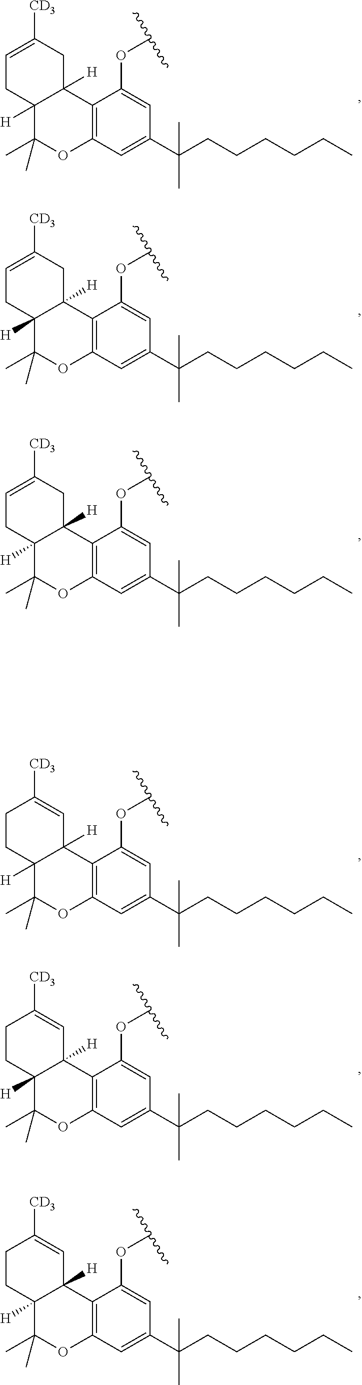

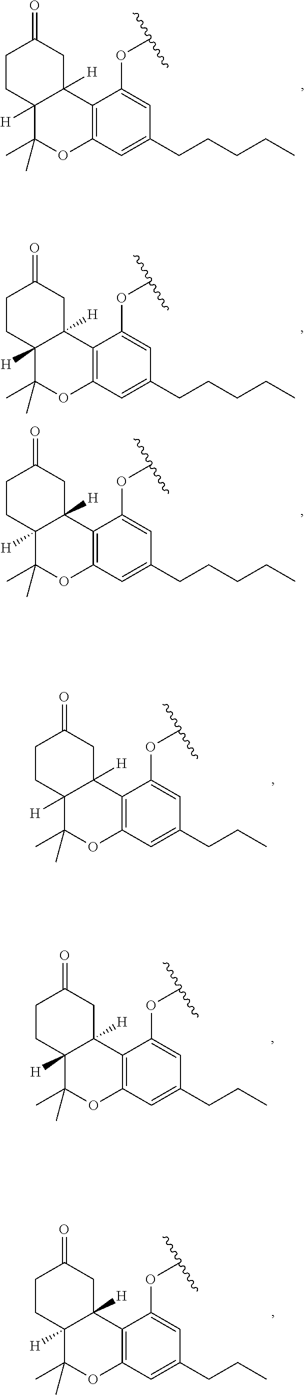

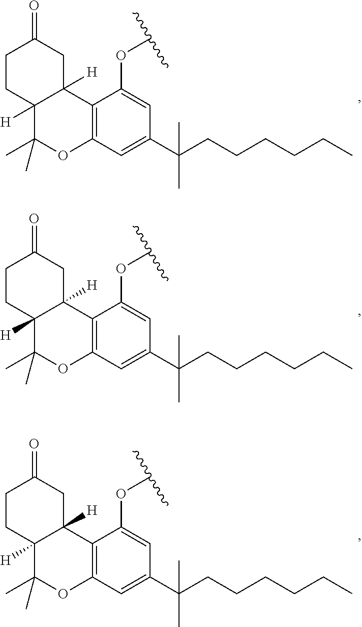

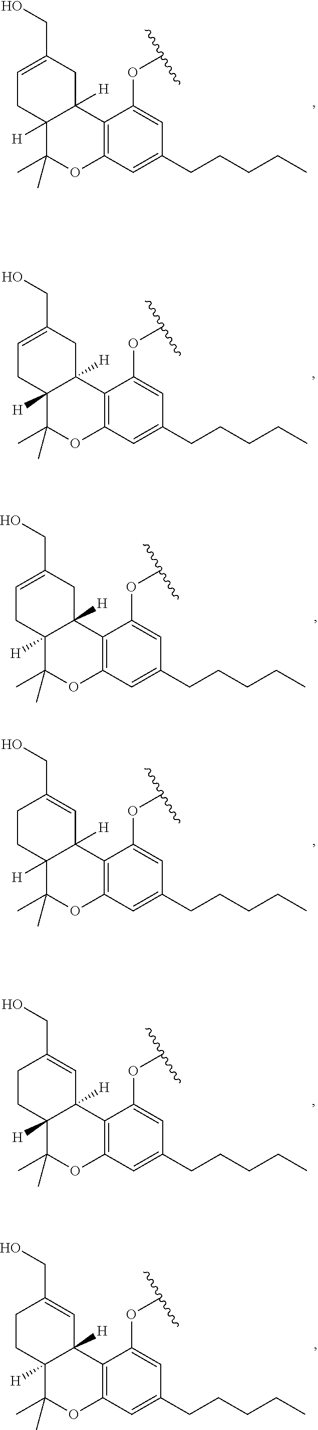

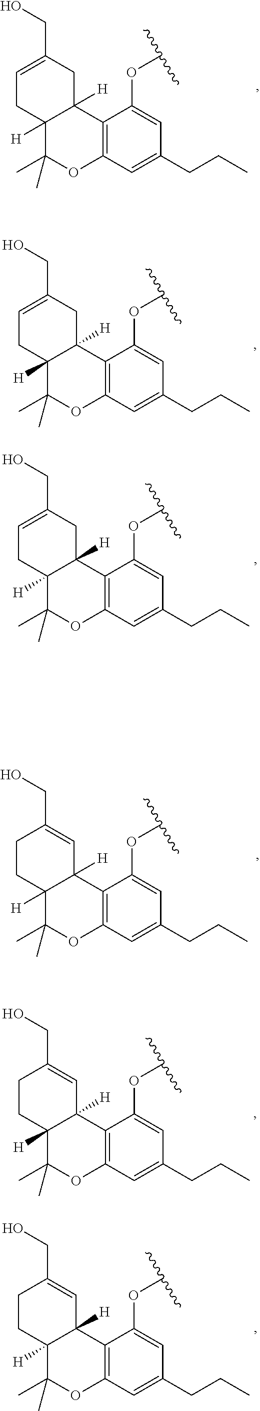

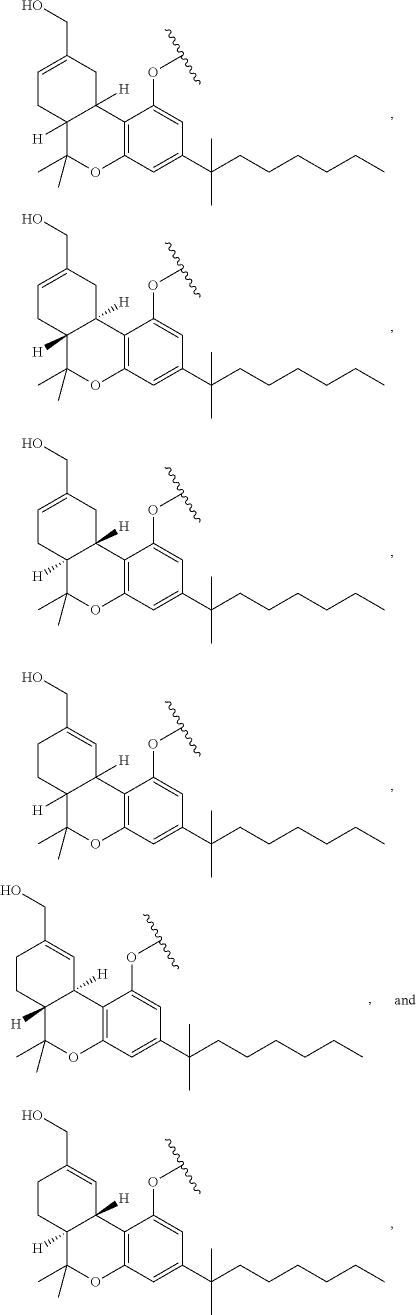

wherein R¹ is the same as described above.

In some embodiments, the compounds are in a non-salt form. In some embodiments, the compounds are in a salt form. In some embodiments, the compounds are in an ammonium salt form.

In some embodiments, the

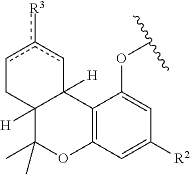

moiety is chosen from

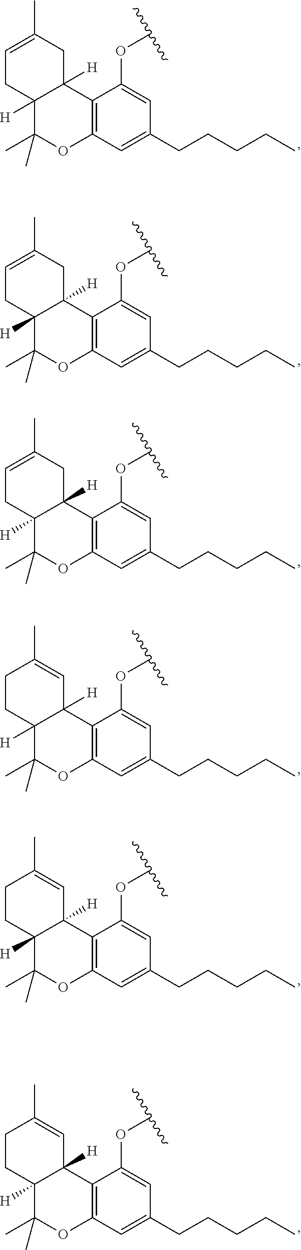

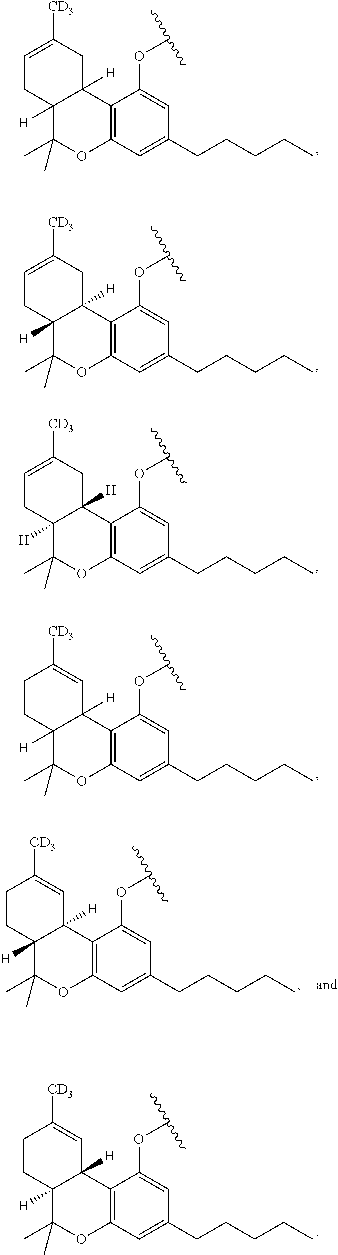

In some embodiments, the

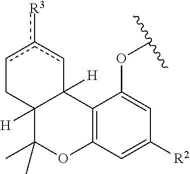

moiety is

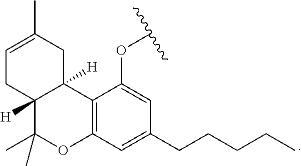 [SMILES: *Oc1cc(CCCCC)cc2c1[C@@H]1CC(C)=CC[C@H]1C(C)(C)O2]

In some embodiments, the

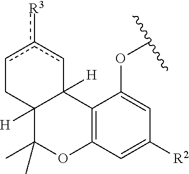

moiety is

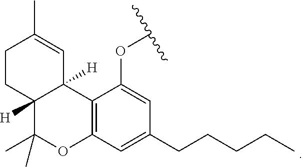 [SMILES: *Oc1cc(CCCCC)cc2c1[C@@H]1C=C(C)CC[C@H]1C(C)(C)O2]

In some embodiments, the

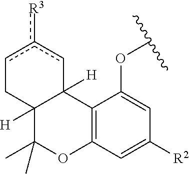

moiety is

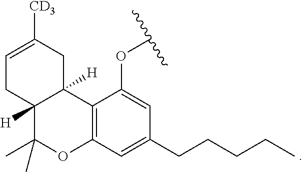 [SMILES: *Oc1cc(CCCCC)cc2c1[C@@H]1CC(C([2H])([2H])[2H])=CC[C@H]1C(C)(C)O2]

In some embodiments, the

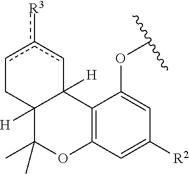

moiety is

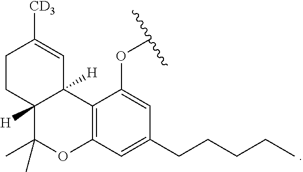 [SMILES: *Oc1cc(CCCCC)cc2c1[C@@H]1C=C(C([2H])([2H])[2H])CC[C@H]1C(C)(C)O2]

In some embodiments, the

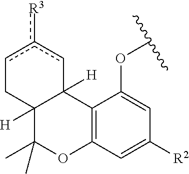

moiety is chosen from

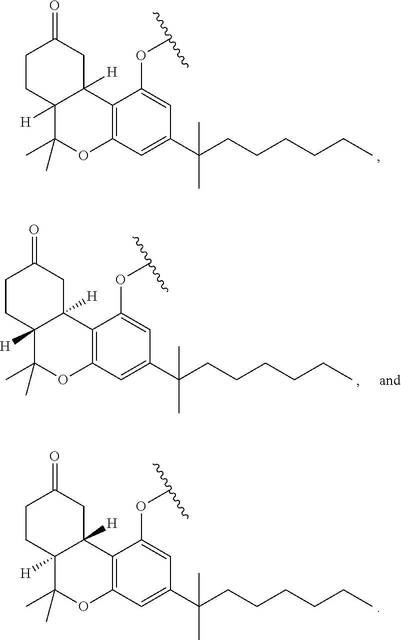 [SMILES: *Oc1cc(C(C)(C)CCCCCC)cc2c1C1CC(=O)CCC1C(C)(C)O2 ; *Oc1cc(C(C)(C)CCCCCC)cc2c1[C@@H]1CC(=O)CC[C@H]1C(C)(C)O2 ; *Oc1cc(C(C)(C)CCCCCC)cc2c1[C@H]1CC(=O)CC[C@@H]1C(C)(C)O2]

In some embodiments, the

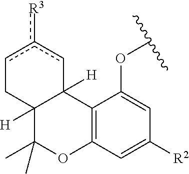

moiety is

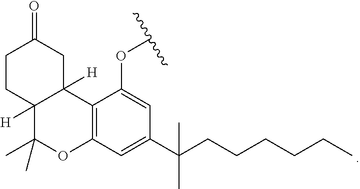 [SMILES: *Oc1cc(C(C)(C)CCCCCC)cc2c1C1CC(=O)CCC1C(C)(C)O2]

In some embodiments, the

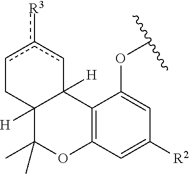

moiety is chosen from

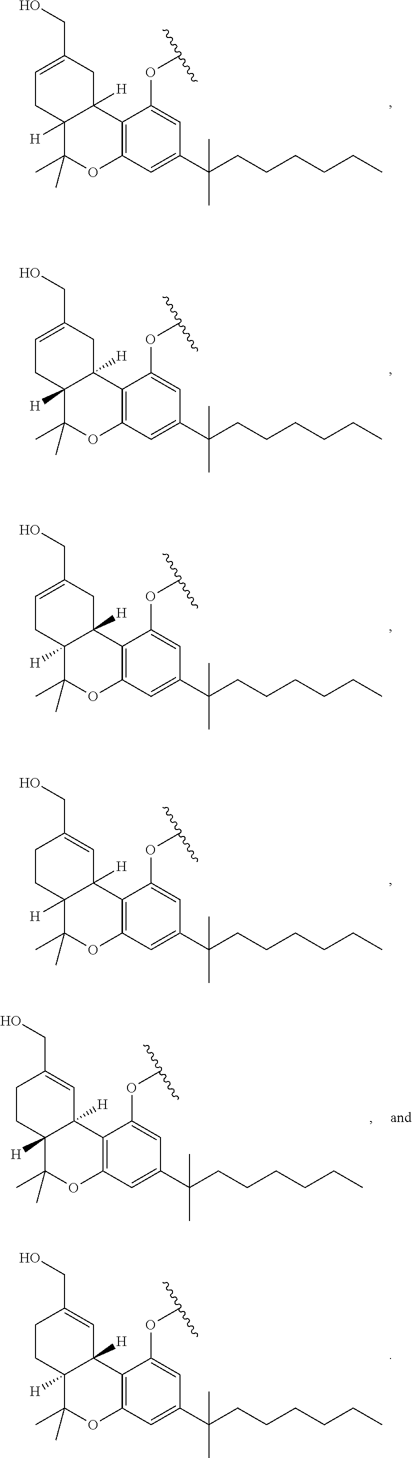 [SMILES: *Oc1cc(C(C)(C)CCCCCC)cc2c1C1CC(CO)=CCC1C(C)(C)O2 ; *Oc1cc(C(C)(C)CCCCCC)cc2c1[C@@H]1CC(CO)=CC[C@H]1C(C)(C)O2 ; *Oc1cc(C(C)(C)CCCCCC)cc2c1[C@H]1CC(CO)=CC[C@@H]1C(C)(C)O2 ; *Oc1cc(C(C)(C)CCCCCC)cc2c1C1C=C(CO)CCC1C(C)(C)O2 ; *Oc1cc(C(C)(C)CCCCCC)cc2c1[C@@H]1C=C(CO)CC[C@H]1C(C)(C)O2 ; *Oc1cc(C(C)(C)CCCCCC)cc2c1[C@H]1C=C(CO)CC[C@@H]1C(C)(C)O2]

In some embodiments, the

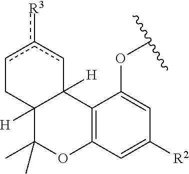

moiety is

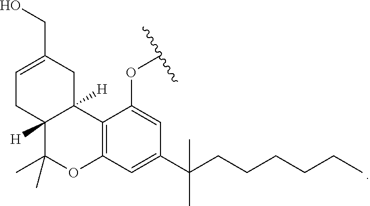 [SMILES: *Oc1cc(C(C)(C)CCCCCC)cc2c1[C@@H]1CC(CO)=CC[C@H]1C(C)(C)O2]

In some embodiments, the

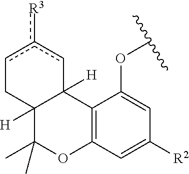

moiety is

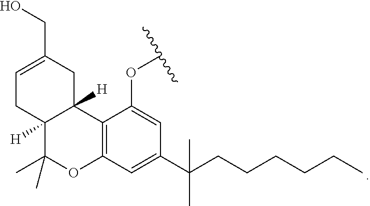 [SMILES: *Oc1cc(C(C)(C)CCCCCC)cc2c1[C@H]1CC(CO)=CC[C@@H]1C(C)(C)O2]

In some embodiments, R¹ is

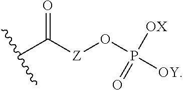

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q. and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

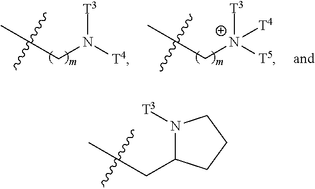

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

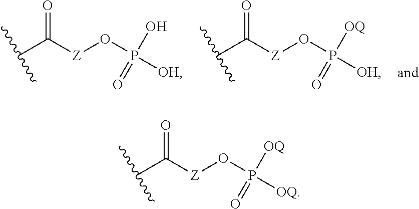

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

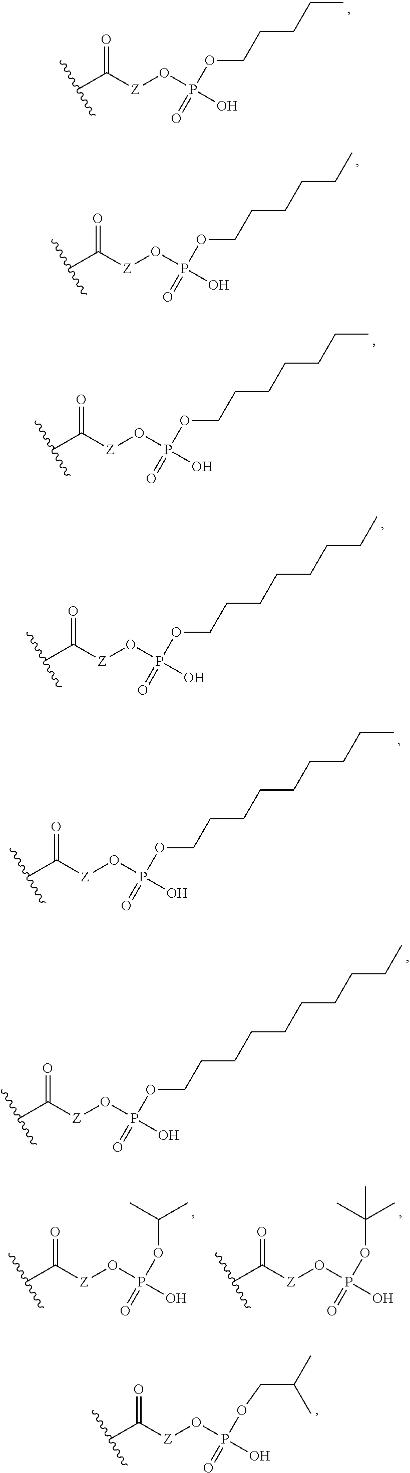

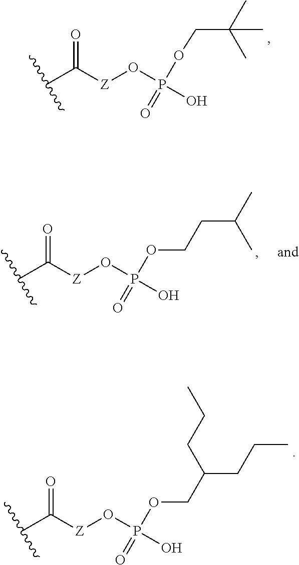

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

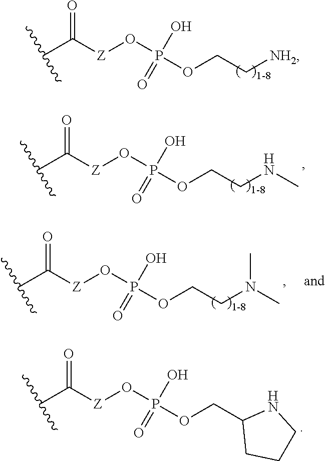

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

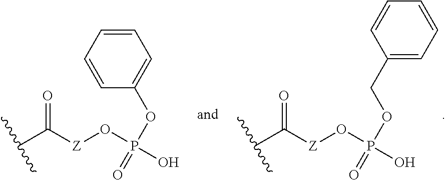

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

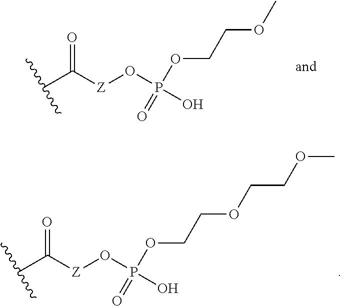

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

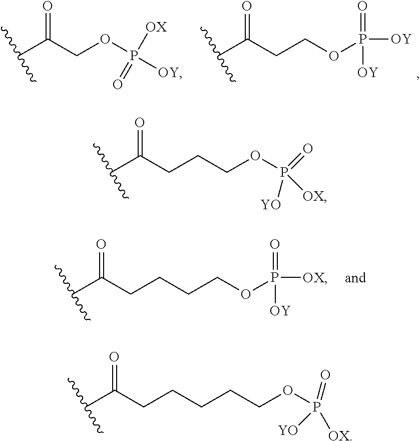

In some embodiments. X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

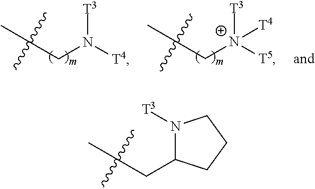

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

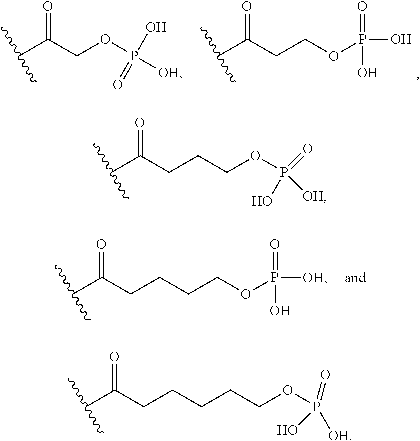

In some embodiments, R¹ is chosen from

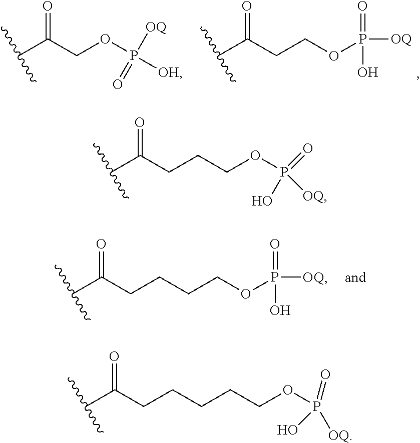

In some embodiments, R¹ is chosen from

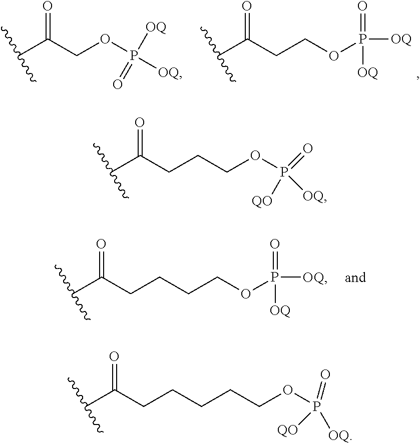

In some embodiments, R¹ is chosen from

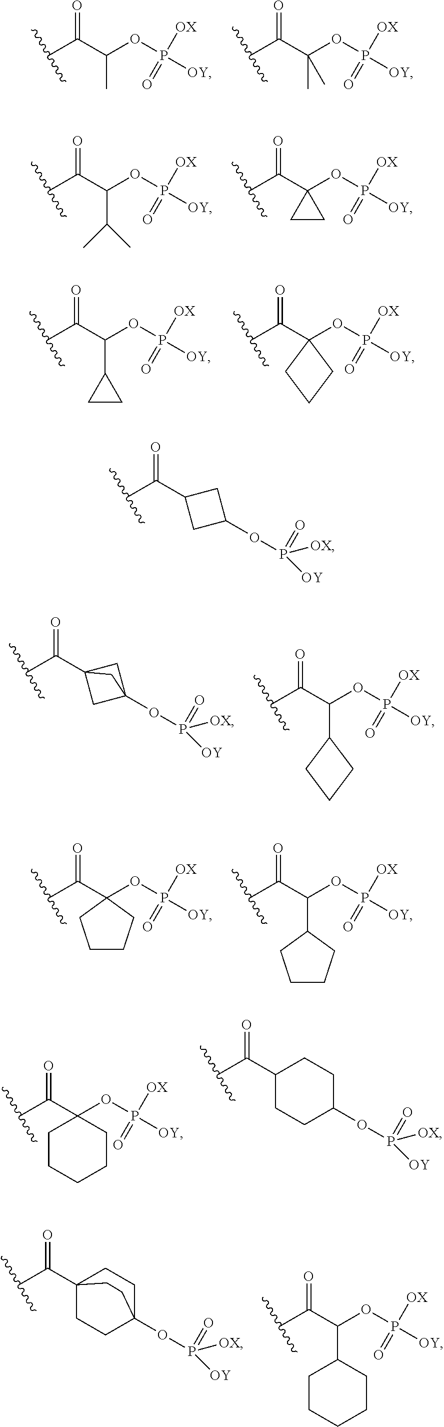

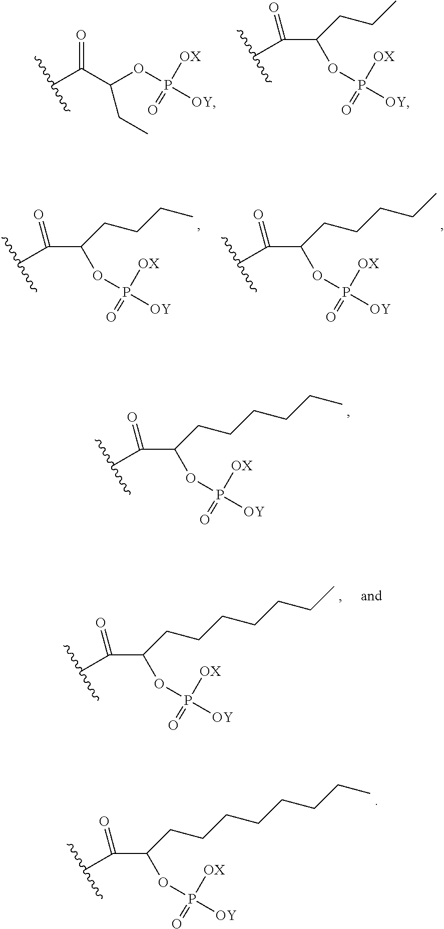

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₈ alkyl, C₁₋₈ haloalkyl, C₆₋₁₈ aryl. C₁₋₁₃ heteroaryl, C₇₋₁₉ arylalkyl, C₂₋₁₄ heteroarylalkyl, —(CH₂CH₂O)ₙCH₃,

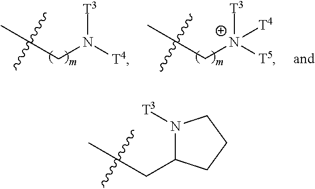

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

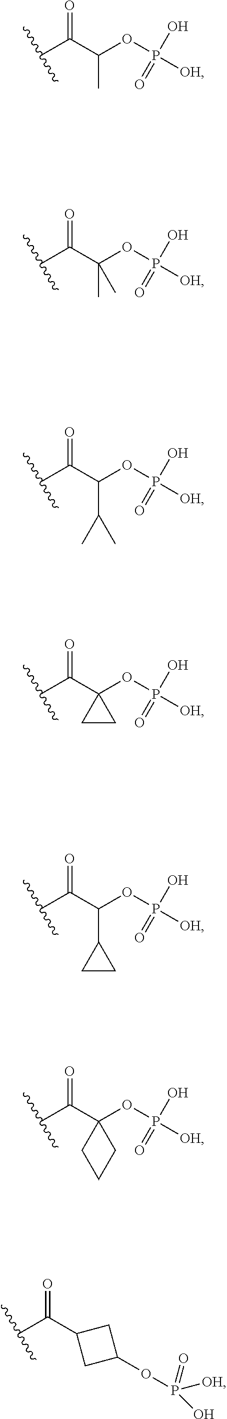

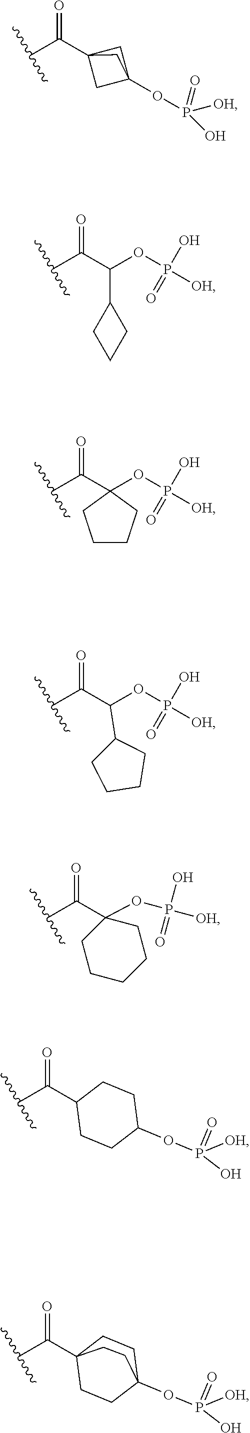

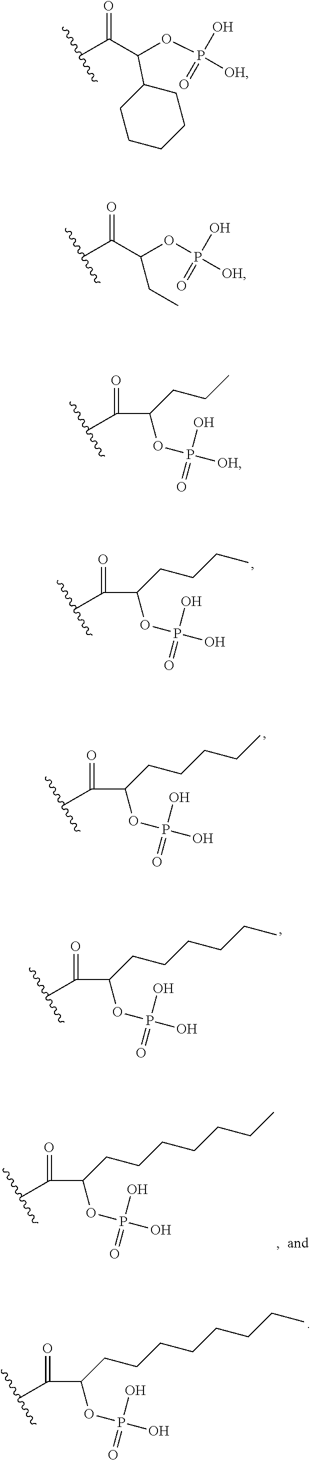

In some embodiments, R¹ is chosen from

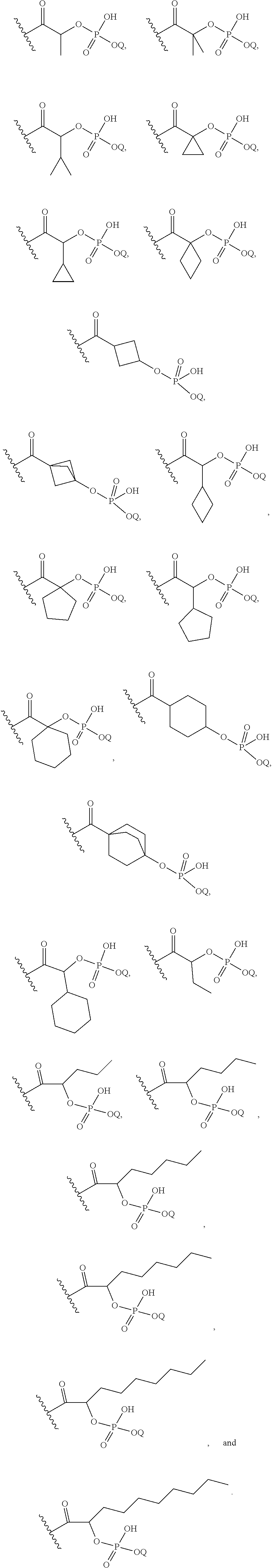

In some embodiments, R¹ is chosen from

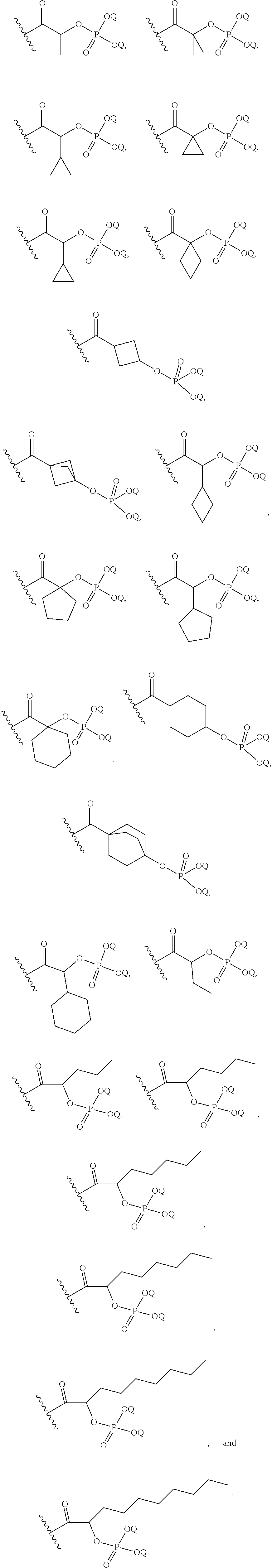

In some embodiments, R¹ is chosen from

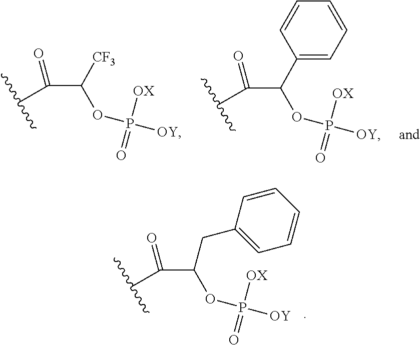

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

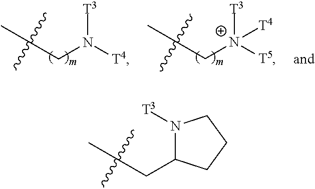

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

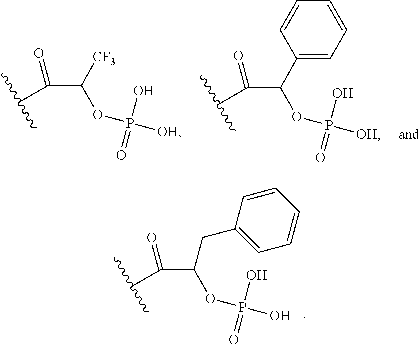 [SMILES: *C(=O)C(OP(=O)(O)O)C(F)(F)F ; *C(=O)C(OP(=O)(O)O)c1ccccc1 ; "and" ; *C(=O)C(Cc1ccccc1)OP(=O)(O)O]

In some embodiments, R¹ is chosen from

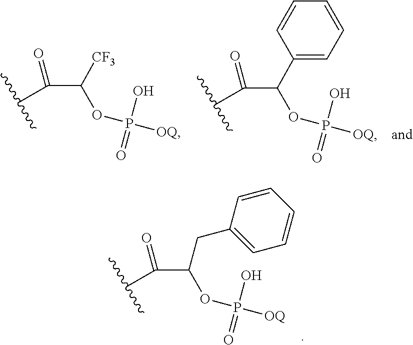

In some embodiments, R¹ is chosen from

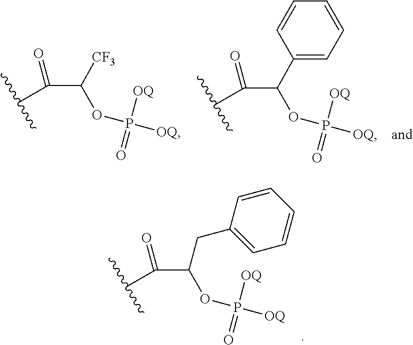

In some embodiments, R¹ is chosen from

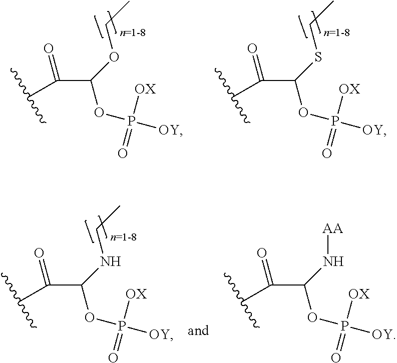

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q. and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

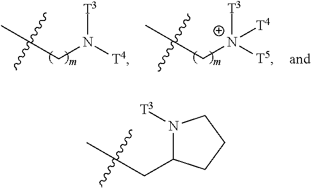

groups. In some embodiments, X is chosen from H and Q, and Y is chosen from phenyl, benzyl, and C₁₋₈ alkyl groups. In some embodiments, X is chosen from H and Q, and Y is chosen from phenyl, benzyl, ethyl, and isopropyl.

In some embodiments, R¹ is chosen from

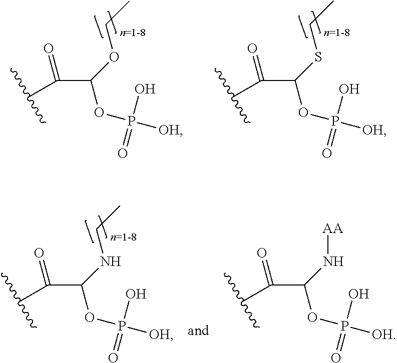

In some embodiments, R¹ is chosen from

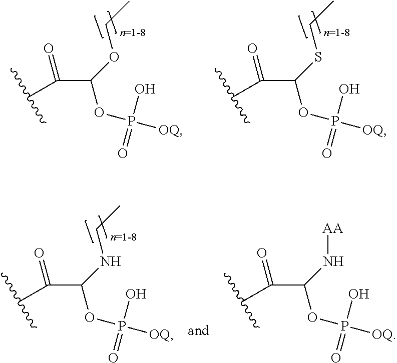

In some embodiments, R¹ is chosen from

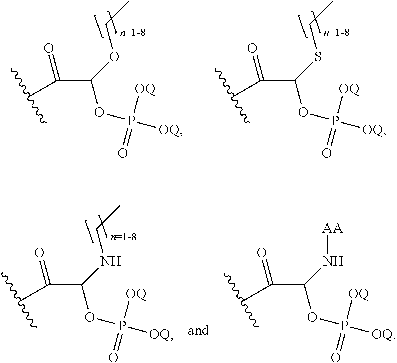

In some embodiments, R¹ is chosen from

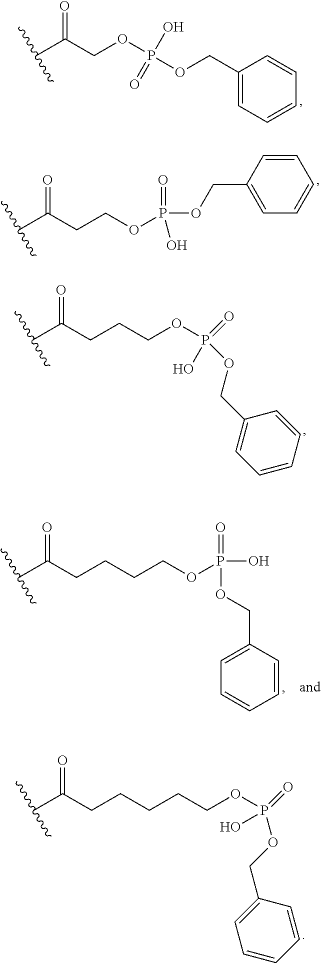

In some embodiments, R¹ is chosen from

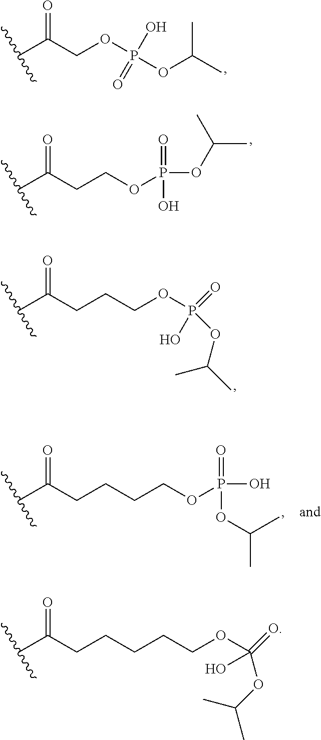

In some embodiments, R¹ is chosen from

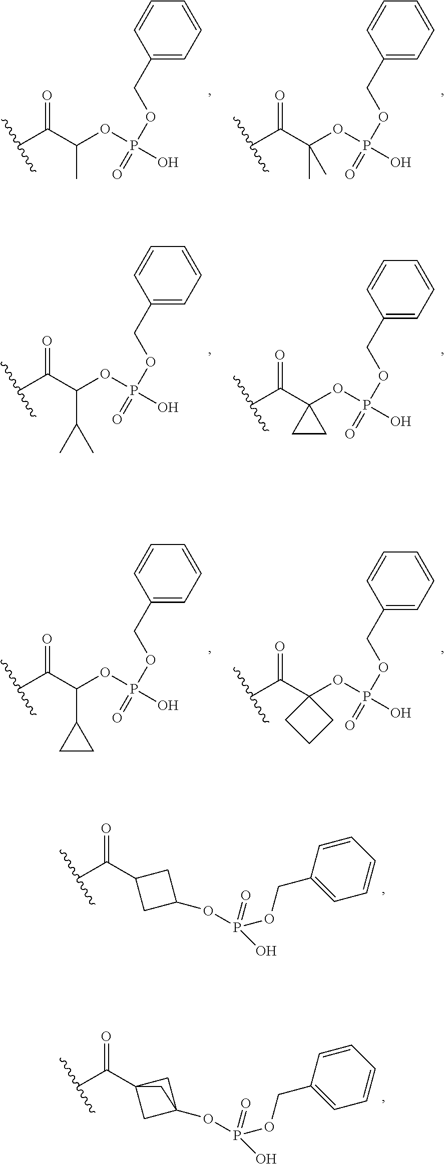

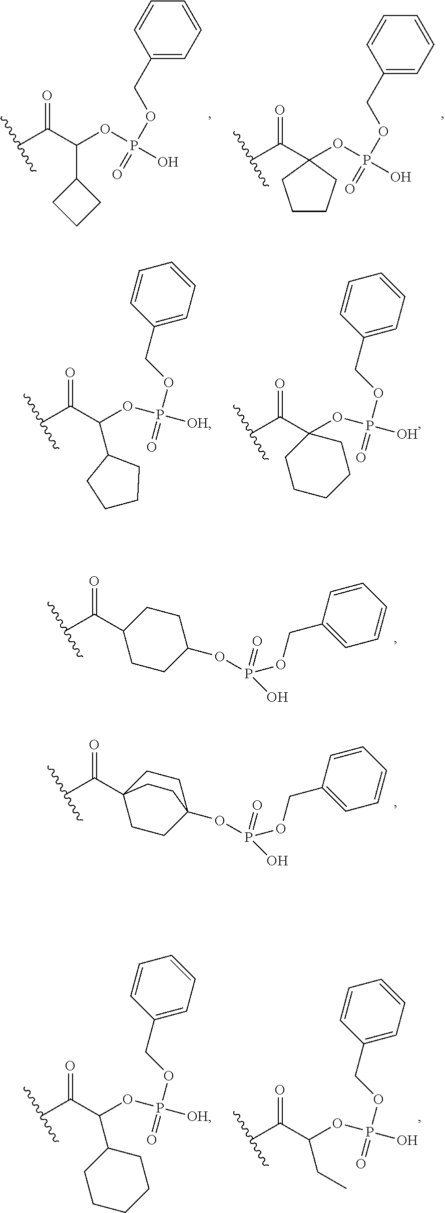

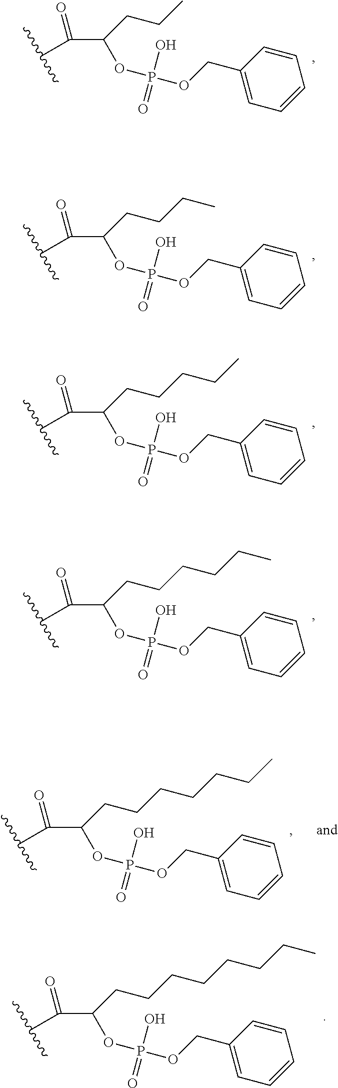 [SMILES: *C(=O)C(CCC)OP(=O)(O)OCc1ccccc1 ; *C(=O)C(CCCC)OP(=O)(O)OCc1ccccc1 ; *C(=O)C(CCCCC)OP(=O)(O)OCc1ccccc1 ; *C(=O)C(CCCCCC)OP(=O)(O)OCc1ccccc1 ; *C(=O)C(CCCCCCC)OP(=O)(O)OCc1ccccc1 ; *C(=O)C(CCCCCCCC)OP(=O)(O)OCc1ccccc1]

In some embodiments, R¹ is chosen from

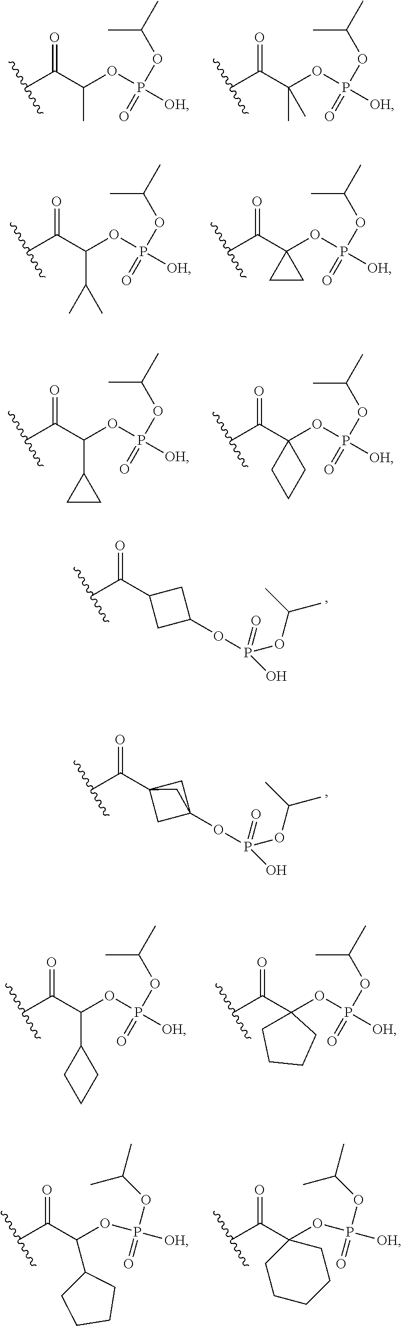

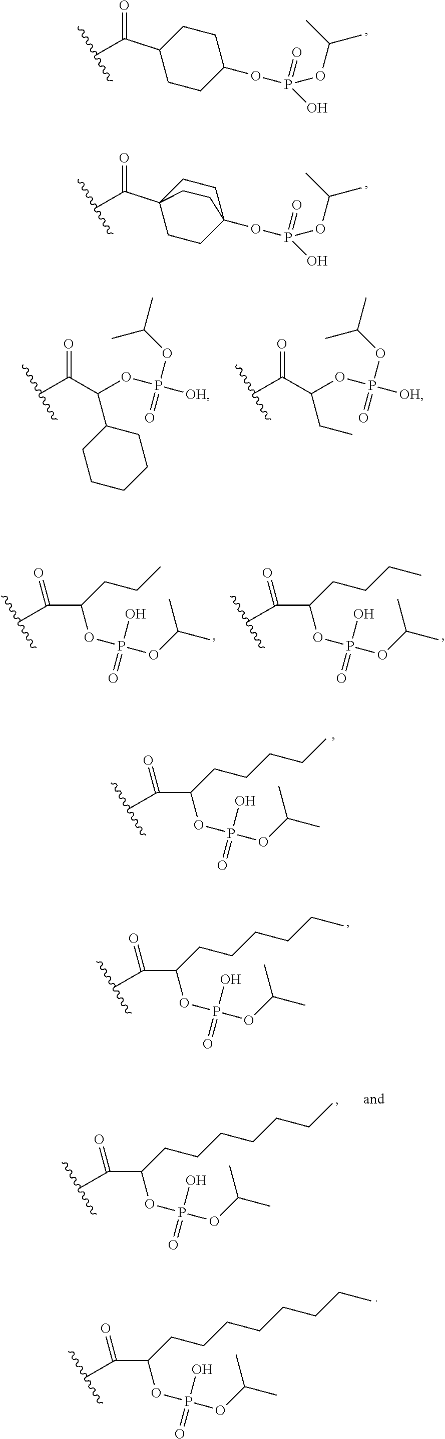

In some embodiments, R¹ is chosen from

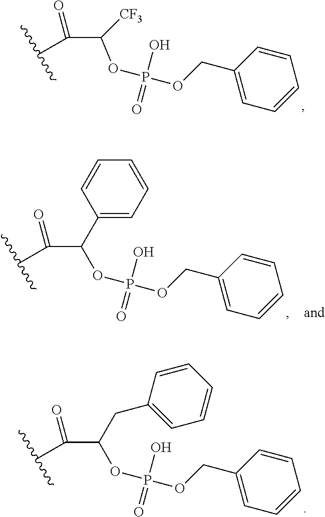 [SMILES: *C(=O)C(OP(=O)(O)OCc1ccccc1)C(F)(F)F ; *C(=O)C(OP(=O)(O)OCc1ccccc1)c1ccccc1 ; *C(=O)C(Cc1ccccc1)OP(=O)(O)OCc1ccccc1]

In some embodiments, R¹ is chosen from

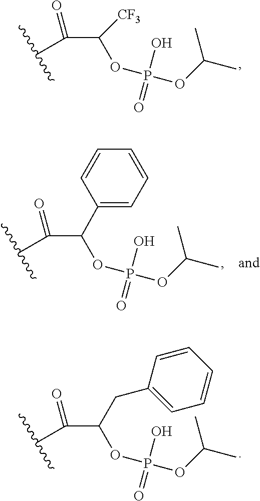

In some embodiments, R¹ is chosen from

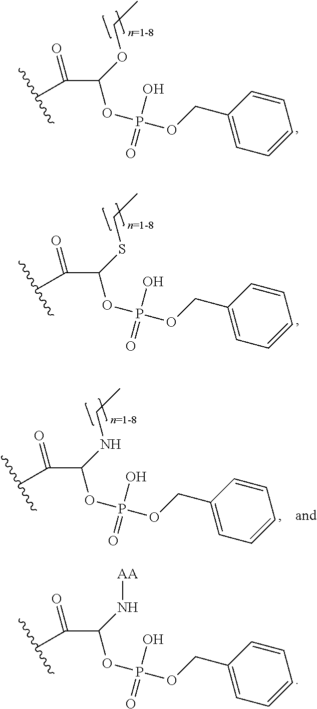

In some embodiments, R¹ is chosen from

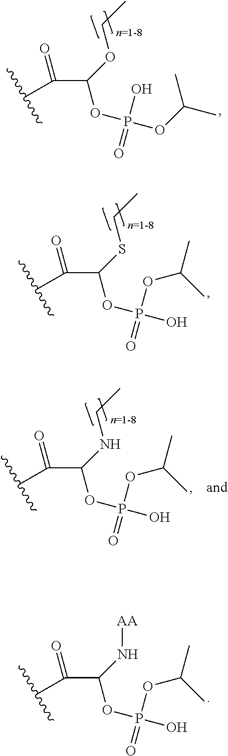

In some embodiments, R¹ is

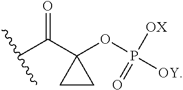

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

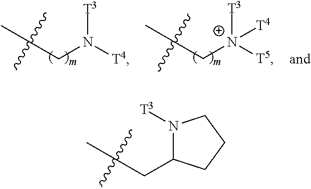

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

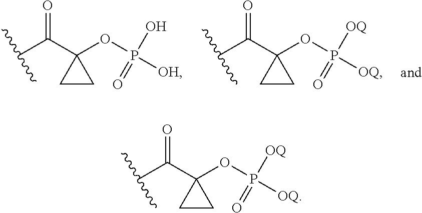

In some embodiments, R¹ is

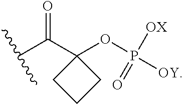

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

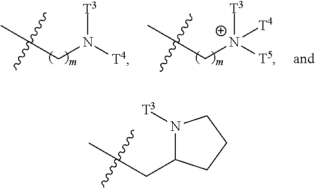

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

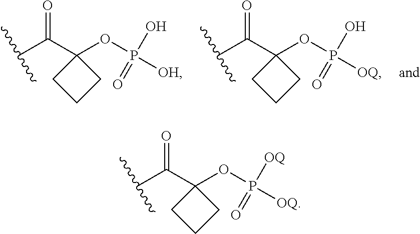

In some embodiments, R¹ is

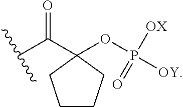

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q. and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

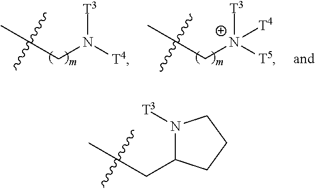

groups. In some embodiments, X is chosen from H and Q. and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

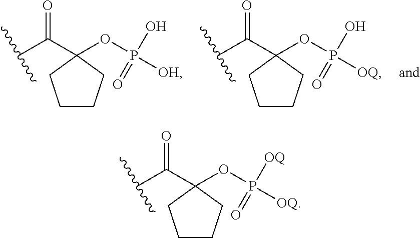

In some embodiments, R¹ is

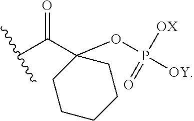

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

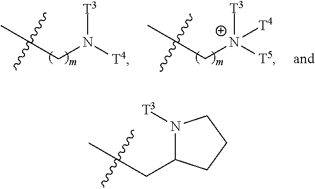

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, R¹ is chosen from

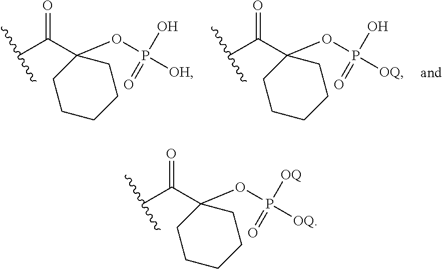

In some embodiments, R¹ is

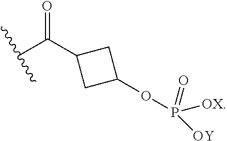

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

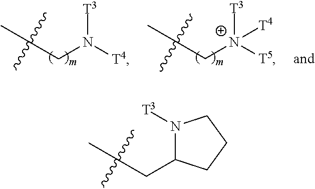

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

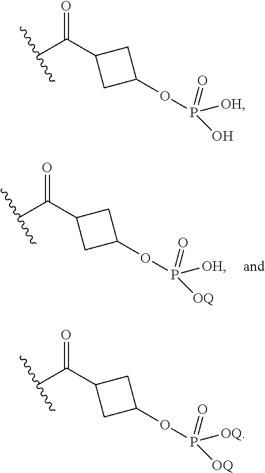

In some embodiments, R¹ is

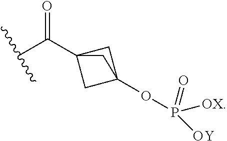

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

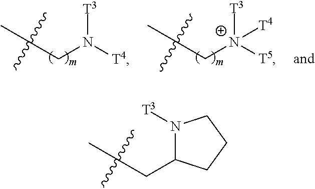

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

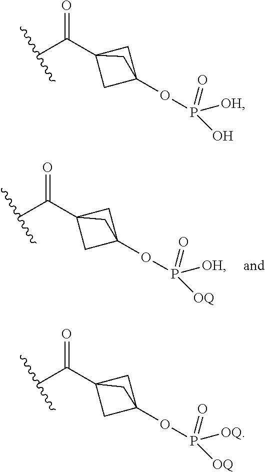

In some embodiments, R¹ is

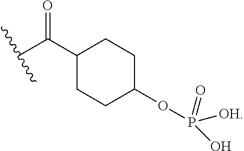 [SMILES: *C(=O)C1CCC(OP(=O)(O)O)CC1]

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

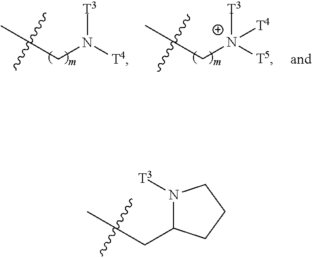

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, R¹ is chosen from

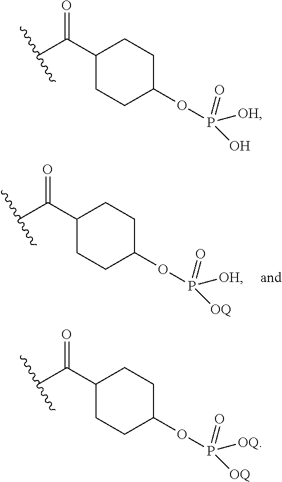

In some embodiments, R¹ is

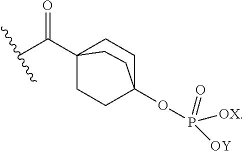

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

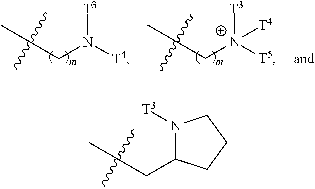

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, R¹ is chosen from

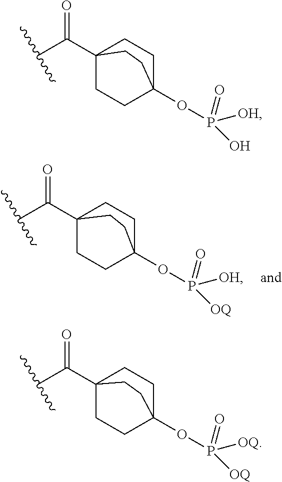

In some embodiments, R¹ is chosen from

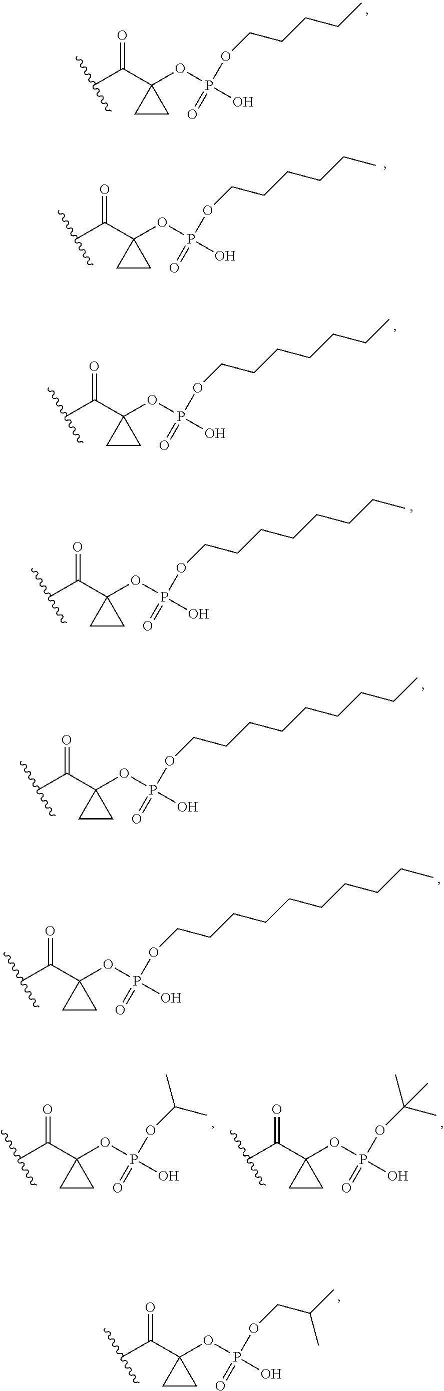

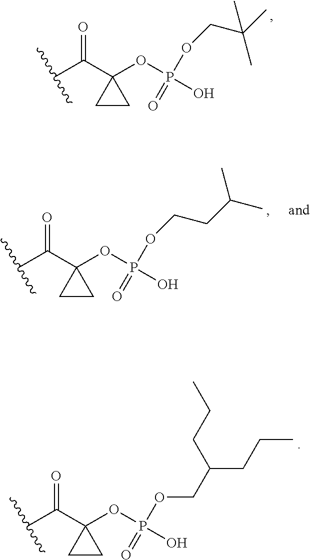 [SMILES: *C(=O)C1(OP(=O)(O)OCC(C)(C)C)CC1 ; *C(=O)C1(OP(=O)(O)OCCC(C)C)CC1 ; *C(=O)C1(OP(=O)(O)OCC(CCC)CCC)CC1]

In some embodiments, R¹ is chosen from

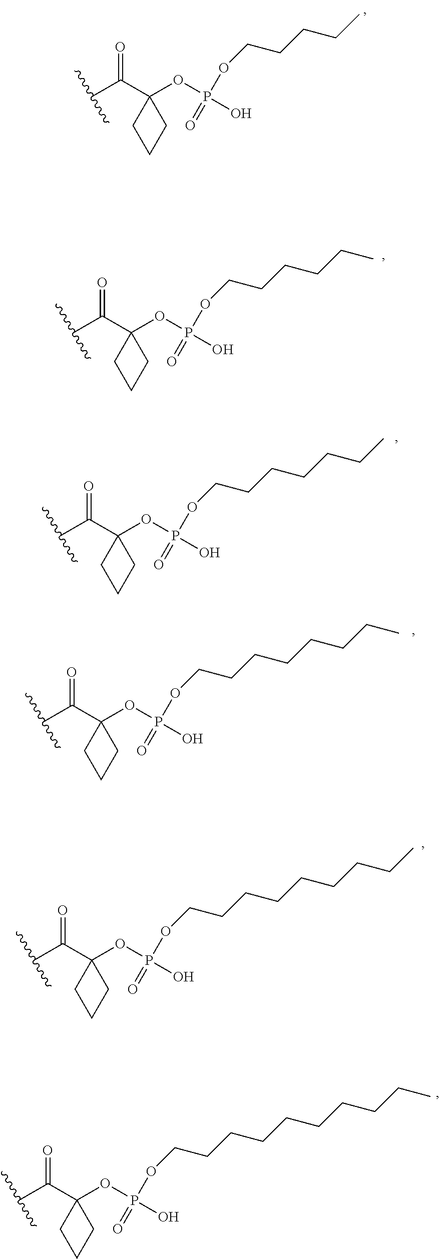 [SMILES: *C(=O)C1(OP(=O)(O)OCCCCC)CCC1 ; *C(=O)C1(OP(=O)(O)OCCCCCC)CCC1 ; *C(=O)C1(OP(=O)(O)OCCCCCCC)CCC1 ; *C(=O)C1(OP(=O)(O)OCCCCCCCC)CCC1 ; *C(=O)C1(OP(=O)(O)OCCCCCCCCC)CCC1 ; *C(=O)C1(OP(=O)(O)OCCCCCCCCCC)CCC1]

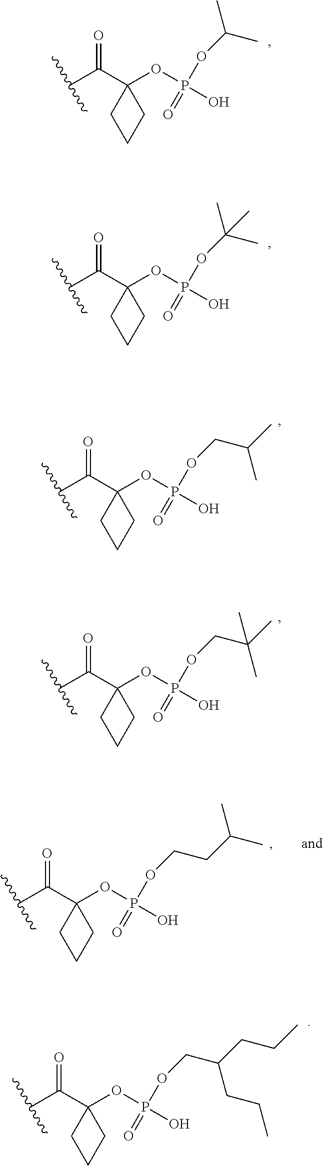 [SMILES: *C(=O)C1(OP(=O)(O)OC(C)C)CCC1 ; *C(=O)C1(OP(=O)(O)OC(C)(C)C)CCC1 ; *C(=O)C1(OP(=O)(O)OCC(C)C)CCC1 ; *C(=O)C1(OP(=O)(O)OCC(C)(C)C)CCC1 ; *C(=O)C1(OP(=O)(O)OCCC(C)C)CCC1 ; *C(=O)C1(OP(=O)(O)OCC(CCC)CCC)CCC1]

In some embodiments, R¹ is chosen from

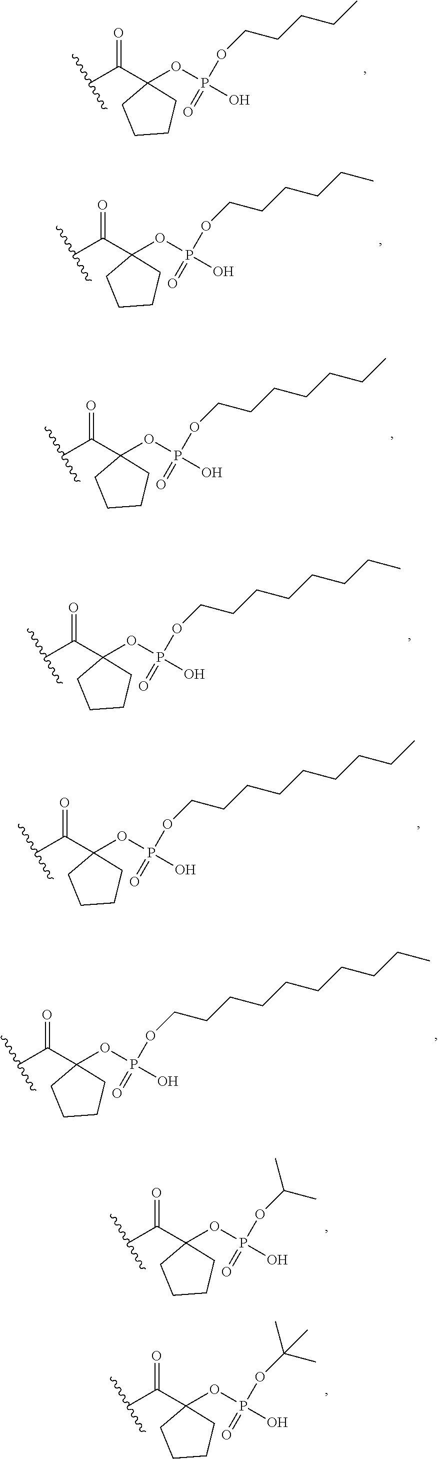

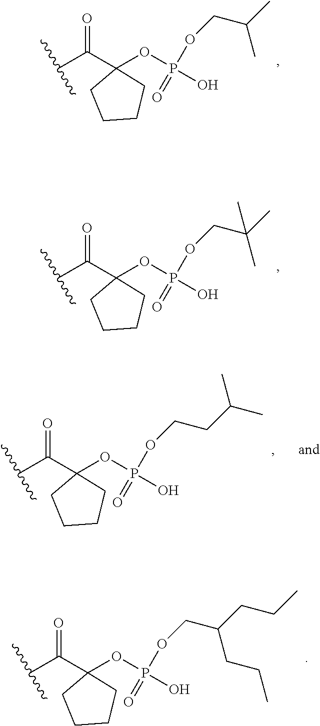 [SMILES: *C(=O)C1(OP(=O)(O)OCC(C)C)CCCC1 ; *C(=O)C1(OP(=O)(O)OCC(C)(C)C)CCCC1 ; *C(=O)C1(OP(=O)(O)OCCC(C)C)CCCC1 ; *C(=O)C1(OP(=O)(O)OCC(CCC)CCC)CCCC1]

In some embodiments, R¹ is chosen from

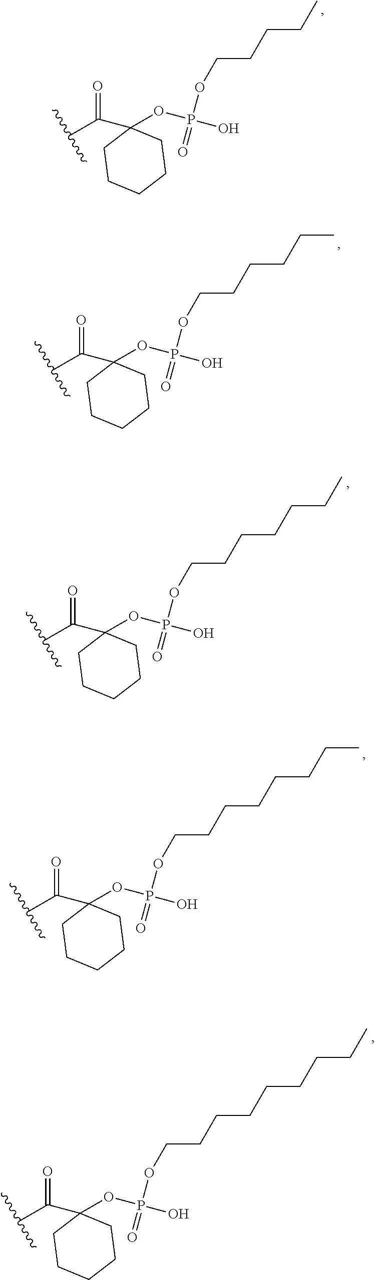 [SMILES: *C(=O)C1(OP(=O)(O)OCCCCC)CCCCC1 ; *C(=O)C1(OP(=O)(O)OCCCCCC)CCCCC1 ; *C(=O)C1(OP(=O)(O)OCCCCCCC)CCCCC1 ; *C(=O)C1(OP(=O)(O)OCCCCCCCC)CCCCC1 ; *C(=O)C1(OP(=O)(O)OCCCCCCCCC)CCCCC1]

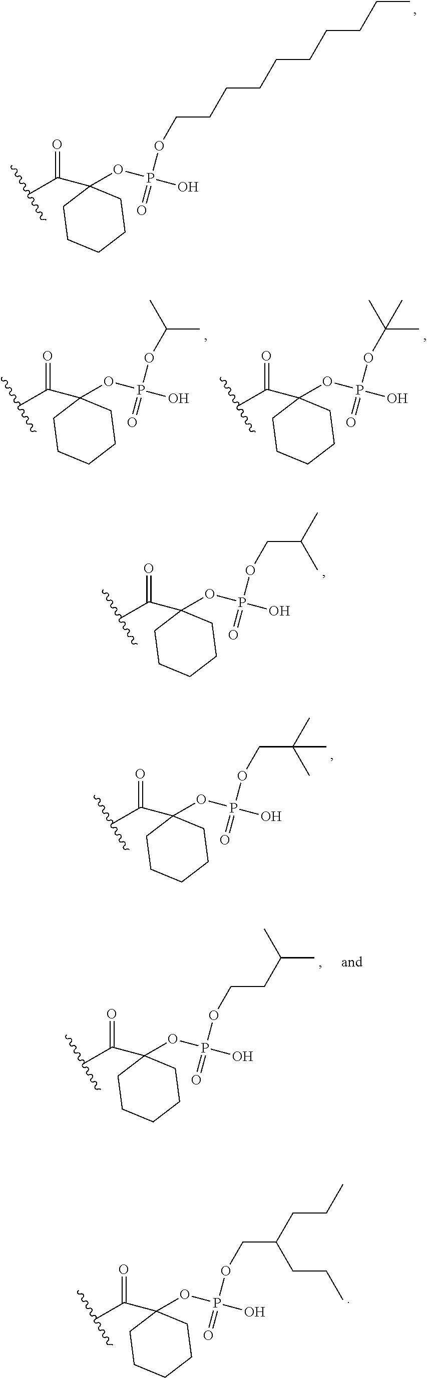

In some embodiments, R¹ is chosen from

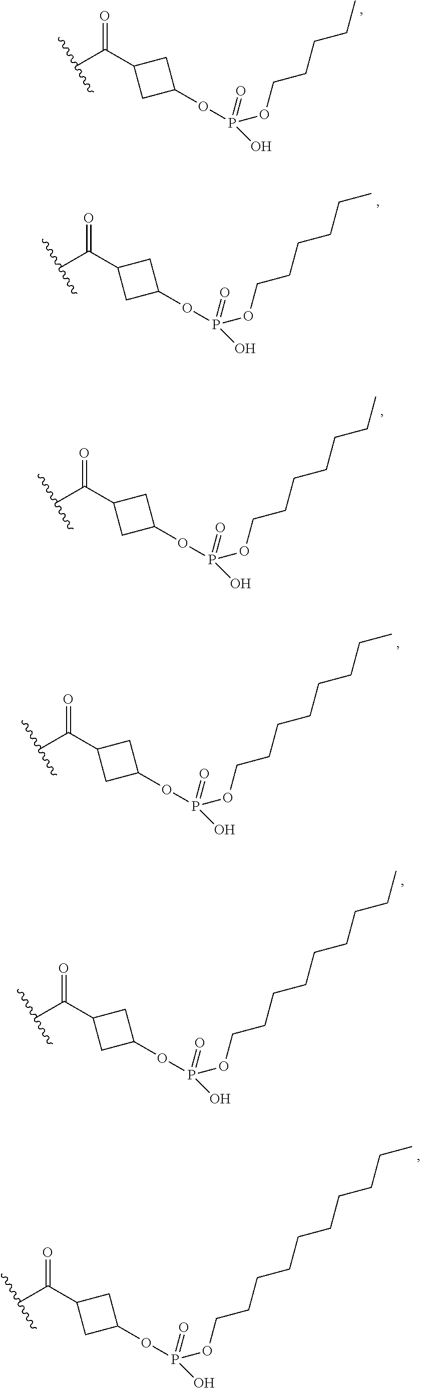 [SMILES: *C(=O)C1CC(OP(=O)(O)OCCCCC)C1 ; *C(=O)C1CC(OP(=O)(O)OCCCCCC)C1 ; *C(=O)C1CC(OP(=O)(O)OCCCCCCC)C1 ; *C(=O)C1CC(OP(=O)(O)OCCCCCCCC)C1 ; *C(=O)C1CC(OP(=O)(O)OCCCCCCCCC)C1 ; *C(=O)C1CC(OP(=O)(O)OCCCCCCCCCC)C1]

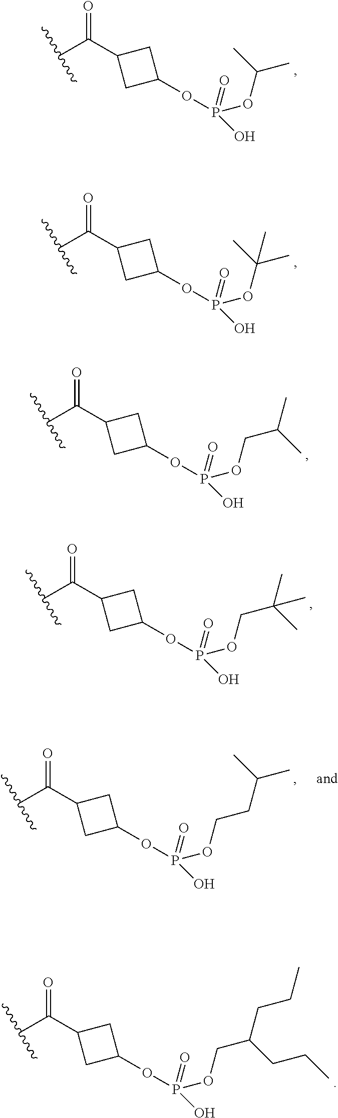 [SMILES: *C(=O)C1CC(OP(=O)(O)OC(C)C)C1 ; *C(=O)C1CC(OP(=O)(O)OC(C)(C)C)C1 ; *C(=O)C1CC(OP(=O)(O)OCC(C)C)C1 ; *C(=O)C1CC(OP(=O)(O)OCC(C)(C)C)C1 ; *C(=O)C1CC(OP(=O)(O)OCCC(C)C)C1 ; *C(=O)C1CC(OP(=O)(O)OCC(CCC)CCC)C1]

In some embodiments, R¹ is chosen from

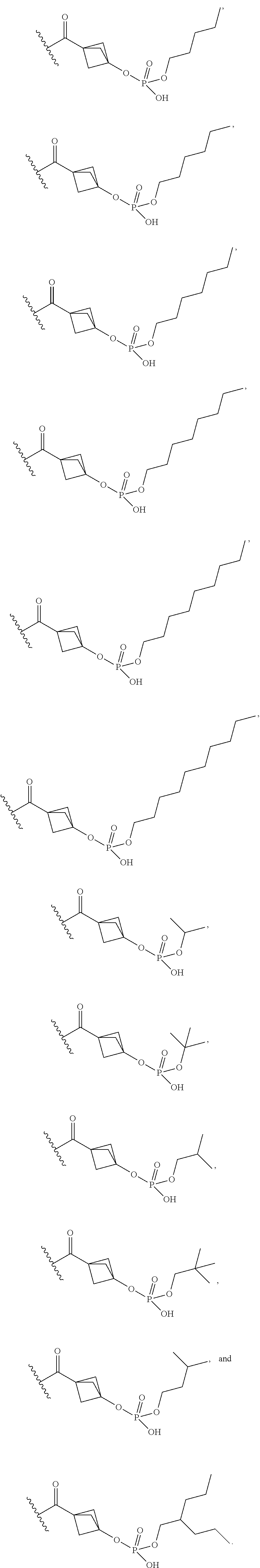

In some embodiments, R¹ is chosen from

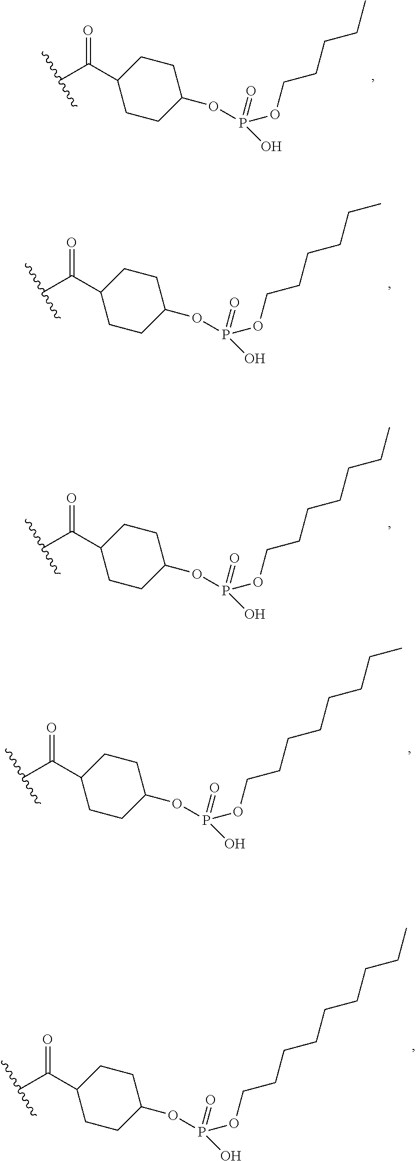 [SMILES: *C(=O)C1CCC(OP(=O)(O)OCCCCC)CC1 ; *C(=O)C1CCC(OP(=O)(O)OCCCCCC)CC1 ; *C(=O)C1CCC(OP(=O)(O)OCCCCCCC)CC1 ; *C(=O)C1CCC(OP(=O)(O)OCCCCCCCC)CC1 ; *C(=O)C1CCC(OP(=O)(O)OCCCCCCCCC)CC1]

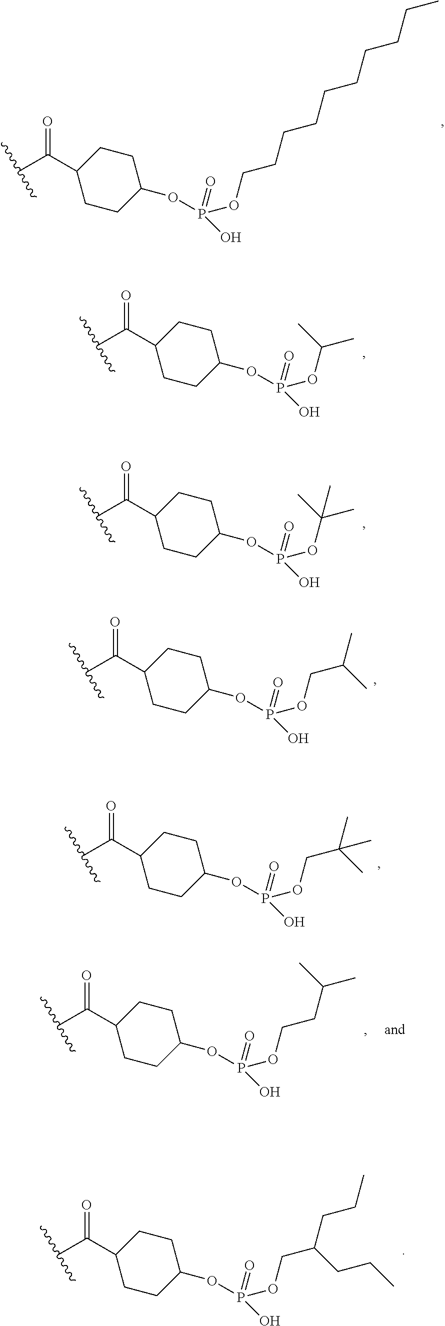

In some embodiments, R¹ is chosen from

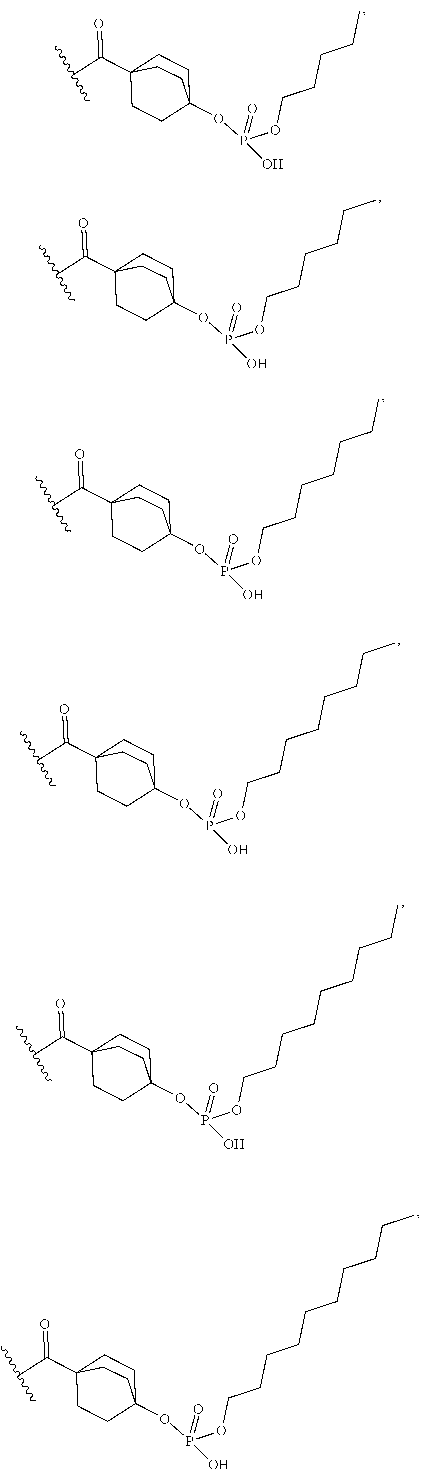 [SMILES: *C(=O)C12CCC(OP(=O)(O)OCCCCC)(CC1)CC2 ; *C(=O)C12CCC(OP(=O)(O)OCCCCCC)(CC1)CC2 ; *C(=O)C12CCC(OP(=O)(O)OCCCCCCC)(CC1)CC2 ; *C(=O)C12CCC(OP(=O)(O)OCCCCCCCC)(CC1)CC2 ; *C(=O)C12CCC(OP(=O)(O)OCCCCCCCCC)(CC1)CC2 ; *C(=O)C12CCC(OP(=O)(O)OCCCCCCCCCC)(CC1)CC2]

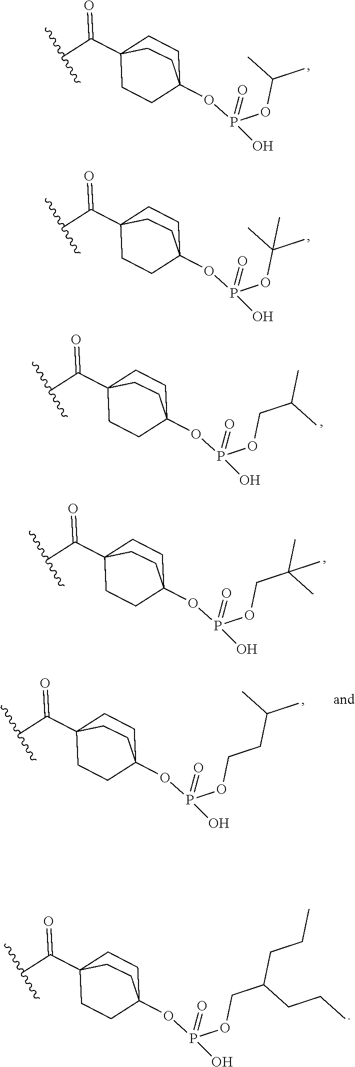

In some embodiments, R¹ is chosen from

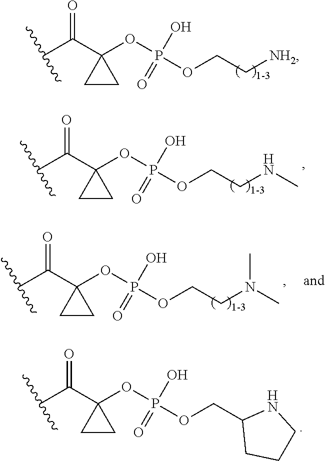

In some embodiments, R¹ is chosen from

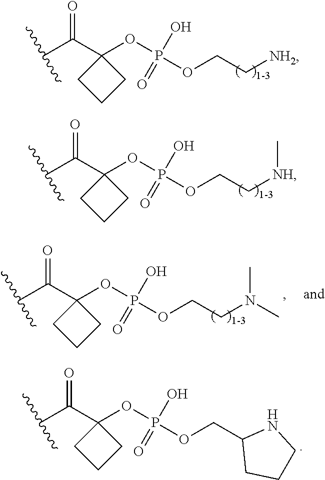

In some embodiments, R¹ is chosen from

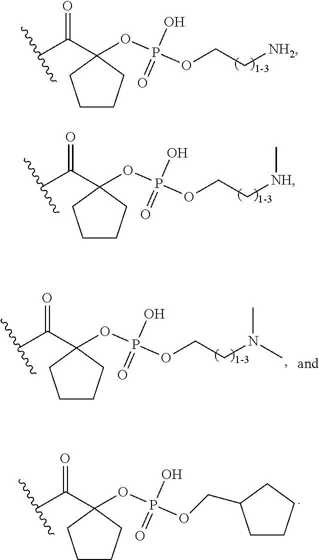

In some embodiments, R¹ is chosen from

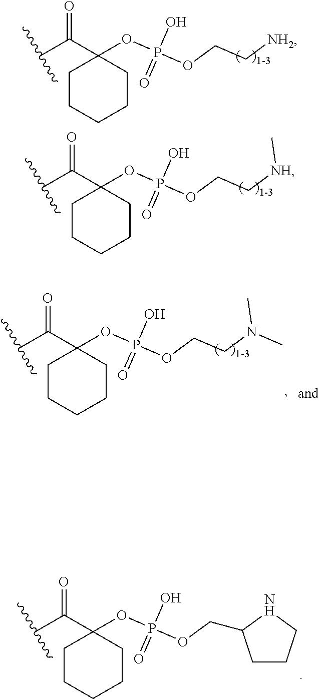

In some embodiments, R¹ is chosen from 1-3

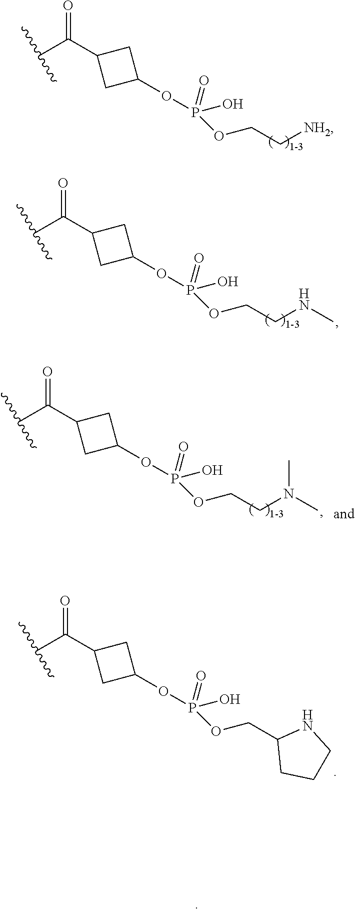

In some embodiments, R¹ is chosen from

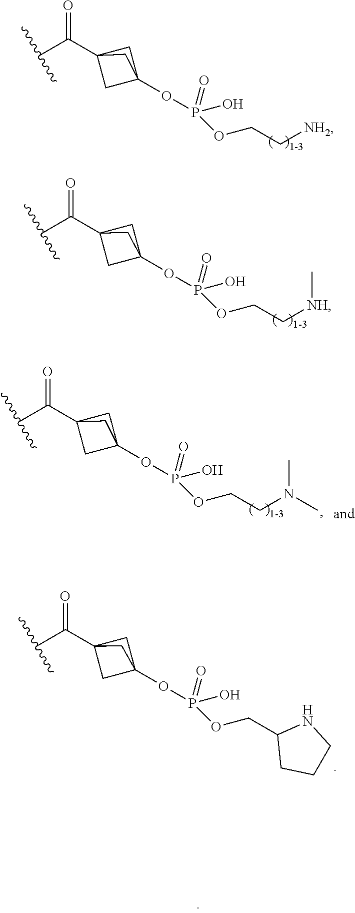

In some embodiments, R¹ is chosen from 3

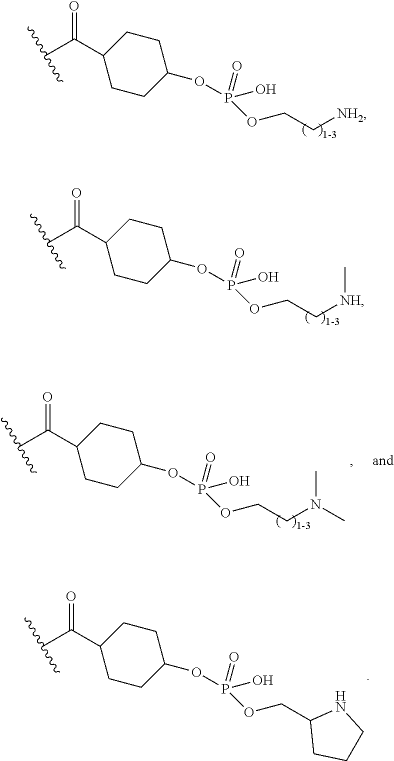

In some embodiments, R¹ is chosen from

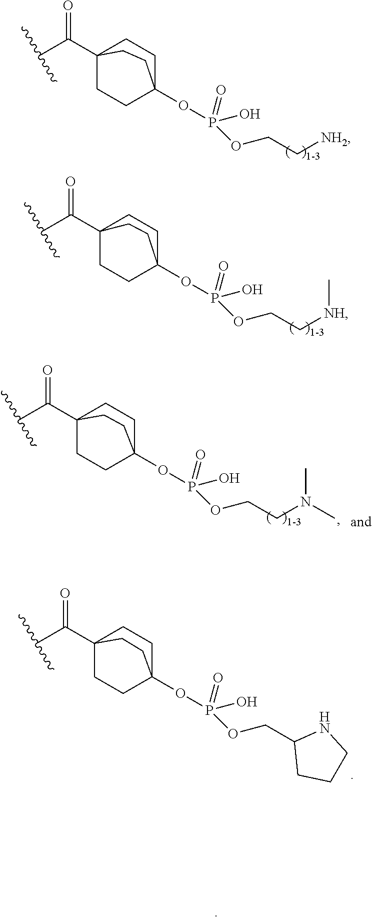

In some embodiments, R¹ is chosen from

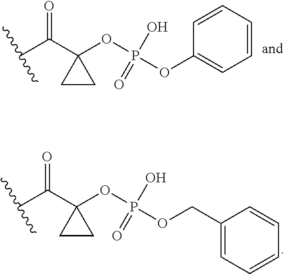 [SMILES: *C(=O)C1(OP(=O)(O)Oc2ccccc2)CC1 ; *C(=O)C1(OP(=O)(O)OCc2ccccc2)CC1]

In some embodiments, R¹ is chosen from

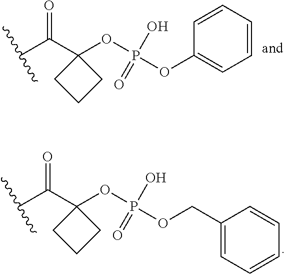 [SMILES: *C(=O)C1(OP(=O)(O)Oc2ccccc2)CCC1 ; *C(=O)C1(OP(=O)(O)OCc2ccccc2)CCC1]

In some embodiments, R¹ is chosen from

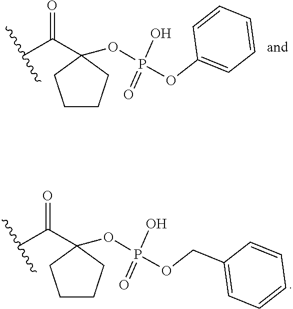 [SMILES: *C(=O)C1(OP(=O)(O)Oc2ccccc2)CCCC1 ; *C(=O)C1(OP(=O)(O)OCc2ccccc2)CCCC1]

In some embodiments, R¹ is chosen from

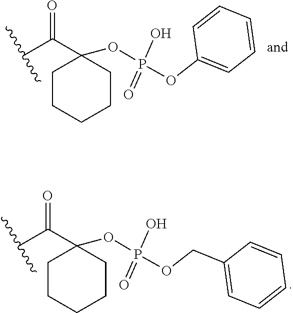 [SMILES: *C(=O)C1(OP(=O)(O)Oc2ccccc2)CCCCC1 ; *C(=O)C1(OP(=O)(O)OCc2ccccc2)CCCCC1]

In some embodiments, R¹ is chosen from

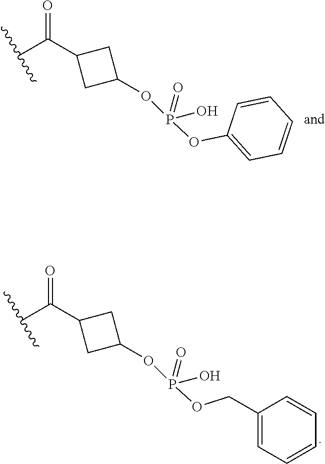 [SMILES: *C(=O)C1CC(OP(=O)(O)Oc2ccccc2)C1 ; *C(=O)C1CC(OP(=O)(O)OCc2ccccc2)C1]

In some embodiments, R¹ is chosen from

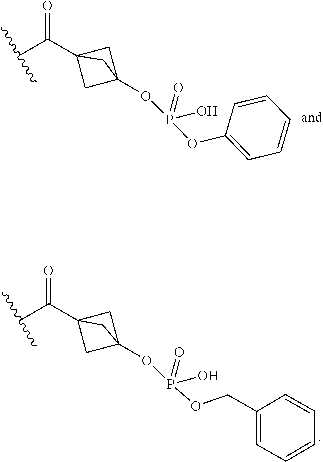 [SMILES: *C(=O)C12CC(OP(=O)(O)Oc3ccccc3)(C1)C2 ; *C(=O)C12CC(OP(=O)(O)OCc3ccccc3)(C1)C2]

In some embodiments, R¹ is chosen from

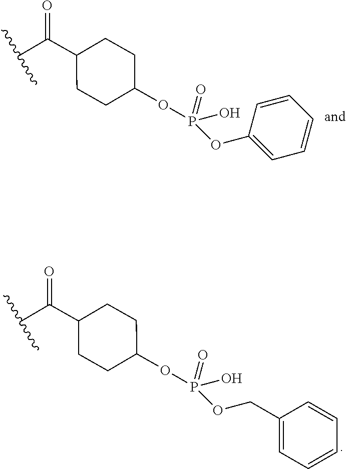 [SMILES: *C(=O)C1CCC(OP(=O)(O)Oc2ccccc2)CC1 ; *C(=O)C1CCC(OP(=O)(O)OCc2ccccc2)CC1]

In some embodiments, R¹ is chosen from

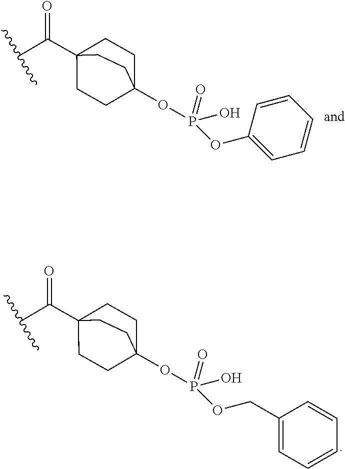 [SMILES: *C(=O)C12CCC(OP(=O)(O)Oc3ccccc3)(CC1)CC2 ; *C(=O)C12CCC(OP(=O)(O)OCc3ccccc3)(CC1)CC2]

In some embodiments, R¹ is chosen from

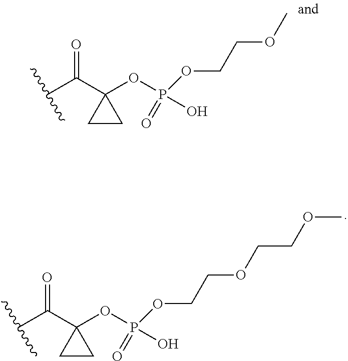 [SMILES: *C(=O)C1(OP(=O)(O)OCCOC)CC1 ; *C(=O)C1(OP(=O)(O)OCCOCCOC)CC1]

In some embodiments, R¹ is chosen from

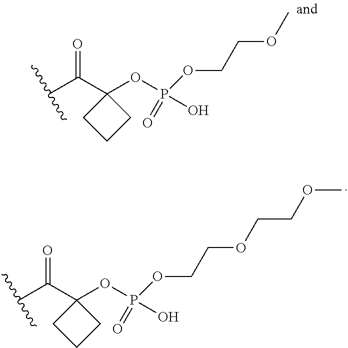 [SMILES: *C(=O)C1(OP(=O)(O)OCCOC)CCC1 ; *C(=O)C1(OP(=O)(O)OCCOCCOC)CCC1]

In some embodiments, R¹ is chosen from

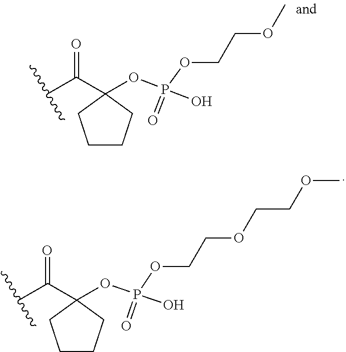 [SMILES: *C(=O)C1(OP(=O)(O)OCCOC)CCCC1 ; *C(=O)C1(OP(=O)(O)OCCOCCOC)CCCC1]

In some embodiments, R¹ is chosen from

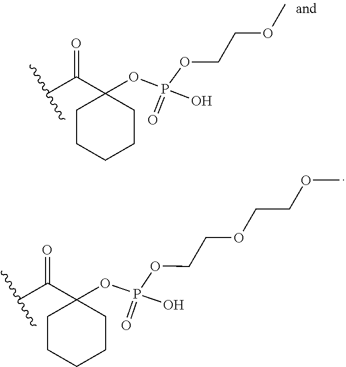 [SMILES: *C(=O)C1(OP(=O)(O)OCCOC)CCCCC1 ; *C(=O)C1(OP(=O)(O)OCCOCCOC)CCCCC1]

In some embodiments, R¹ is chosen from

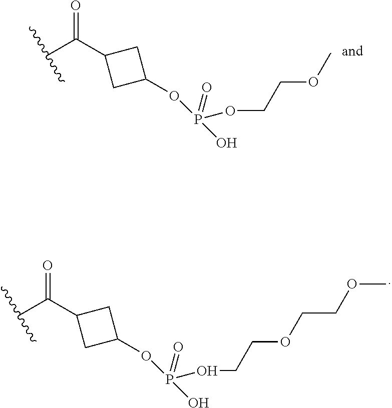 [SMILES: *C(=O)C1CC(OP(=O)(O)OCCOC)C1 ; *C(=O)C1CC(OP(=O)(O)OCCOCCOC)C1]

In some embodiments, R¹ is chosen from OH and

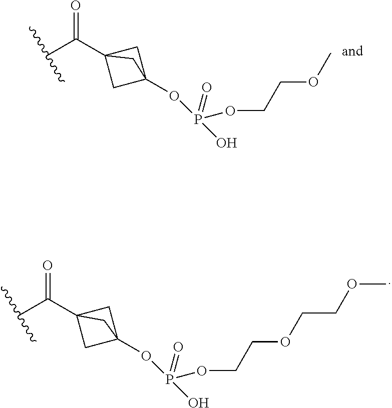 [SMILES: *C(=O)C12CC(OP(=O)(O)OCCOC)(C1)C2 ; *C(=O)C12CC(OP(=O)(O)OCCOCCOC)(C1)C2]

In some embodiments, R¹ is chosen from

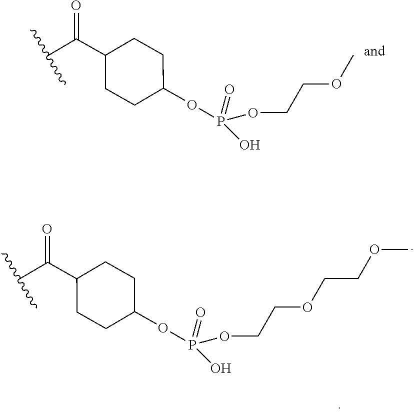 [SMILES: *C(=O)C1CCC(OP(=O)(O)OCCOC)CC1 ; *C(=O)C1CCC(OP(=O)(O)OCCOCCOC)CC1]

In some embodiments, R¹ is chosen from

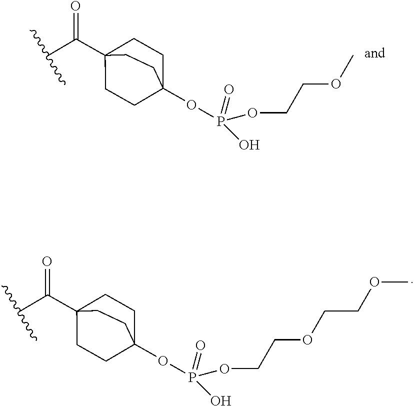 [SMILES: *C(=O)C12CCC(OP(=O)(O)OCCOC)(CC1)CC2 ; *C(=O)C12CCC(OP(=O)(O)OCCOCCOC)(CC1)CC2]

In some embodiments R¹ is

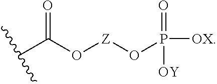

In some embodiments, the carbon in Z that is next to the carbonyloxy group

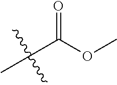 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

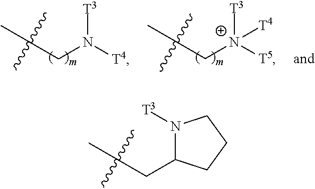

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

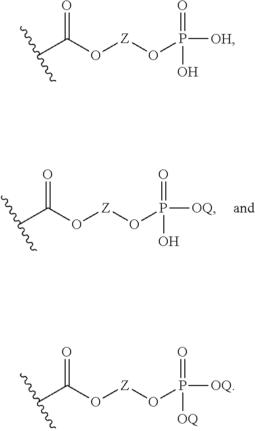

In some embodiments, the carbon in Z that is next to the carbonyloxy group

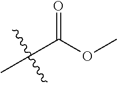 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon.

In some embodiments, R¹ is chosen from

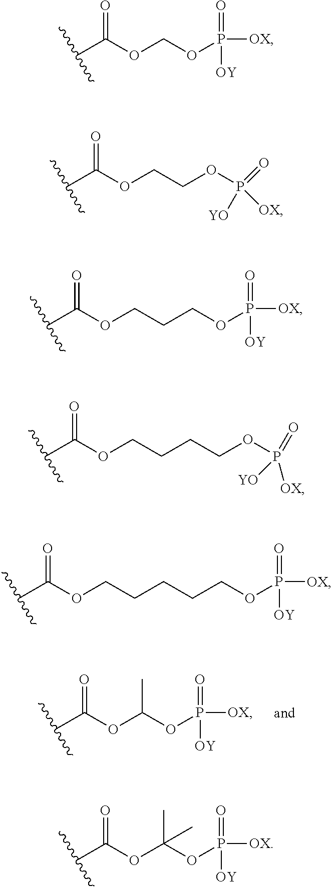

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

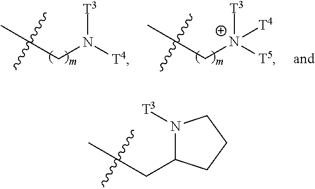

and groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, R¹ is chosen from

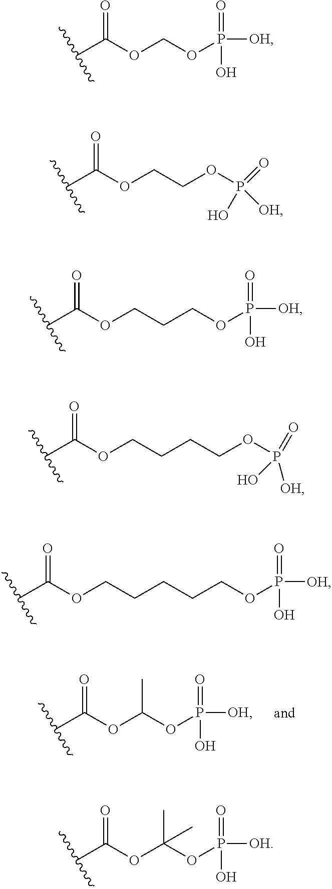

In some embodiments, R¹ is chosen from

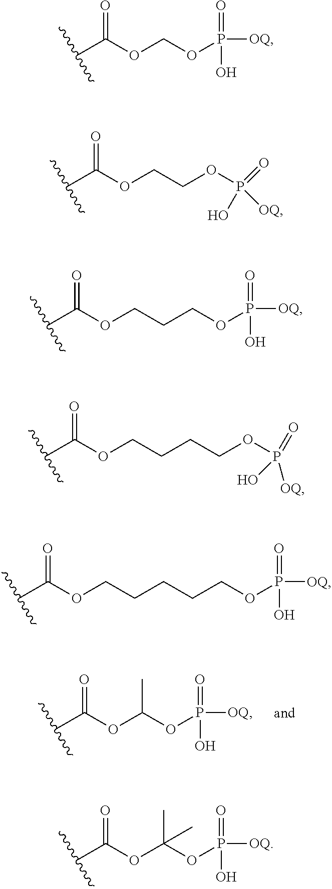

In some embodiments, R¹ is chosen from

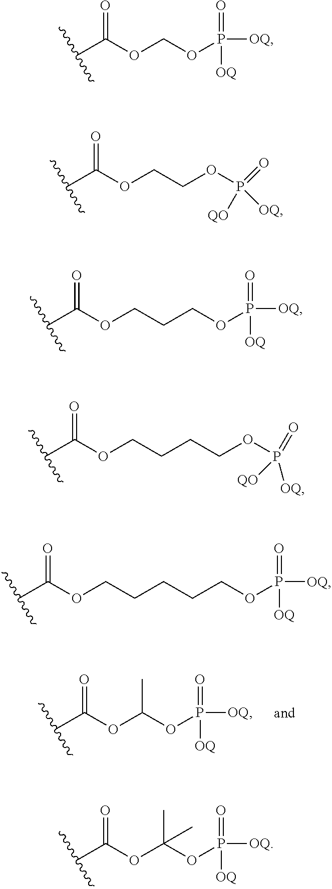

In some embodiments, R¹ is chosen from

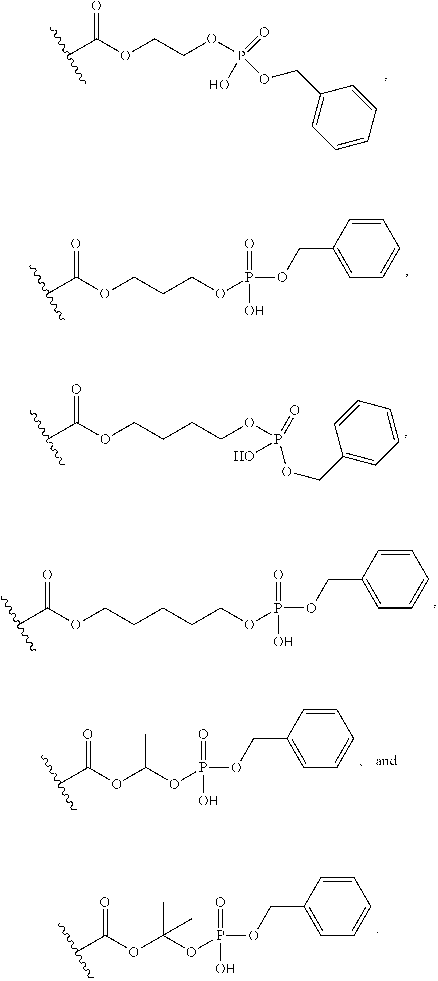 [SMILES: *C(=O)OCCOP(=O)(O)OCc1ccccc1 ; *C(=O)OCCCOP(=O)(O)OCc1ccccc1 ; *C(=O)OCCCCOP(=O)(O)OCc1ccccc1 ; *C(=O)OCCCCCOP(=O)(O)OCc1ccccc1 ; *C(=O)OC(C)OP(=O)(O)OCc1ccccc1 ; *C(=O)OC(C)(C)OP(=O)(O)OCc1ccccc1]

In some embodiments, R¹ is chosen from

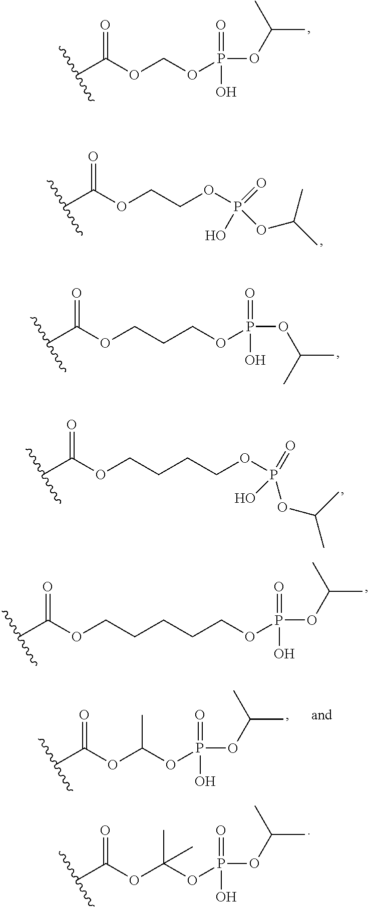

In some embodiments, R¹ is

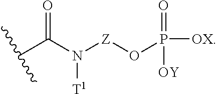

In some embodiments, the carbon in Z that is next to the carbonylamino group

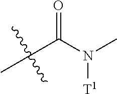

(i.e. the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, T¹ is H. In some embodiments, T¹ is methyl. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

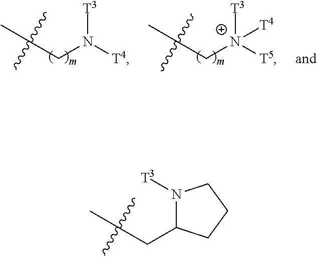

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, R¹ is chosen from

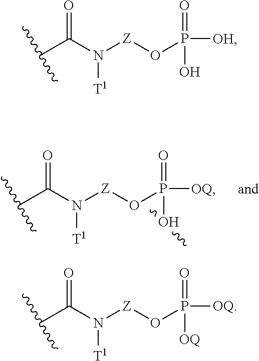

In some embodiments, the carbon in Z that is next to the carbonylamino group

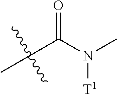

(i.e., the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, T¹ is H. In some embodiments, T¹ is methyl.

In some embodiments, R¹ is chosen from:

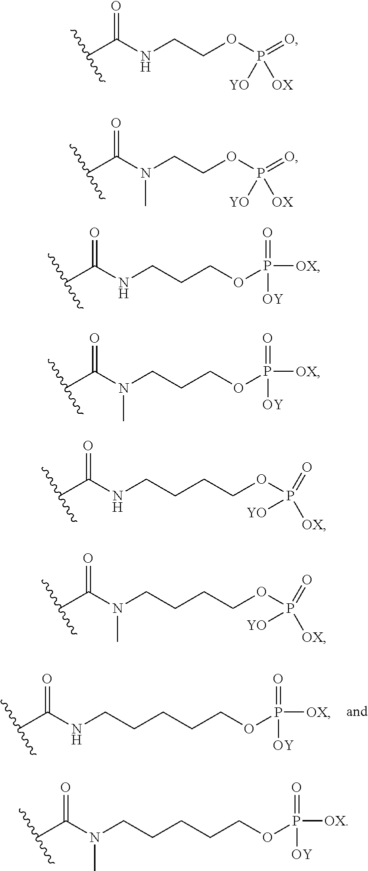

In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

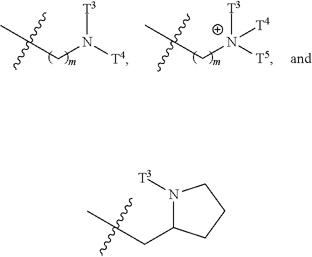

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments R¹ is chosen from

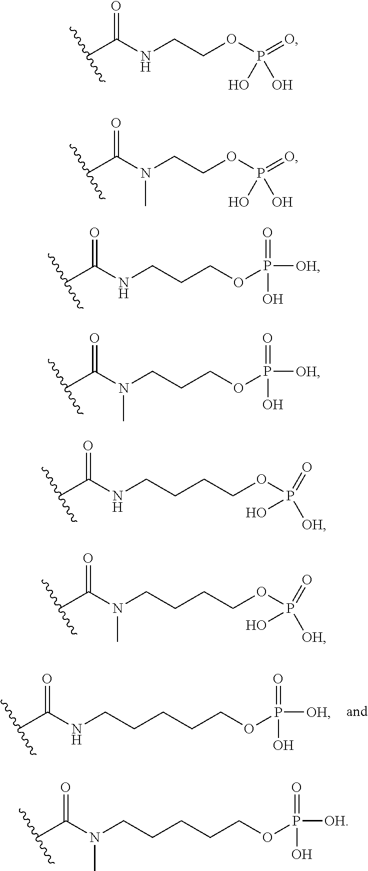

In some embodiments, R¹ is chosen from

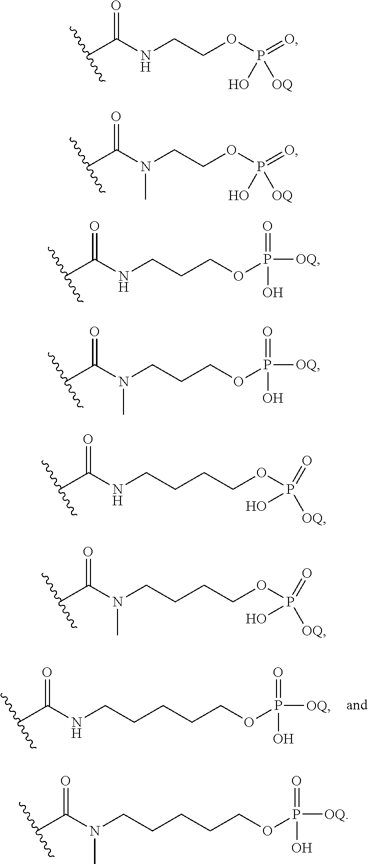

In some embodiments, R¹ is chosen from

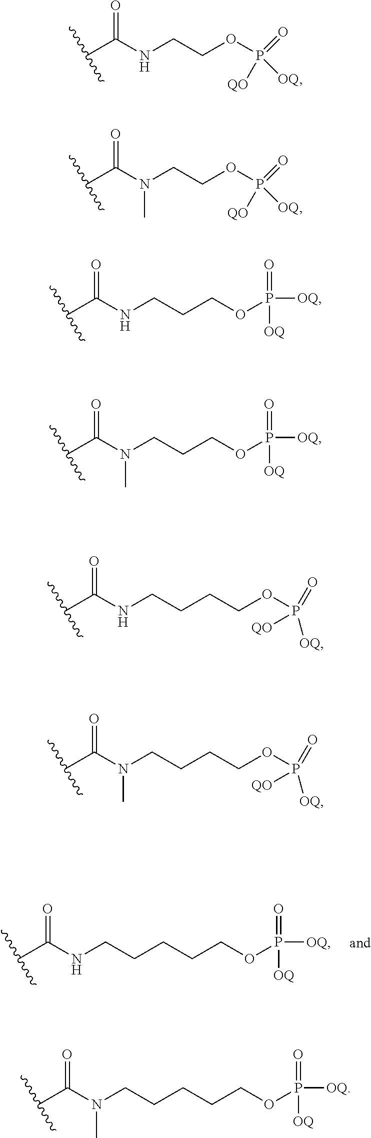

In some embodiments, R¹ is chosen from

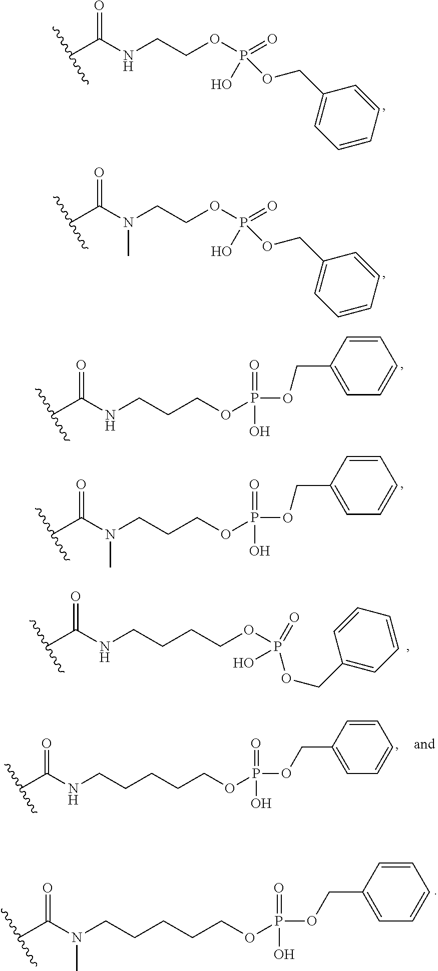

In some embodiments, R¹ is chosen from

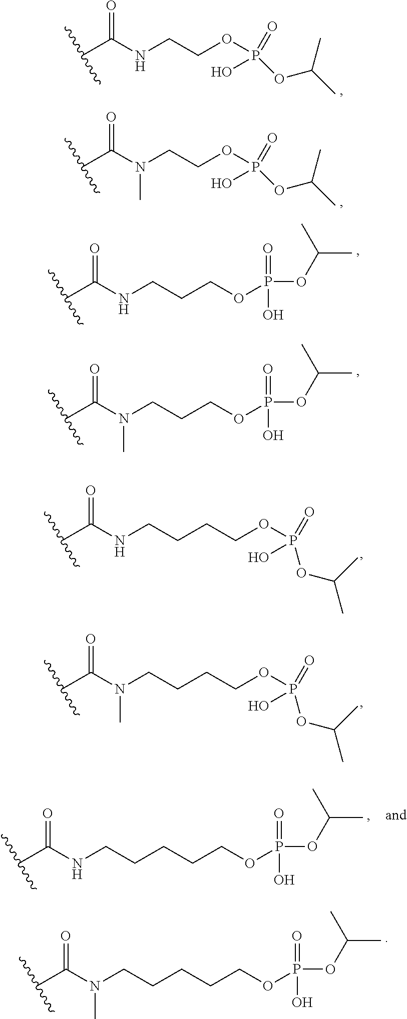

In some embodiments, R¹ is

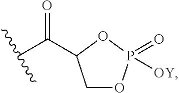

In some embodiments, Y is H. In some embodiments. Y is Q. In some embodiments, Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

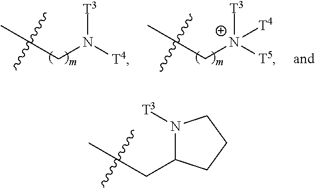

groups. In some embodiments, Y is chosen from phenyl, benzyl, and C₁₋₅ alkyl groups. In some embodiments, Y is chosen from phenyl, benzyl, ethyl, and isopropyl.

In some embodiments, R¹ is

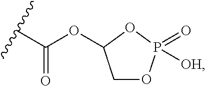 [SMILES: *C(=O)OC1COP(=O)(O)O1]

In some embodiments, R¹ is

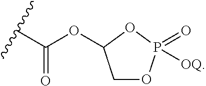

In some embodiments, R¹ is

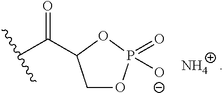 [SMILES: *C(=O)C1COP(=O)([O-])O1 ; [NH4+]]

In some embodiments, R¹ is chosen from

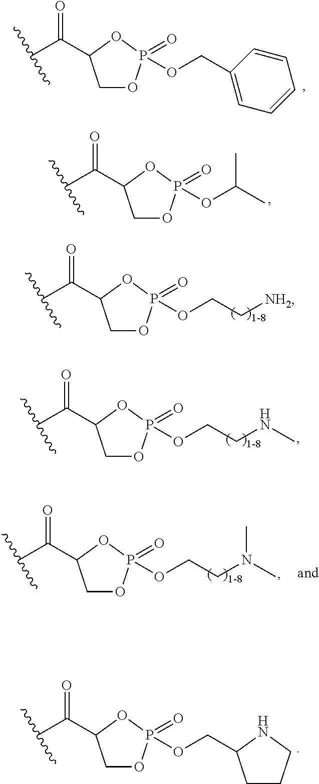

### 2. Formula (II) and its Sub-Formulas

Optionally, the compounds have a structure of Formula (II) or a pharmaceutically acceptable salt thereof:

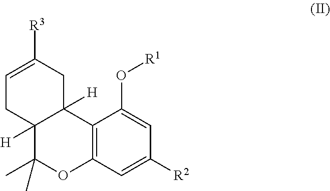

wherein R¹, R², and R³ are the same as described above for Formula (I), except that R³ cannot be ═O.

In some embodiments, the compounds are in a non-salt form. In some embodiments, the compounds are in a salt form. In some embodiments, the compounds are in an ammonium salt form.

In some embodiments, Formula (II) is in the form of Formula (II)-1.

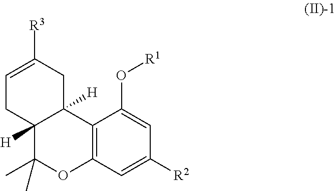

In some embodiments, Formula (II) is in the form of Formula (II)-2.

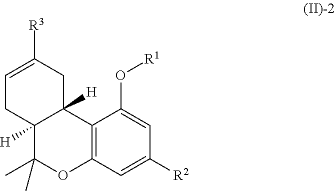

In some embodiments, R² is chosen from C₂₋₉ alkyl groups, and R³ is chosen from —CH₃, —CD₃, —CH₂OH, and —CH₂OR^{a}. In some embodiments, R² is chosen from C₂₋₉ alkyl groups, and R³ is —CH₃. In some embodiments, R² is chosen from C₂₋₉ alkyl groups, and R³ is —CD₃. In some embodiments, R² is chosen from C₂₋₉ alkyl groups, and R³ is —CH₂OH. In some embodiments, R² is chosen from C₂₋₉ alkyl groups, and R³ is —CH₂OR^{a}.

In some embodiments, R² is chosen from n-propyl, n-pentyl, and 1,1-dimethylheptyl, and R³ is chosen from —CH₃, —CD₃, —CH₂OH, and —CH₂OR^{a}. In some embodiments, R² is chosen from n-propyl, n-pentyl, and 1,1-dimethylheptyl, and R³ is —CH₃. In some embodiments, R² is chosen from n-propyl, n-pentyl, and 1,1-dimethylheptyl, and R³ is —CD₃. In some embodiments, R² is chosen from n-propyl, n-pentyl, and 1,1-dimethylheptyl, and R³ is —CH₂OH. In some embodiments, R² is chosen from n-propyl, n-pentyl, and 1,1-dimethylheptyl, and R³ is —CH₂OR^{a}.

In some embodiments, R² is n-propyl, and R³ is chosen from —CH₃, —CD₃, —CH₂OH, and —CH₂OR^{a}. In some embodiments, R² is n-propyl, and R³ is —CH₃. In some embodiments, R² is n-propyl, and R³ is —CD₃. In some embodiments, R² is n-propyl, and R³ is —CH₂OH. In some embodiments, R² is n-propyl, and R³ is —CH₂OR^{a}.

In some embodiments, R² is n-pentyl, and R³ is chosen from —CH₃, —CD₃, —CH₂OH, and —CH₂OR^{a}. In some embodiments, R² is n-pentyl, and R³ is —CH₃. In some embodiments, R² is n-pentyl, and R³ is —CD₃. In some embodiments, R² is n-pentyl, and R³ is —CH₂OH. In some embodiments, R² is n-pentyl, and R³ is —CH₂OR^{a}.

In some embodiments, R² is 1,1-dimethylheptyl, and R³ is chosen from —CH₃, —CD₃, —CH₂OH, and —CH₂OR^{a}. In some embodiments, R² is 1,1-dimethylheptyl, and R³ is —CH₃. In some embodiments, R² is 1,1-dimethylheptyl, and R³ is —CD₃. In some embodiments, R² is 1,1-dimethylheptyl, and R³ is —CH₂OH. In some embodiments, R² is 1,1-dimethylheptyl, and R³ is —CH₂OR^{a}.

### Formula (II)-1A

In some embodiments, Formula (II)-1 is in the form of Formula (II)-1A. i.e., the compounds are prodrugs of (−)trans-Δ⁸-THC.

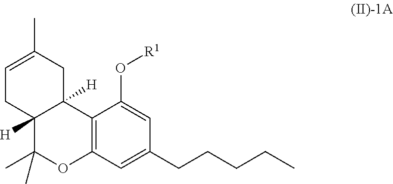

In some embodiments, the compounds of Formula (II)-1A are chosen from compounds having the following formula:

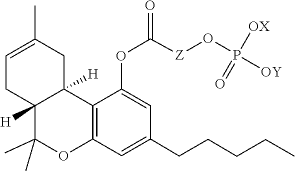

and pharmaceutically acceptable salts thereof. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl. C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

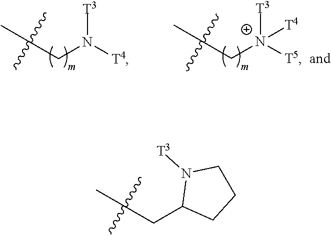

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, the compound of Formula (II)-1A is chosen from compounds having the following formulae:

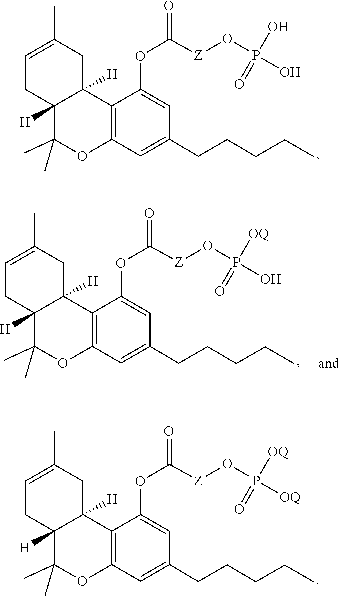

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (II)-1A is chosen from compounds having the following formulae:

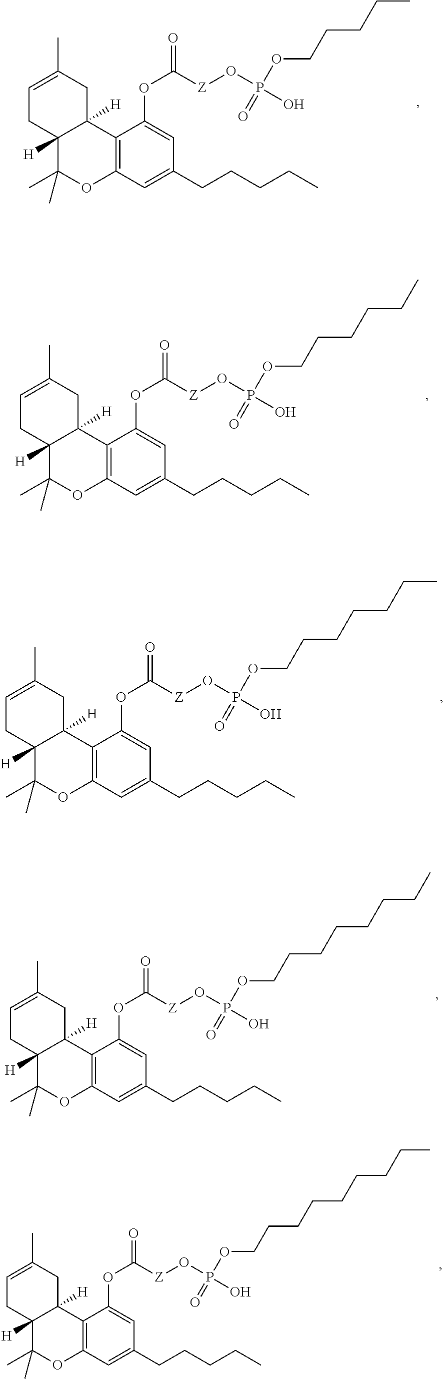

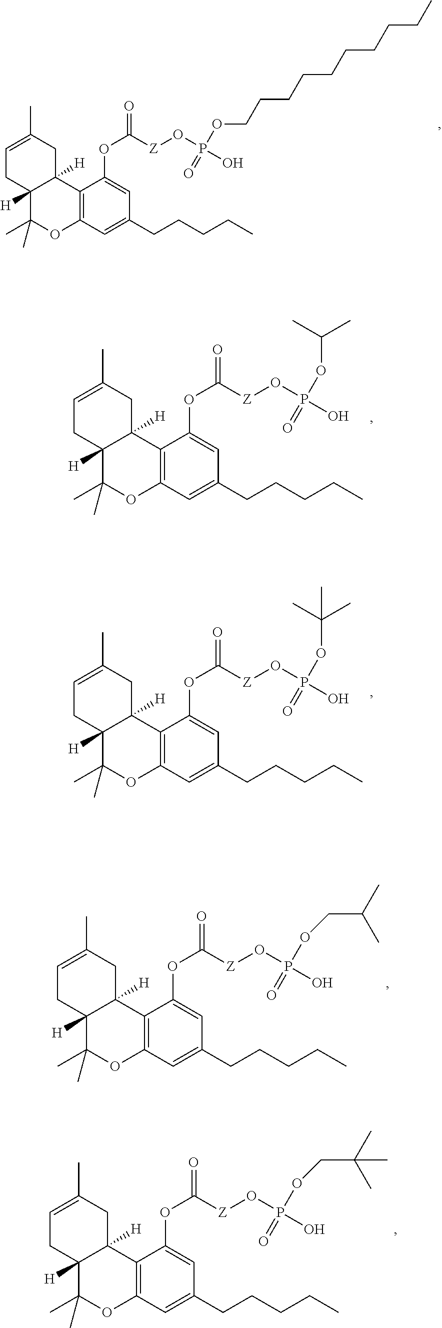

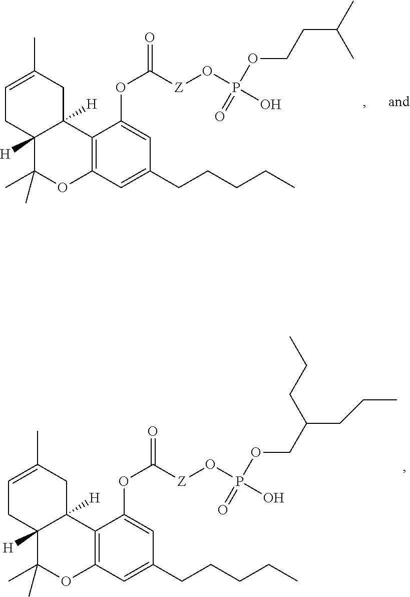

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (II)-1A is chosen from compounds having the following formulae:

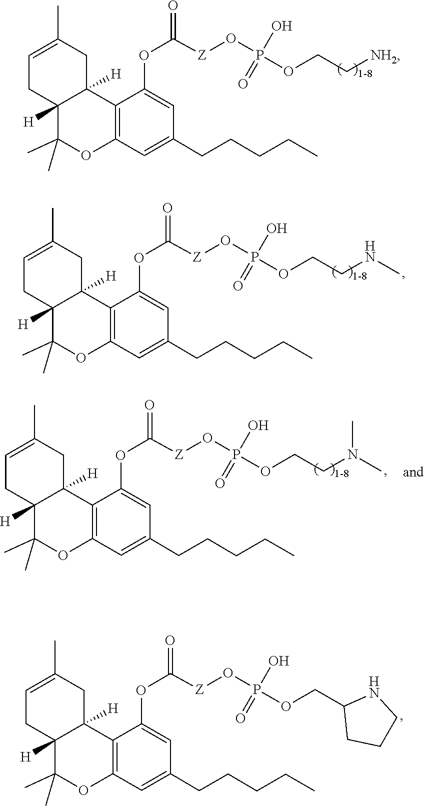

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (II)-1A is chosen from compounds having the following formulae:

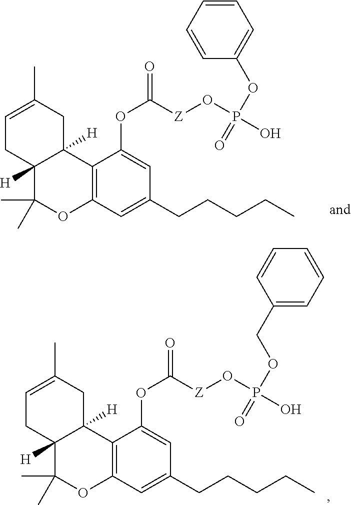

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (II)-1A is chosen from compounds having the following formulae:

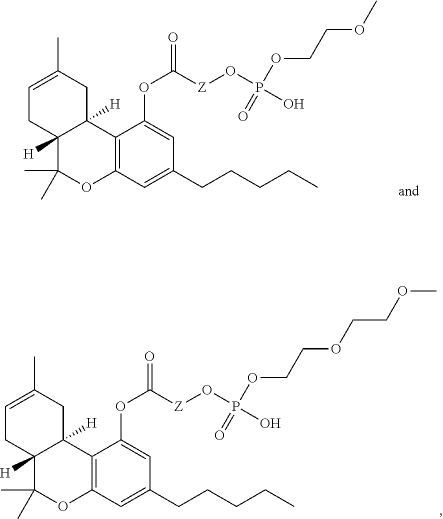

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (II)-1A is chosen from compounds having the following formulae:

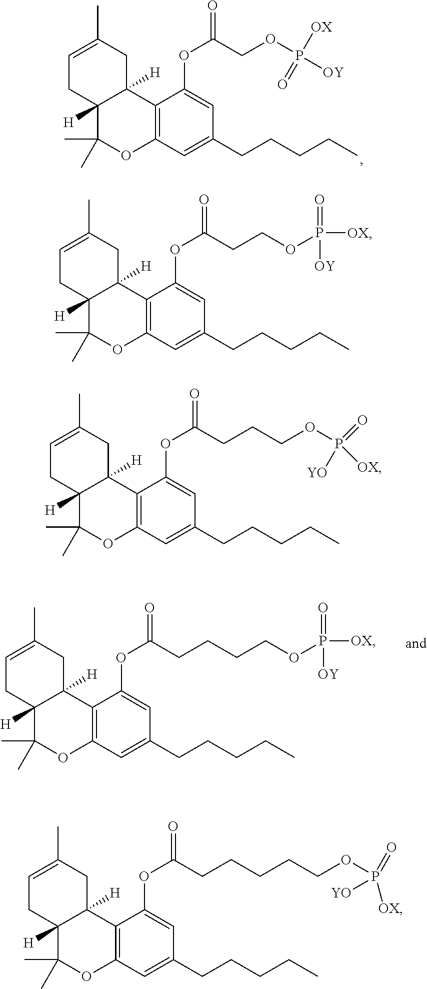

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

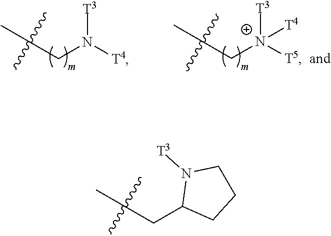

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

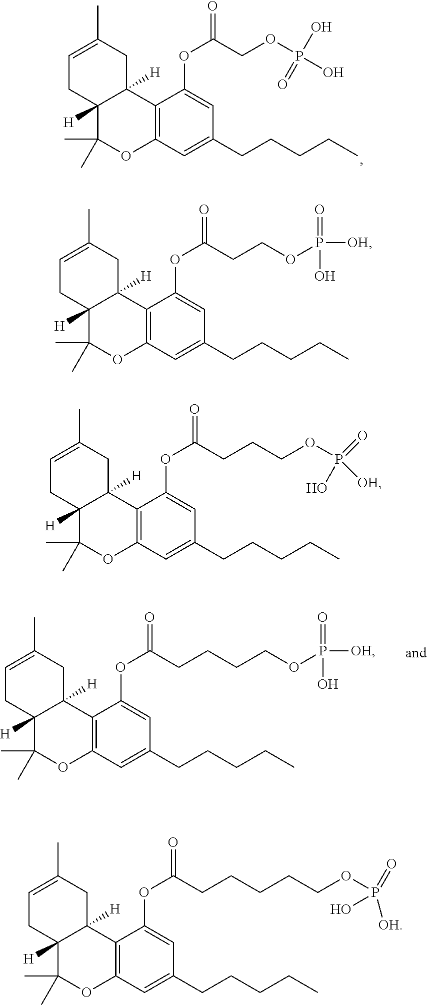

In some embodiments, the compound of Formula (II)-1A is chosen from compounds having the following formulae:

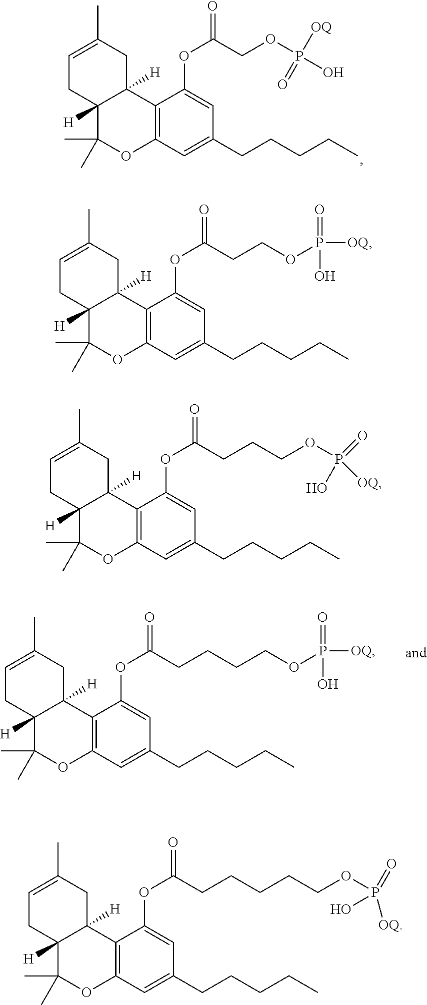

In some embodiments, the compound of Formula (II)-1A is chosen from compounds having the following formulae:

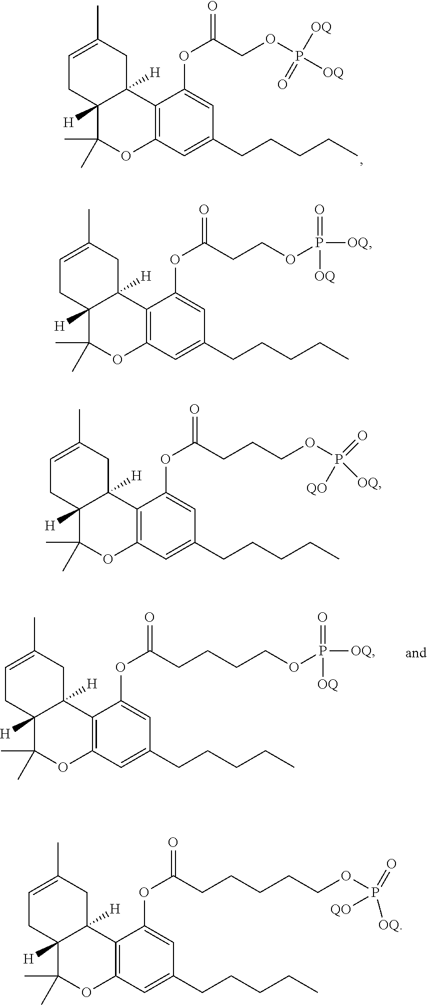

In some embodiments, the compound of Formula (II)-1A is chosen from compounds having the following formulae:

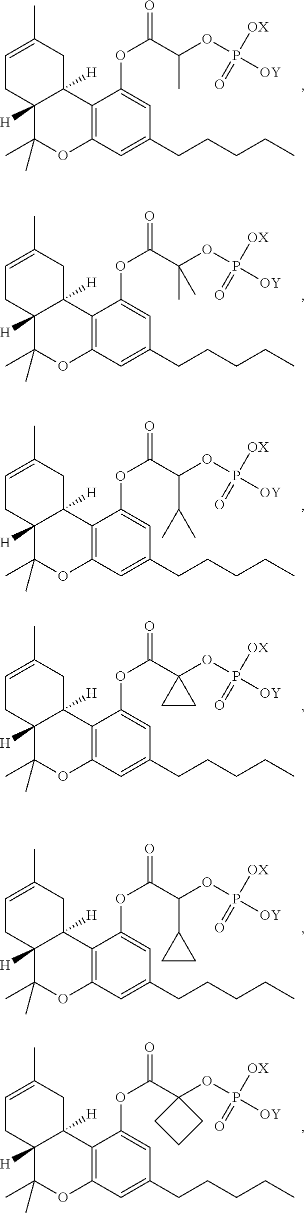

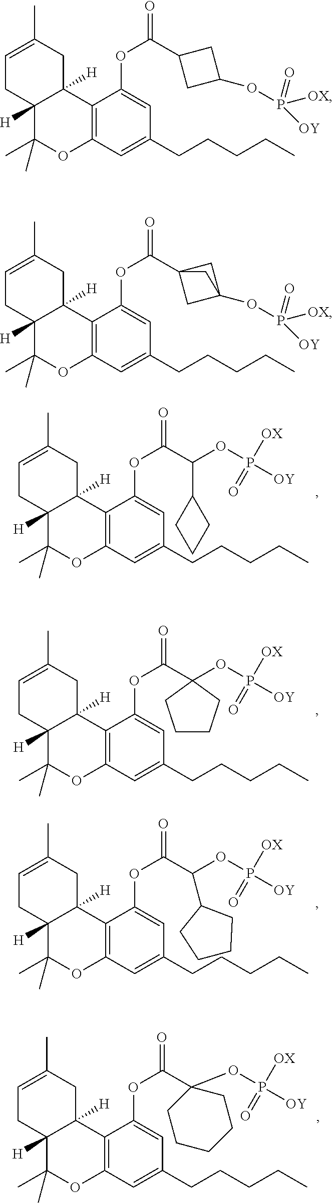

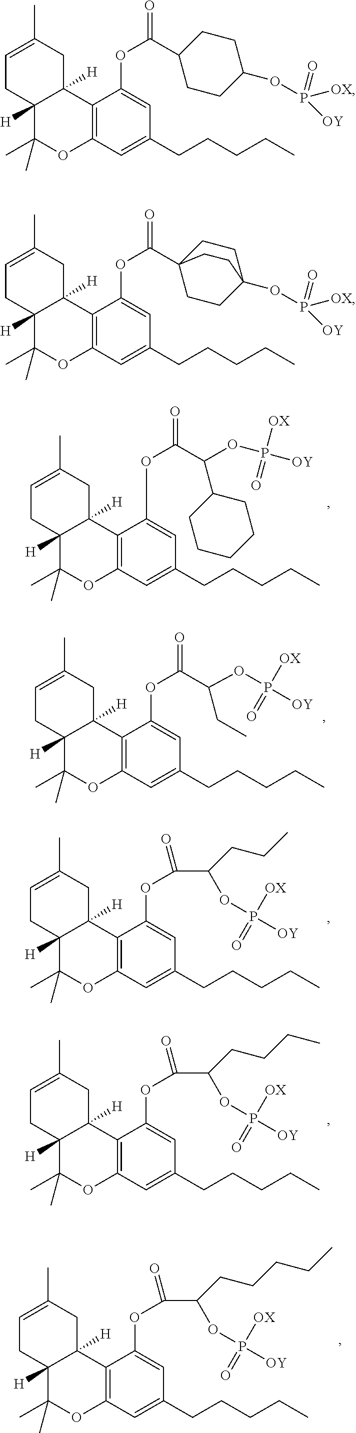

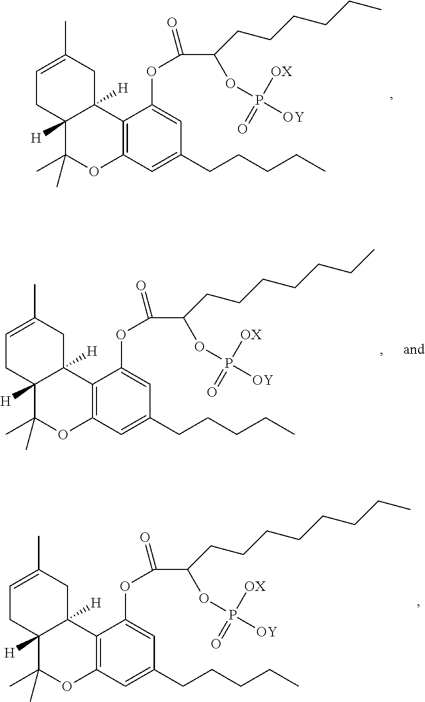

and pharmaceutically acceptable salts of any of the foregoing. In some embodiments, the pharmaceutically acceptable salts are ammonium salts. In some embodiments, X is H. In some embodiments. X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q. and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

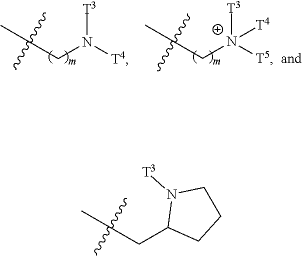

groups. In some embodiments, X is chosen from H and Q. and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

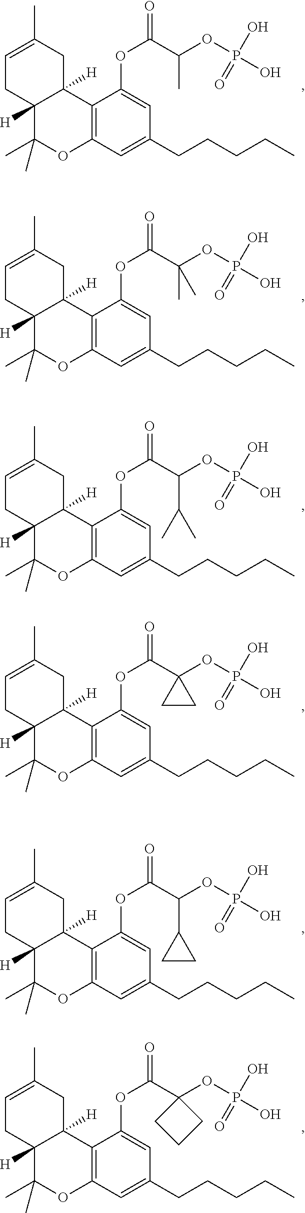

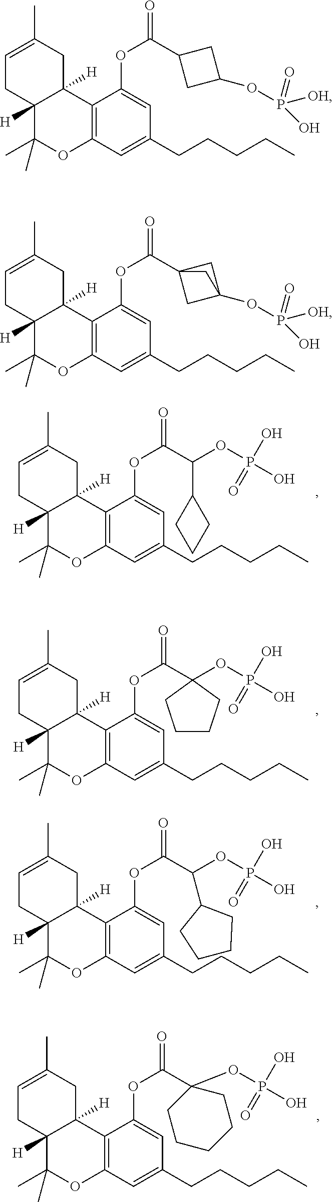

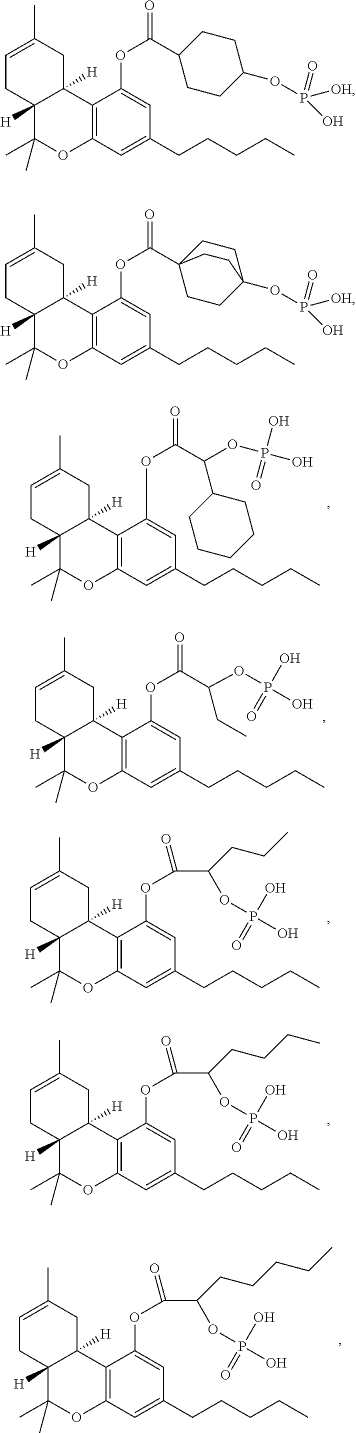

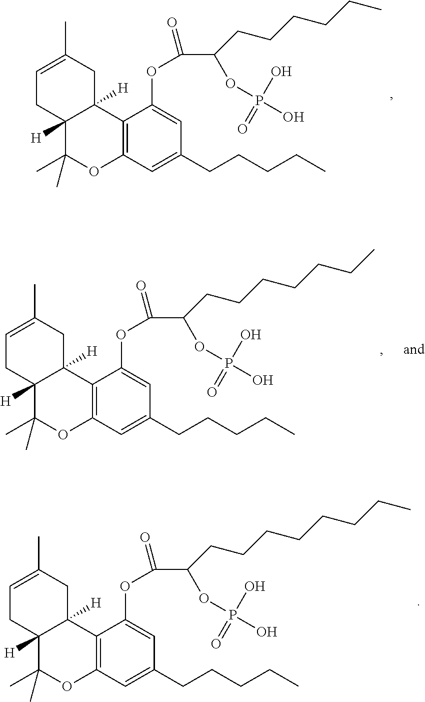 [SMILES: CCCCCCC(OP(=O)(O)O)C(=O)Oc1cc(CCCCC)cc2c1[C@@H]1CC(C)=CC[C@H]1C(C)(C)O2 ; CCCCCCCC(OP(=O)(O)O)C(=O)Oc1cc(CCCCC)cc2c1[C@@H]1CC(C)=CC[C@H]1C(C)(C)O2 ; CCCCCCCCC(OP(=O)(O)O)C(=O)Oc1cc(CCCCC)cc2c1[C@@H]1CC(C)=CC[C@H]1C(C)(C)O2]

In some embodiments, the compound of Formula (II)-1A is chosen from compounds having the following formulae:

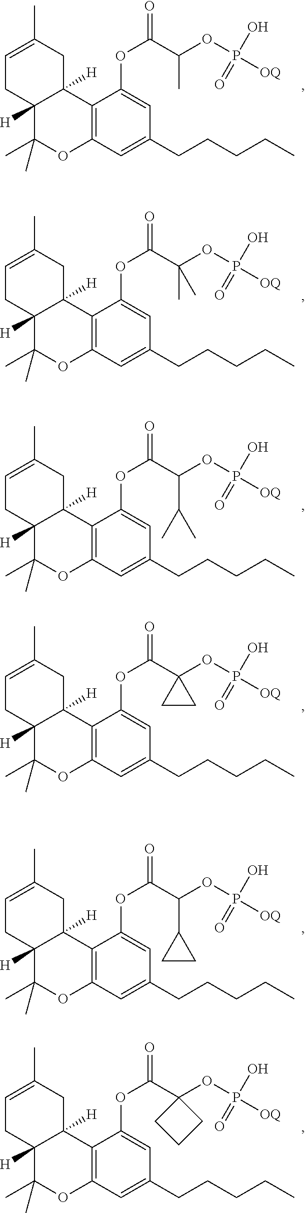

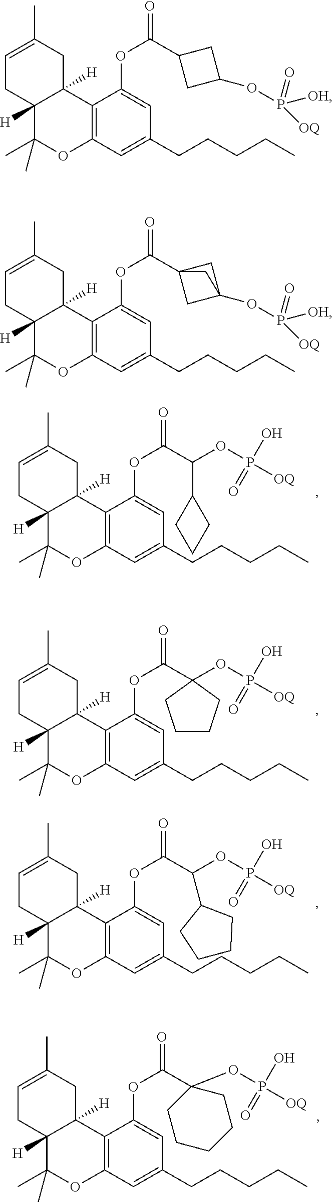

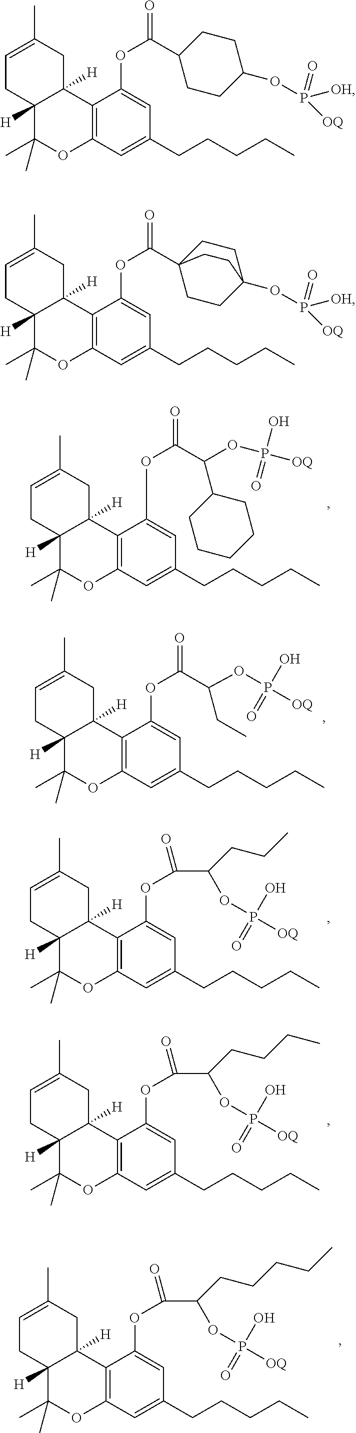

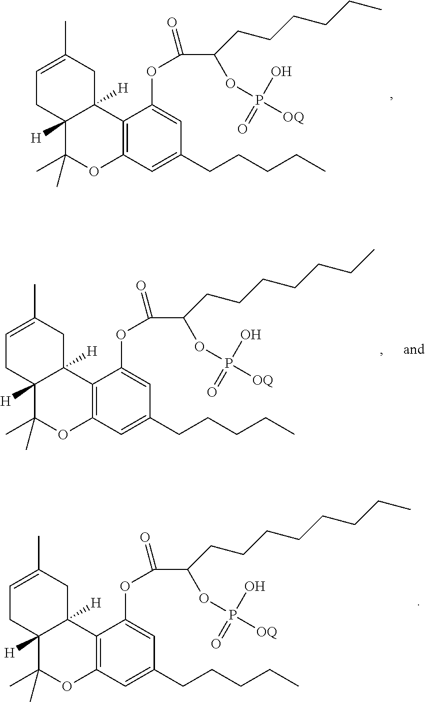

In some embodiments, the compound of Formula (II)-1A is chosen from compounds having the following formulae:

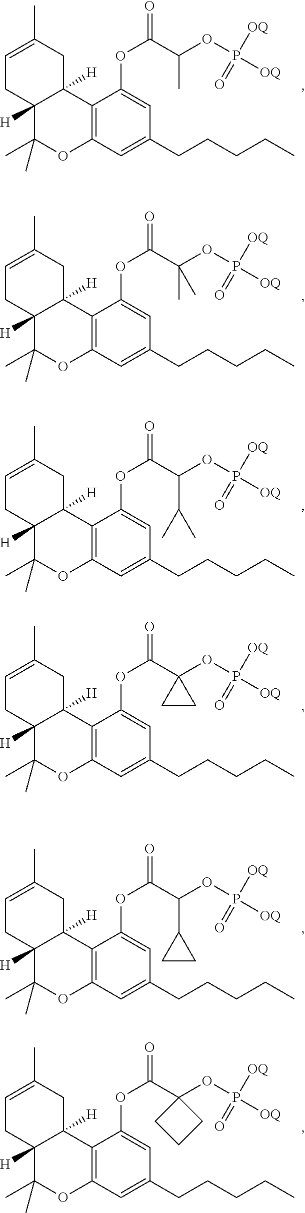

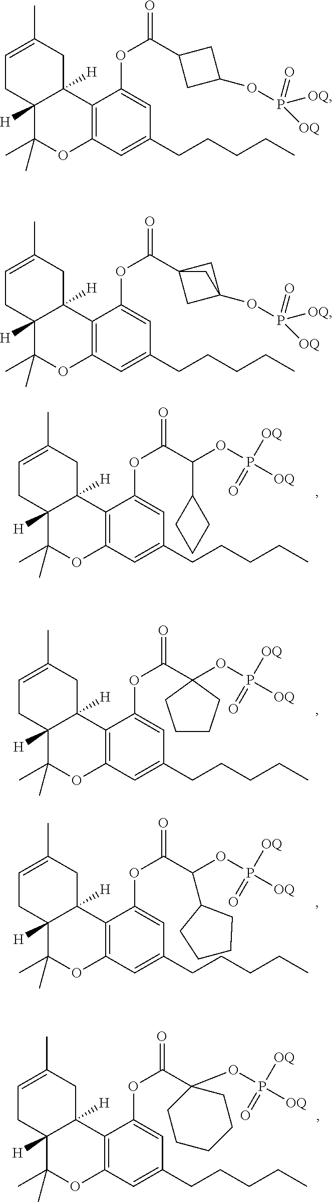

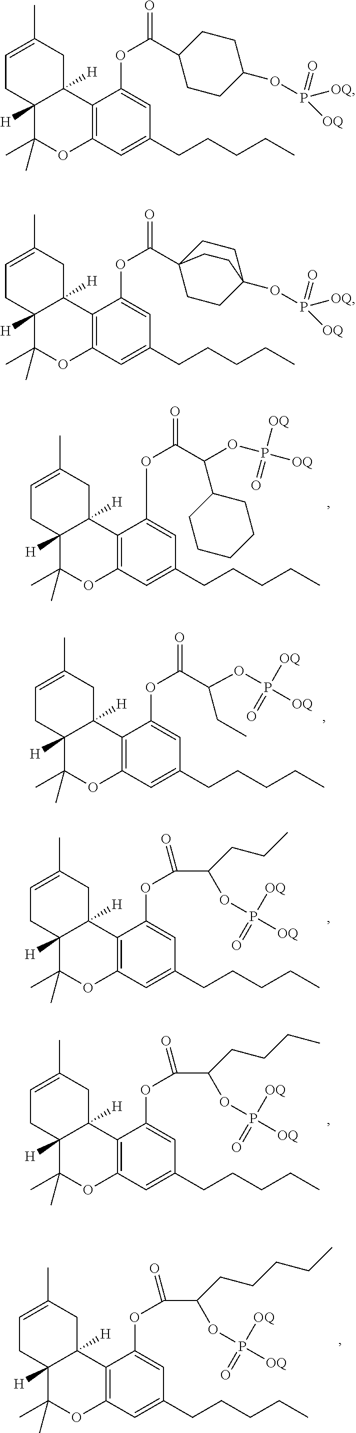

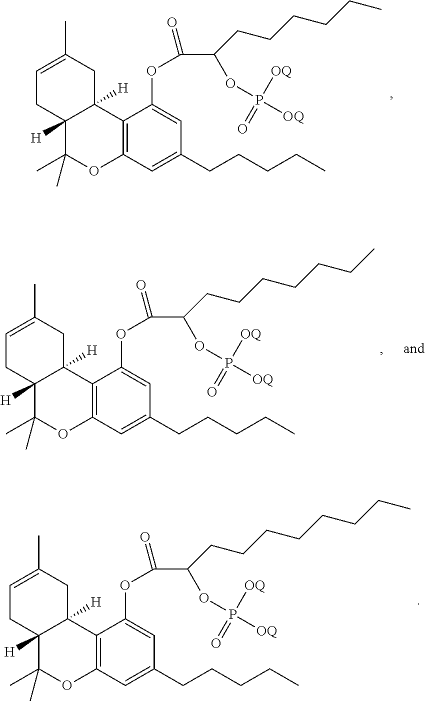

In some embodiments, the compound of Formula (II)-1A is chosen from compounds having the following formulae:

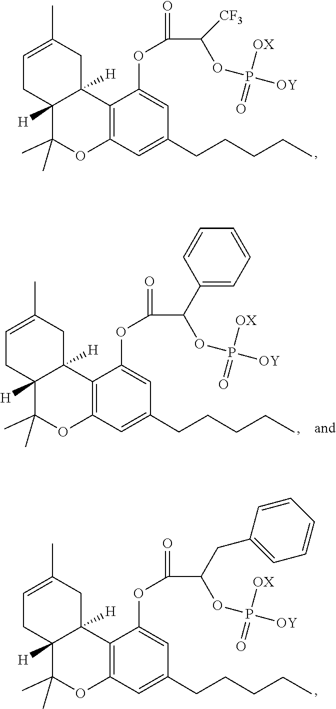

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

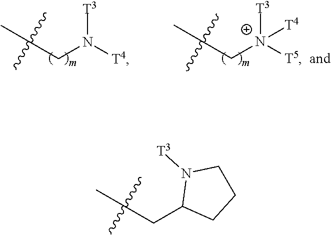

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

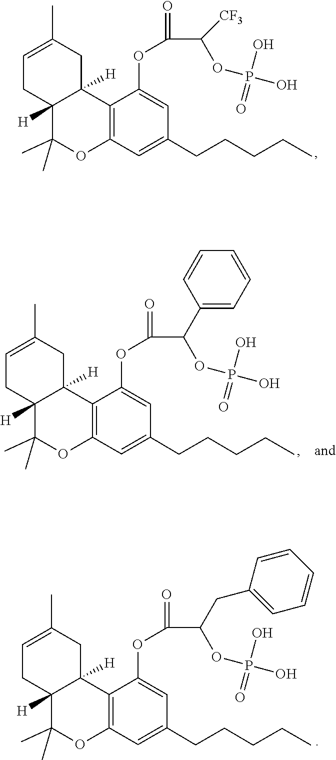

In some embodiments, the compound of Formula (II)-1A is chosen from compounds having the following formulae:

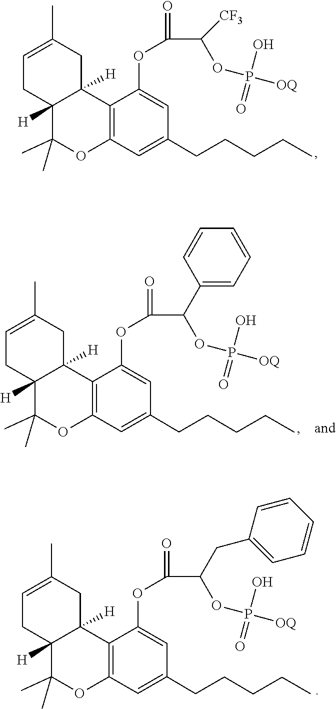

In some embodiments, the compound of Formula (II)-1A is chosen from compounds having the following formula:

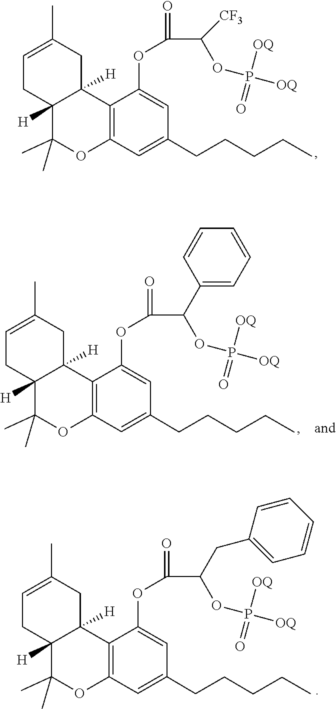

In some embodiments, the compound of Formula (II)-1A is chosen from compounds having the following formulae:

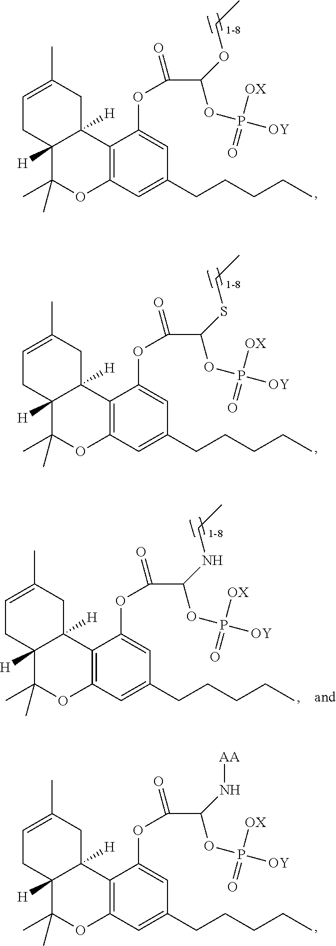

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

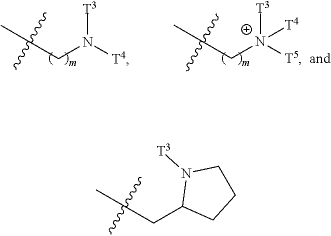

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, the compound of Formula (II)-1A is chosen from compounds having the following formulae:

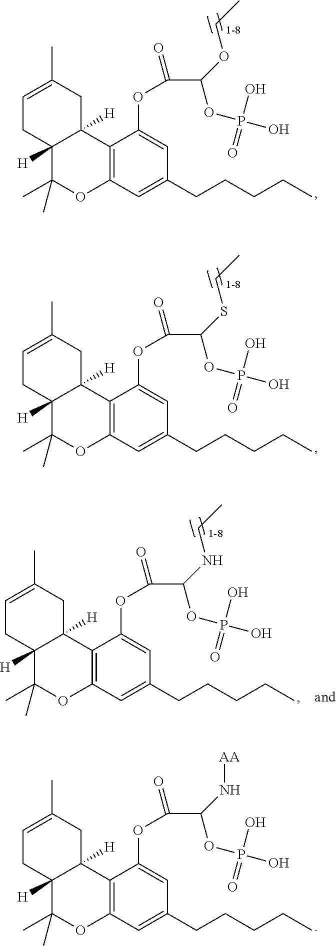

In some embodiments, the compound of Formula (II)-1A is chosen from compounds having the following formulae:

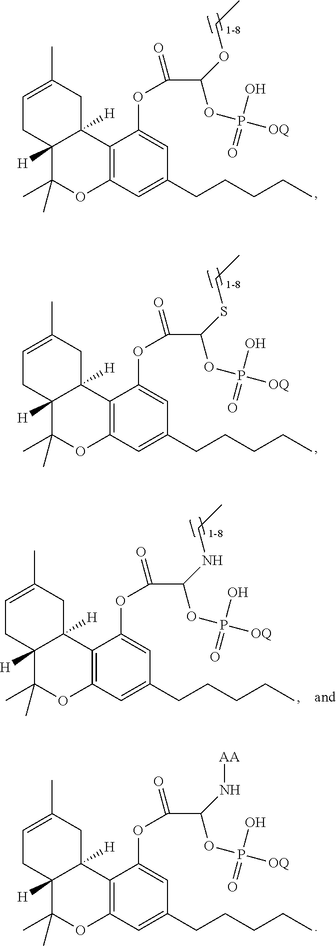

In some embodiments, the compound of Formula (II)-1A is chosen from compounds having the following formulae:

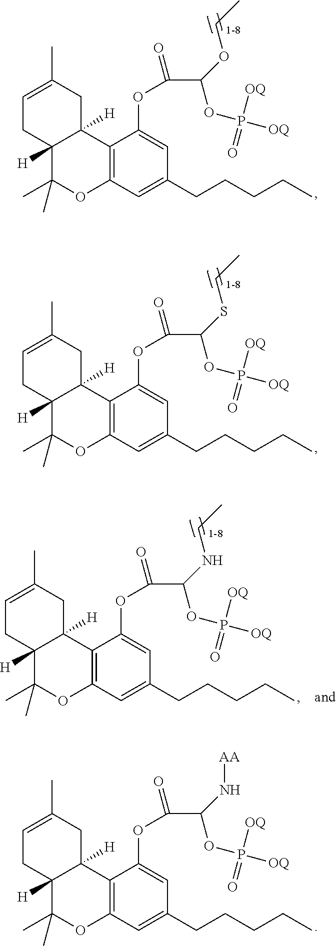

In some embodiments, the compound of Formula (II)-1A is chosen from compounds having the following formulae:

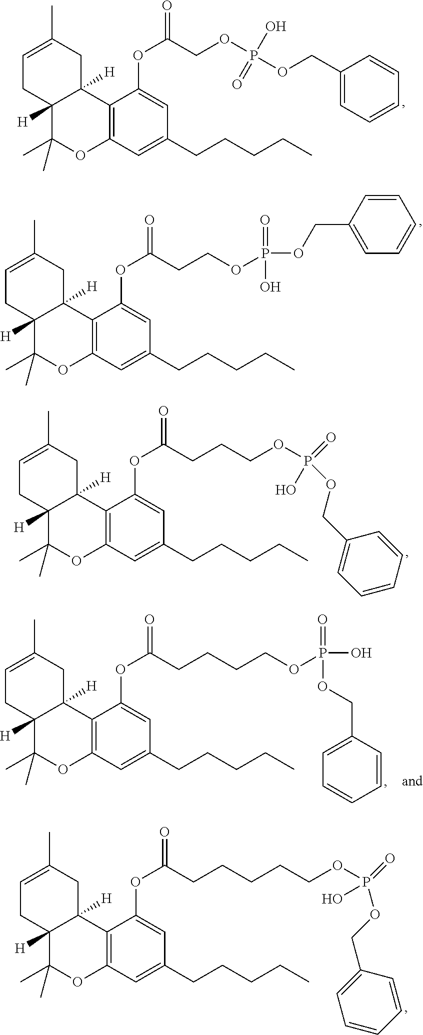

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

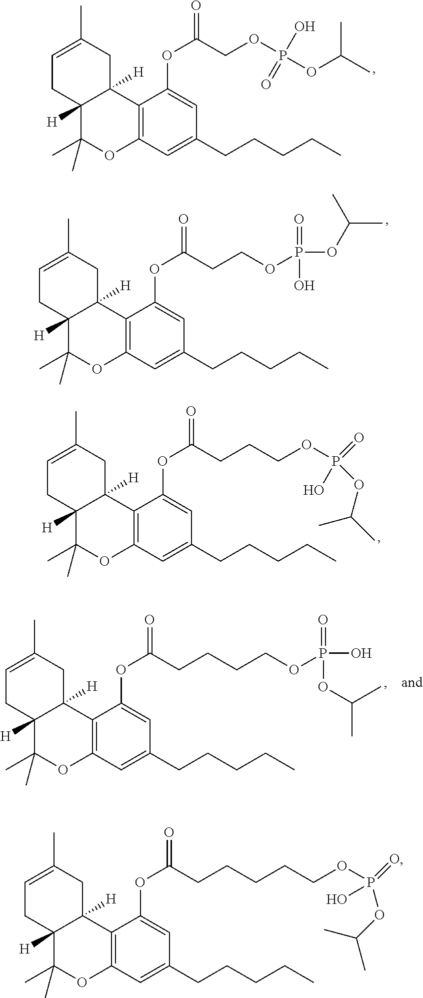

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

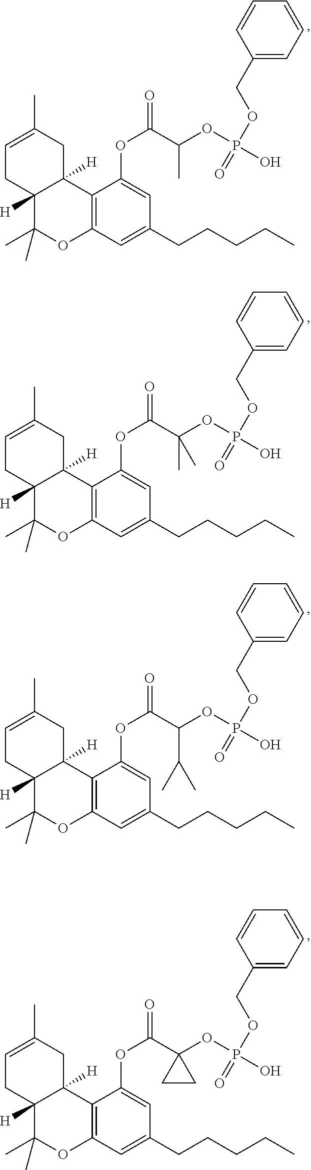 [SMILES: CCCCCc1cc(OC(=O)C(C)OP(=O)(O)OCc2ccccc2)c2c(c1)OC(C)(C)[C@@H]1CC=C(C)C[C@@H]21 ; CCCCCc1cc(OC(=O)C(C)(C)OP(=O)(O)OCc2ccccc2)c2c(c1)OC(C)(C)[C@@H]1CC=C(C)C[C@@H]21 ; CCCCCc1cc(OC(=O)C(OP(=O)(O)OCc2ccccc2)C(C)C)c2c(c1)OC(C)(C)[C@@H]1CC=C(C)C[C@@H]21 ; CCCCCc1cc(OC(=O)C2(OP(=O)(O)OCc3ccccc3)CC2)c2c(c1)OC(C)(C)[C@@H]1CC=C(C)C[C@@H]21]

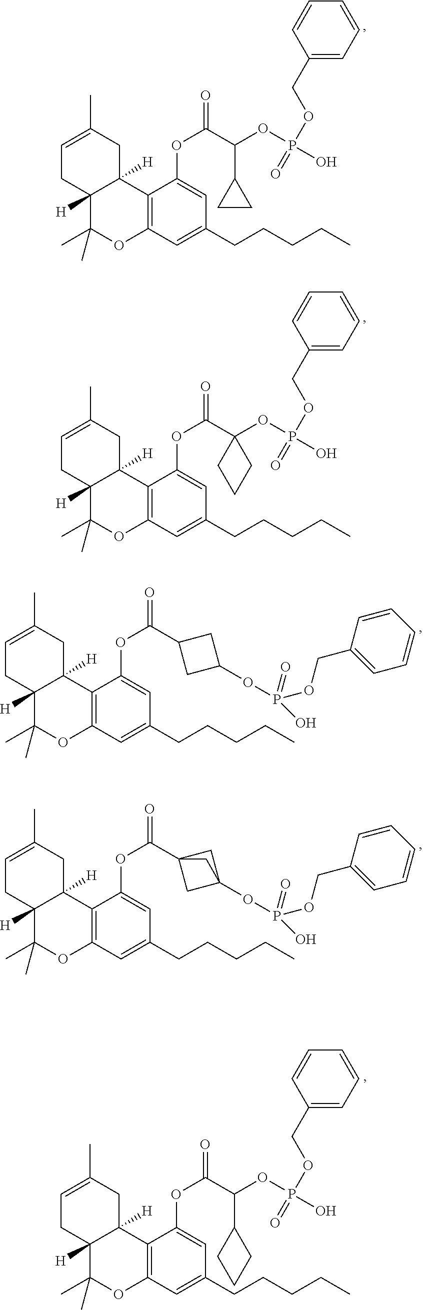 [SMILES: CCCCCc1cc(OC(=O)C(OP(=O)(O)OCc2ccccc2)C2CC2)c2c(c1)OC(C)(C)[C@@H]1CC=C(C)C[C@@H]21 ; CCCCCc1cc(OC(=O)C2(OP(=O)(O)OCc3ccccc3)CCC2)c2c(c1)OC(C)(C)[C@@H]1CC=C(C)C[C@@H]21 ; CCCCCc1cc(OC(=O)C2CC(OP(=O)(O)OCc3ccccc3)C2)c2c(c1)OC(C)(C)[C@@H]1CC=C(C)C[C@@H]21 ; CCCCCc1cc(OC(=O)C23CC(OP(=O)(O)OCc4ccccc4)(C2)C3)c2c(c1)OC(C)(C)[C@@H]1CC=C(C)C[C@@H]21 ; CCCCCc1cc(OC(=O)C(OP(=O)(O)OCc2ccccc2)C2CCC2)c2c(c1)OC(C)(C)[C@@H]1CC=C(C)C[C@@H]21]

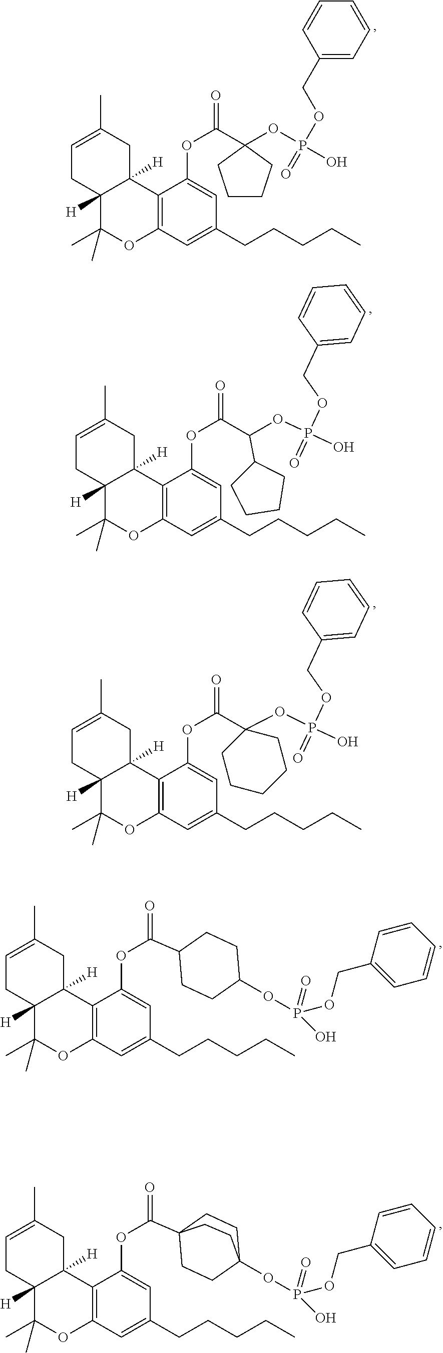

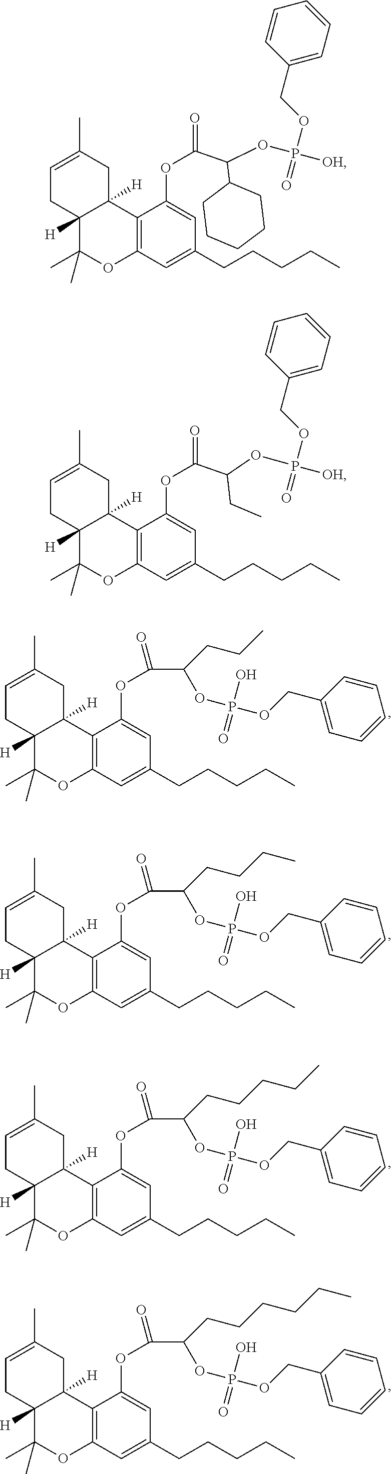

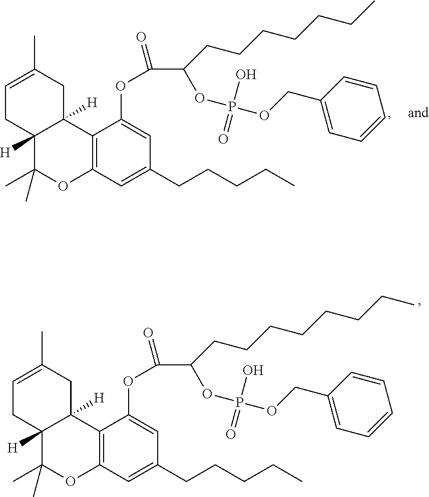 [SMILES: CCCCCCCC(OP(=O)(O)OCc1ccccc1)C(=O)Oc1cc(CCCCC)cc2c1[C@@H]1CC(C)=CC[C@H]1C(C)(C)O2 ; CCCCCCCCC(OP(=O)(O)OCc1ccccc1)C(=O)Oc1cc(CCCCC)cc2c1[C@@H]1CC(C)=CC[C@H]1C(C)(C)O2]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

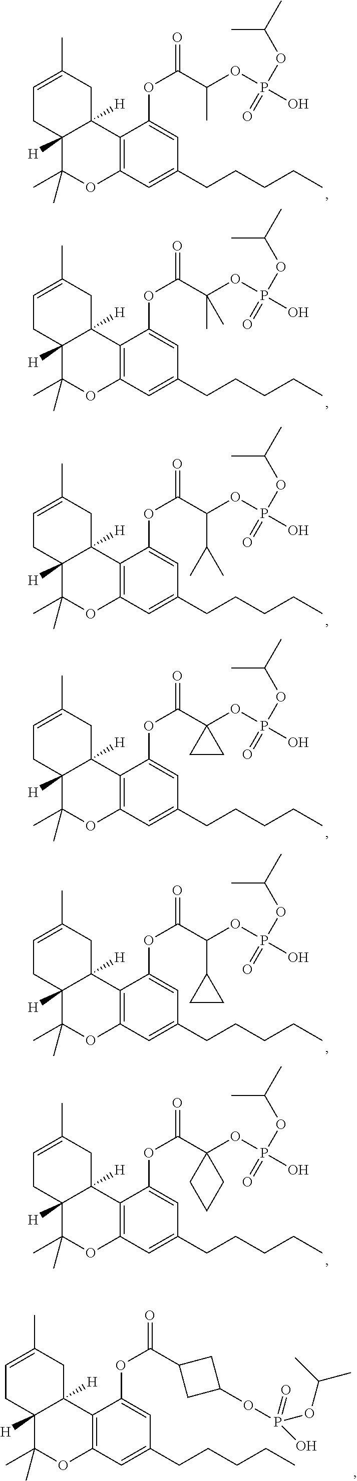

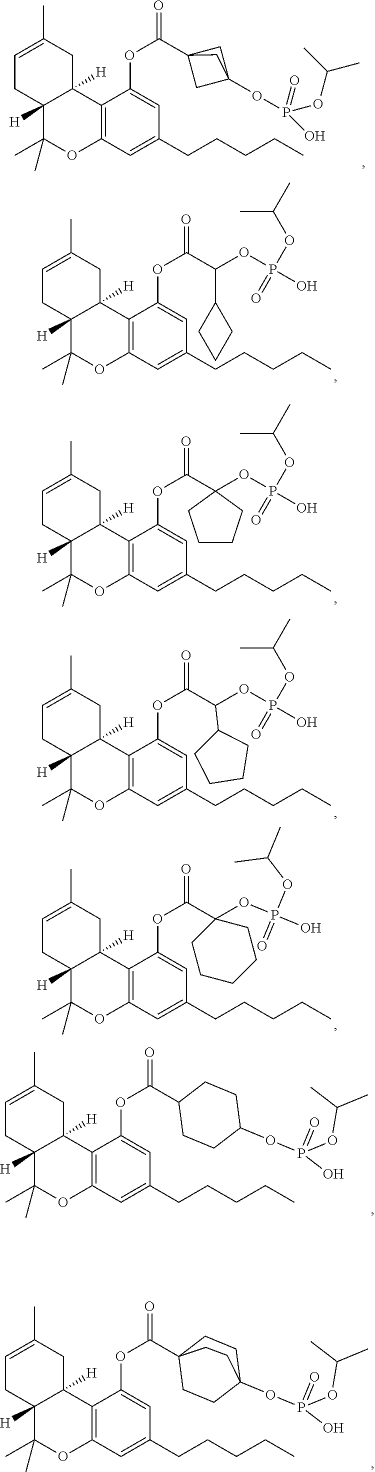

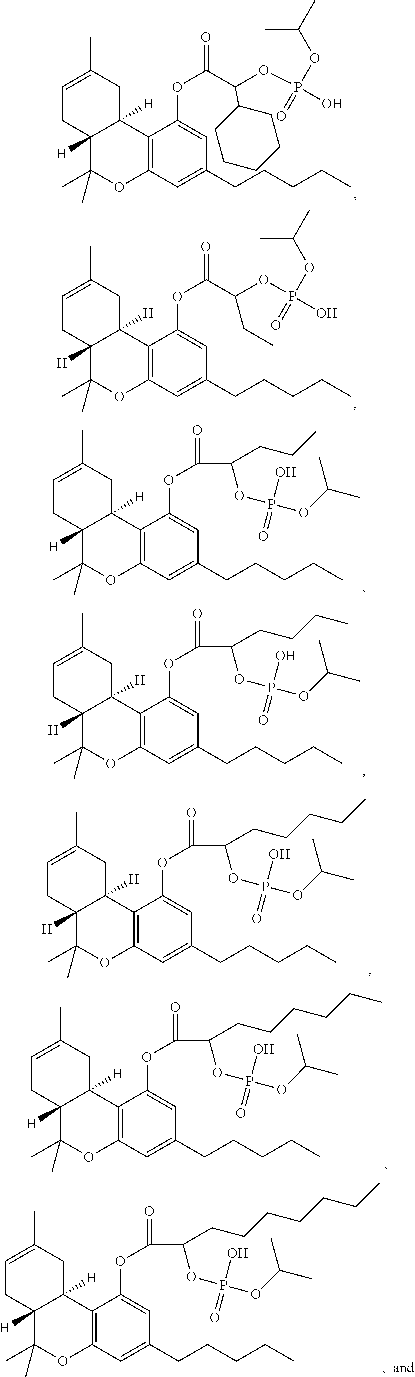

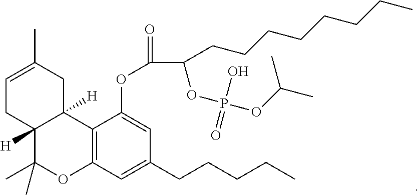 [SMILES: CCCCCCCCC(OP(=O)(O)OC(C)C)C(=O)Oc1cc(CCCCC)cc2c1[C@@H]1CC(C)=CC[C@H]1C(C)(C)O2]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

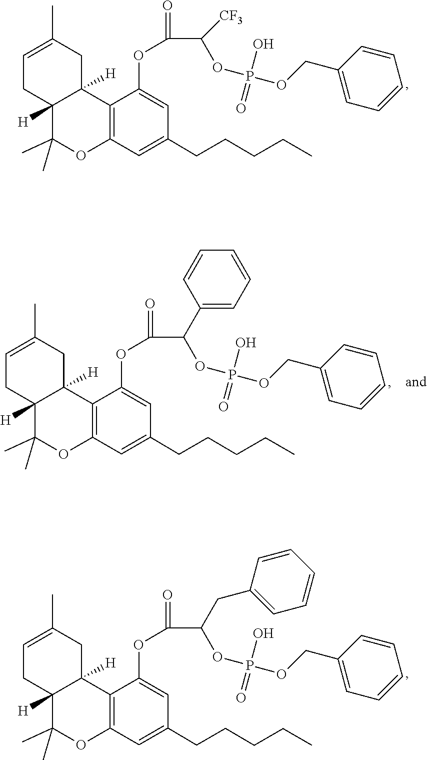 [SMILES: CCCCCc1cc(OC(=O)C(OP(=O)(O)OCc2ccccc2)C(F)(F)F)c2c(c1)OC(C)(C)[C@@H]1CC=C(C)C[C@@H]21 ; CCCCCc1cc(OC(=O)C(OP(=O)(O)OCc2ccccc2)c2ccccc2)c2c(c1)OC(C)(C)[C@@H]1CC=C(C)C[C@@H]21 ; CCCCCc1cc(OC(=O)C(Cc2ccccc2)OP(=O)(O)OCc2ccccc2)c2c(c1)OC(C)(C)[C@@H]1CC=C(C)C[C@@H]21]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

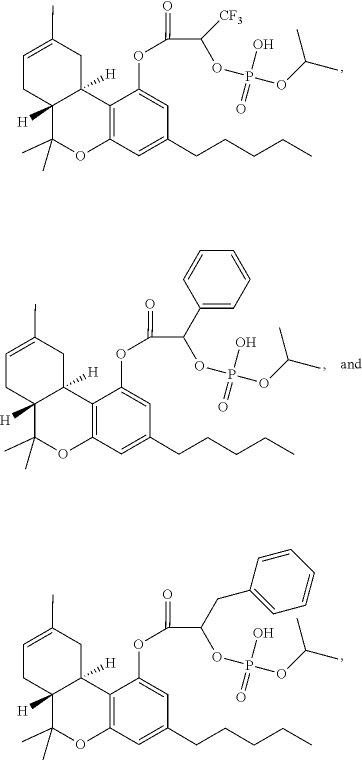

and pharmaceutically acceptable salts of any of the foregoing. In some embodiments, the pharmaceutically acceptable salts are ammonium salts.

In some embodiments, the compound of Formula (II)-1A is chosen from compound having the following formulae:

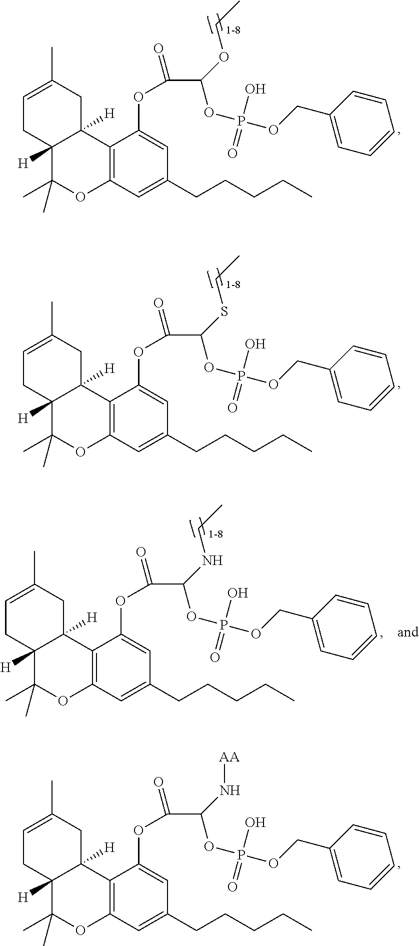

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

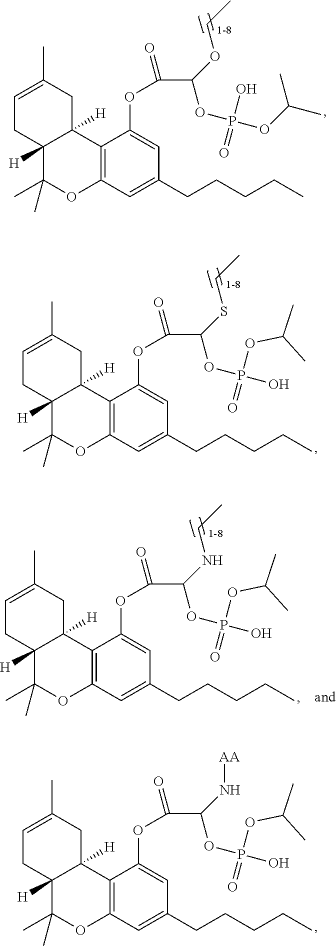

and pharmaceutically acceptable salts of any of the foregoing. In some embodiments the pharmaceutically acceptable salts are ammonium salts.

In some embodiments, the compound of Formula (II)-1A is chosen from compounds having the following formula:

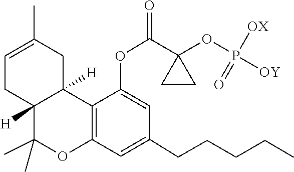

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

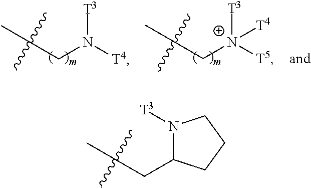

groups. In some embodiments, X is chosen from H and Q, and Y is chosen from phenyl, benzyl, and C₁₋₈ alkyl groups. In some embodiments, X is chosen from H and Q, and Y is chosen from phenyl, benzyl, ethyl, and isopropyl.

In some embodiments, the compound of Formula (II)-1A is chosen from compounds having the following formula:

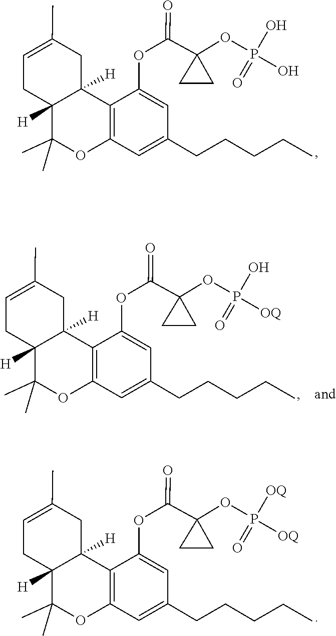

In some embodiments, the compound of Formula (II)-1A is chosen from compounds having the following formula:

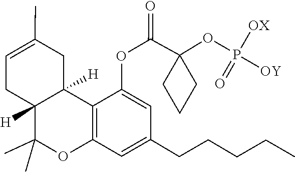

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

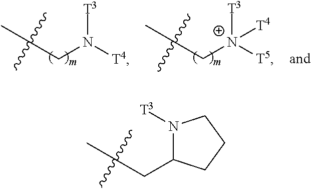

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

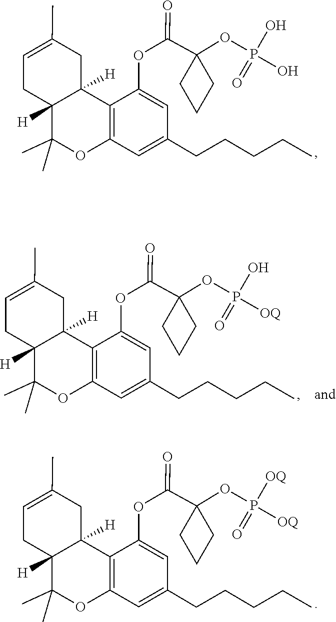

In some embodiments, the compound of Formula (II)-1A is chosen from compounds having the following formula:

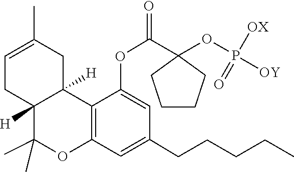

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl C₇₋₁₉ arylalkyl, C₂₋₁₄ heteroarylalkyl, —(CH₂CH₂O)ₙCH₃,

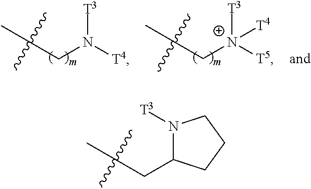

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

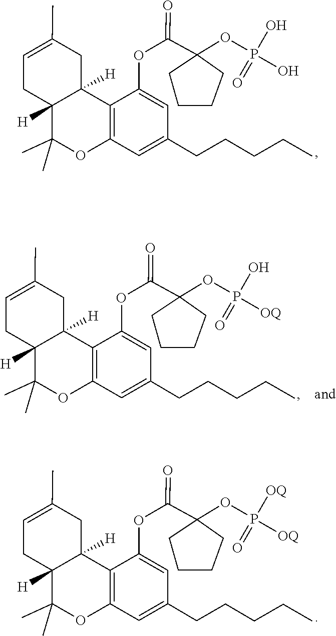

In some embodiments, the compound of Formula (II)-1A is chosen from compounds having the following formula:

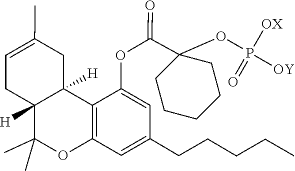

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

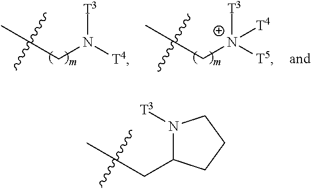

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

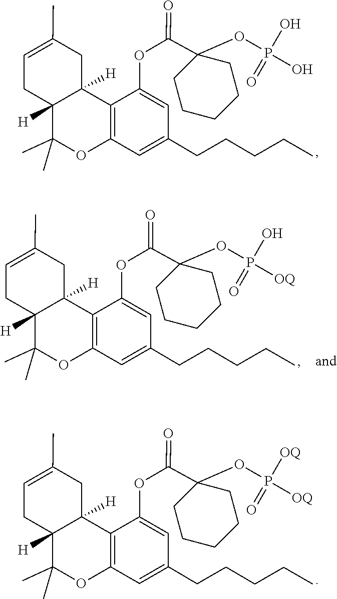

In some embodiments, the compound of Formula (II)-1A is chosen from compounds having the following formula:

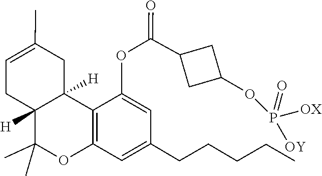

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q. and Y is chosen from C₁₋₁₈ alkyl, C₁₋₁₈ haloalkyl, C₆₋₁₈ aryl, C₁₋₁₃ heteroaryl, C₇₋₁₉ arylalkyl C₂₋₁₄ heteroarylalkyl, —(CH₂CH₂O)ₙCH₃,

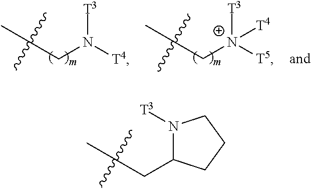

groups. In some embodiments, X is chosen from H and Q. and Y is chosen from phenyl, benzyl, and C₁₋₈ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

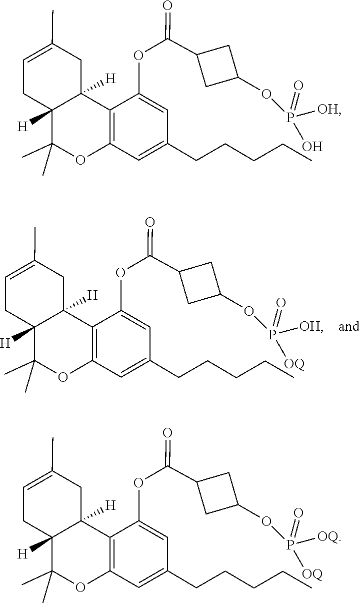

In some embodiments, the compound of Formula (II)-1A is chosen from compounds having the following formula:

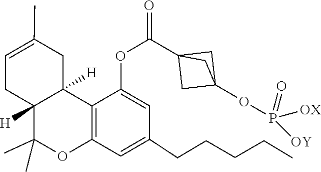

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

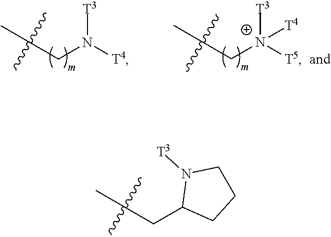

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, the compound of Formula (II)-1A is chosen from compounds having the following formulae:

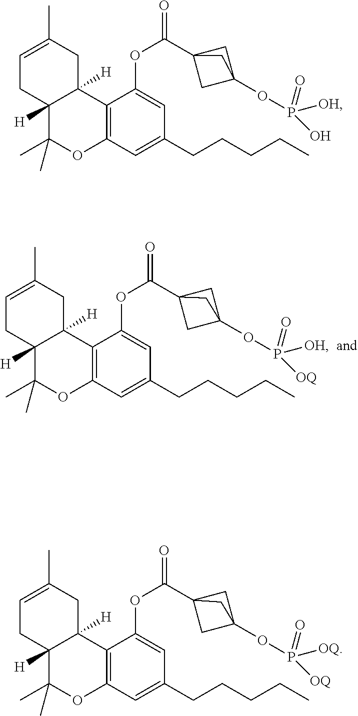

In some embodiments, the compound of Formula (II)-1A is chosen from compounds having the following formula:

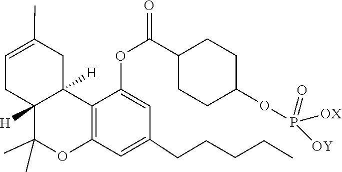

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

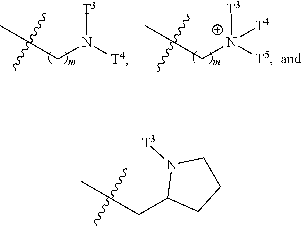

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

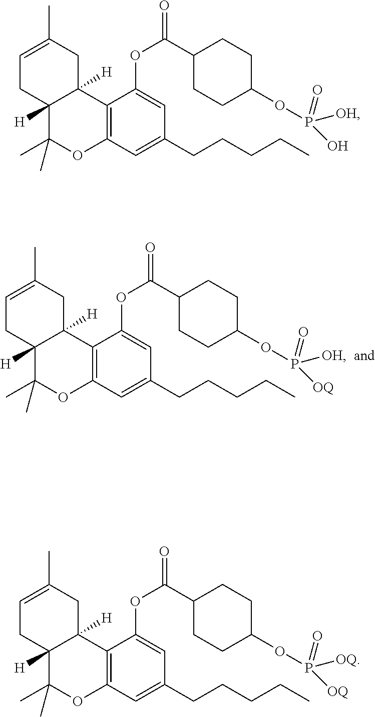

In some embodiments, the compound of Formula (II)-1A is chosen from compounds having the following formula:

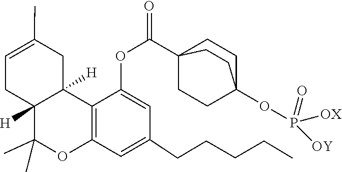

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

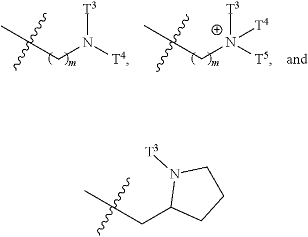

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

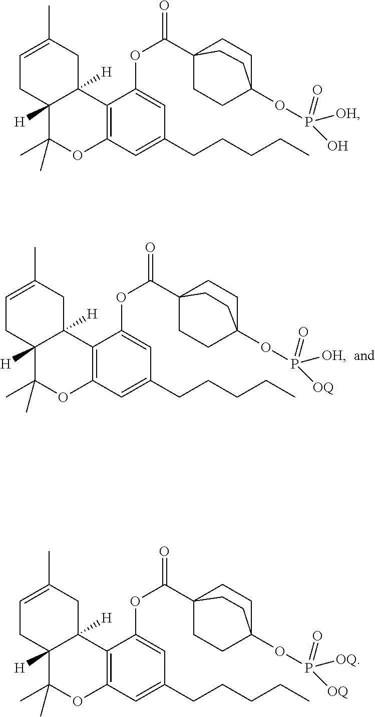

In some embodiments, the compound of Formula (II)-1A is chosen from compounds having the following formulae:

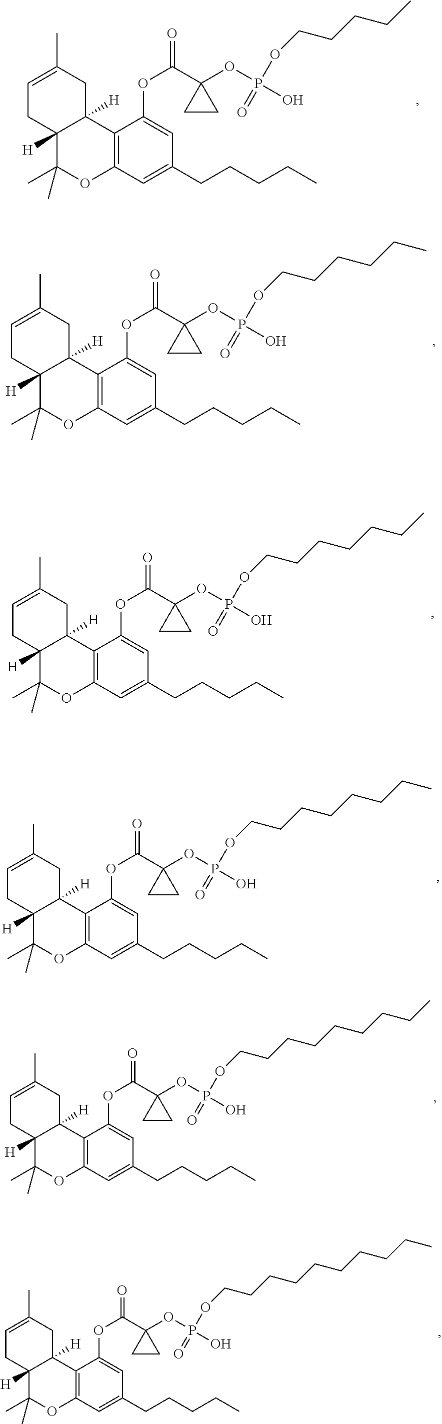 [SMILES: CCCCCOP(=O)(O)OC1(C(=O)Oc2cc(CCCCC)cc3c2[C@@H]2CC(C)=CC[C@H]2C(C)(C)O3)CC1 ; CCCCCCOP(=O)(O)OC1(C(=O)Oc2cc(CCCCC)cc3c2[C@@H]2CC(C)=CC[C@H]2C(C)(C)O3)CC1 ; CCCCCCCOP(=O)(O)OC1(C(=O)Oc2cc(CCCCC)cc3c2[C@@H]2CC(C)=CC[C@H]2C(C)(C)O3)CC1 ; CCCCCCCCOP(=O)(O)OC1(C(=O)Oc2cc(CCCCC)cc3c2[C@@H]2CC(C)=CC[C@H]2C(C)(C)O3)CC1 ; CCCCCCCCCOP(=O)(O)OC1(C(=O)Oc2cc(CCCCC)cc3c2[C@@H]2CC(C)=CC[C@H]2C(C)(C)O3)CC1 ; CCCCCCCCCCOP(=O)(O)OC1(C(=O)Oc2cc(CCCCC)cc3c2[C@@H]2CC(C)=CC[C@H]2C(C)(C)O3)CC1]

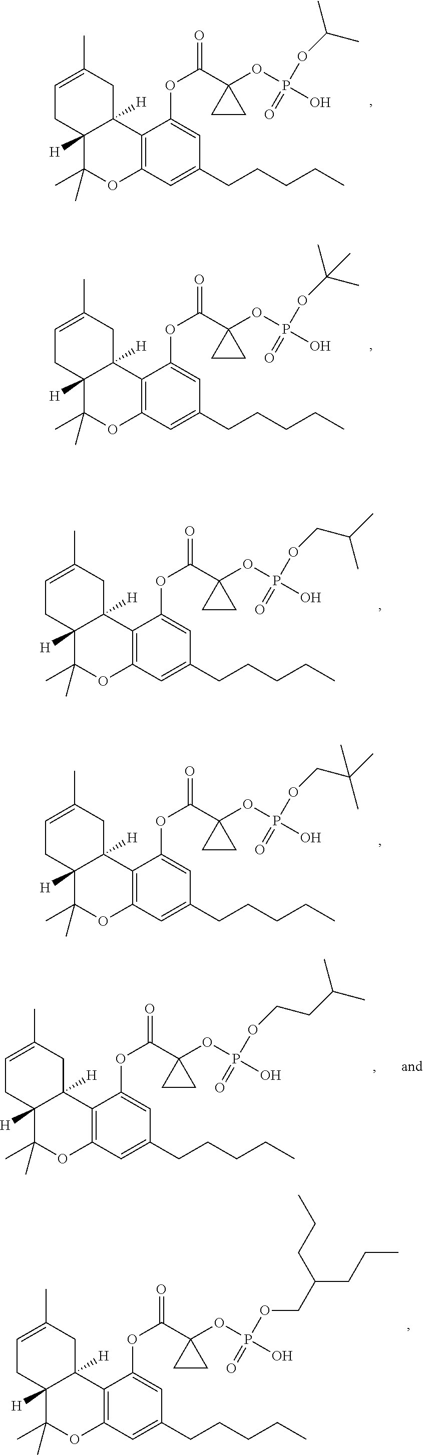

and pharmaceutically acceptable salts of any of the foregoing. In some embodiments the pharmaceutically acceptable salts are ammonium salts.

In some embodiments, the compound of Formula (II)-1A is chosen from compounds having the following formulae:

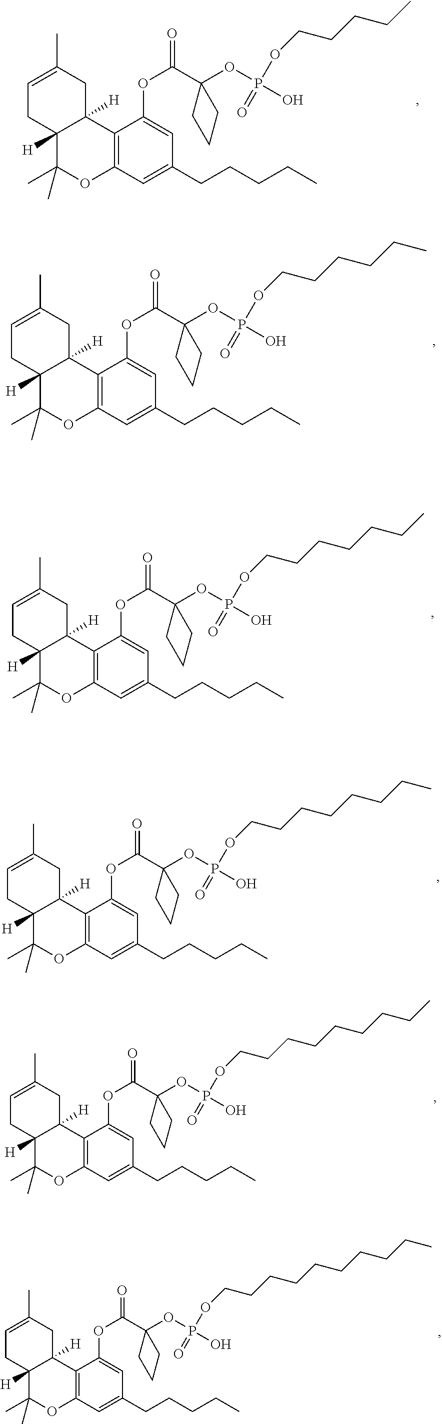 [SMILES: CCCCCOP(=O)(O)OC1(C(=O)Oc2cc(CCCCC)cc3c2[C@@H]2CC(C)=CC[C@H]2C(C)(C)O3)CCC1 ; CCCCCCOP(=O)(O)OC1(C(=O)Oc2cc(CCCCC)cc3c2[C@@H]2CC(C)=CC[C@H]2C(C)(C)O3)CCC1 ; CCCCCCCOP(=O)(O)OC1(C(=O)Oc2cc(CCCCC)cc3c2[C@@H]2CC(C)=CC[C@H]2C(C)(C)O3)CCC1 ; CCCCCCCCOP(=O)(O)OC1(C(=O)Oc2cc(CCCCC)cc3c2[C@@H]2CC(C)=CC[C@H]2C(C)(C)O3)CCC1 ; CCCCCCCCCOP(=O)(O)OC1(C(=O)Oc2cc(CCCCC)cc3c2[C@@H]2CC(C)=CC[C@H]2C(C)(C)O3)CCC1 ; CCCCCCCCCCOP(=O)(O)OC1(C(=O)Oc2cc(CCCCC)cc3c2[C@@H]2CC(C)=CC[C@H]2C(C)(C)O3)CCC1]

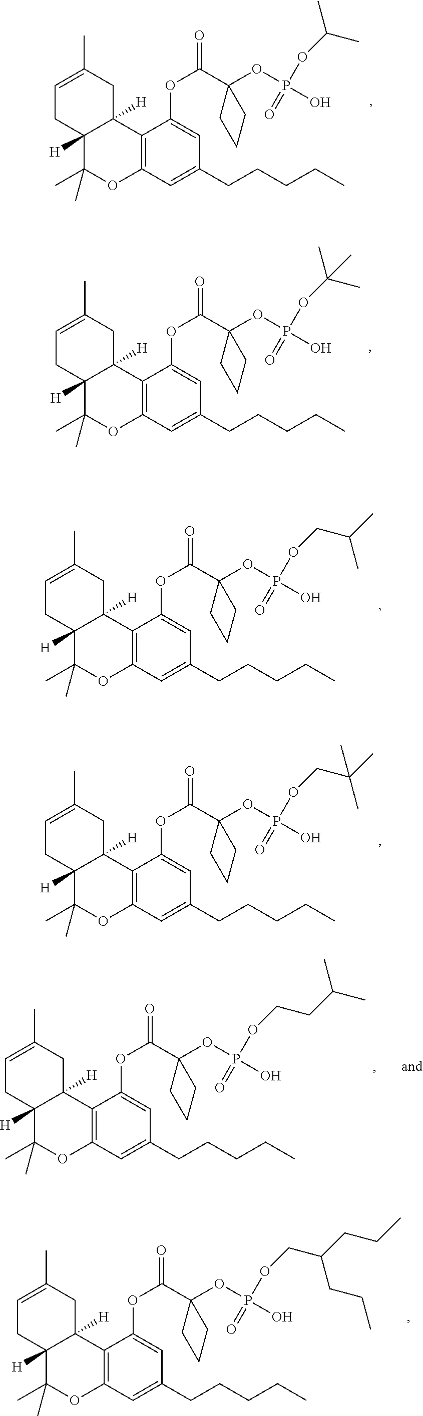

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

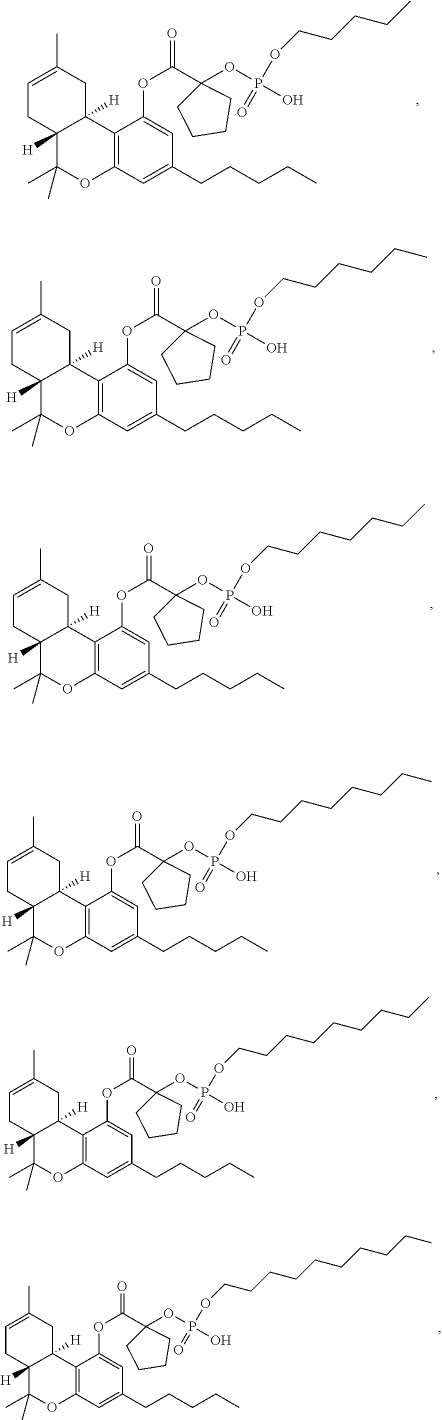 [SMILES: CCCCCOP(=O)(O)OC1(C(=O)Oc2cc(CCCCC)cc3c2[C@@H]2CC(C)=CC[C@H]2C(C)(C)O3)CCCC1 ; CCCCCCOP(=O)(O)OC1(C(=O)Oc2cc(CCCCC)cc3c2[C@@H]2CC(C)=CC[C@H]2C(C)(C)O3)CCCC1 ; CCCCCCCOP(=O)(O)OC1(C(=O)Oc2cc(CCCCC)cc3c2[C@@H]2CC(C)=CC[C@H]2C(C)(C)O3)CCCC1 ; CCCCCCCCOP(=O)(O)OC1(C(=O)Oc2cc(CCCCC)cc3c2[C@@H]2CC(C)=CC[C@H]2C(C)(C)O3)CCCC1 ; CCCCCCCCCOP(=O)(O)OC1(C(=O)Oc2cc(CCCCC)cc3c2[C@@H]2CC(C)=CC[C@H]2C(C)(C)O3)CCCC1 ; CCCCCCCCCCOP(=O)(O)OC1(C(=O)Oc2cc(CCCCC)cc3c2[C@@H]2CC(C)=CC[C@H]2C(C)(C)O3)CCCC1]

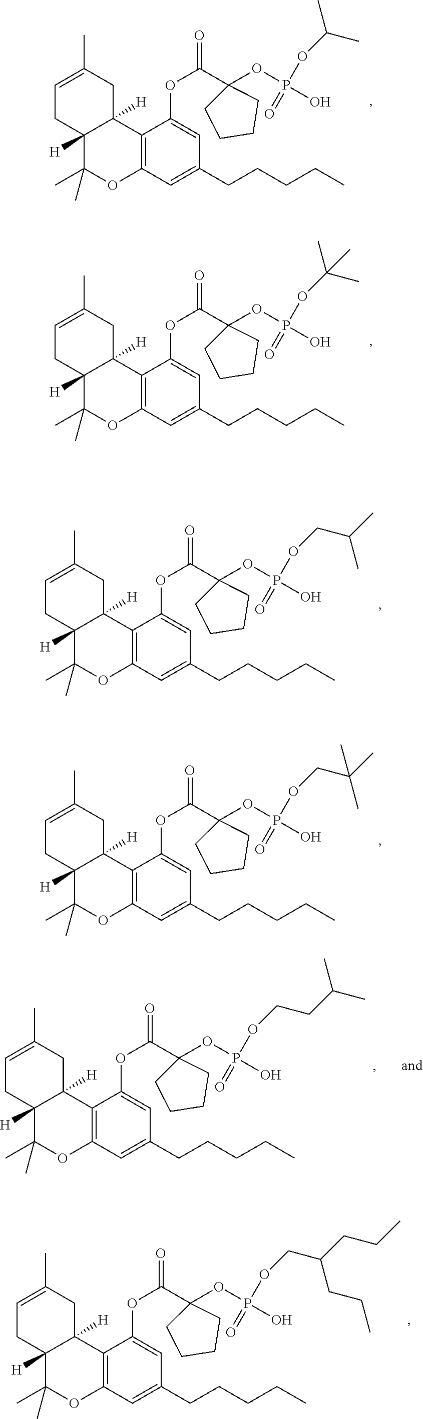

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

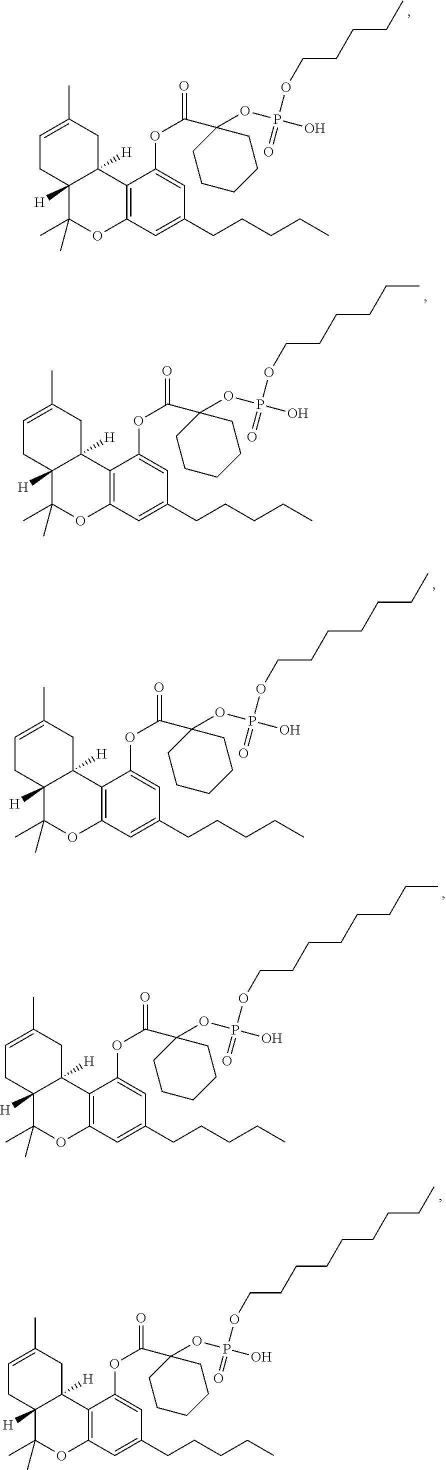 [SMILES: CCCCCOP(=O)(O)OC1(C(=O)Oc2cc(CCCCC)cc3c2[C@@H]2CC(C)=CC[C@H]2C(C)(C)O3)CCCCC1 ; CCCCCCOP(=O)(O)OC1(C(=O)Oc2cc(CCCCC)cc3c2[C@@H]2CC(C)=CC[C@H]2C(C)(C)O3)CCCCC1 ; CCCCCCCOP(=O)(O)OC1(C(=O)Oc2cc(CCCCC)cc3c2[C@@H]2CC(C)=CC[C@H]2C(C)(C)O3)CCCCC1 ; CCCCCCCCOP(=O)(O)OC1(C(=O)Oc2cc(CCCCC)cc3c2[C@@H]2CC(C)=CC[C@H]2C(C)(C)O3)CCCCC1 ; CCCCCCCCCOP(=O)(O)OC1(C(=O)Oc2cc(CCCCC)cc3c2[C@@H]2CC(C)=CC[C@H]2C(C)(C)O3)CCCCC1]

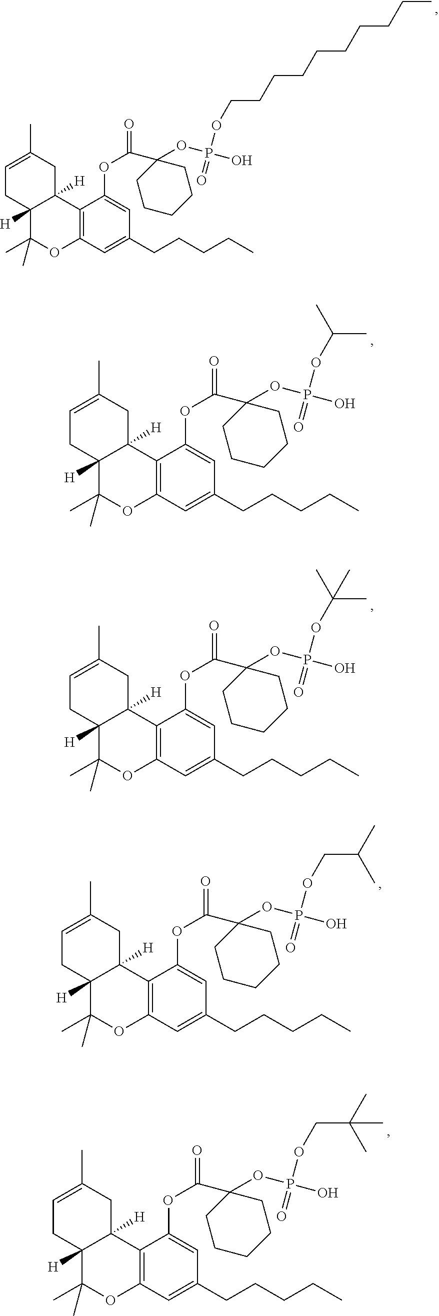 [SMILES: CCCCCCCCCCOP(=O)(O)OC1(C(=O)Oc2cc(CCCCC)cc3c2[C@@H]2CC(C)=CC[C@H]2C(C)(C)O3)CCCCC1 ; CCCCCc1cc(OC(=O)C2(OP(=O)(O)OC(C)C)CCCCC2)c2c(c1)OC(C)(C)[C@@H]1CC=C(C)C[C@@H]21 ; CCCCCc1cc(OC(=O)C2(OP(=O)(O)OC(C)(C)C)CCCCC2)c2c(c1)OC(C)(C)[C@@H]1CC=C(C)C[C@@H]21 ; CCCCCc1cc(OC(=O)C2(OP(=O)(O)OCC(C)C)CCCCC2)c2c(c1)OC(C)(C)[C@@H]1CC=C(C)C[C@@H]21 ; CCCCCc1cc(OC(=O)C2(OP(=O)(O)OCC(C)(C)C)CCCCC2)c2c(c1)OC(C)(C)[C@@H]1CC=C(C)C[C@@H]21]

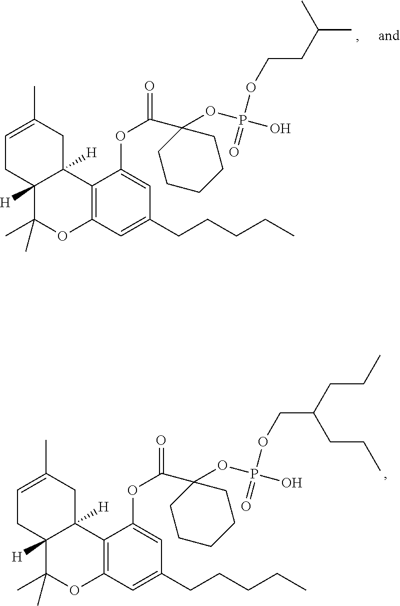 [SMILES: CCCCCc1cc(OC(=O)C2(OP(=O)(O)OCCC(C)C)CCCCC2)c2c(c1)OC(C)(C)[C@@H]1CC=C(C)C[C@@H]21 ; CCCCCc1cc(OC(=O)C2(OP(=O)(O)OCC(CCC)CCC)CCCCC2)c2c(c1)OC(C)(C)[C@@H]1CC=C(C)C[C@@H]21]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

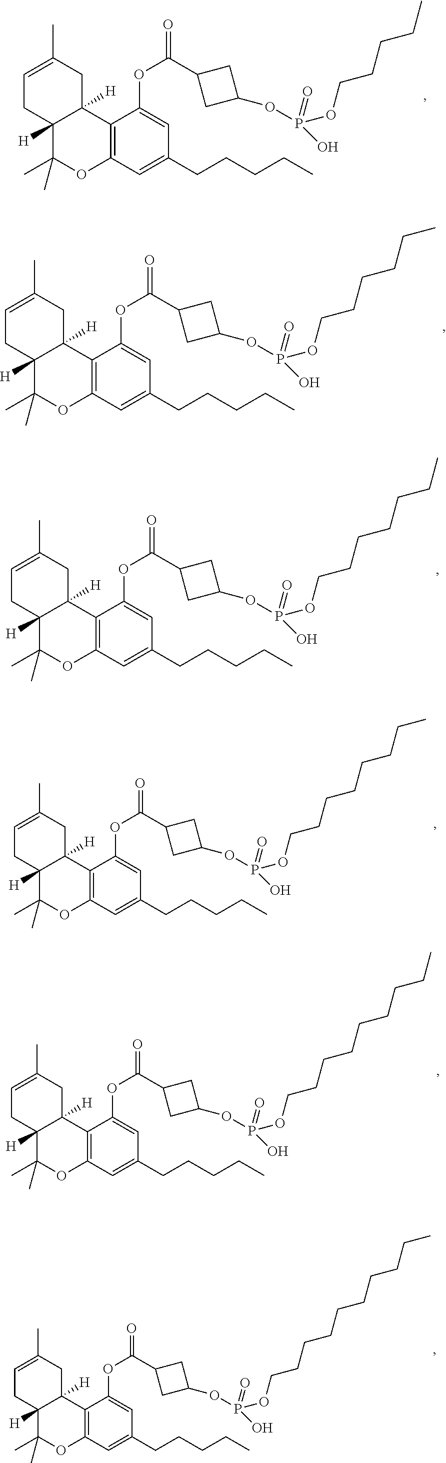 [SMILES: CCCCCOP(=O)(O)OC1CC(C(=O)Oc2cc(CCCCC)cc3c2[C@@H]2CC(C)=CC[C@H]2C(C)(C)O3)C1 ; CCCCCCOP(=O)(O)OC1CC(C(=O)Oc2cc(CCCCC)cc3c2[C@@H]2CC(C)=CC[C@H]2C(C)(C)O3)C1 ; CCCCCCCOP(=O)(O)OC1CC(C(=O)Oc2cc(CCCCC)cc3c2[C@@H]2CC(C)=CC[C@H]2C(C)(C)O3)C1 ; CCCCCCCCOP(=O)(O)OC1CC(C(=O)Oc2cc(CCCCC)cc3c2[C@@H]2CC(C)=CC[C@H]2C(C)(C)O3)C1 ; CCCCCCCCCOP(=O)(O)OC1CC(C(=O)Oc2cc(CCCCC)cc3c2[C@@H]2CC(C)=CC[C@H]2C(C)(C)O3)C1 ; CCCCCCCCCCOP(=O)(O)OC1CC(C(=O)Oc2cc(CCCCC)cc3c2[C@@H]2CC(C)=CC[C@H]2C(C)(C)O3)C1]

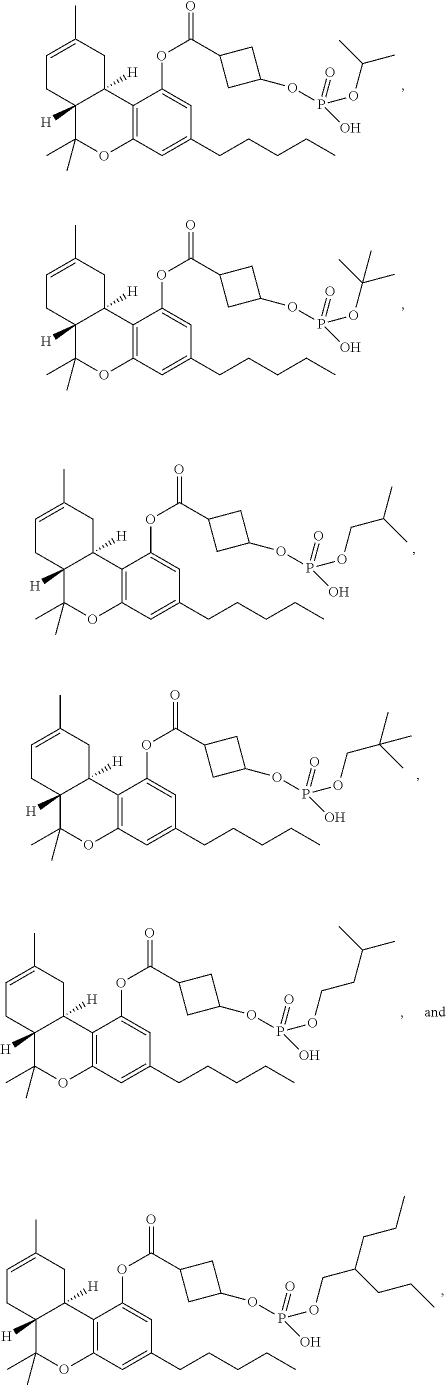 [SMILES: CCCCCc1cc(OC(=O)C2CC(OP(=O)(O)OC(C)C)C2)c2c(c1)OC(C)(C)[C@@H]1CC=C(C)C[C@@H]21 ; CCCCCc1cc(OC(=O)C2CC(OP(=O)(O)OC(C)(C)C)C2)c2c(c1)OC(C)(C)[C@@H]1CC=C(C)C[C@@H]21 ; CCCCCc1cc(OC(=O)C2CC(OP(=O)(O)OCC(C)C)C2)c2c(c1)OC(C)(C)[C@@H]1CC=C(C)C[C@@H]21 ; CCCCCc1cc(OC(=O)C2CC(OP(=O)(O)OCC(C)(C)C)C2)c2c(c1)OC(C)(C)[C@@H]1CC=C(C)C[C@@H]21 ; CCCCCc1cc(OC(=O)C2CC(OP(=O)(O)OCCC(C)C)C2)c2c(c1)OC(C)(C)[C@@H]1CC=C(C)C[C@@H]21 ; CCCCCc1cc(OC(=O)C2CC(OP(=O)(O)OCC(CCC)CCC)C2)c2c(c1)OC(C)(C)[C@@H]1CC=C(C)C[C@@H]21]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

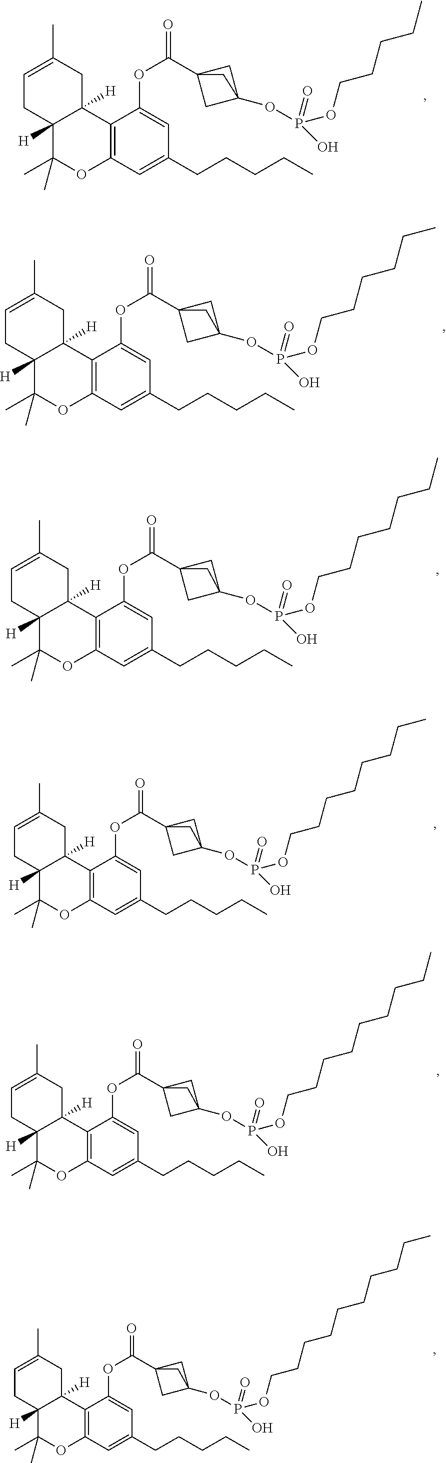

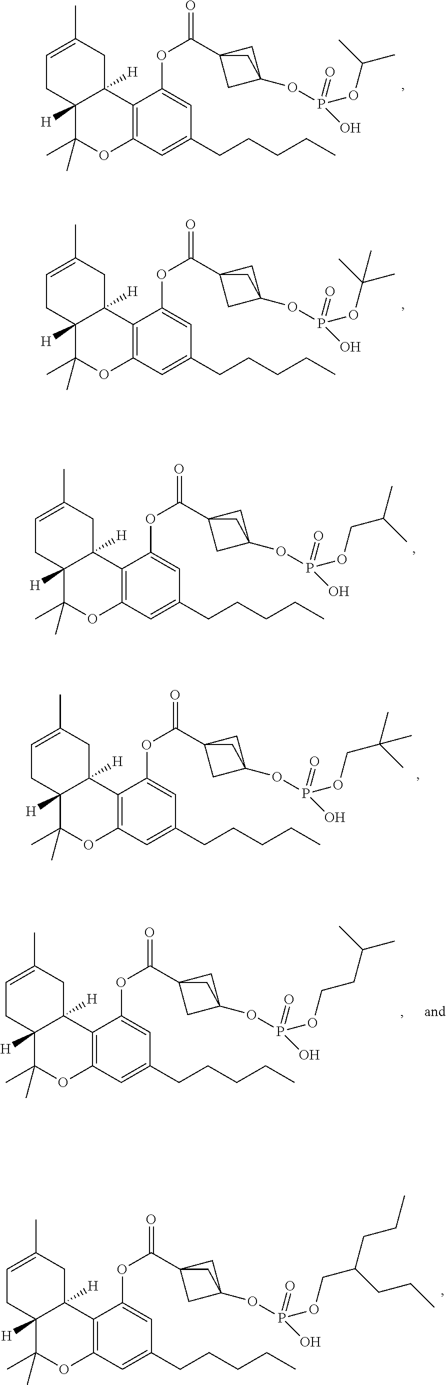

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

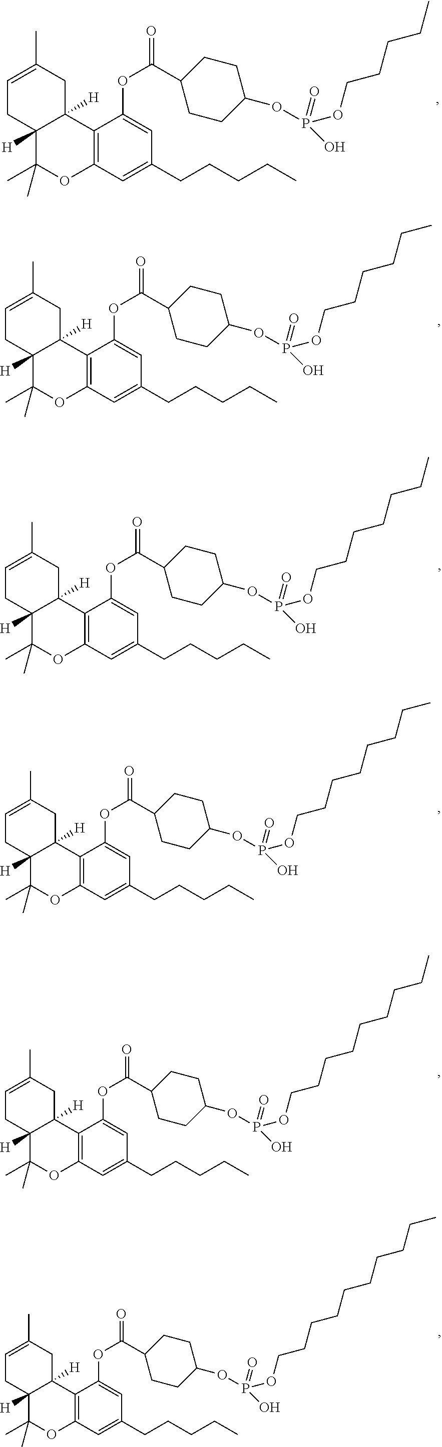 [SMILES: CCCCCOP(=O)(O)OC1CCC(C(=O)Oc2cc(CCCCC)cc3c2[C@@H]2CC(C)=CC[C@H]2C(C)(C)O3)CC1 ; CCCCCCOP(=O)(O)OC1CCC(C(=O)Oc2cc(CCCCC)cc3c2[C@@H]2CC(C)=CC[C@H]2C(C)(C)O3)CC1 ; CCCCCCCOP(=O)(O)OC1CCC(C(=O)Oc2cc(CCCCC)cc3c2[C@@H]2CC(C)=CC[C@H]2C(C)(C)O3)CC1 ; CCCCCCCCOP(=O)(O)OC1CCC(C(=O)Oc2cc(CCCCC)cc3c2[C@@H]2CC(C)=CC[C@H]2C(C)(C)O3)CC1 ; CCCCCCCCCOP(=O)(O)OC1CCC(C(=O)Oc2cc(CCCCC)cc3c2[C@@H]2CC(C)=CC[C@H]2C(C)(C)O3)CC1 ; CCCCCCCCCCOP(=O)(O)OC1CCC(C(=O)Oc2cc(CCCCC)cc3c2[C@@H]2CC(C)=CC[C@H]2C(C)(C)O3)CC1]

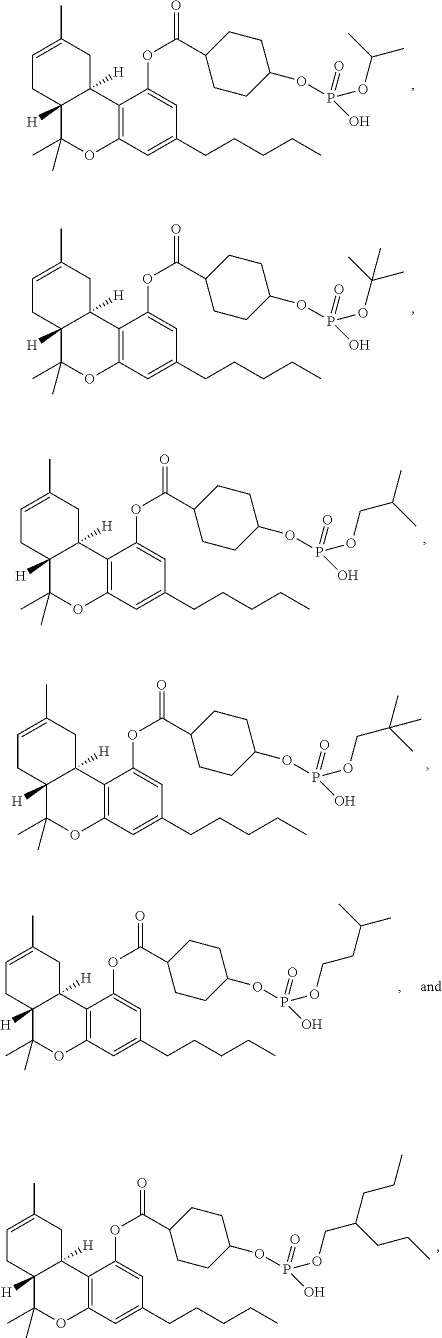

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

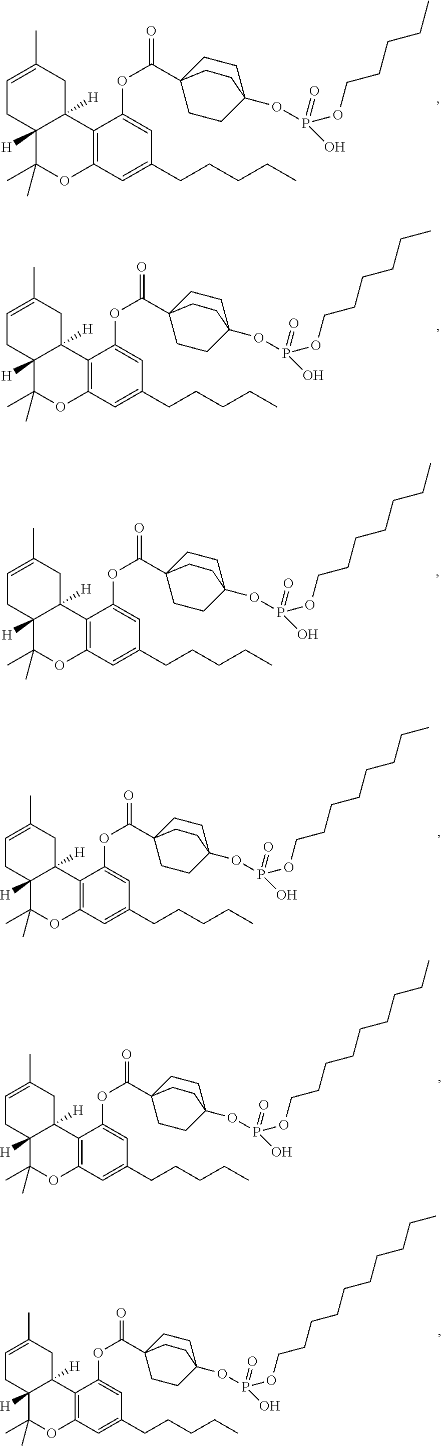

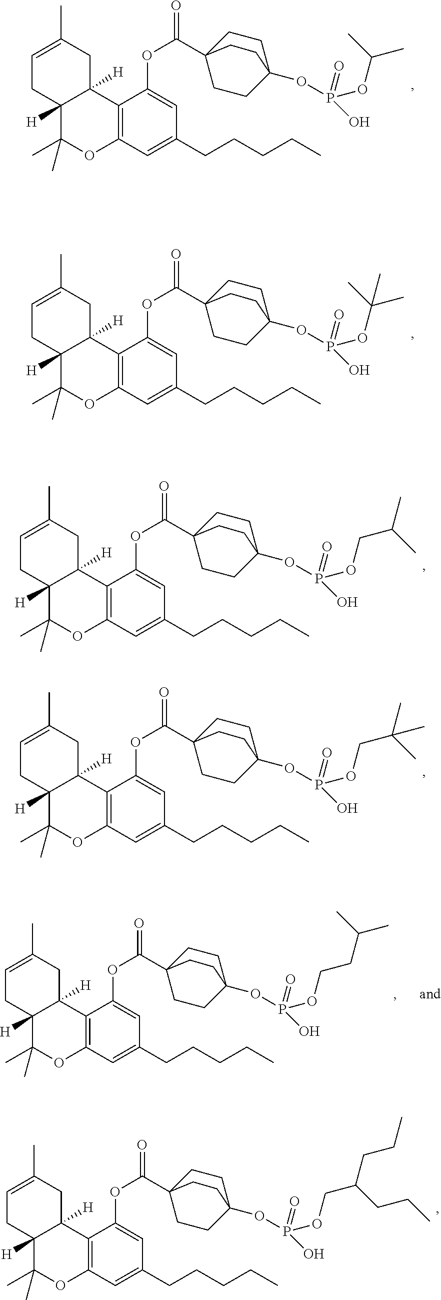

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

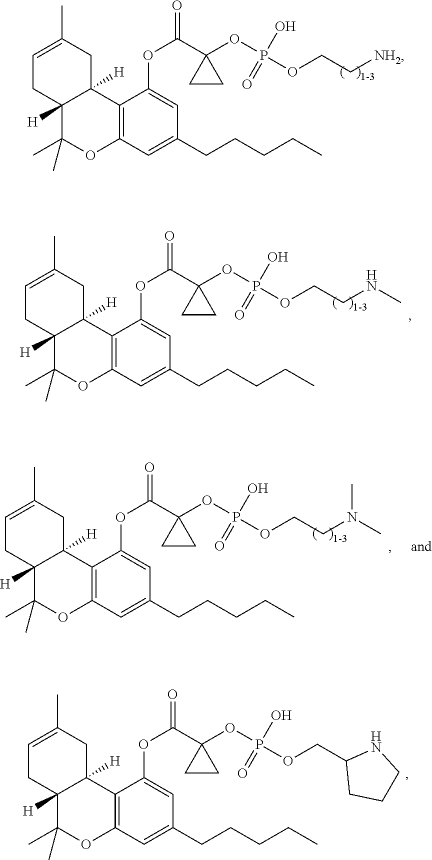

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

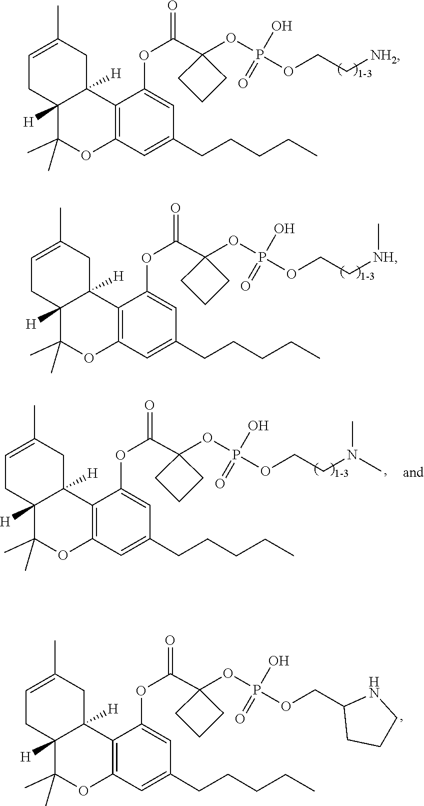

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

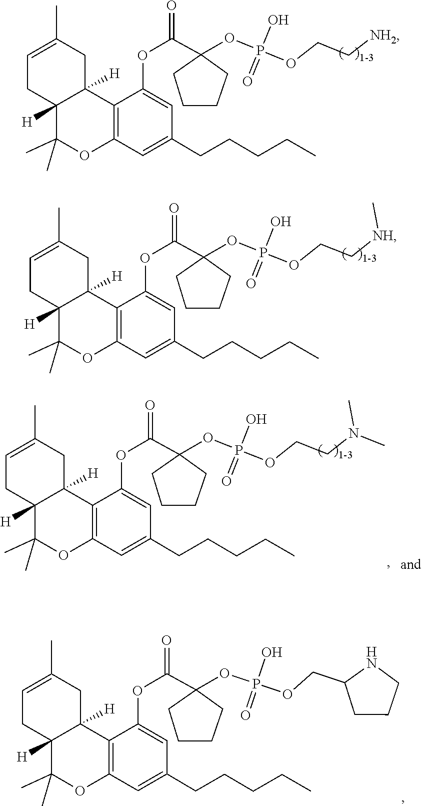

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

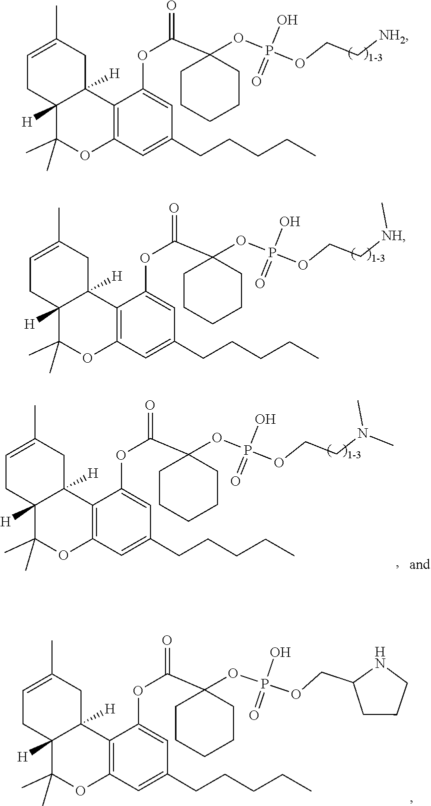

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

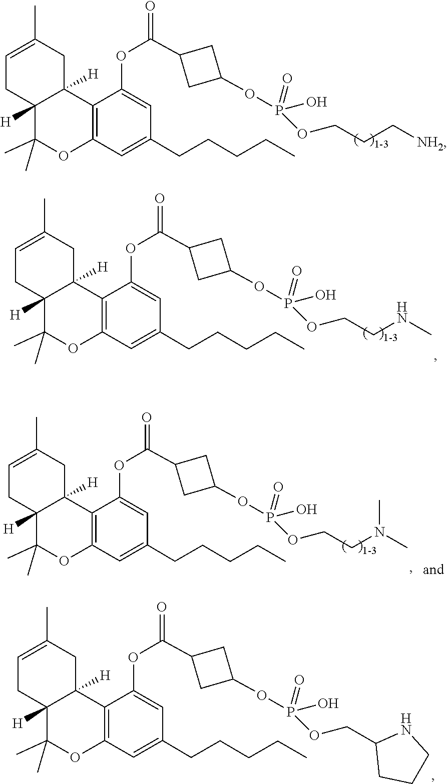

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

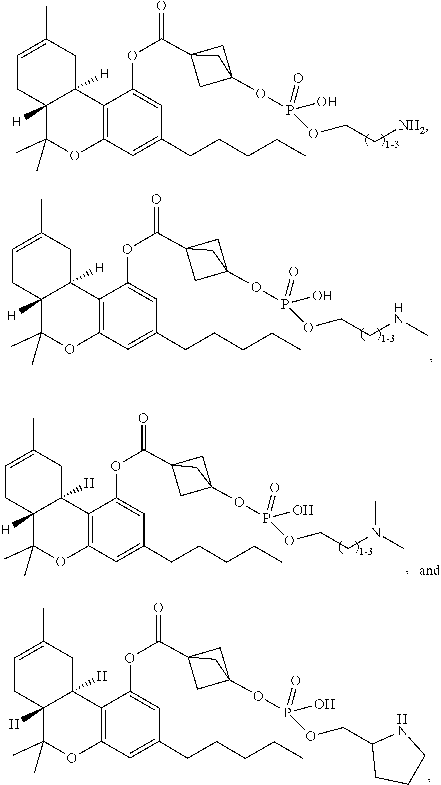

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

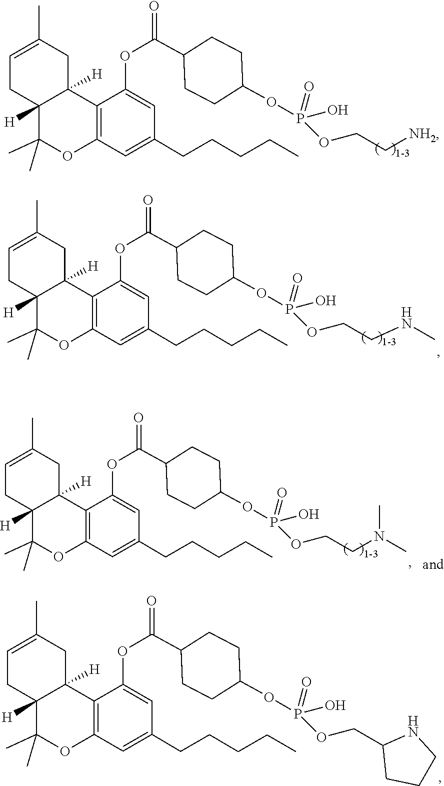

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

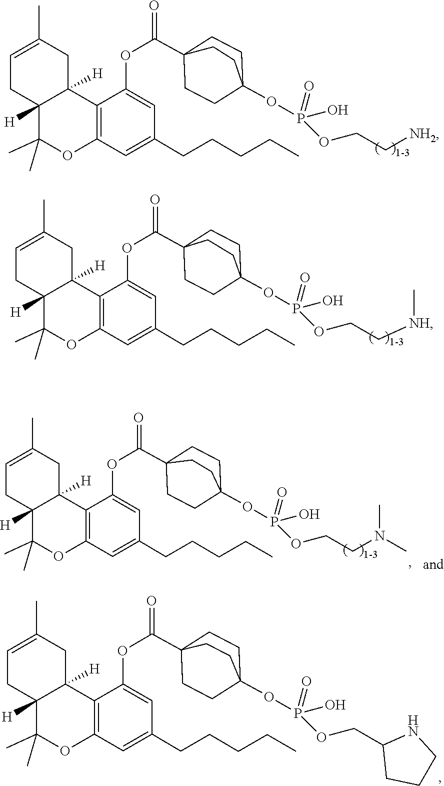

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

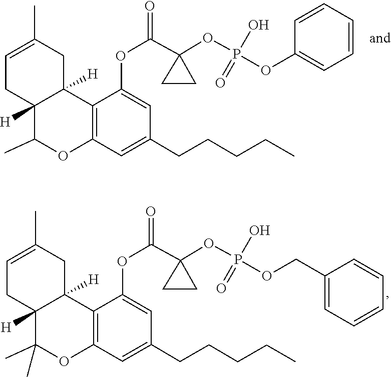

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

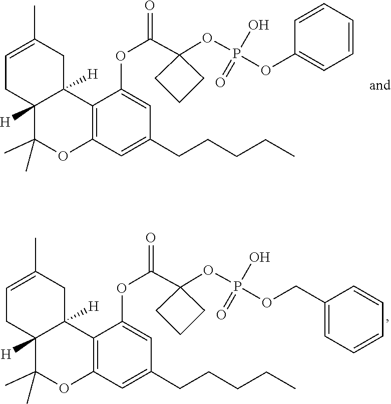

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

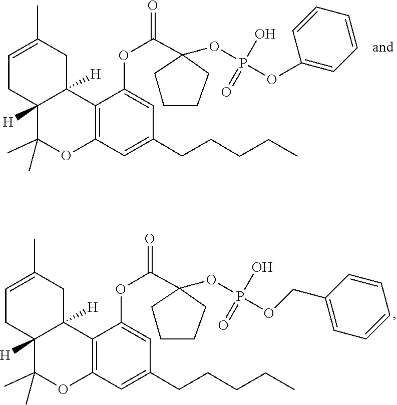

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

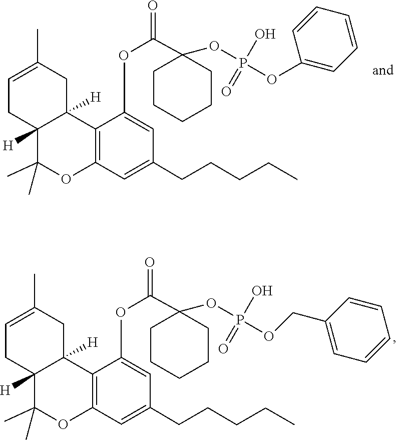

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

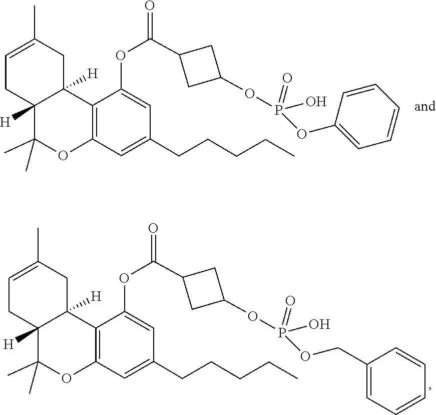

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

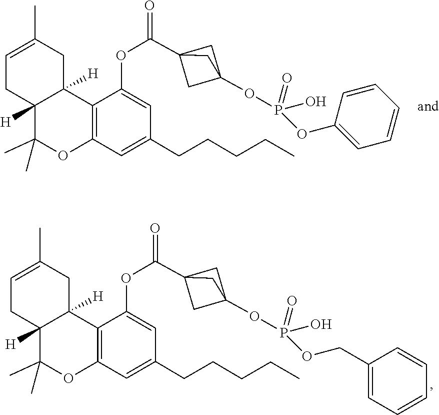

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

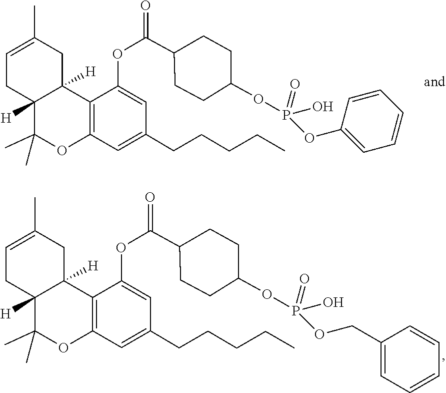

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

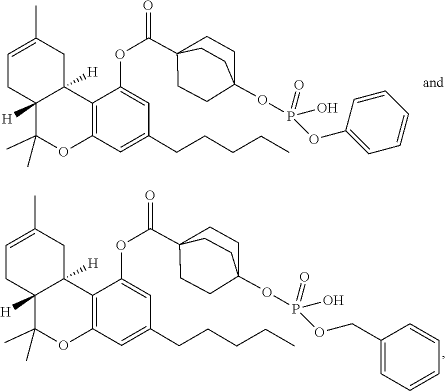

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

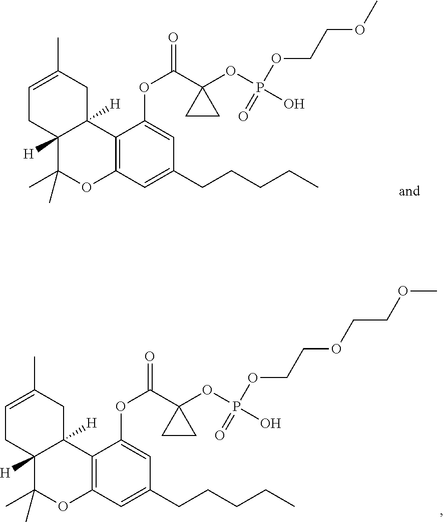 [SMILES: CCCCCc1cc(OC(=O)C2(OP(=O)(O)OCCOC)CC2)c2c(c1)OC(C)(C)[C@@H]1CC=C(C)C[C@@H]21 ; CCCCCc1cc(OC(=O)C2(OP(=O)(O)OCCOCCOC)CC2)c2c(c1)OC(C)(C)[C@@H]1CC=C(C)C[C@@H]21]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

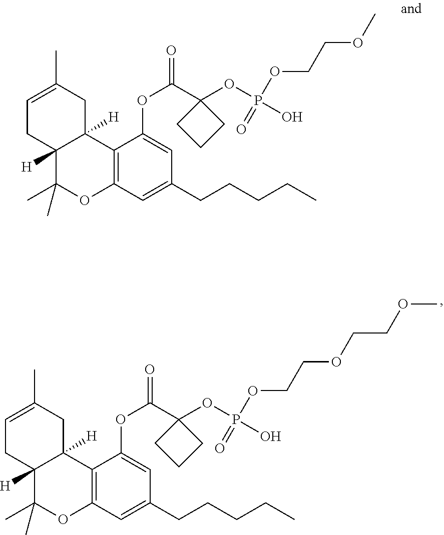

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

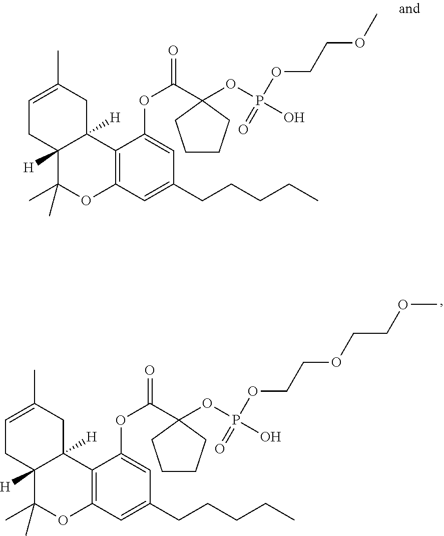

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

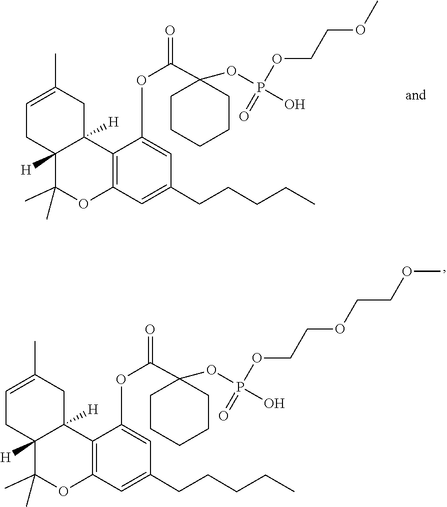

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

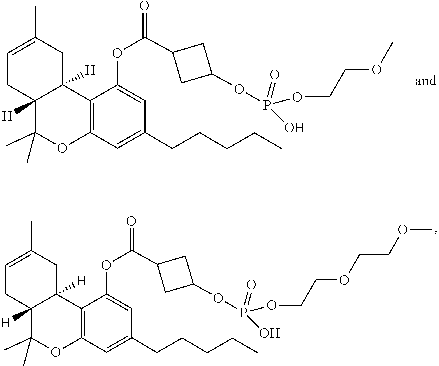

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

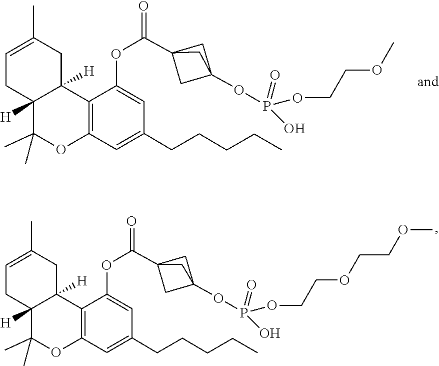

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

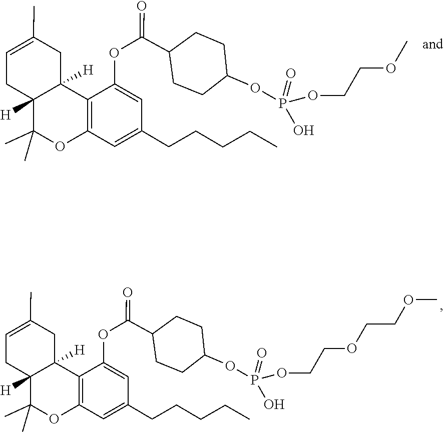

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

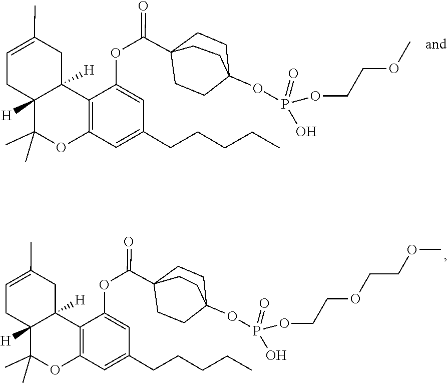

and pharmaceutically acceptable salts of any of the foregoing. In some embodiments, the pharmaceutically acceptable salts are ammonium salts.

In some embodiments, the compound of Formula (II)-1A is chosen from compounds having the following formula:

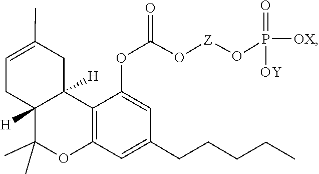

and pharmaceutically acceptable salts thereof. In some embodiments, the carbon in Z that is next to the carbonyloxy group

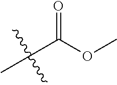 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q. and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

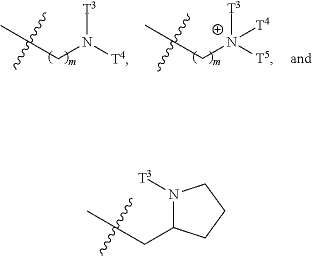

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

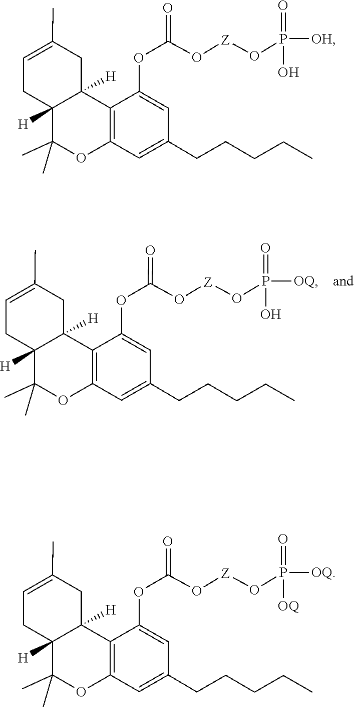

In some embodiments, the carbon in Z that is next to the carbonyloxy group

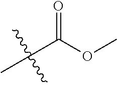 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (II)-1A is chosen from compounds having the following formulae:

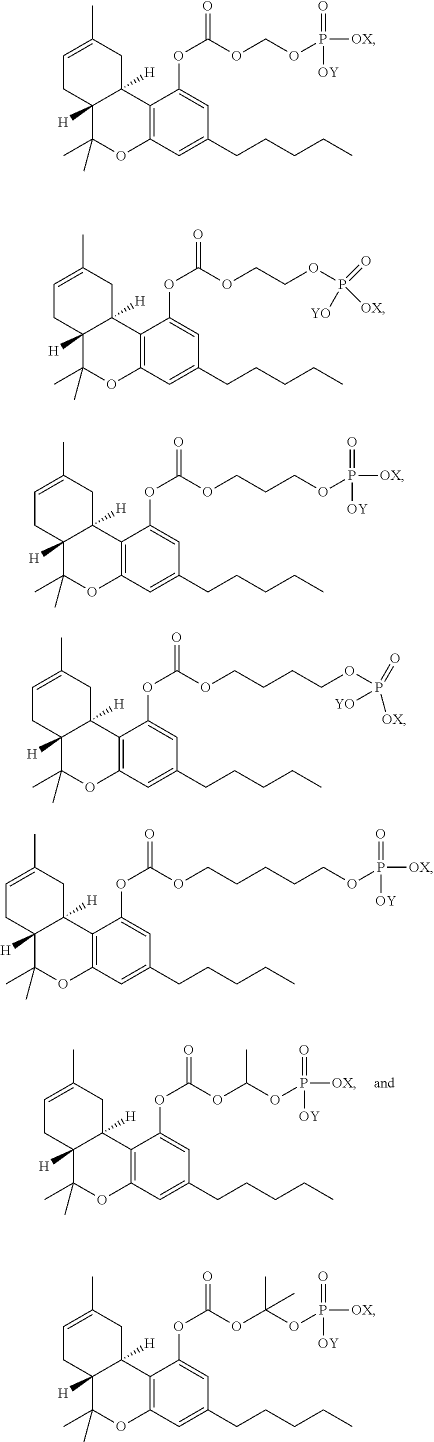

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

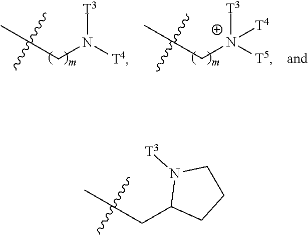

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

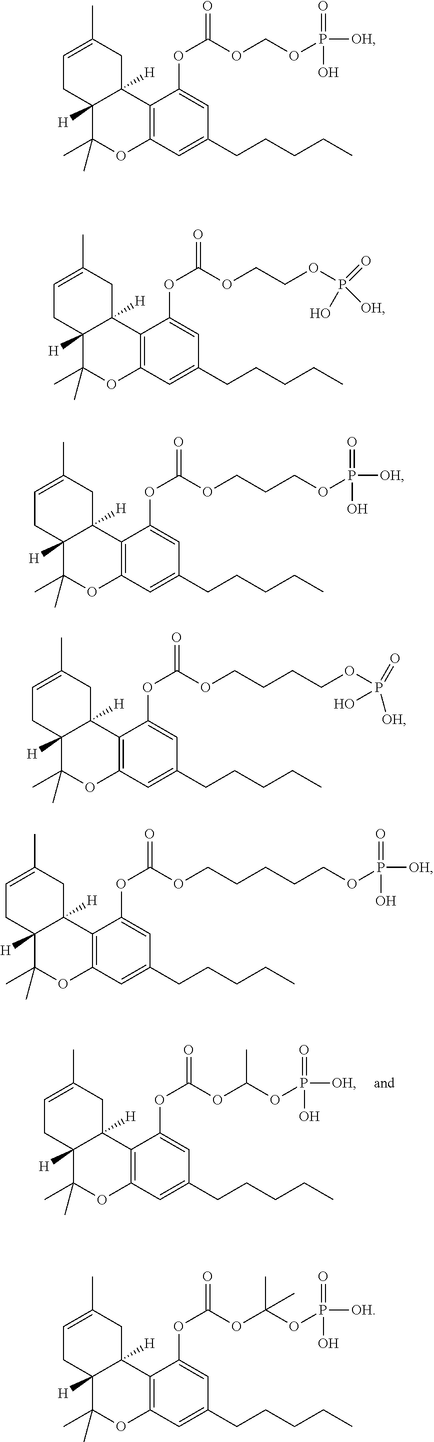

In some embodiments, the compound of Formula (II)-1A is chosen from compounds having the following formulae:

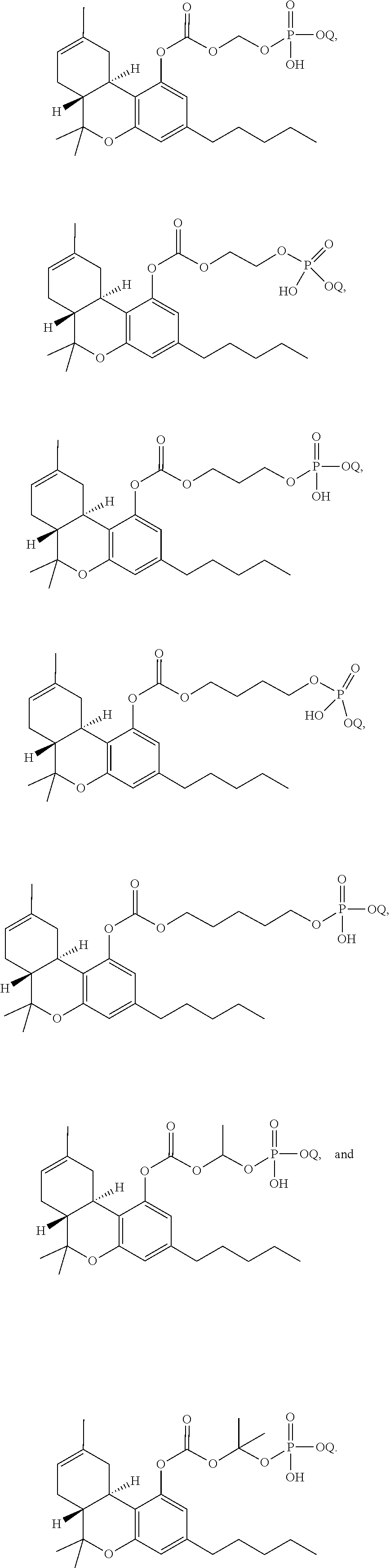

In some embodiments, the compound of Formula (II)-1A is chosen from compounds having the following formulae:

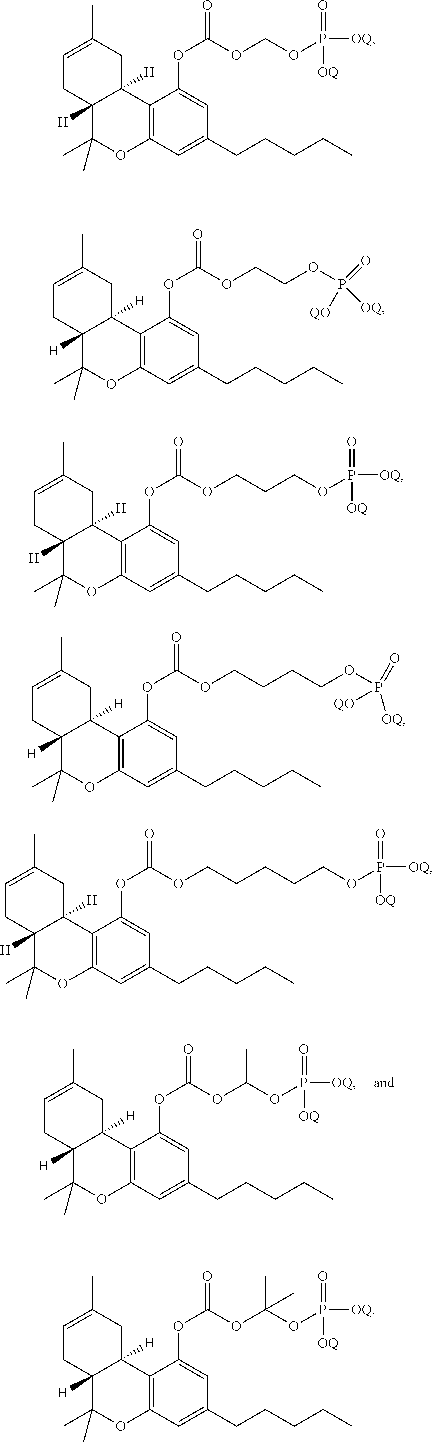

In some embodiments, the compound of Formula (II)-1A is chosen from compounds having the following formulae:

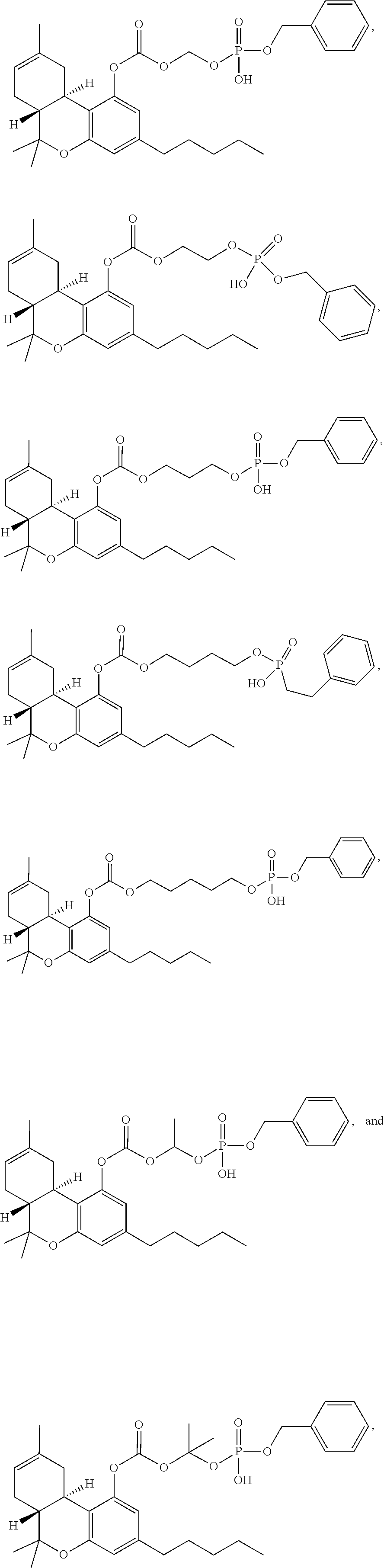

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

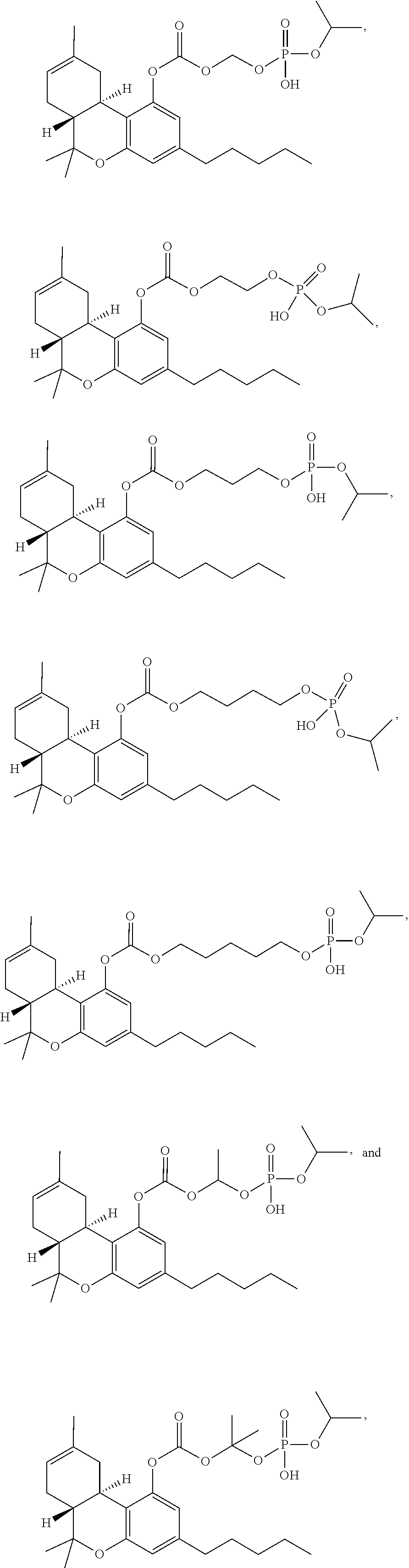

and pharmaceutically acceptable salts of any of the foregoing. In some embodiments, the pharmaceutically acceptable salts are ammonium salts.

In some embodiments, the compound of Formula (II)-1A is chosen from compounds having the following formula:

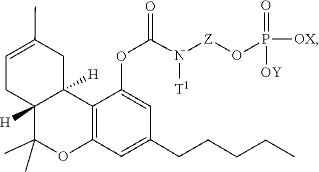

and pharmaceutically acceptable salts thereof. In some embodiments, the carbon in Z that is next to the carbonylamino group

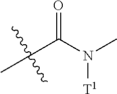

(i.e., the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, the pharmaceutically acceptable salts are ammonium salts. In some embodiments, T¹ is H. In some embodiments, T¹ is methyl. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

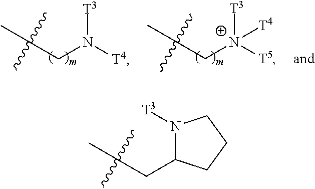

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

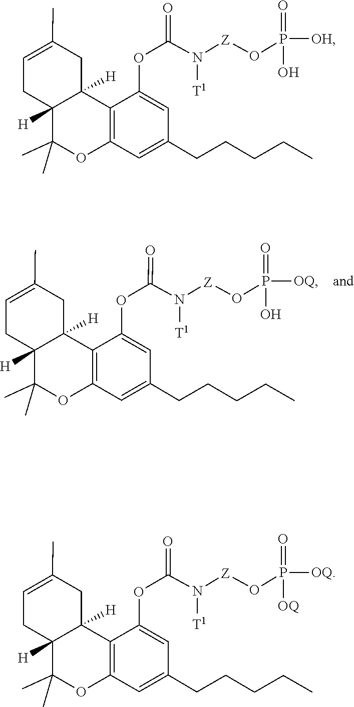

In some embodiments, the carbon in Z that is next to the carbonylamino group

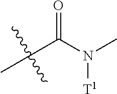

(i.e., the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, T¹ is H. In some embodiments, T¹ is methyl.

In some embodiments, the compound of Formula (II)-1A is chosen from compounds having the following formulae:

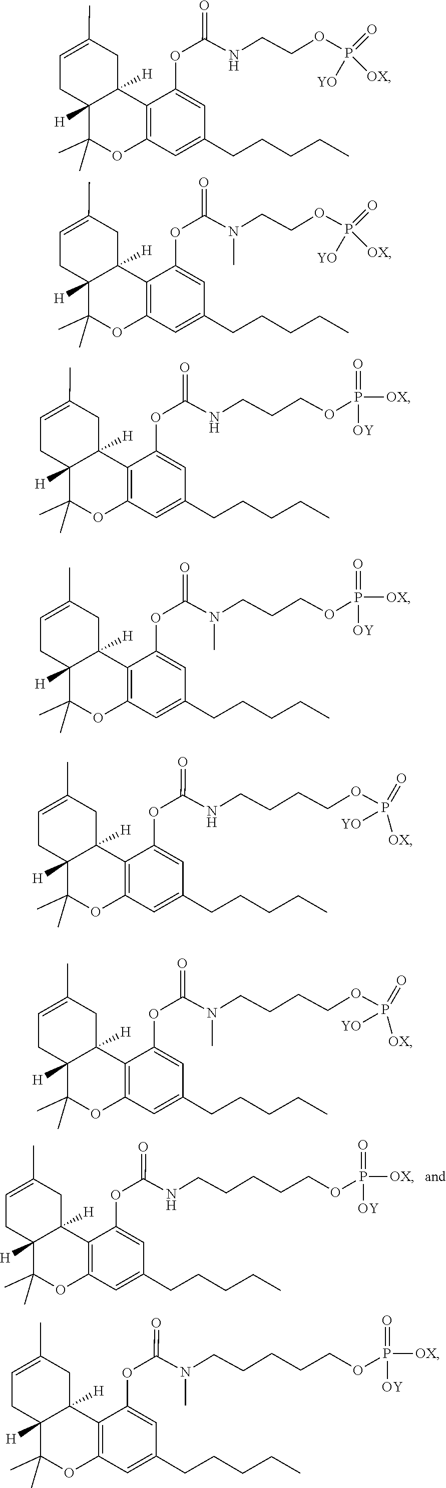

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

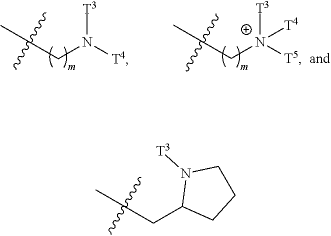

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (II)-1A is chosen from compounds having the following formulae:

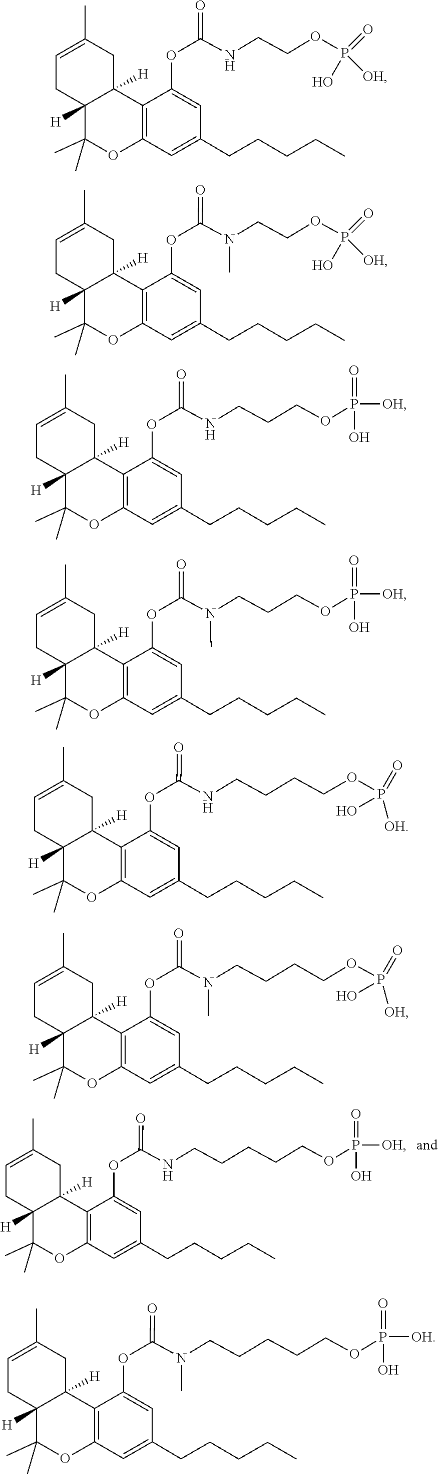

In some embodiments, the compound of Formula (II)-1A is chosen from compounds having the following formulae:

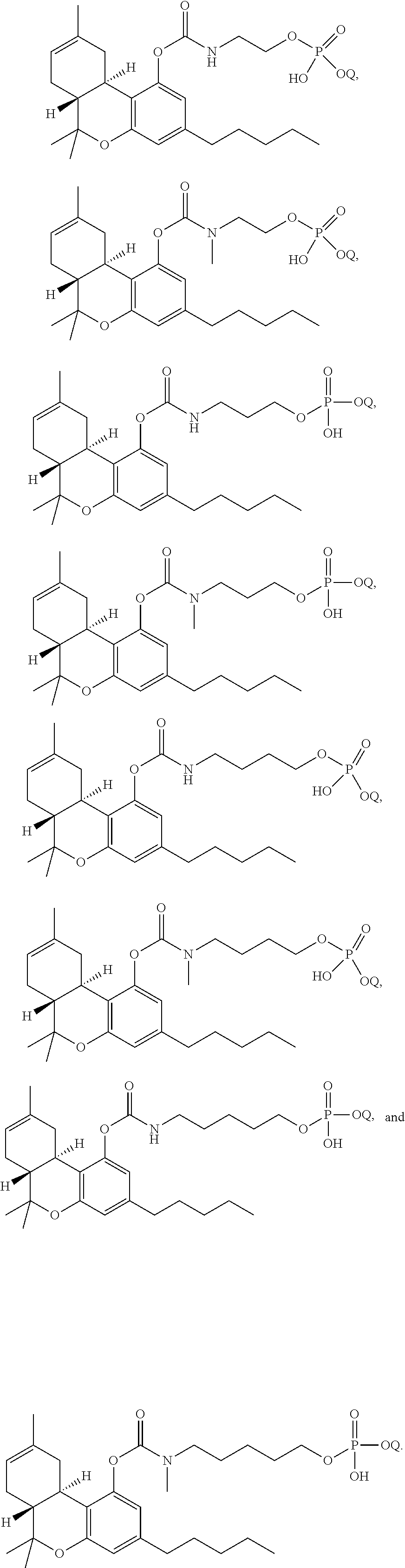

In some embodiments, the compound of Formula (II)-1A is chosen from compounds having the following formulae:

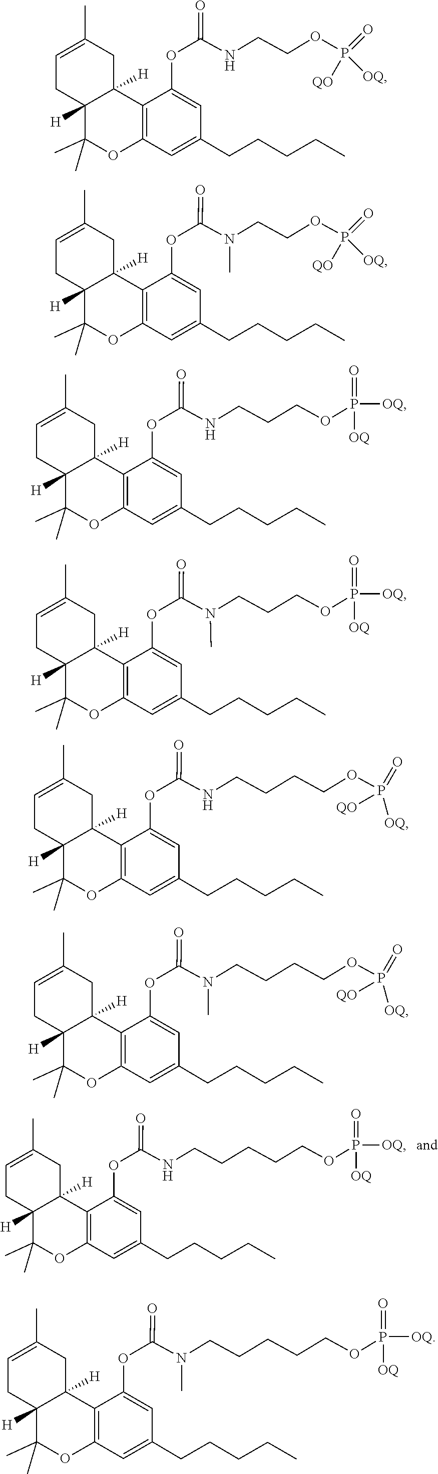

In some embodiments, the compound of Formula (II)-1A is chosen from compounds having the following formulae:

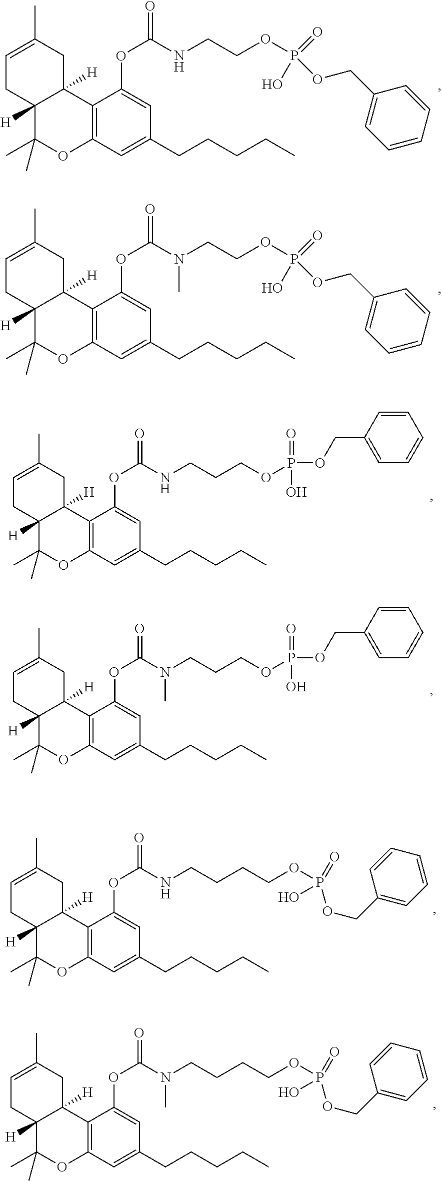

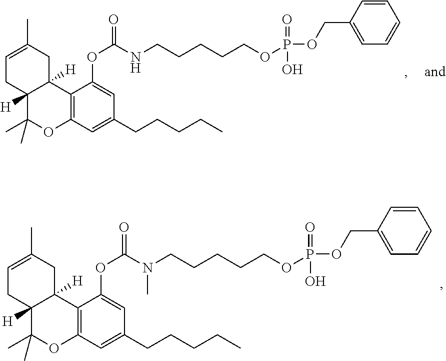 [SMILES: CCCCCc1cc(OC(=O)NCCCCCOP(=O)(O)OCc2ccccc2)c2c(c1)OC(C)(C)[C@@H]1CC=C(C)C[C@@H]21 ; CCCCCc1cc(OC(=O)N(C)CCCCCOP(=O)(O)OCc2ccccc2)c2c(c1)OC(C)(C)[C@@H]1CC=C(C)C[C@@H]21]

and pharmaceutically acceptable salts of any of the foregoing. In some embodiments, the pharmaceutically acceptable salts are ammonium salts.

In some embodiments, the compound of Formula (II)-1A is chosen from compounds having the following formulae:

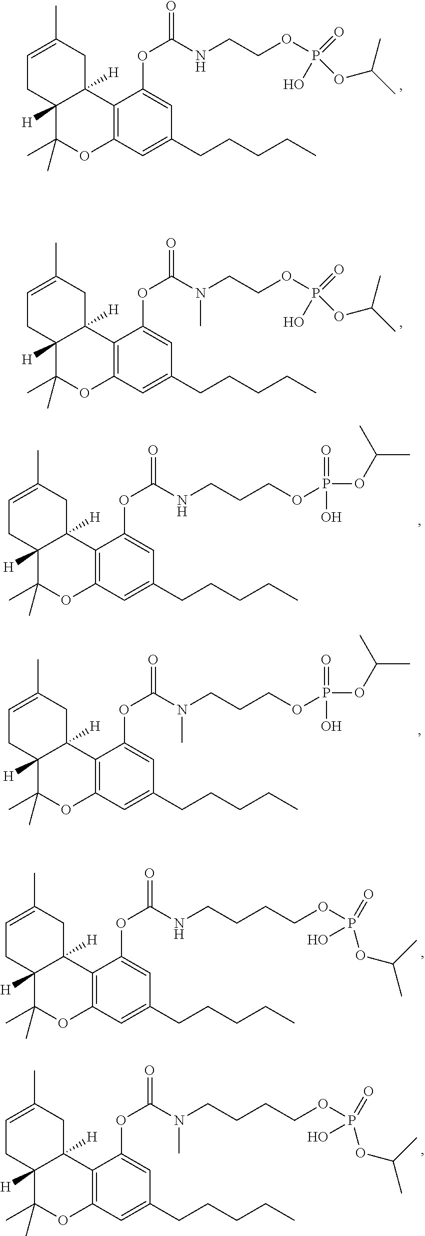

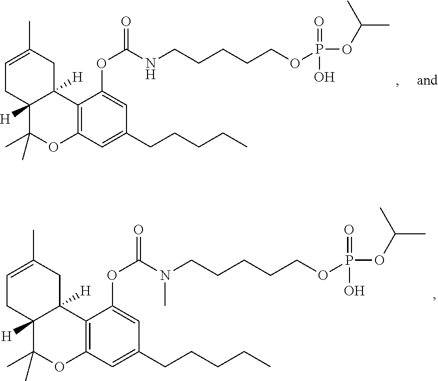 [SMILES: CCCCCc1cc(OC(=O)NCCCCCOP(=O)(O)OC(C)C)c2c(c1)OC(C)(C)[C@@H]1CC=C(C)C[C@@H]21 ; CCCCCc1cc(OC(=O)N(C)CCCCCOP(=O)(O)OC(C)C)c2c(c1)OC(C)(C)[C@@H]1CC=C(C)C[C@@H]21]

and pharmaceutically acceptable salts of any of the foregoing. In some embodiments, the pharmaceutically acceptable salts are ammonium salts.

In some embodiments, the compound of Formula (II)-1A is chosen from compounds having the following formula:

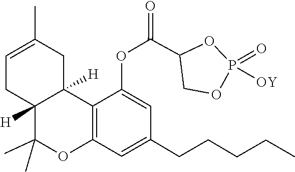

and pharmaceutically acceptable salts thereof. In some embodiments, the pharmaceutically acceptable salts are ammonium salts. In some embodiments, Y is H. In some embodiments, Y is Q. In some embodiments, Y is chosen from C₁₋₁₈ alkyl, C₁₋₁₈ haloalkyl. C₆₋₁₈ aryl, C₁₋₁₃ heteroaryl, C₇₋₁₉ arylalkyl, C₂₋₁₄ heteroarylalkyl, —(CH₂CH₂O)ₙCH₃,

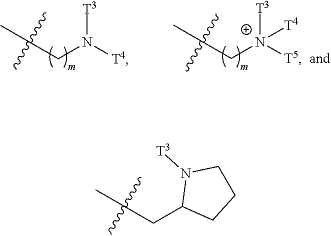

groups. In some embodiments, Y is chosen from phenyl, benzyl and C₁₋₅ alkyl groups. In some embodiments, Y is chosen from phenyl, benzyl, ethyl, and isopropyl.

In some embodiments, the compound of Formula (II)-1A has the following formula:

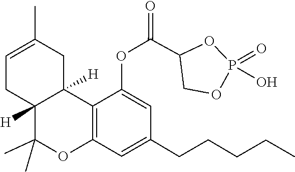 [SMILES: CCCCCc1cc(OC(=O)C2COP(=O)(O)O2)c2c(c1)OC(C)(C)[C@@H]1CC=C(C)C[C@@H]21]

and pharmaceutically acceptable salts thereof. In some embodiments, the pharmaceutically acceptable salts are ammonium salts.

In some embodiments, the compound of Formula (II)-1A has the following formula:

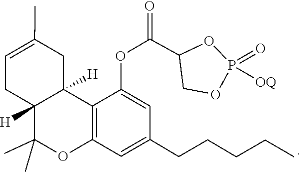

In some embodiments, the compound of Formula (II)-1A has the following formula:

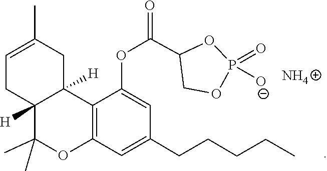 [SMILES: CCCCCc1cc(OC(=O)C2COP(=O)([O-])O2)c2c(c1)OC(C)(C)[C@@H]1CC=C(C)C[C@@H]21 ; [NH4+]]

In some embodiments, the compound of Formula (II)-1A is chosen from compounds having the following formulae:

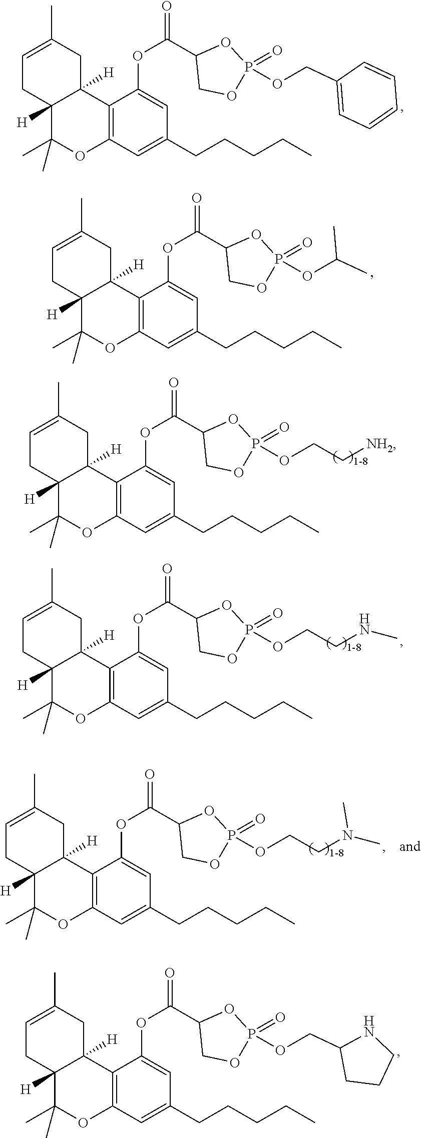

and pharmaceutically acceptable salts of any of the foregoing. In some embodiments, the pharmaceutically acceptable salts are ammonium salts.

### Formula (II)-1B

In some embodiments, Formula (II)-1 is in the form of Formula (II)-1B. i.e., the compounds are prodrugs of HU-210. R¹ is the same as described above. For example, exemplary embodiments of R¹ are described in Formula (II)-1A.

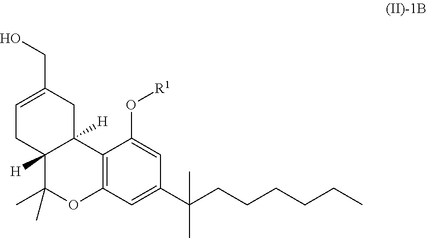

### Formula (II)-2A

In some embodiments, Formula (II)-2 is in the form of Formula (II)-2A. i.e., the compounds are prodrugs of (+)trans-Δ⁸-THC. R¹ is the same as described above. For example, exemplary embodiments of R¹ are described in Formula (II)-1A.

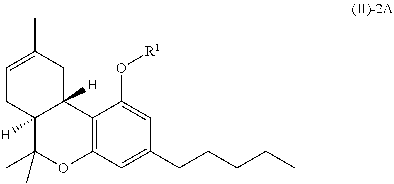

### Formula (II)-2B

In some embodiments, Formula (II)-2 is in the form of Formula (II)-2B, i.e., the compounds are prodrugs of dexanabinol. R¹ is the same as described above. For example, exemplary embodiments of R¹ are described in Formula (II)-1A.

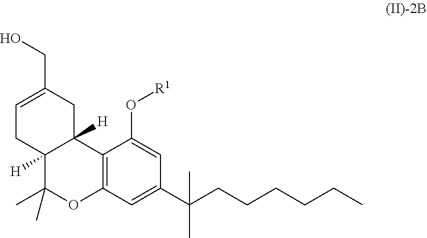

### 3. Formula (III) and its Sub-Formulas

Optionally, the compounds have a structure of Formula (III) or a pharmaceutically acceptable salt thereof:

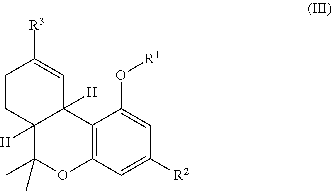

wherein R¹, R², and R³ are the same as described above for Formula (I), except that R³ cannot be ═O.

In some embodiments, the compounds are in a non-salt form. In some embodiments, the compounds are in a salt form. In some embodiments, the compounds are in an ammonium salt form.

In some embodiments, Formula (III) is in the form of Formula (III)-1.

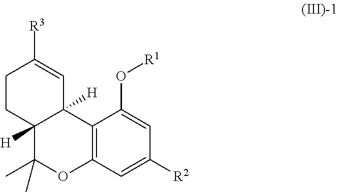

In some embodiments, Formula (III) is in the form of Formula (III)-2.

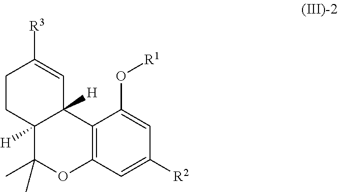

In some embodiments, R² is chosen from C₂₋₉ alkyl groups, and R³ is chosen from —CH₃, —CD₃, —CH₂OH, and —CH₂OR^{a}. In some embodiments, R² is chosen from C₂₋₉ alkyl groups, and R³ is —CH₃. In some embodiments, R² is chosen from C₂₋₉ alkyl groups, and R³ is —CD₃. In some embodiments, R² is chosen from C₂₋₉ alkyl groups, and R³ is —CH₂OH. In some embodiments, R² is chosen from C₂₋₉ alkyl groups, and R³ is —CH₂OR^{a}.

In some embodiments, R² is chosen from n-propyl, n-pentyl, and 1,1-dimethylheptyl, and R³ is chosen from —CH₃, —CD₃, —CH₂OH, and —CH₂OR^{a}. In some embodiments, R² is chosen from n-propyl, n-pentyl, and 1,1-dimethylheptyl, and R³ is —CH₃. In some embodiments, R² is chosen from n-propyl, n-pentyl, and 1,1-dimethylheptyl, and R³ is —CD₃. In some embodiments, R² is chosen from n-propyl, n-pentyl, and 1,1 -dimethylheptyl, and R³ is —CH₂OH. In some embodiments, R² is chosen from n-propyl, n-pentyl, and 1,1-dimethylheptyl, and R³ is —CH₂OR^{a}.

In some embodiments, R² is n-propyl, and R³ is chosen from —CH₃, —CD₃, —CH₂OH, and —CH₂OR^{a}. In some embodiments, R² is n-propyl, and R³ is —CH₃. In some embodiments, R² is n-propyl, and R³ is —CD₃. In some embodiments, R² is n-propyl, and R³ is —CH₂OH. In some embodiments, R² is n-propyl, and R³ is —CH₂OR^{a}.

In some embodiments, R² is n-pentyl, and R³ is chosen from —CH₃, —CD₃. —CH₂OH, and —CH₂OR^{a}. In some embodiments, R² is n-pentyl, and R³ is —CH₃. In some embodiments, R² is n-pentyl, and R³ is —CD₃. In some embodiments, R² is n-pentyl, and R³ is —CH₂OH. In some embodiments, R² is n-pentyl, and R³ is —CH₂OR.

In some embodiments, R² is 1,1-dimethylheptyl, and R³ is chosen from —CH₃. —CD₃, —CH₂OH, and —CH₂OR^{a}. In some embodiments, R² is 1,1-dimethylheptyl, and R³ is —CH₃. In some embodiments, R² is 1,1-dimethylheptyl, and R³ is —CD₃. In some embodiments, R² is 1,1-dimethylheptyl, and R³ is —CH₂OH. In some embodiments, R² is 1,1-dimethylheptyl, and R³ is —CH₂OR^{a}.

### Formula (II)-1A

In some embodiments, Formula (III)-1 is in the form of Formula (III)-1A, i.e., the compounds are prodrugs of (−)trans-Δ⁹-THC.

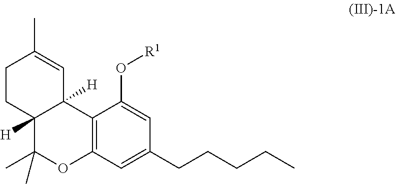

In some embodiments, the compounds of Formula (III)-1A are chosen from compounds having the following formula:

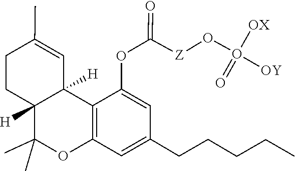

and pharmaceutically acceptable salts thereof. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

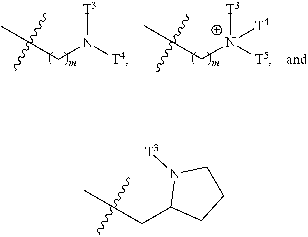

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

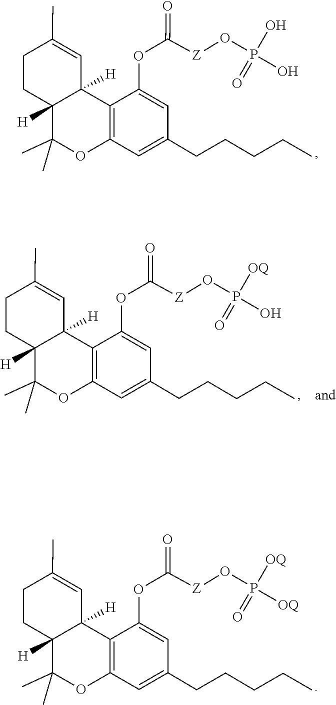

In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (III)-1A is chosen from compounds having the following formulae:

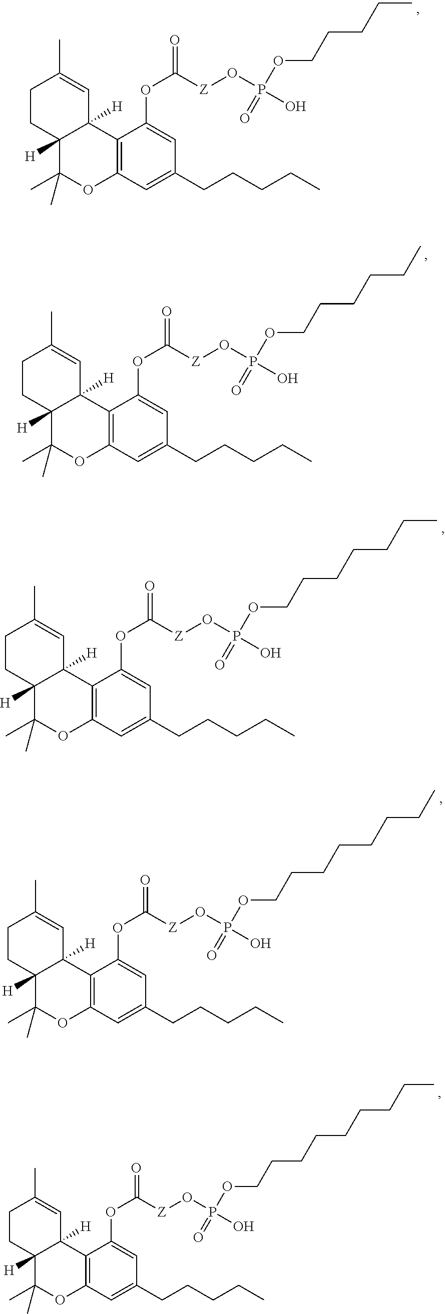

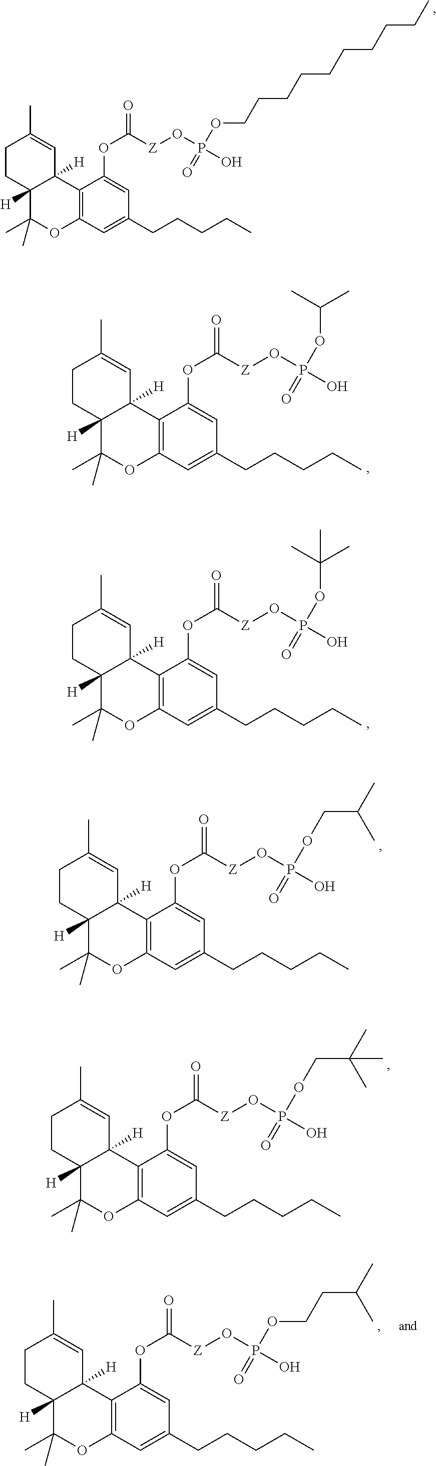

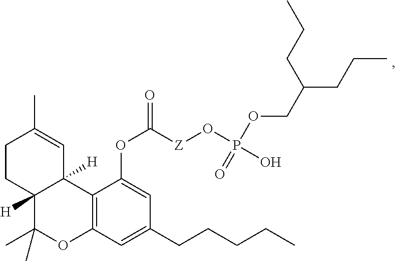

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (III)-1A is chosen from compounds having the following formulae:

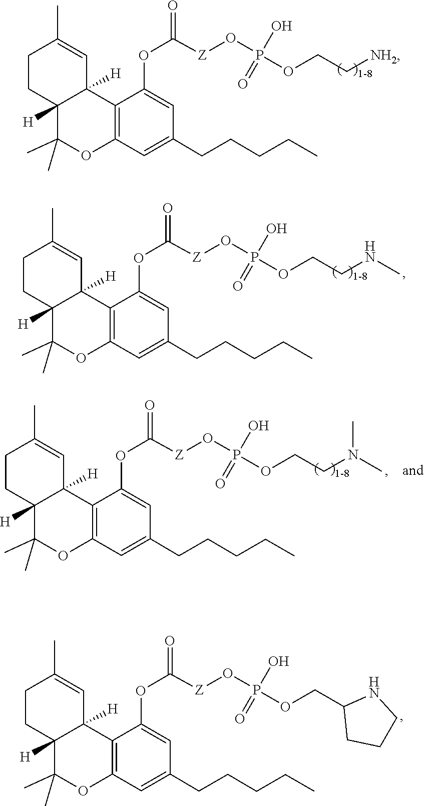

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (III)-1A is chosen from compounds having the following formulae:

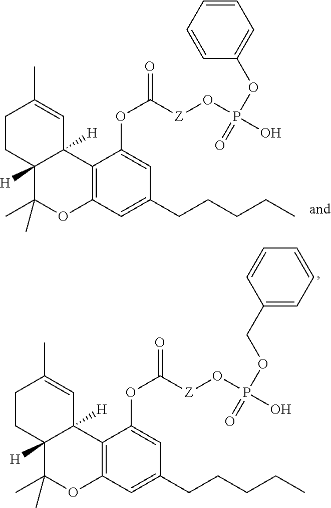

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (III)-1A is chosen from compounds having the following formulae:

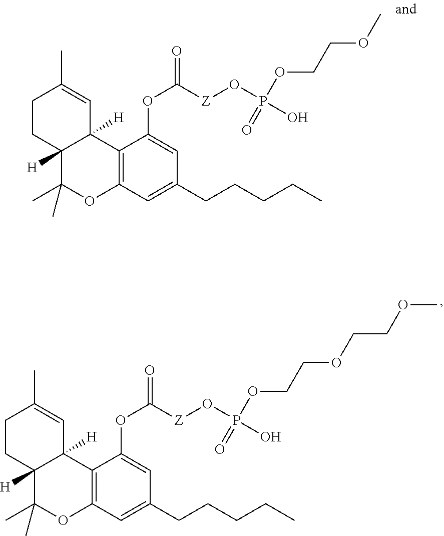

and pharmaceutically acceptable salts of any of the foregoing. In some embodiments, the pharmaceutically acceptable salts are ammonium salts. In some embodiments, the carbon in Z that is next to the carbonyl group (i.e., the alpha carbon) has no hydrogen atoms. In some embodiments, the carbon in Z that is next to the carbonyl group (i.e., the alpha carbon) has one hydrogen atom. In some embodiments, the carbon in Z that is next to the carbonyl group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (III)-1A is chosen from compounds having the following formulae:

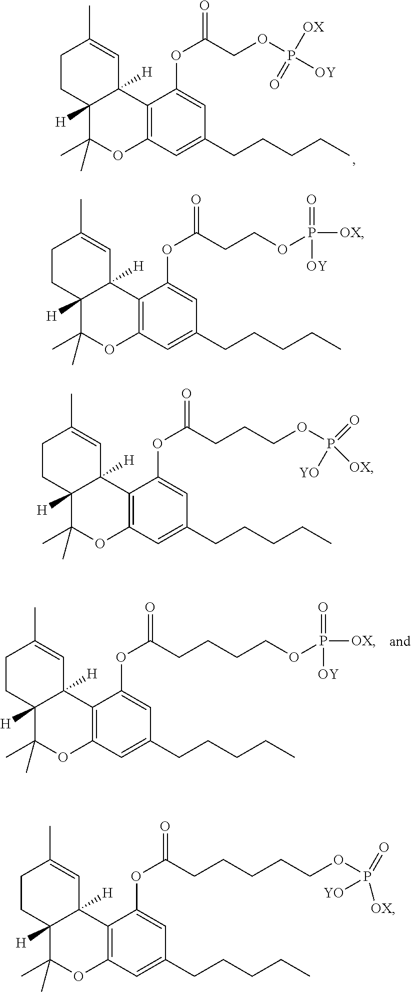

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

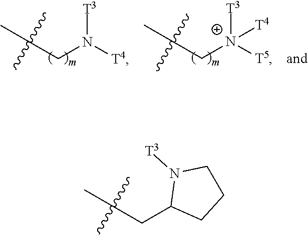

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

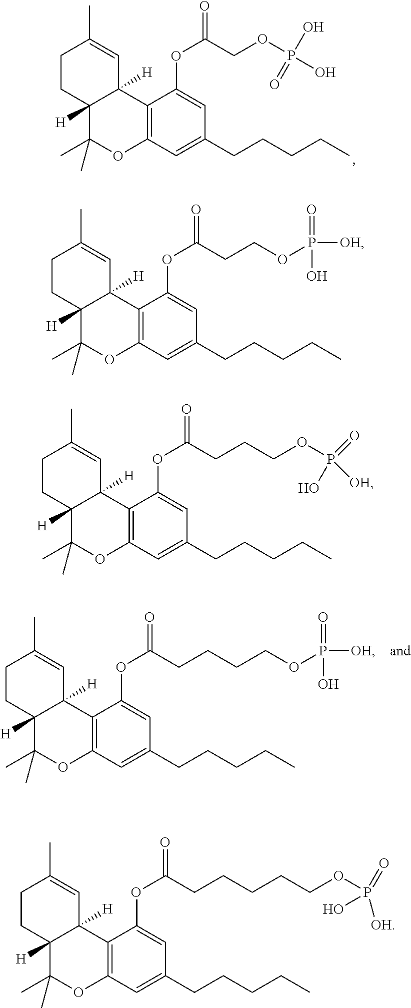

In some embodiments, the compound of Formula (III)-1A is chosen from compounds having the following formulae:

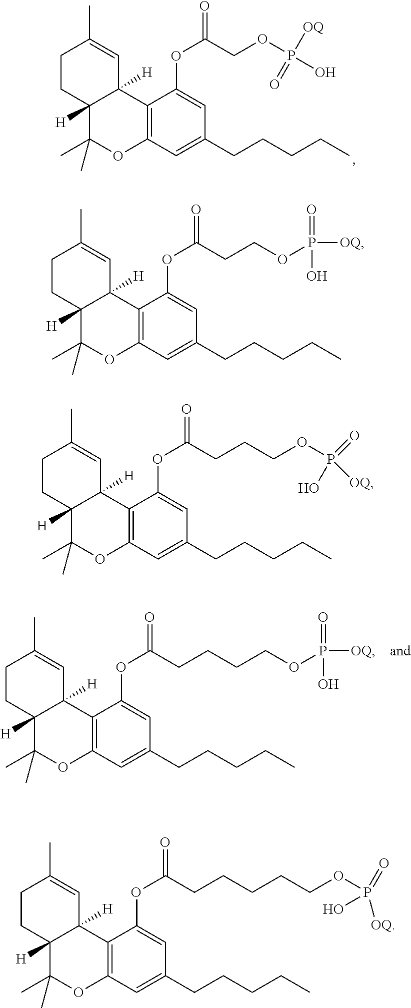

In some embodiments, the compound of Formula (III)-1A is chosen from compounds having the following formulae:

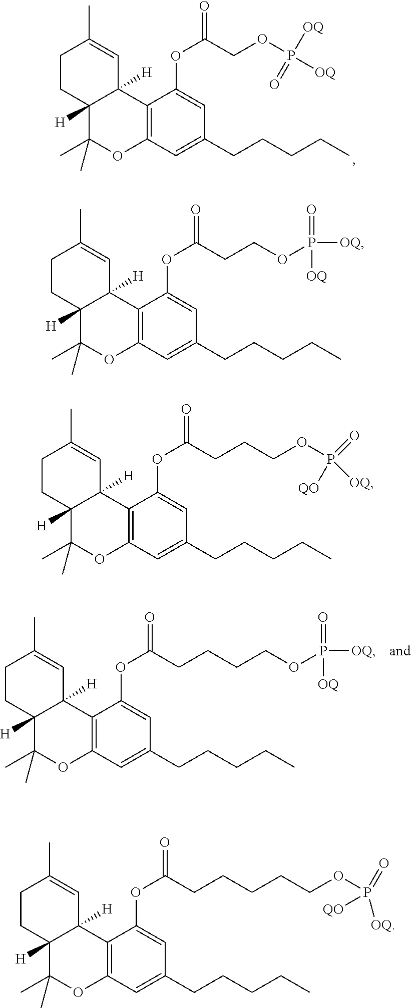

In some embodiments, the compound of Formula (III)-1A is chosen from compounds having the following formulae:

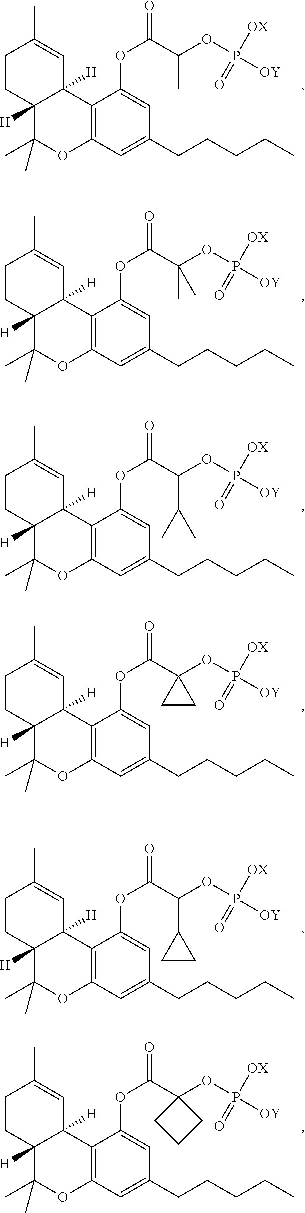

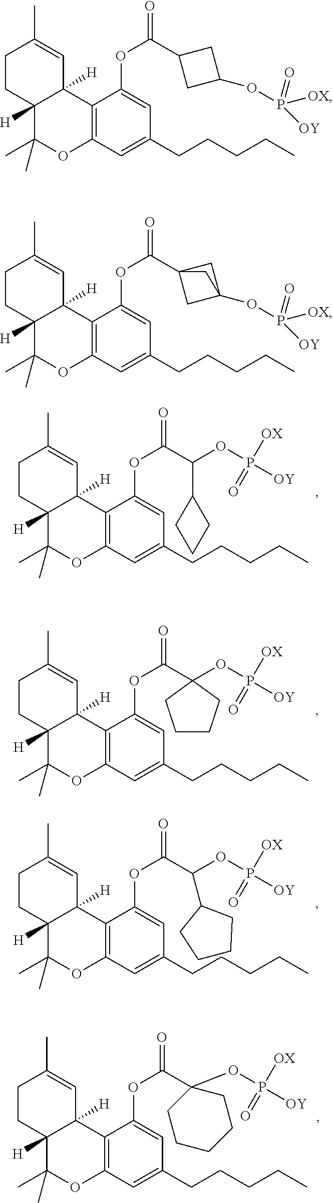

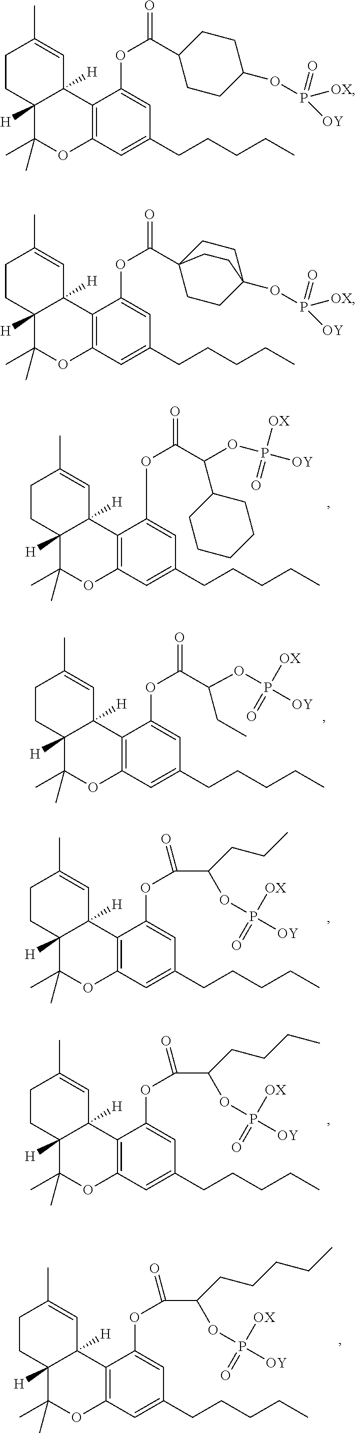

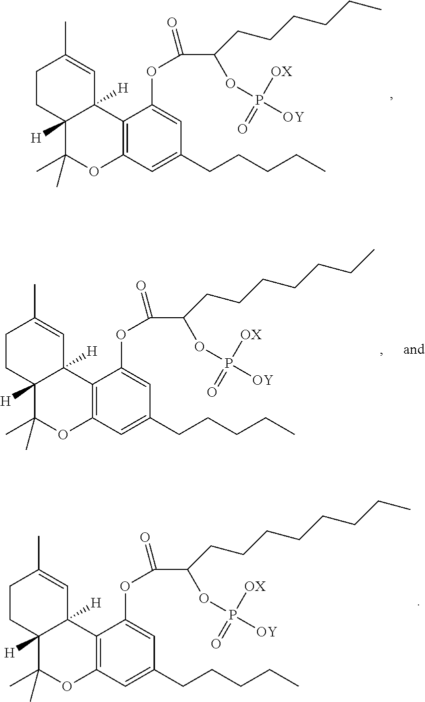

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl C₇₋₁₉ arylalkyl C₂₋₁₄ heteroarylalkyl, —(CH₂CH₂O)ₙCH₃,

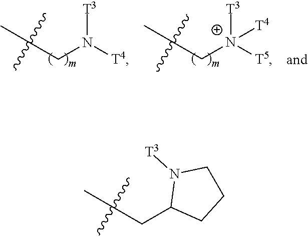

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

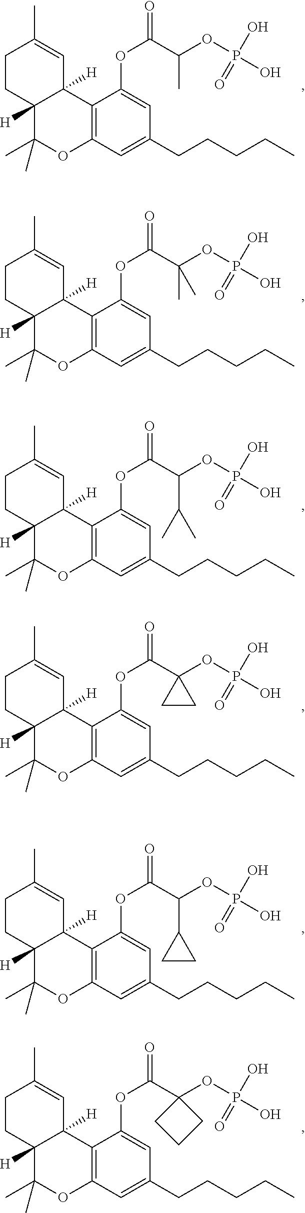

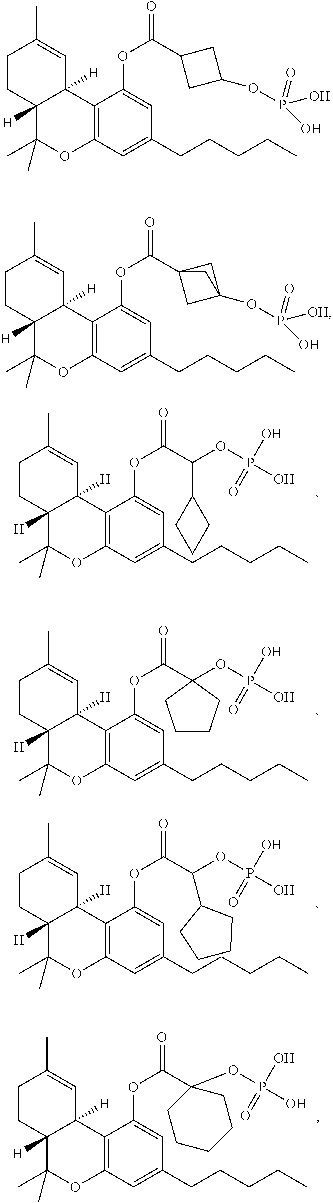

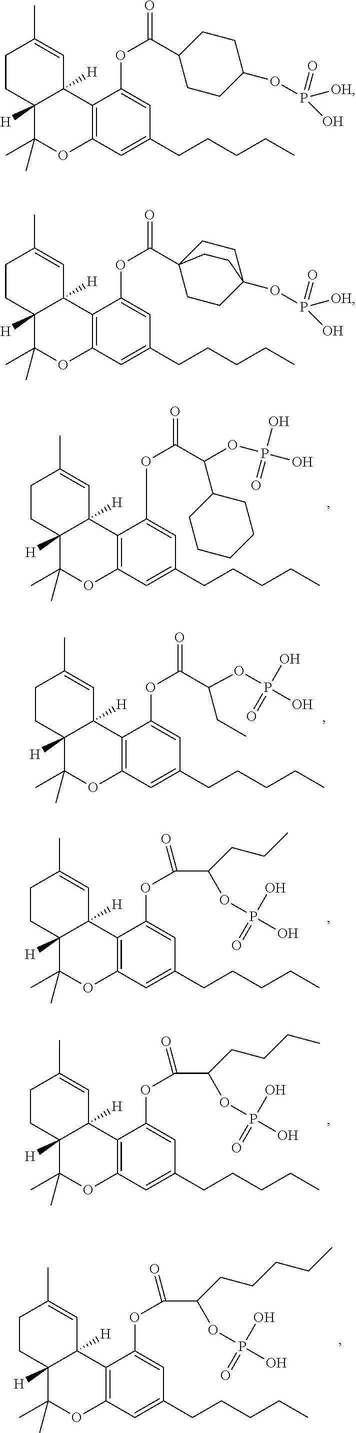

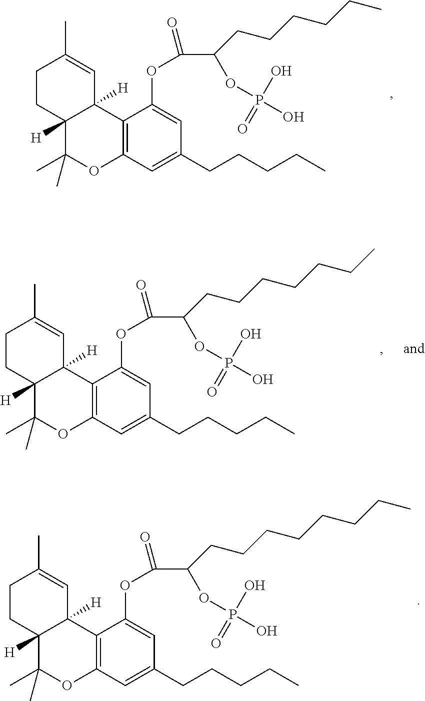 [SMILES: CCCCCCC(OP(=O)(O)O)C(=O)Oc1cc(CCCCC)cc2c1[C@@H]1C=C(C)CC[C@H]1C(C)(C)O2 ; CCCCCCCC(OP(=O)(O)O)C(=O)Oc1cc(CCCCC)cc2c1[C@@H]1C=C(C)CC[C@H]1C(C)(C)O2 ; CCCCCCCCC(OP(=O)(O)O)C(=O)Oc1cc(CCCCC)cc2c1[C@@H]1C=C(C)CC[C@H]1C(C)(C)O2]

In some embodiments, the compound of Formula (III)-1A is chosen from compounds having the following formulae:

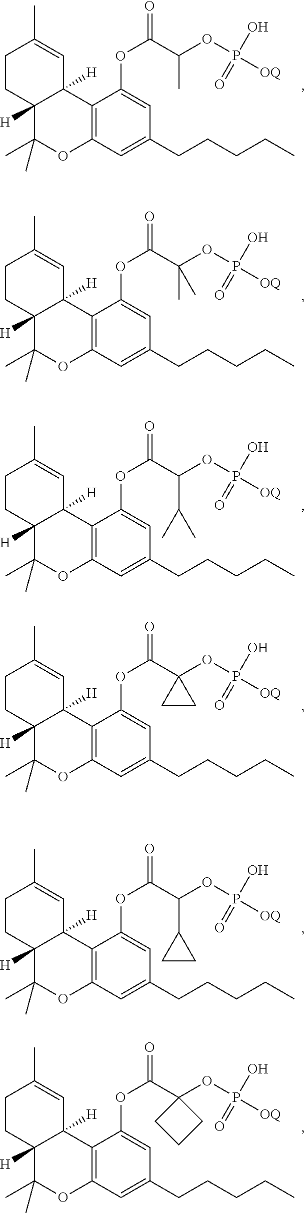

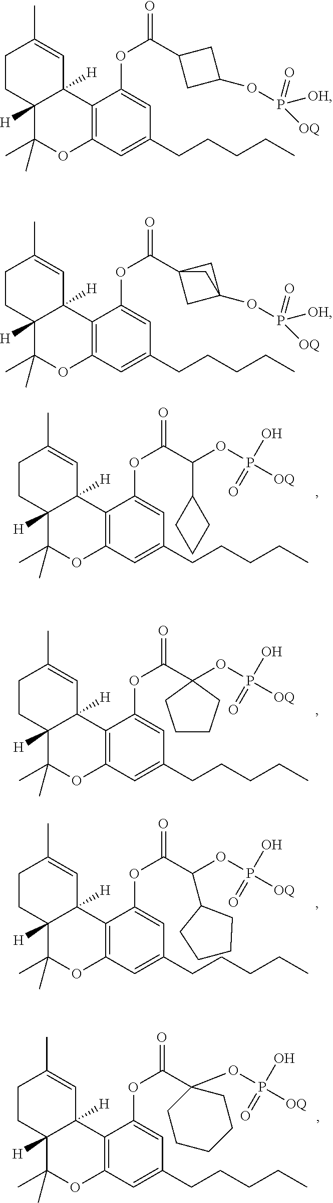

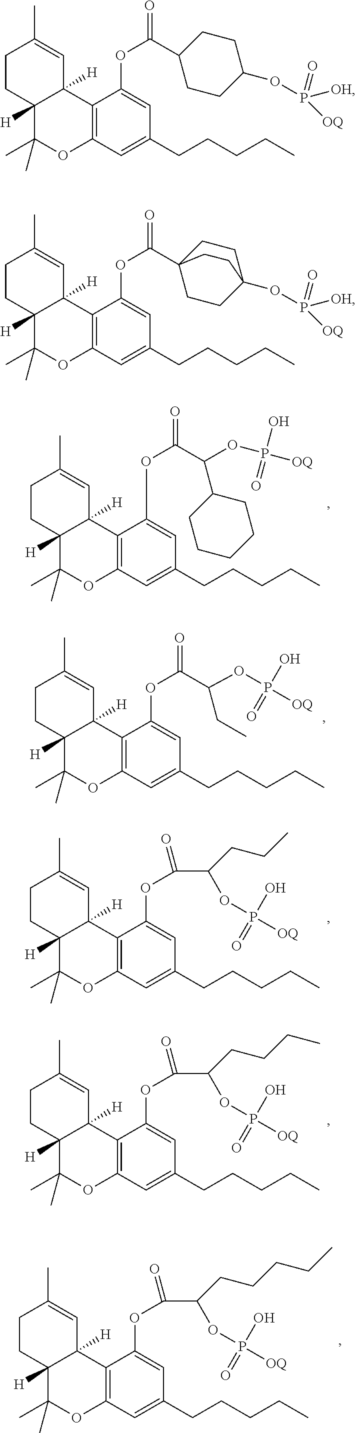

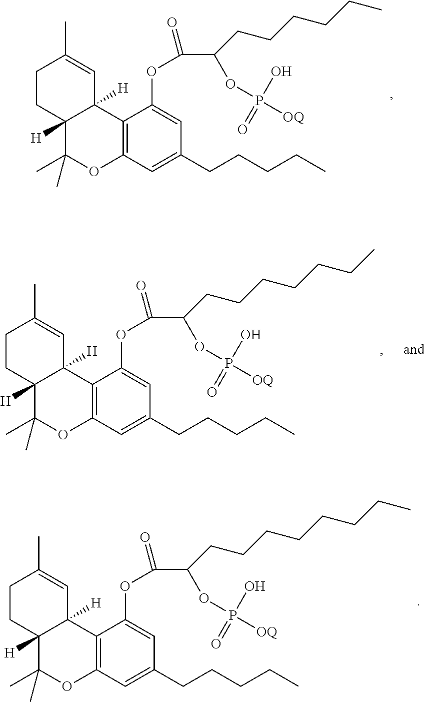

In some embodiments, the compound of Formula (III)-1A is chosen from compounds having the following formulae:

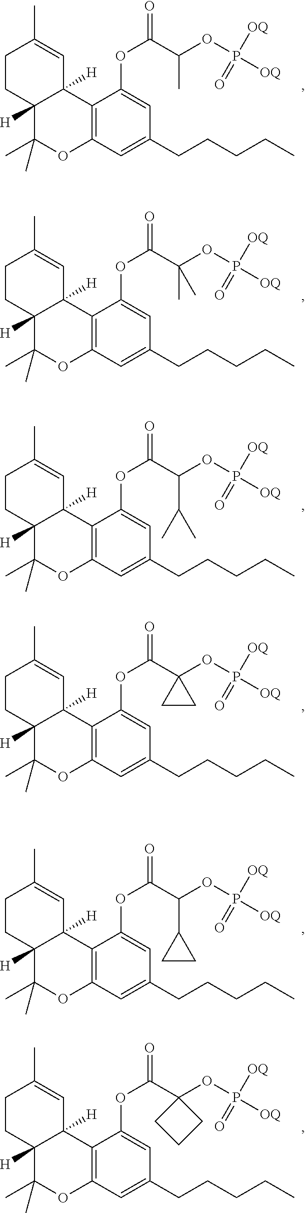

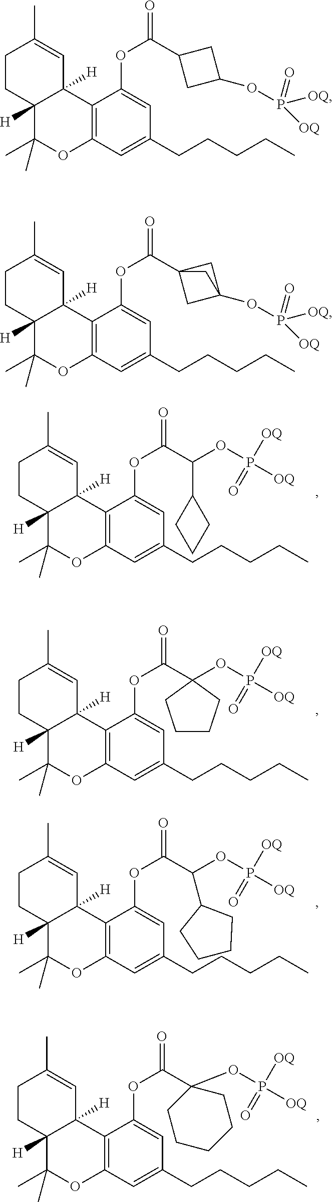

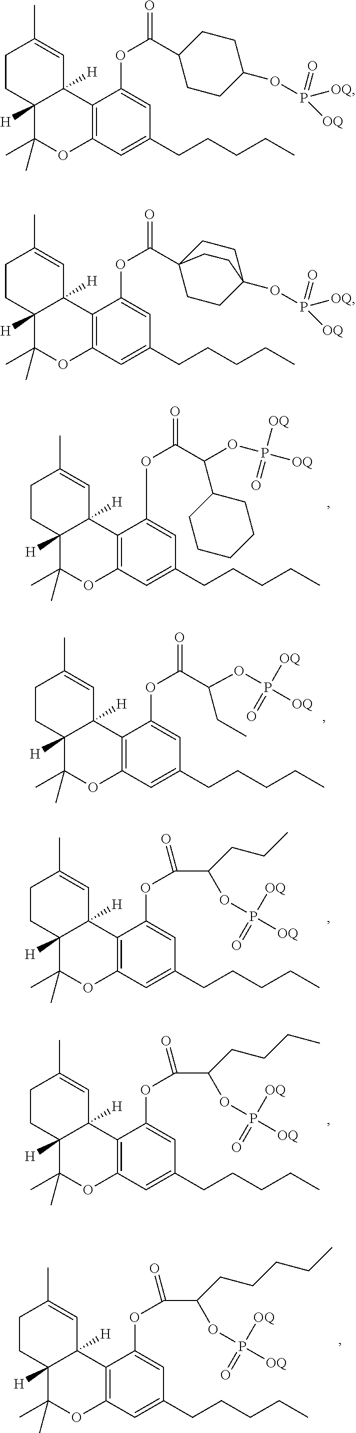

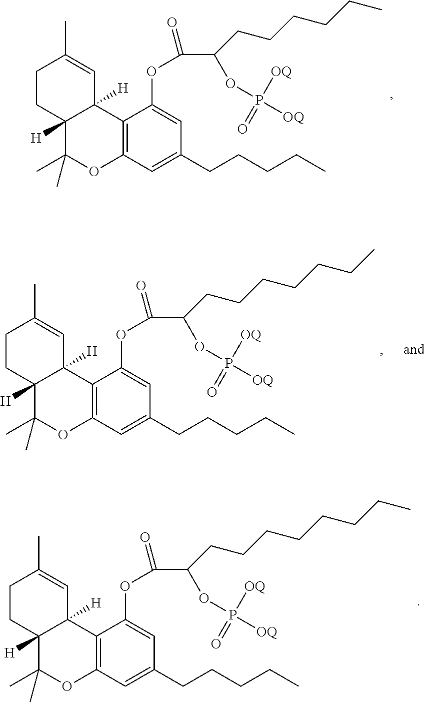

In some embodiments the compound of Formula (III)-1A is chosen from compounds having the following formulae:

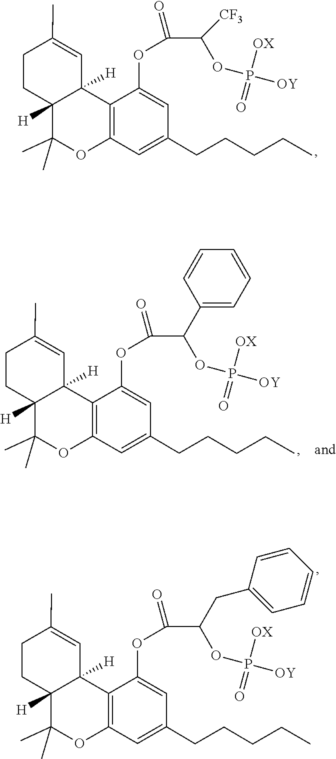

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

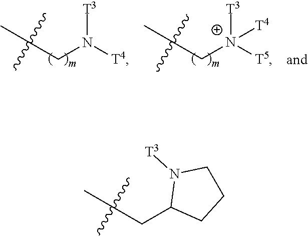

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

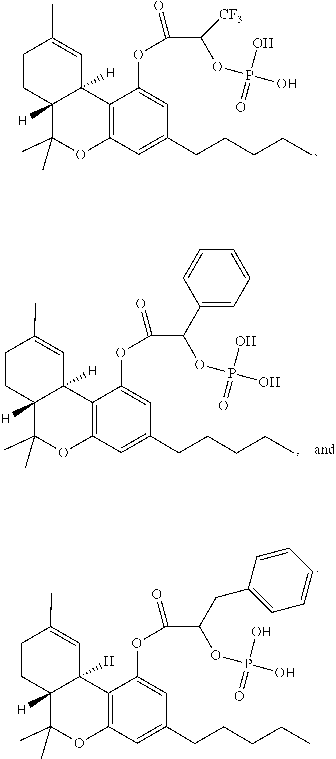

In some embodiments, the compound of Formula (III)-1A is chosen from compounds having the following formulae:

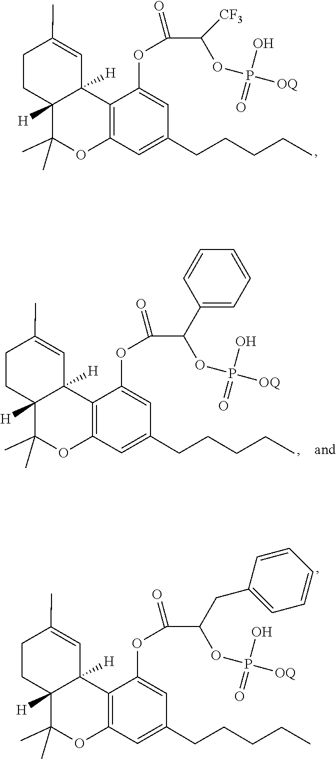

In some embodiments, the compound of Formula (III)-1A is chosen from compounds having the following formulae:

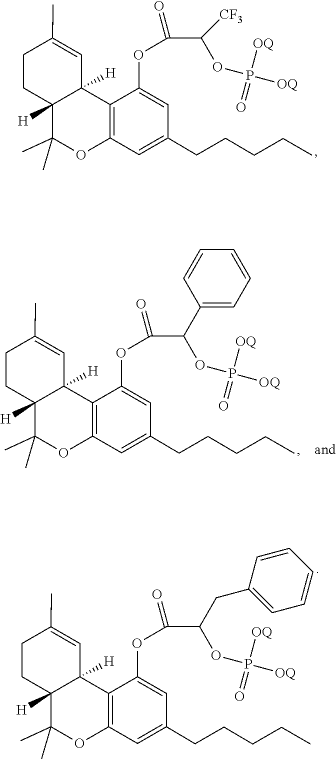

In some embodiments, the compound of Formula (III)-1A is chosen from compounds having the following formulae:

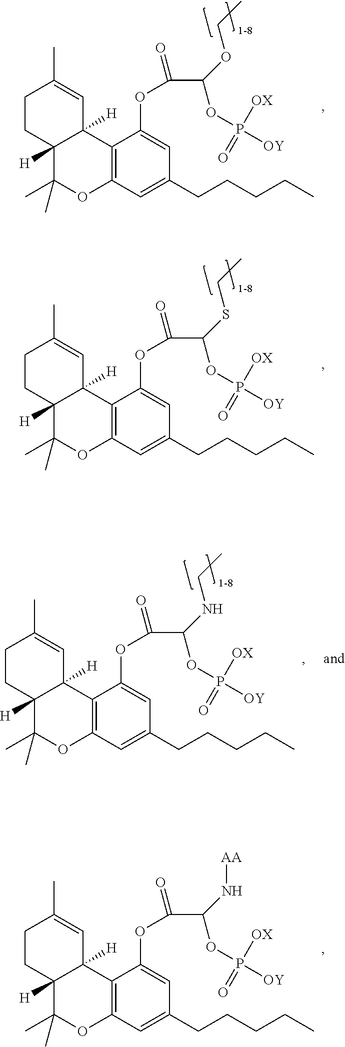

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂)ₙCH₃,

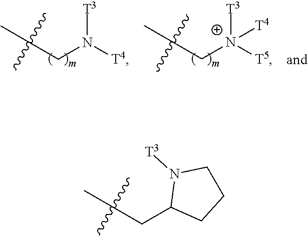

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

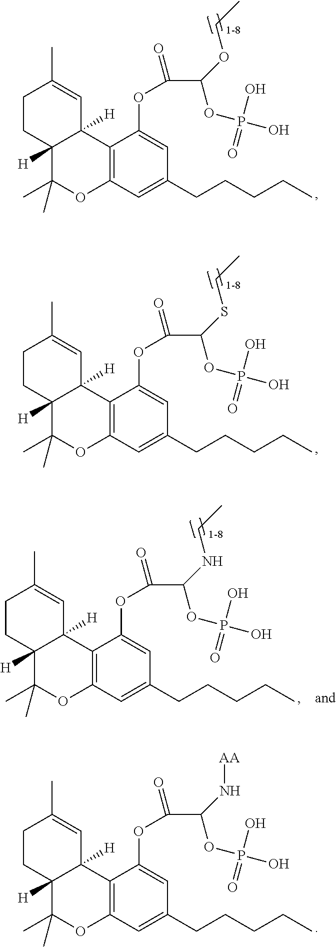

In some embodiments, the compound of Formula (III)-1A is chosen from compounds having the following formulae:

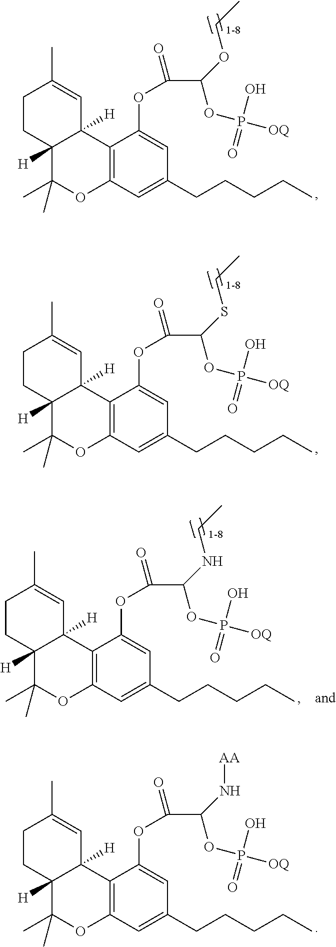

In some embodiments, the compound of Formula (III)-1A is chosen from compounds having the following formulae:

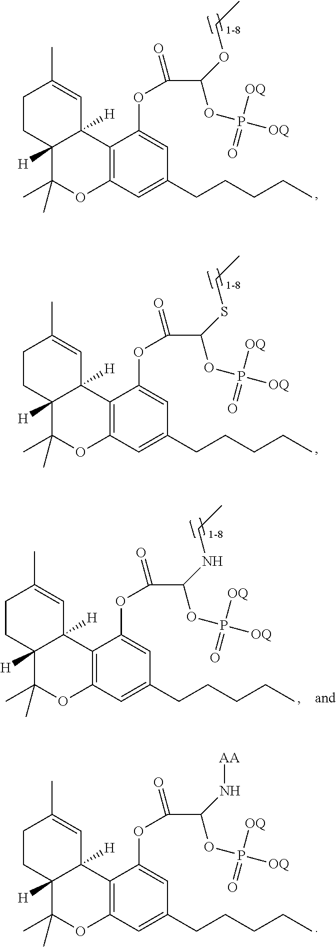

In some embodiments, the compound of Formula (III)-1A is chosen from compounds having the following formulae:

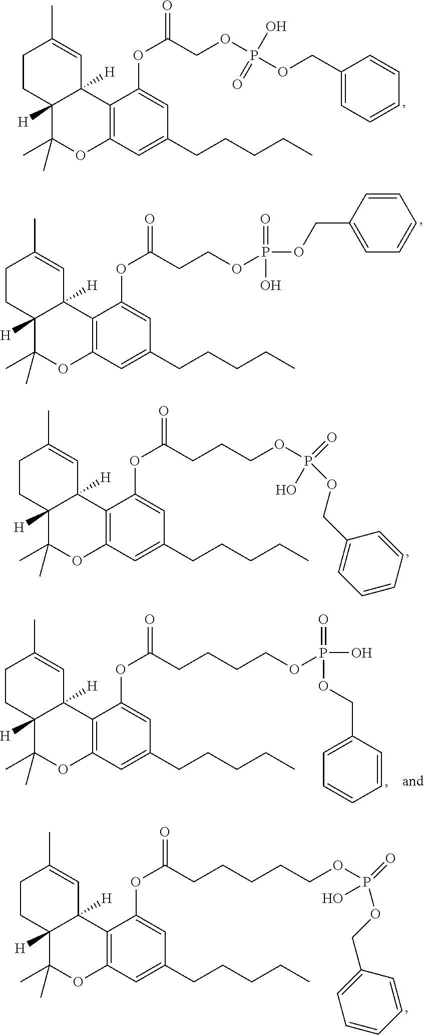

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

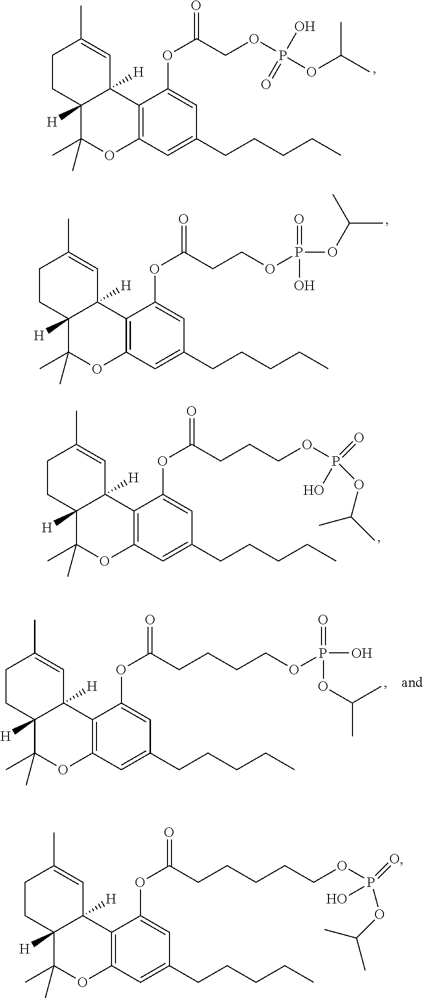

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

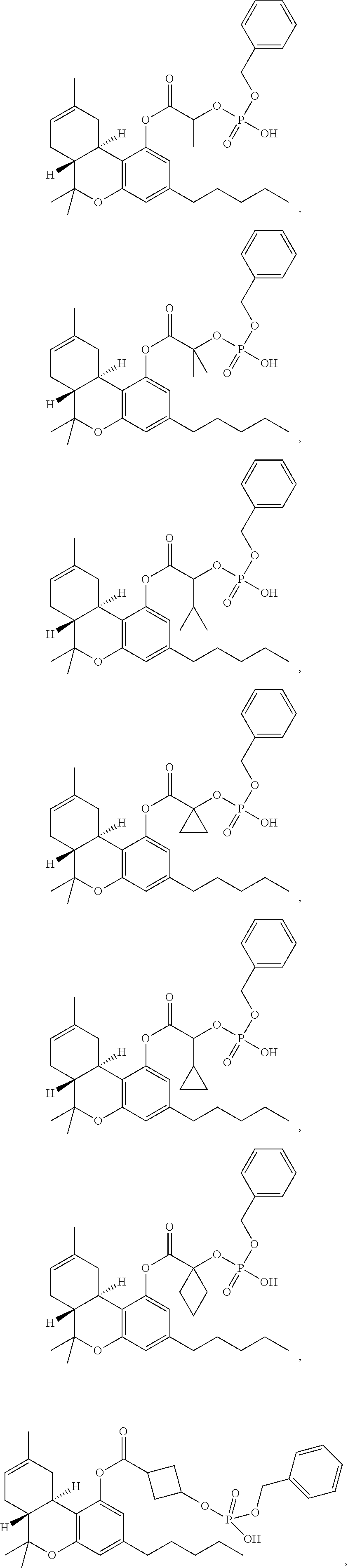

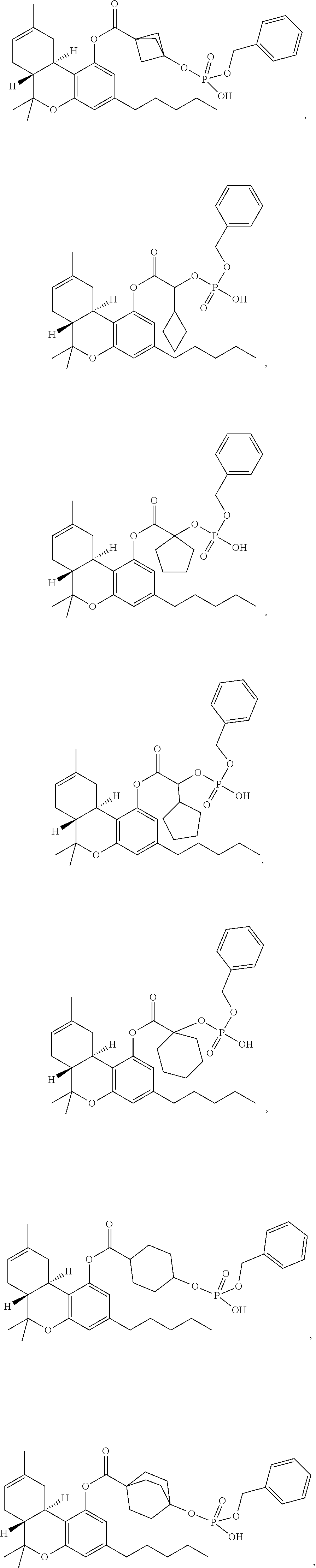

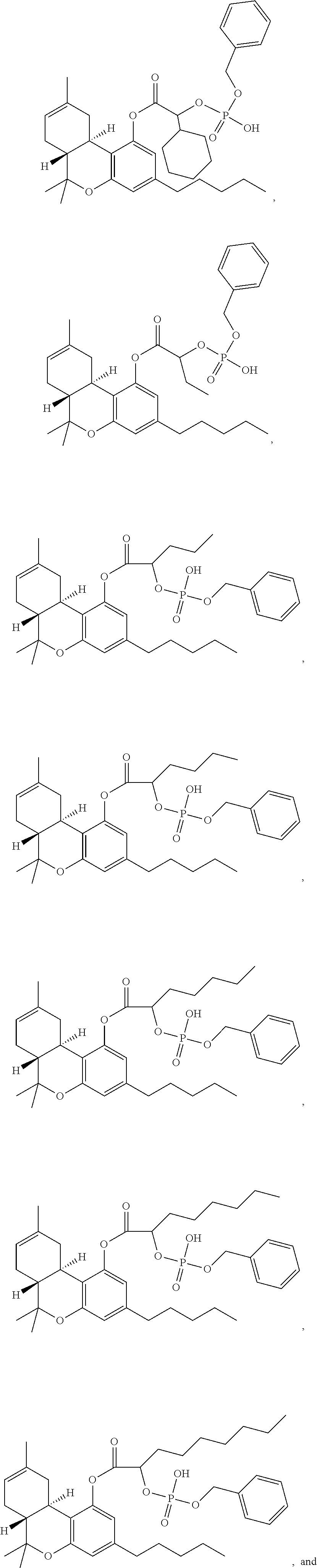

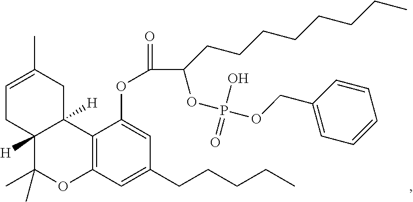 [SMILES: CCCCCCCCC(OP(=O)(O)OCc1ccccc1)C(=O)Oc1cc(CCCCC)cc2c1[C@@H]1CC(C)=CC[C@H]1C(C)(C)O2]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

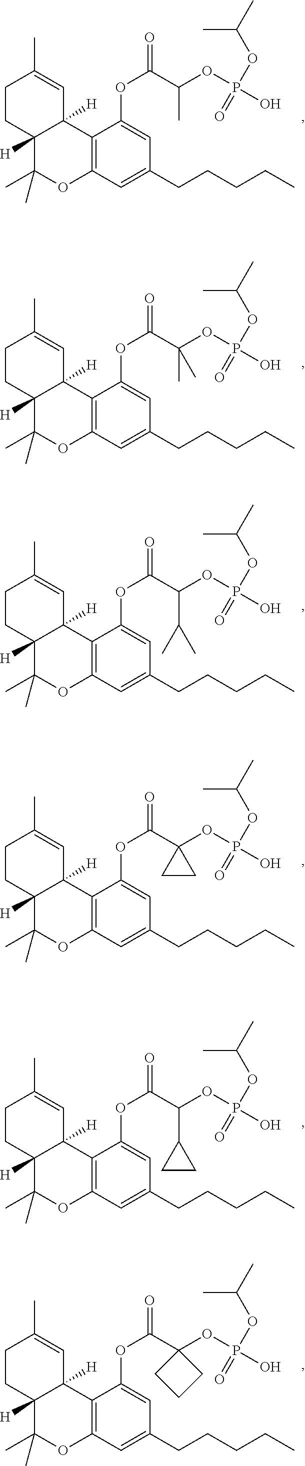 [SMILES: CCCCCc1cc(OC(=O)C(C)OP(=O)(O)OC(C)C)c2c(c1)OC(C)(C)[C@@H]1CCC(C)=C[C@@H]21 ; CCCCCc1cc(OC(=O)C(C)(C)OP(=O)(O)OC(C)C)c2c(c1)OC(C)(C)[C@@H]1CCC(C)=C[C@@H]21 ; CCCCCc1cc(OC(=O)C(OP(=O)(O)OC(C)C)C(C)C)c2c(c1)OC(C)(C)[C@@H]1CCC(C)=C[C@@H]21 ; CCCCCc1cc(OC(=O)C2(OP(=O)(O)OC(C)C)CC2)c2c(c1)OC(C)(C)[C@@H]1CCC(C)=C[C@@H]21 ; CCCCCc1cc(OC(=O)C(OP(=O)(O)OC(C)C)C2CC2)c2c(c1)OC(C)(C)[C@@H]1CCC(C)=C[C@@H]21 ; CCCCCc1cc(OC(=O)C2(OP(=O)(O)OC(C)C)CCC2)c2c(c1)OC(C)(C)[C@@H]1CCC(C)=C[C@@H]21]

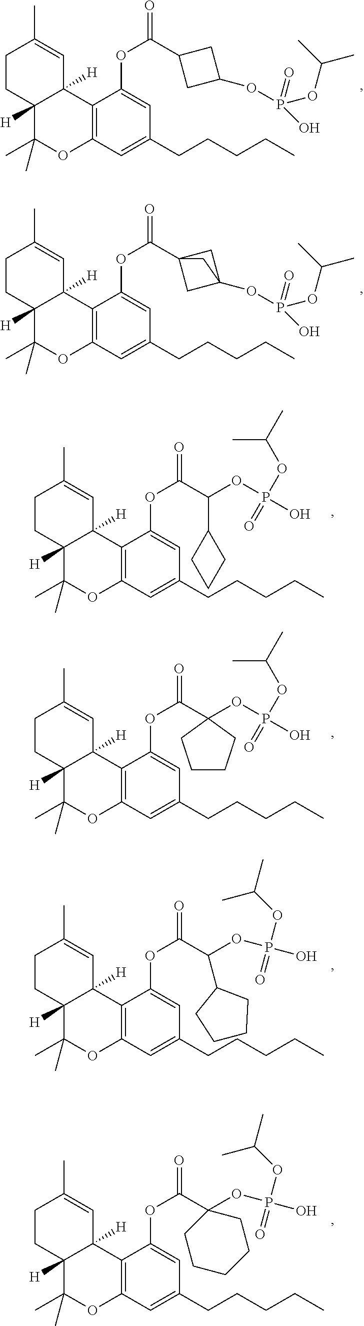

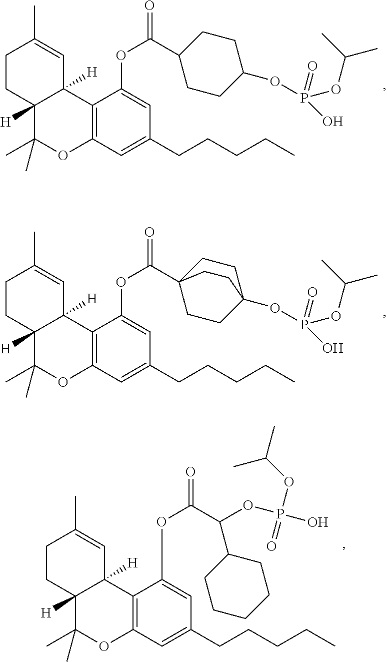 [SMILES: CCCCCc1cc(OC(=O)C2CCC(OP(=O)(O)OC(C)C)CC2)c2c(c1)OC(C)(C)[C@@H]1CCC(C)=C[C@@H]21 ; CCCCCc1cc(OC(=O)C23CCC(OP(=O)(O)OC(C)C)(CC2)CC3)c2c(c1)OC(C)(C)[C@@H]1CCC(C)=C[C@@H]21 ; CCCCCc1cc(OC(=O)C(OP(=O)(O)OC(C)C)C2CCCCC2)c2c(c1)OC(C)(C)[C@@H]1CCC(C)=C[C@@H]21]

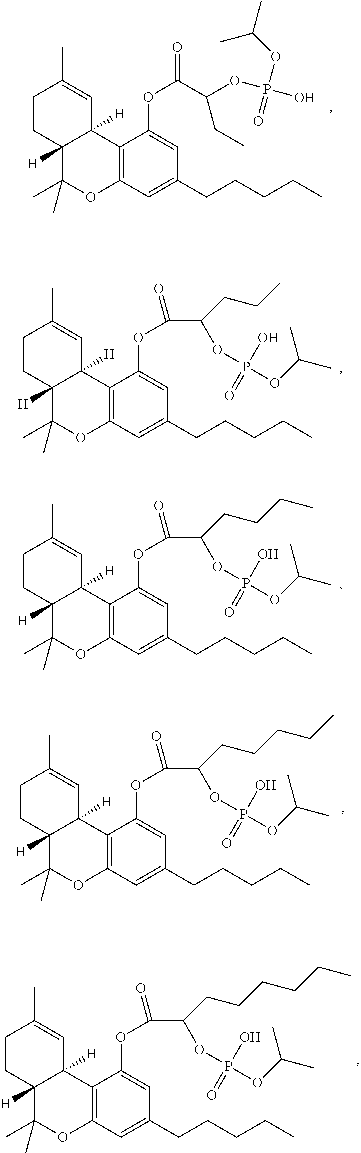 [SMILES: CCCCCc1cc(OC(=O)C(CC)OP(=O)(O)OC(C)C)c2c(c1)OC(C)(C)[C@@H]1CCC(C)=C[C@@H]21 ; CCCCCc1cc(OC(=O)C(CCC)OP(=O)(O)OC(C)C)c2c(c1)OC(C)(C)[C@@H]1CCC(C)=C[C@@H]21 ; CCCCCc1cc(OC(=O)C(CCCC)OP(=O)(O)OC(C)C)c2c(c1)OC(C)(C)[C@@H]1CCC(C)=C[C@@H]21 ; CCCCCc1cc(OC(=O)C(CCCCC)OP(=O)(O)OC(C)C)c2c(c1)OC(C)(C)[C@@H]1CCC(C)=C[C@@H]21 ; CCCCCCC(OP(=O)(O)OC(C)C)C(=O)Oc1cc(CCCCC)cc2c1[C@@H]1C=C(C)CC[C@H]1C(C)(C)O2]

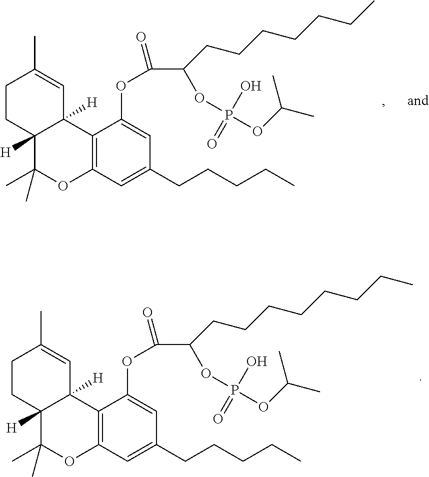 [SMILES: CCCCCCCC(OP(=O)(O)OC(C)C)C(=O)Oc1cc(CCCCC)cc2c1[C@@H]1C=C(C)CC[C@H]1C(C)(C)O2 ; CCCCCCCCC(OP(=O)(O)OC(C)C)C(=O)Oc1cc(CCCCC)cc2c1[C@@H]1C=C(C)CC[C@H]1C(C)(C)O2]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

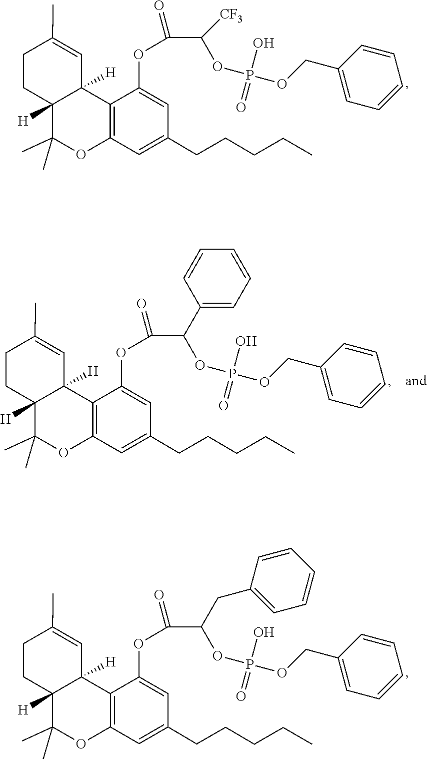 [SMILES: CCCCCc1cc(OC(=O)C(OP(=O)(O)OCc2ccccc2)C(F)(F)F)c2c(c1)OC(C)(C)[C@@H]1CCC(C)=C[C@@H]21 ; CCCCCc1cc(OC(=O)C(OP(=O)(O)OCc2ccccc2)c2ccccc2)c2c(c1)OC(C)(C)[C@@H]1CCC(C)=C[C@@H]21 ; CCCCCc1cc(OC(=O)C(Cc2ccccc2)OP(=O)(O)OCc2ccccc2)c2c(c1)OC(C)(C)[C@@H]1CCC(C)=C[C@@H]21]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

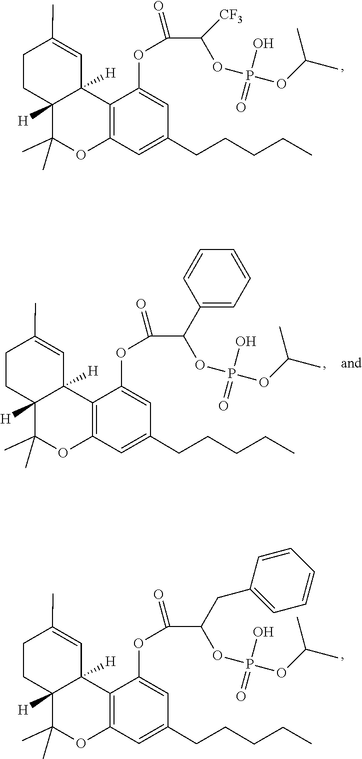

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

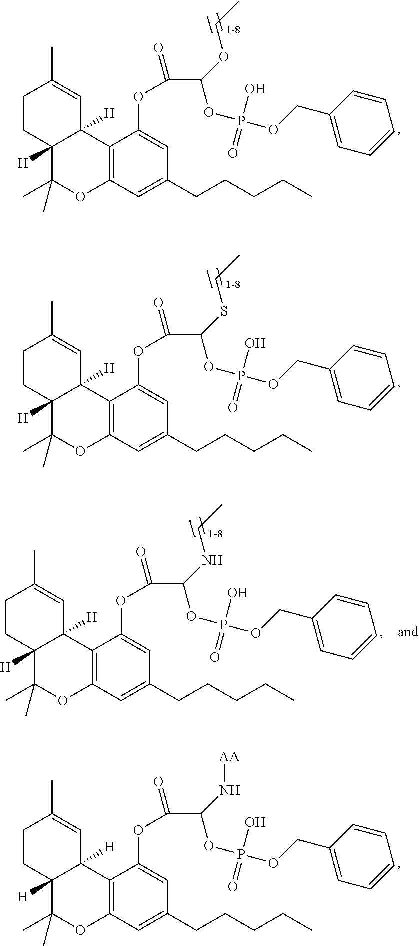

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

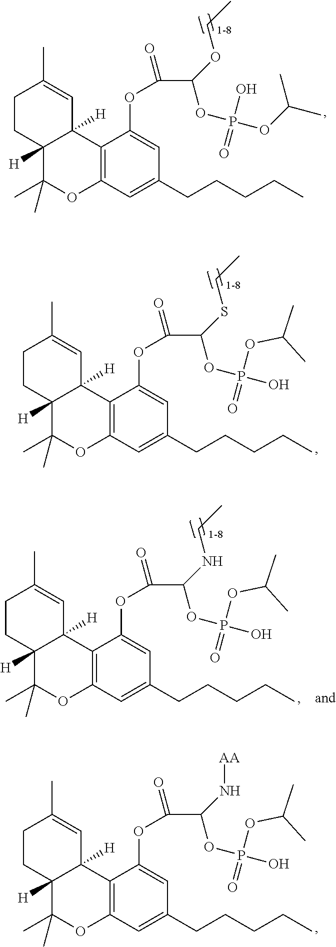

and pharmaceutically acceptable salts of any of the foregoing. In some embodiments, the pharmaceutically acceptable salts are ammonium salts.

In some embodiments, the compound of Formula (III)-1A is chosen from compounds having the following formula:

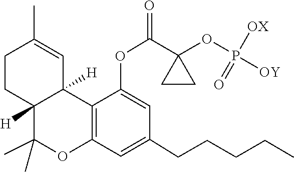

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

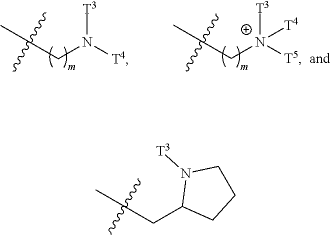

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formula:

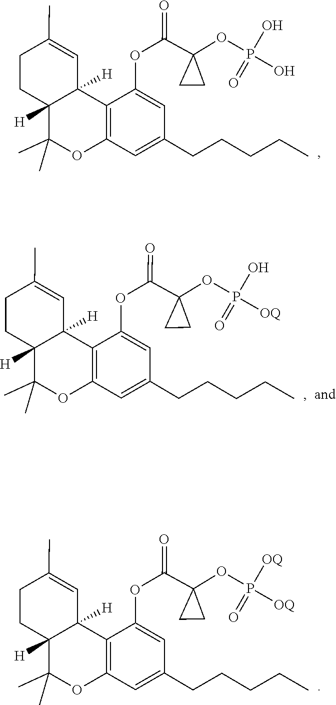

In some embodiments, the compound of Formula (III)-1A is chosen from compounds having the following formula:

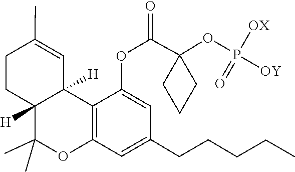

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₃ heteroaryl, C₇₋₁₉ arylalkyl, C₂₋₁₄ heteroarylalkyl, —(CH₂CH₂O)ₙCH₃,

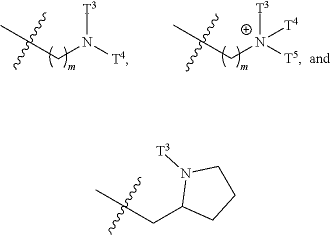

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

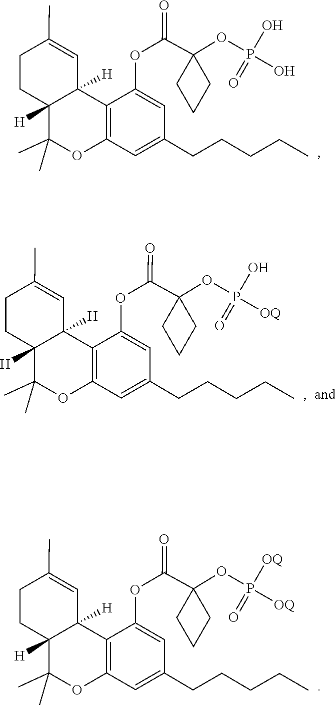

In some embodiments, the compound of Formula (III)-1A is chosen from compounds having the following formula:

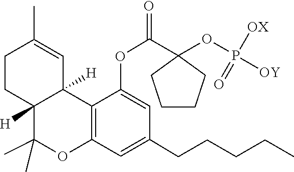

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ is alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

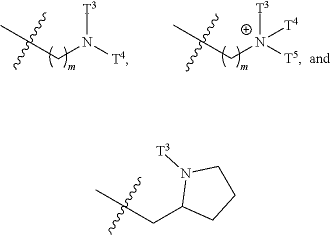

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

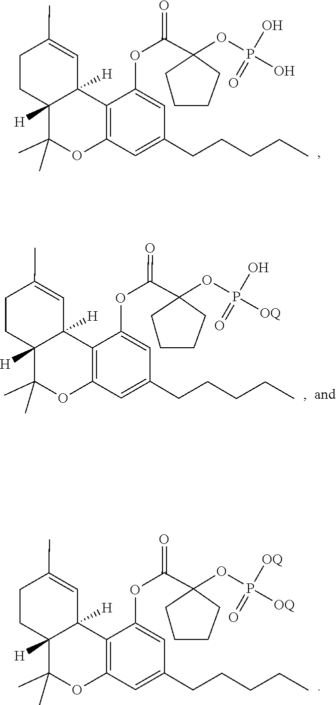

In some embodiments, the compound of Formula (III)-1A is chosen from compounds having the following formula:

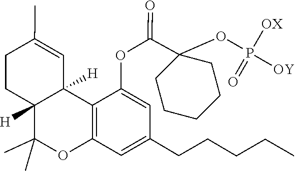

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

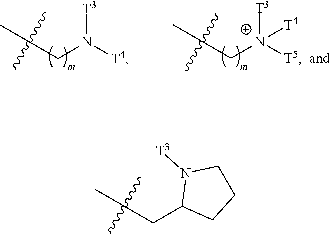

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

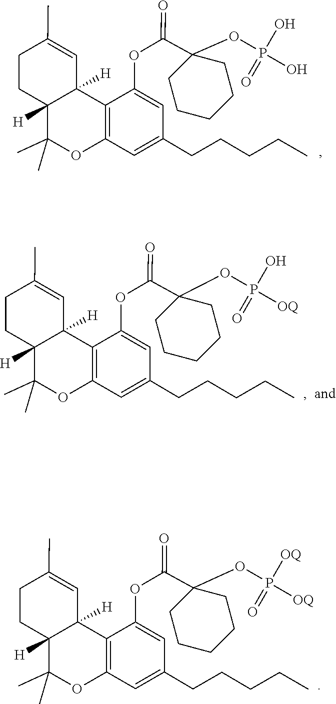

In some embodiments, the compound of Formula (III)-1A is chosen from compounds having the following formula:

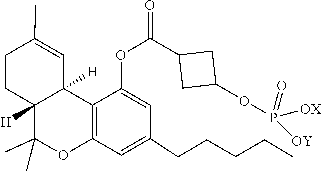

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂)ₙCH₃,

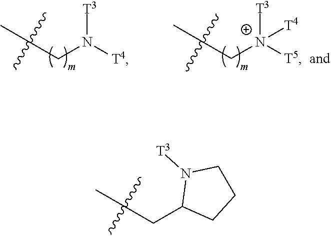

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

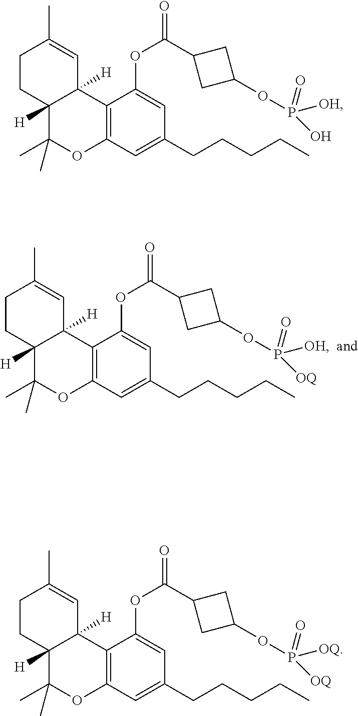

In some embodiments, the compound of Formula (III)-1A is chosen from compounds having the following formula:

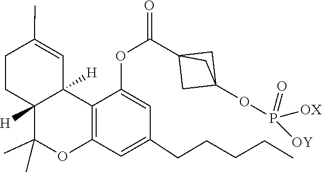

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

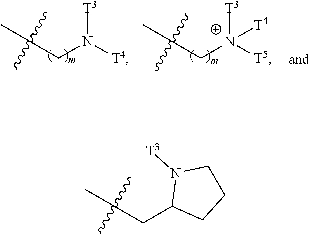

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

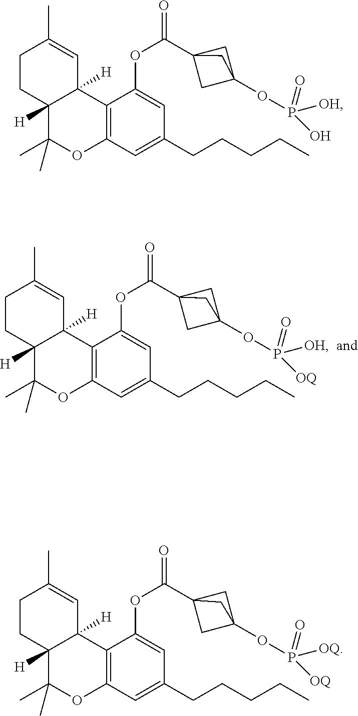

In some embodiments, the compound of Formula (III)-1A is chosen from compounds having the following formula:

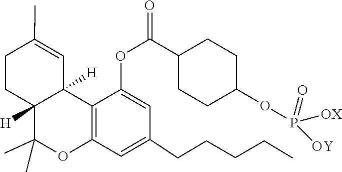

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

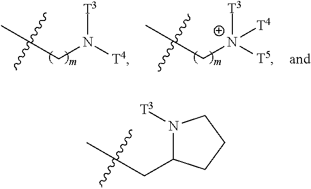

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae

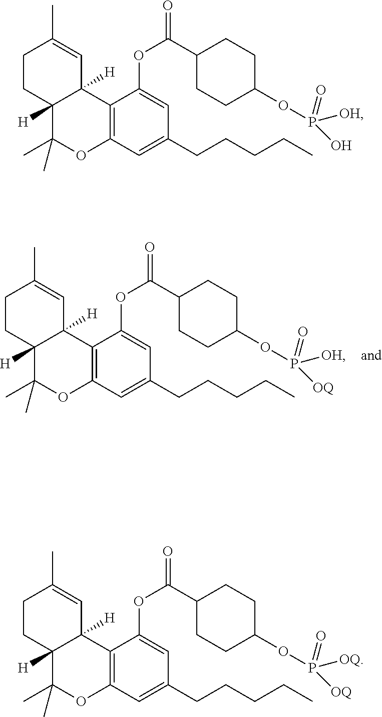

In some embodiments, the compound of Formula (III)-1A is chosen from compounds having the following formula:

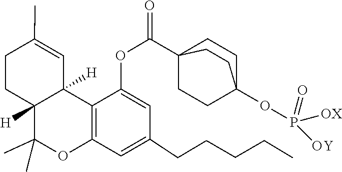

and pharmaceutically acceptable salts thereof. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

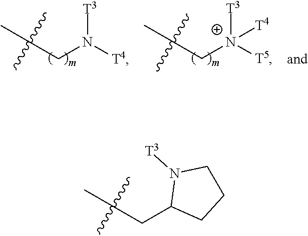

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

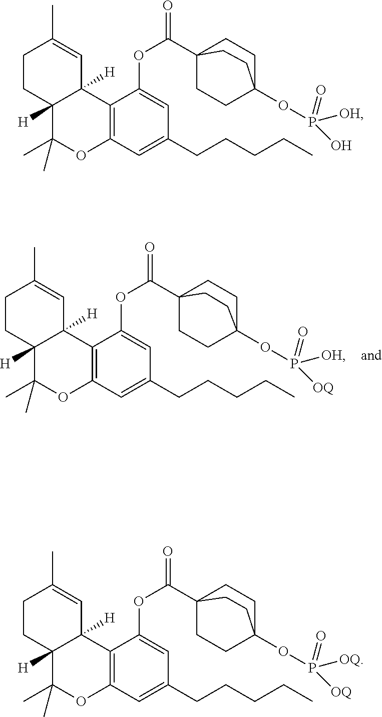

In some embodiments, the compound of Formula (III)-1A is chosen from compounds having the following formulae:

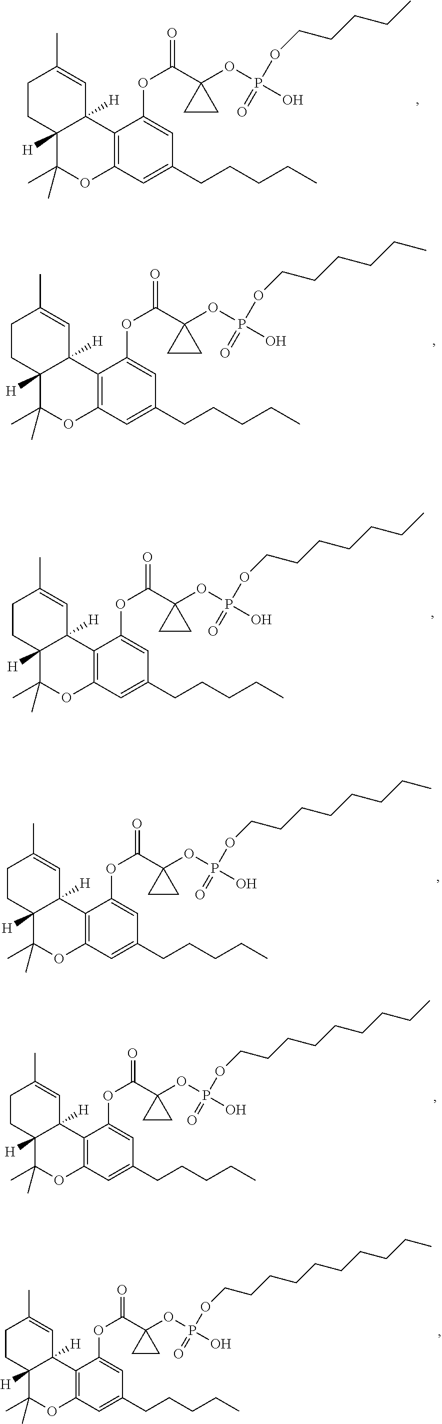 [SMILES: CCCCCOP(=O)(O)OC1(C(=O)Oc2cc(CCCCC)cc3c2[C@@H]2C=C(C)CC[C@H]2C(C)(C)O3)CC1 ; CCCCCCOP(=O)(O)OC1(C(=O)Oc2cc(CCCCC)cc3c2[C@@H]2C=C(C)CC[C@H]2C(C)(C)O3)CC1 ; CCCCCCCOP(=O)(O)OC1(C(=O)Oc2cc(CCCCC)cc3c2[C@@H]2C=C(C)CC[C@H]2C(C)(C)O3)CC1 ; CCCCCCCCOP(=O)(O)OC1(C(=O)Oc2cc(CCCCC)cc3c2[C@@H]2C=C(C)CC[C@H]2C(C)(C)O3)CC1 ; CCCCCCCCCOP(=O)(O)OC1(C(=O)Oc2cc(CCCCC)cc3c2[C@@H]2C=C(C)CC[C@H]2C(C)(C)O3)CC1 ; CCCCCCCCCCOP(=O)(O)OC1(C(=O)Oc2cc(CCCCC)cc3c2[C@@H]2C=C(C)CC[C@H]2C(C)(C)O3)CC1]

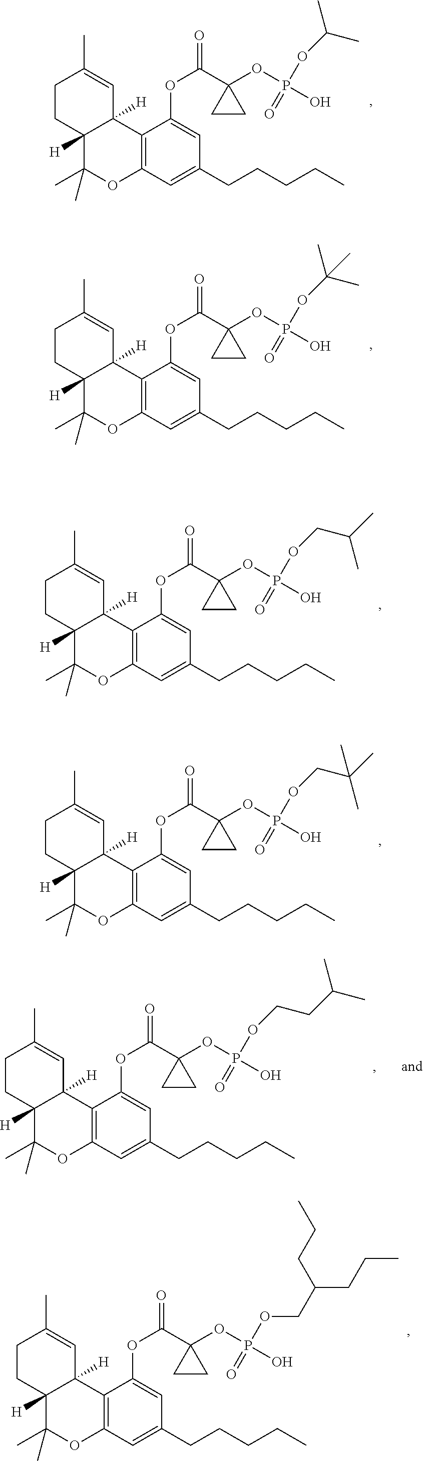

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

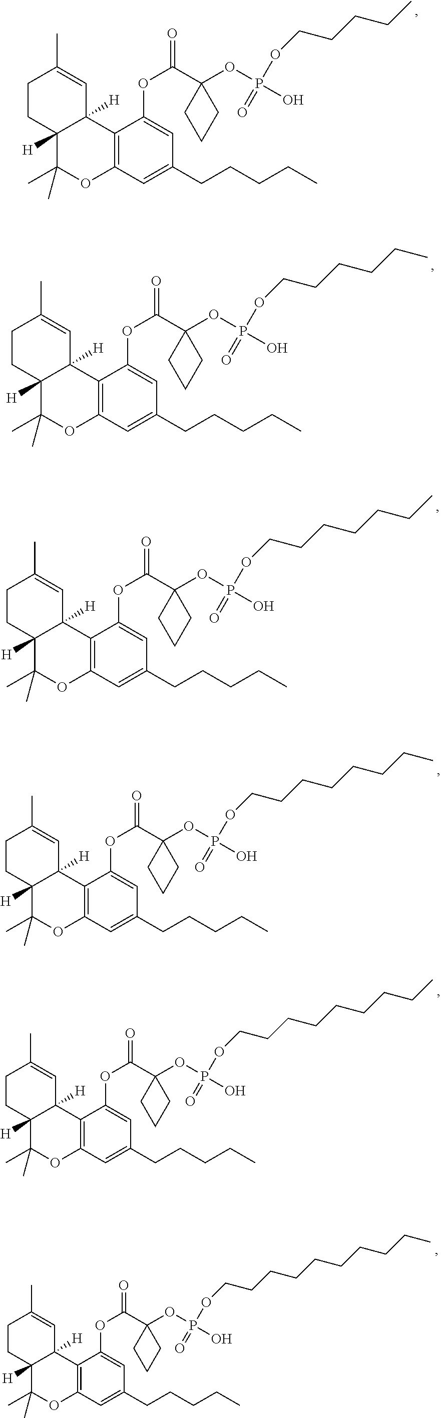 [SMILES: CCCCCOP(=O)(O)OC1(C(=O)Oc2cc(CCCCC)cc3c2[C@@H]2C=C(C)CC[C@H]2C(C)(C)O3)CCC1 ; CCCCCCOP(=O)(O)OC1(C(=O)Oc2cc(CCCCC)cc3c2[C@@H]2C=C(C)CC[C@H]2C(C)(C)O3)CCC1 ; CCCCCCCOP(=O)(O)OC1(C(=O)Oc2cc(CCCCC)cc3c2[C@@H]2C=C(C)CC[C@H]2C(C)(C)O3)CCC1 ; CCCCCCCCOP(=O)(O)OC1(C(=O)Oc2cc(CCCCC)cc3c2[C@@H]2C=C(C)CC[C@H]2C(C)(C)O3)CCC1 ; CCCCCCCCCOP(=O)(O)OC1(C(=O)Oc2cc(CCCCC)cc3c2[C@@H]2C=C(C)CC[C@H]2C(C)(C)O3)CCC1 ; CCCCCCCCCCOP(=O)(O)OC1(C(=O)Oc2cc(CCCCC)cc3c2[C@@H]2C=C(C)CC[C@H]2C(C)(C)O3)CCC1]

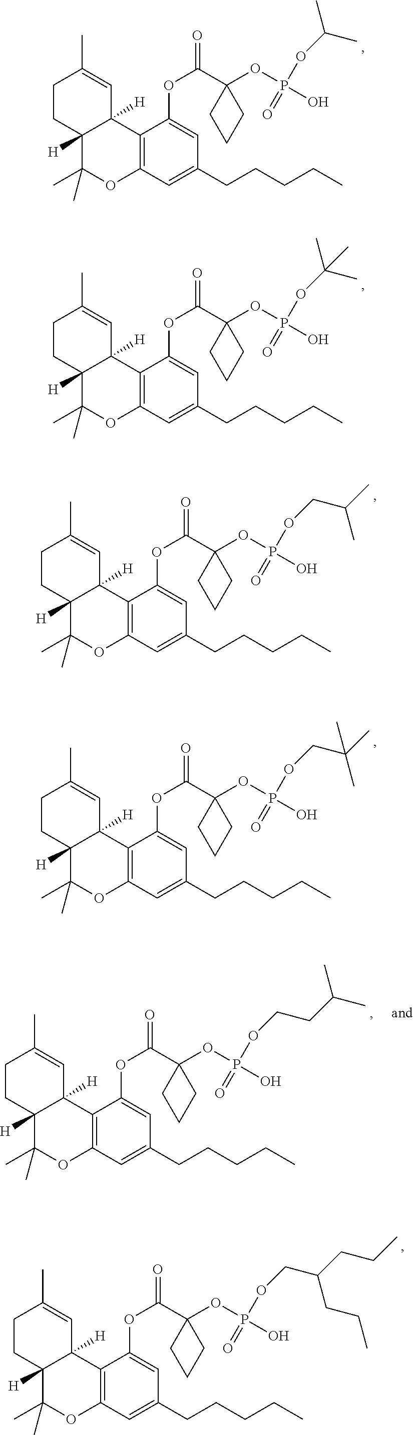 [SMILES: CCCCCc1cc(OC(=O)C2(OP(=O)(O)OC(C)C)CCC2)c2c(c1)OC(C)(C)[C@@H]1CCC(C)=C[C@@H]21 ; CCCCCc1cc(OC(=O)C2(OP(=O)(O)OC(C)(C)C)CCC2)c2c(c1)OC(C)(C)[C@@H]1CCC(C)=C[C@@H]21 ; CCCCCc1cc(OC(=O)C2(OP(=O)(O)OCC(C)C)CCC2)c2c(c1)OC(C)(C)[C@@H]1CCC(C)=C[C@@H]21 ; CCCCCc1cc(OC(=O)C2(OP(=O)(O)OCC(C)(C)C)CCC2)c2c(c1)OC(C)(C)[C@@H]1CCC(C)=C[C@@H]21 ; CCCCCc1cc(OC(=O)C2(OP(=O)(O)OCCC(C)C)CCC2)c2c(c1)OC(C)(C)[C@@H]1CCC(C)=C[C@@H]21 ; CCCCCc1cc(OC(=O)C2(OP(=O)(O)OCC(CCC)CCC)CCC2)c2c(c1)OC(C)(C)[C@@H]1CCC(C)=C[C@@H]21]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

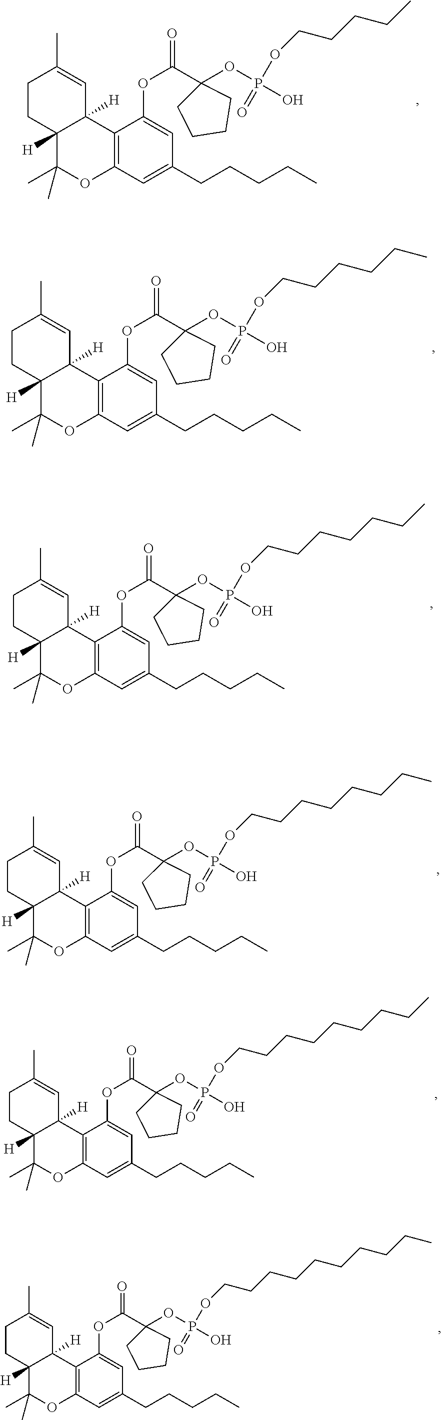 [SMILES: CCCCCOP(=O)(O)OC1(C(=O)Oc2cc(CCCCC)cc3c2[C@@H]2C=C(C)CC[C@H]2C(C)(C)O3)CCCC1 ; CCCCCCOP(=O)(O)OC1(C(=O)Oc2cc(CCCCC)cc3c2[C@@H]2C=C(C)CC[C@H]2C(C)(C)O3)CCCC1 ; CCCCCCCOP(=O)(O)OC1(C(=O)Oc2cc(CCCCC)cc3c2[C@@H]2C=C(C)CC[C@H]2C(C)(C)O3)CCCC1 ; CCCCCCCCOP(=O)(O)OC1(C(=O)Oc2cc(CCCCC)cc3c2[C@@H]2C=C(C)CC[C@H]2C(C)(C)O3)CCCC1 ; CCCCCCCCCOP(=O)(O)OC1(C(=O)Oc2cc(CCCCC)cc3c2[C@@H]2C=C(C)CC[C@H]2C(C)(C)O3)CCCC1 ; CCCCCCCCCCOP(=O)(O)OC1(C(=O)Oc2cc(CCCCC)cc3c2[C@@H]2C=C(C)CC[C@H]2C(C)(C)O3)CCCC1]

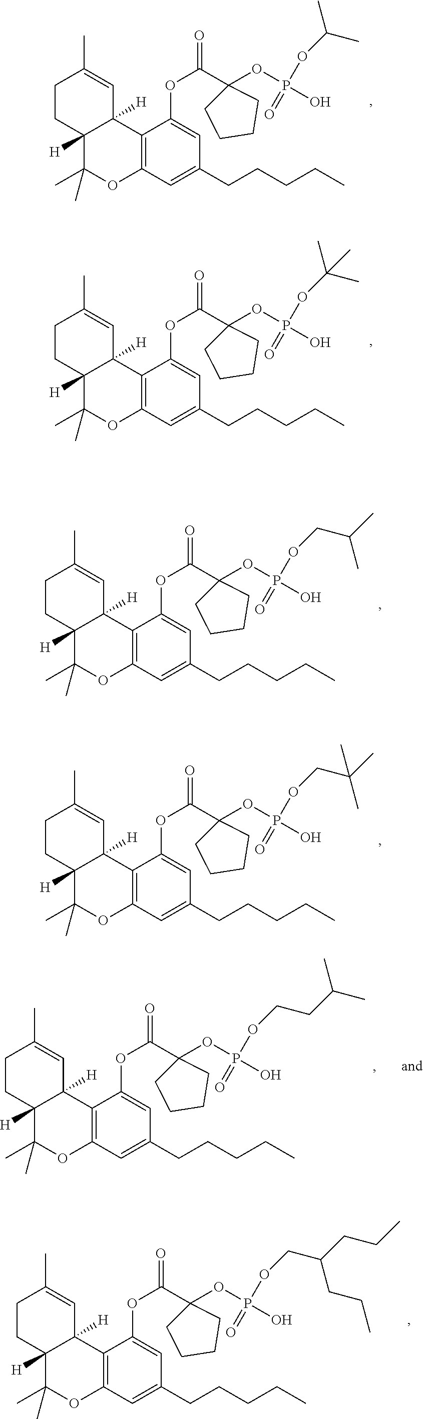

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

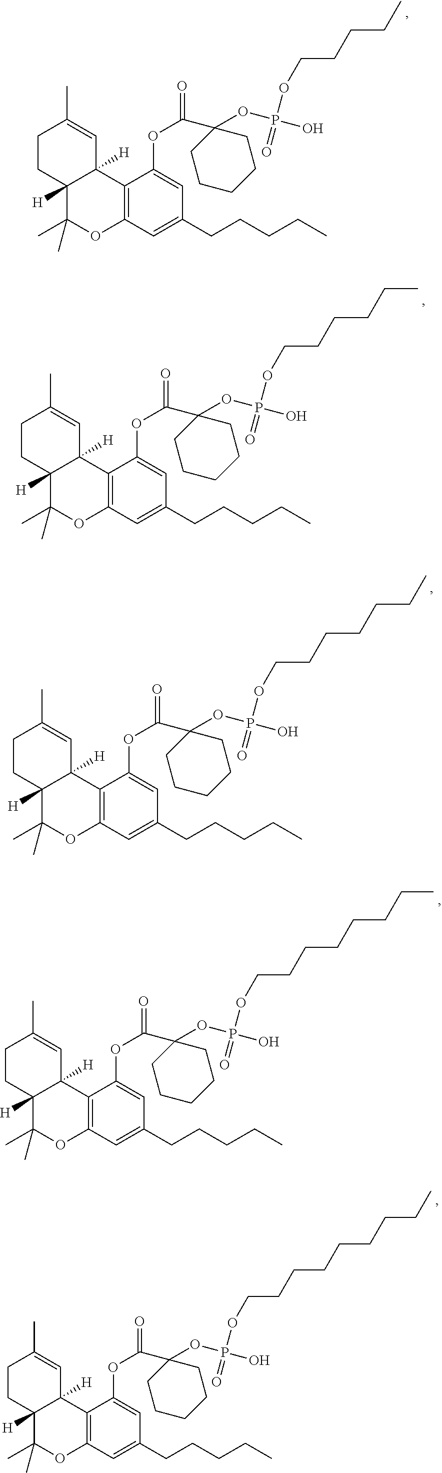 [SMILES: CCCCCOP(=O)(O)OC1(C(=O)Oc2cc(CCCCC)cc3c2[C@@H]2C=C(C)CC[C@H]2C(C)(C)O3)CCCCC1 ; CCCCCCOP(=O)(O)OC1(C(=O)Oc2cc(CCCCC)cc3c2[C@@H]2C=C(C)CC[C@H]2C(C)(C)O3)CCCCC1 ; CCCCCCCOP(=O)(O)OC1(C(=O)Oc2cc(CCCCC)cc3c2[C@@H]2C=C(C)CC[C@H]2C(C)(C)O3)CCCCC1 ; CCCCCCCCOP(=O)(O)OC1(C(=O)Oc2cc(CCCCC)cc3c2[C@@H]2C=C(C)CC[C@H]2C(C)(C)O3)CCCCC1 ; CCCCCCCCCOP(=O)(O)OC1(C(=O)Oc2cc(CCCCC)cc3c2[C@@H]2C=C(C)CC[C@H]2C(C)(C)O3)CCCCC1]

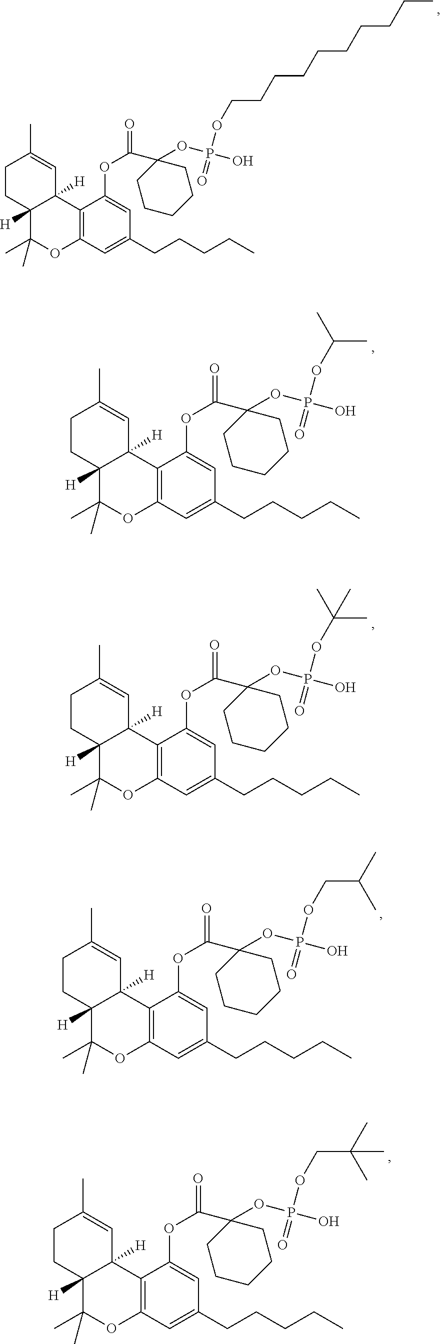 [SMILES: CCCCCCCCCCOP(=O)(O)OC1(C(=O)Oc2cc(CCCCC)cc3c2[C@@H]2C=C(C)CC[C@H]2C(C)(C)O3)CCCCC1 ; CCCCCc1cc(OC(=O)C2(OP(=O)(O)OC(C)C)CCCCC2)c2c(c1)OC(C)(C)[C@@H]1CCC(C)=C[C@@H]21 ; CCCCCc1cc(OC(=O)C2(OP(=O)(O)OC(C)(C)C)CCCCC2)c2c(c1)OC(C)(C)[C@@H]1CCC(C)=C[C@@H]21 ; CCCCCc1cc(OC(=O)C2(OP(=O)(O)OCC(C)C)CCCCC2)c2c(c1)OC(C)(C)[C@@H]1CCC(C)=C[C@@H]21 ; CCCCCc1cc(OC(=O)C2(OP(=O)(O)OCC(C)(C)C)CCCCC2)c2c(c1)OC(C)(C)[C@@H]1CCC(C)=C[C@@H]21]

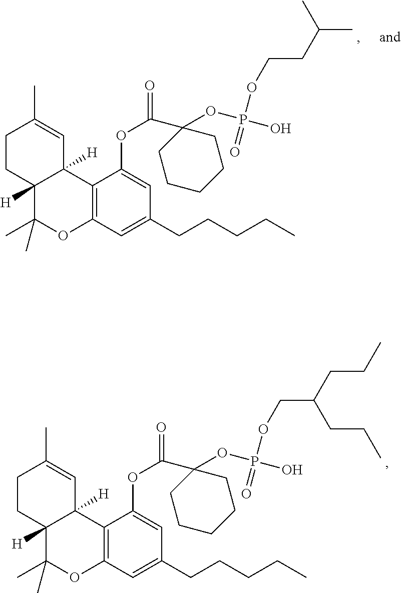 [SMILES: CCCCCc1cc(OC(=O)C2(OP(=O)(O)OCCC(C)C)CCCCC2)c2c(c1)OC(C)(C)[C@@H]1CCC(C)=C[C@@H]21 ; CCCCCc1cc(OC(=O)C2(OP(=O)(O)OCC(CCC)CCC)CCCCC2)c2c(c1)OC(C)(C)[C@@H]1CCC(C)=C[C@@H]21]

and pharmaceutically acceptable salts of any of the foregoing. In some embodiments, the pharmaceutically acceptable salts are ammonium salts.

In some embodiments, the compound of Formula (III)-1 A is chosen from compounds having the following formulae:

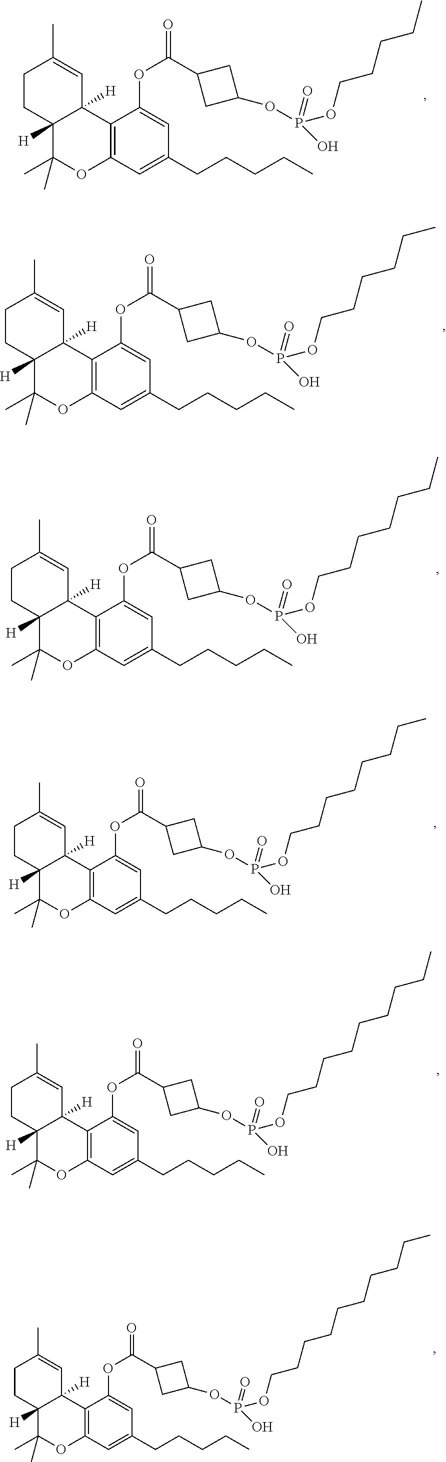 [SMILES: CCCCCOP(=O)(O)OC1CC(C(=O)Oc2cc(CCCCC)cc3c2[C@@H]2C=C(C)CC[C@H]2C(C)(C)O3)C1 ; CCCCCCOP(=O)(O)OC1CC(C(=O)Oc2cc(CCCCC)cc3c2[C@@H]2C=C(C)CC[C@H]2C(C)(C)O3)C1 ; CCCCCCCOP(=O)(O)OC1CC(C(=O)Oc2cc(CCCCC)cc3c2[C@@H]2C=C(C)CC[C@H]2C(C)(C)O3)C1 ; CCCCCCCCOP(=O)(O)OC1CC(C(=O)Oc2cc(CCCCC)cc3c2[C@@H]2C=C(C)CC[C@H]2C(C)(C)O3)C1 ; CCCCCCCCCOP(=O)(O)OC1CC(C(=O)Oc2cc(CCCCC)cc3c2[C@@H]2C=C(C)CC[C@H]2C(C)(C)O3)C1 ; CCCCCCCCCCOP(=O)(O)OC1CC(C(=O)Oc2cc(CCCCC)cc3c2[C@@H]2C=C(C)CC[C@H]2C(C)(C)O3)C1]

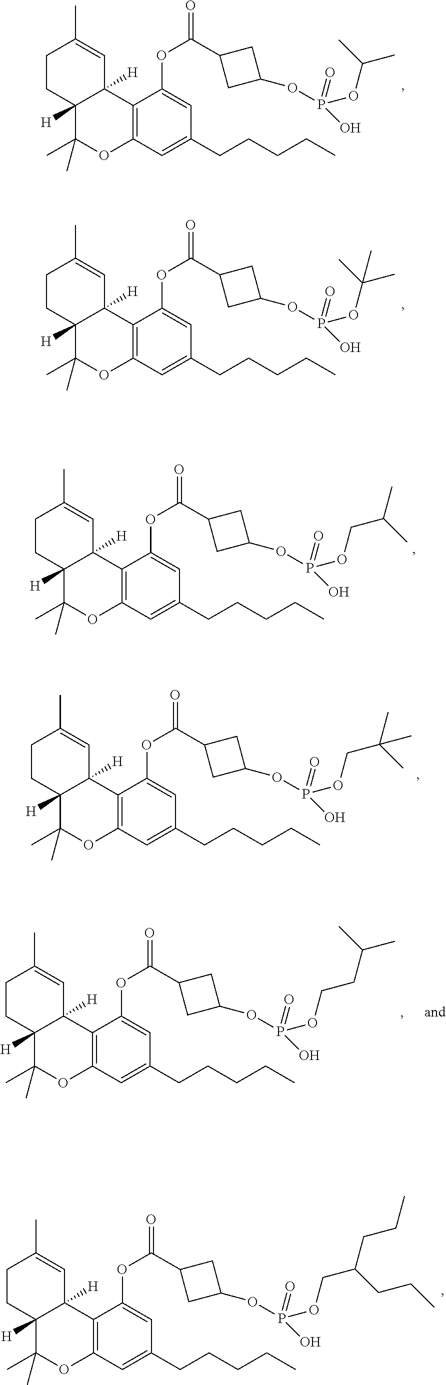 [SMILES: CCCCCc1cc(OC(=O)C2CC(OP(=O)(O)OC(C)C)C2)c2c(c1)OC(C)(C)[C@@H]1CCC(C)=C[C@@H]21 ; CCCCCc1cc(OC(=O)C2CC(OP(=O)(O)OC(C)(C)C)C2)c2c(c1)OC(C)(C)[C@@H]1CCC(C)=C[C@@H]21 ; CCCCCc1cc(OC(=O)C2CC(OP(=O)(O)OCC(C)C)C2)c2c(c1)OC(C)(C)[C@@H]1CCC(C)=C[C@@H]21 ; CCCCCc1cc(OC(=O)C2CC(OP(=O)(O)OCC(C)(C)C)C2)c2c(c1)OC(C)(C)[C@@H]1CCC(C)=C[C@@H]21 ; CCCCCc1cc(OC(=O)C2CC(OP(=O)(O)OCCC(C)C)C2)c2c(c1)OC(C)(C)[C@@H]1CCC(C)=C[C@@H]21 ; CCCCCc1cc(OC(=O)C2CC(OP(=O)(O)OCC(CCC)CCC)C2)c2c(c1)OC(C)(C)[C@@H]1CCC(C)=C[C@@H]21]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

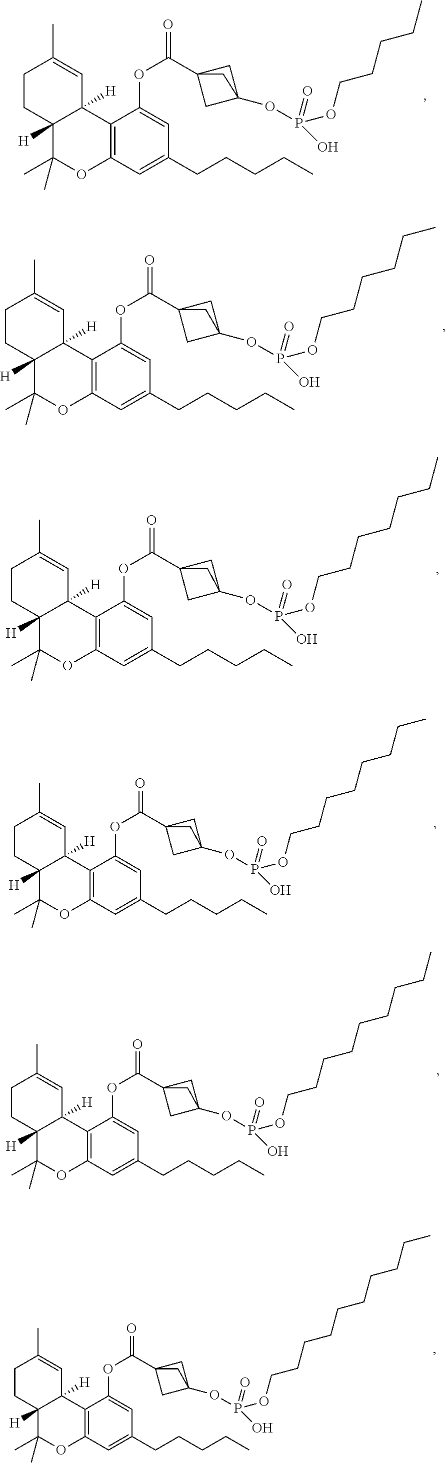

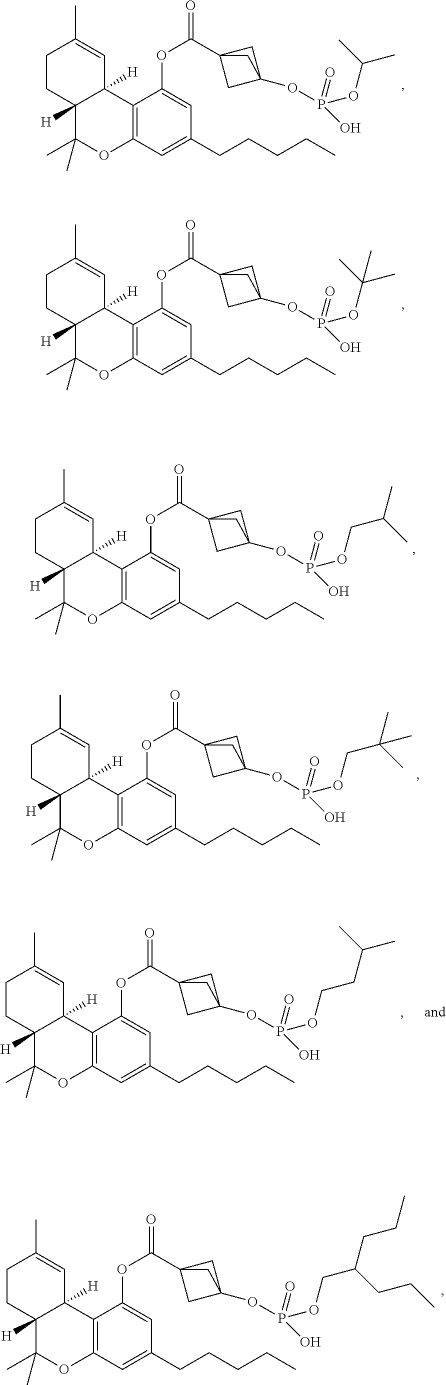

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

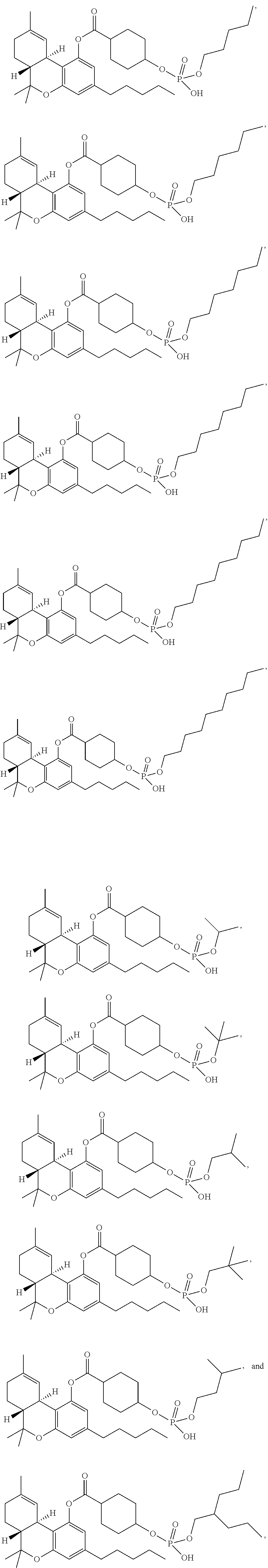

and pharmaceutically acceptable salts of any of the foregoing. In some embodiments the pharmaceutically acceptable salts are ammonium salts.

In some embodiments, the compound of Formula (III)-1A is chosen from compounds having the following formulae:

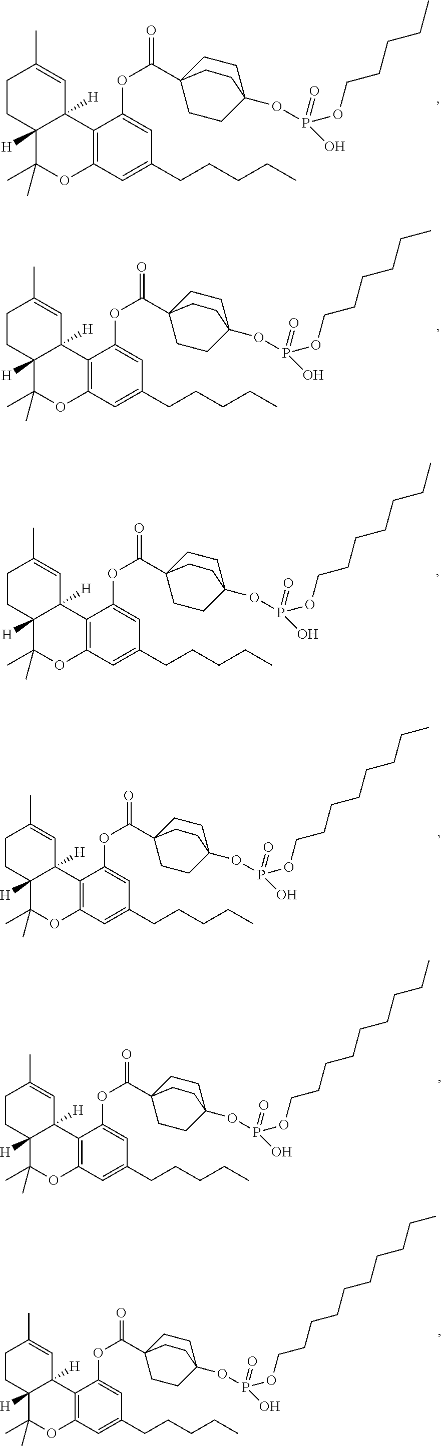

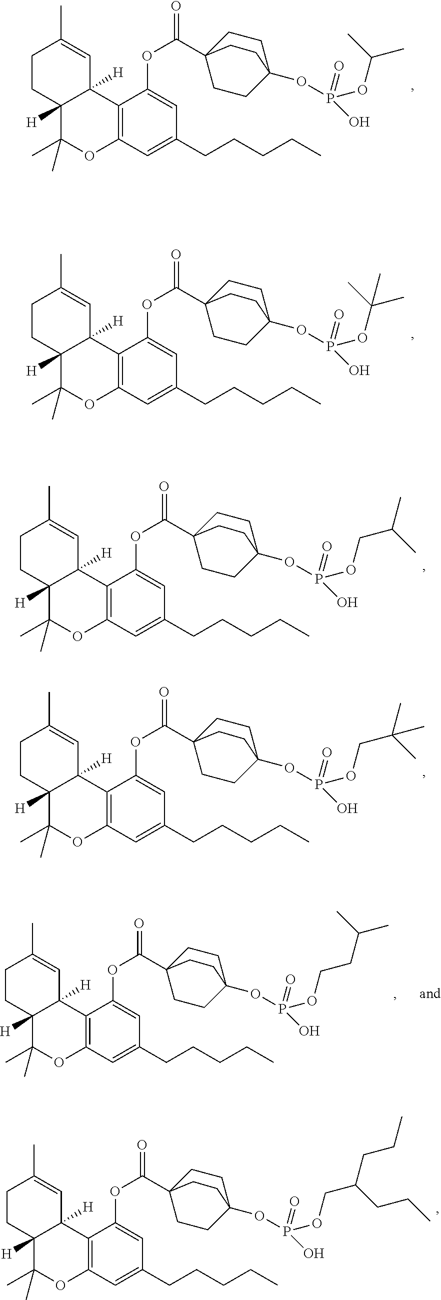

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

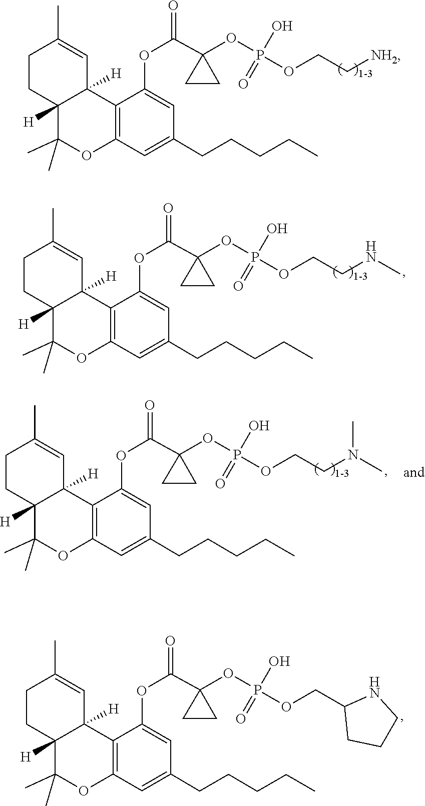

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

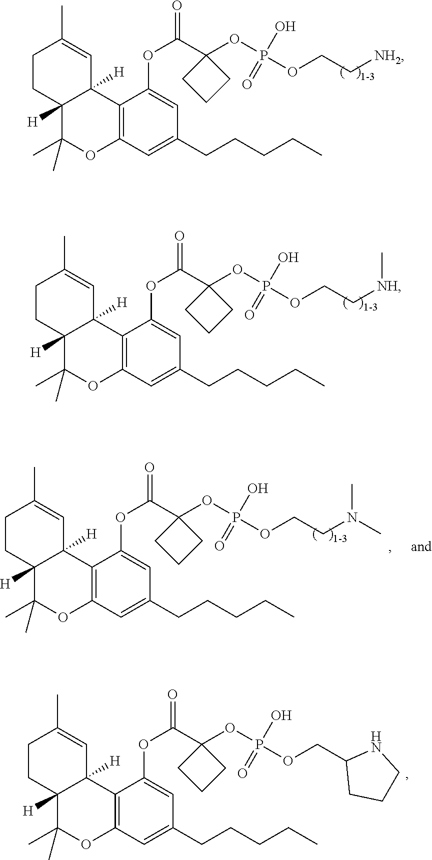

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

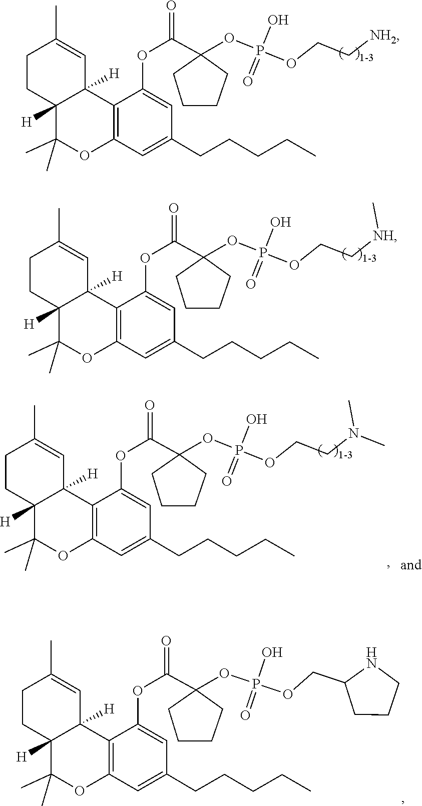

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

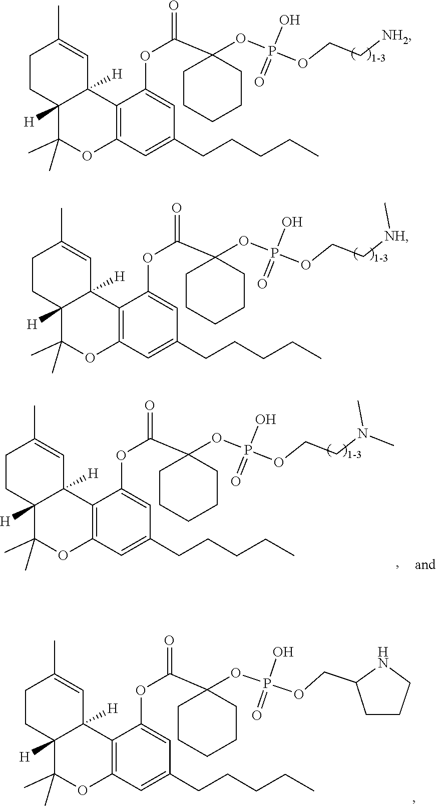

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

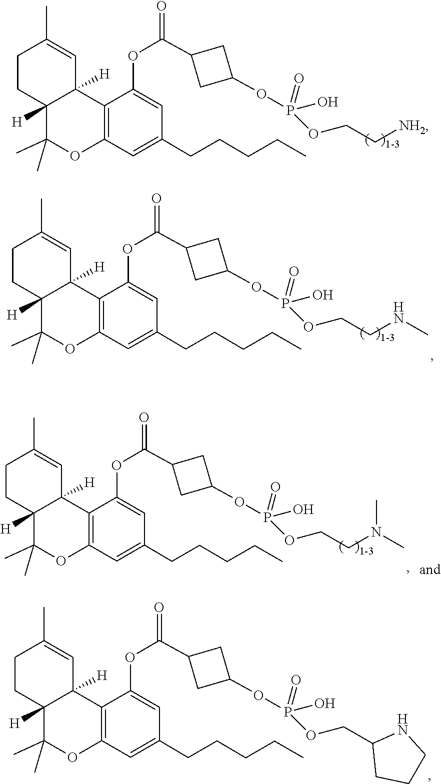

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

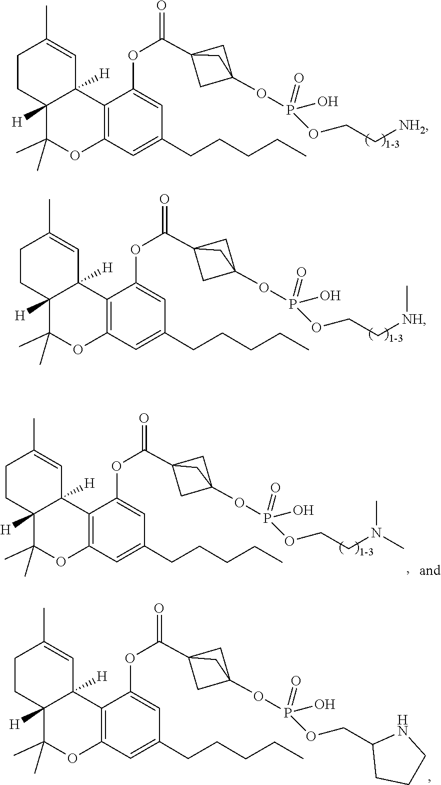

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

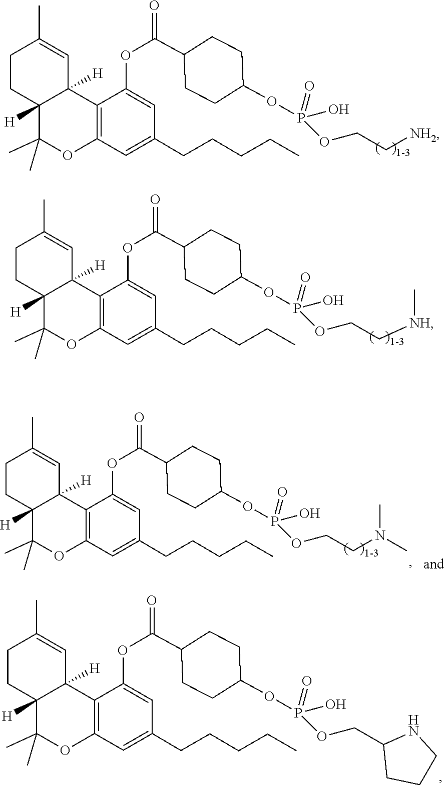

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

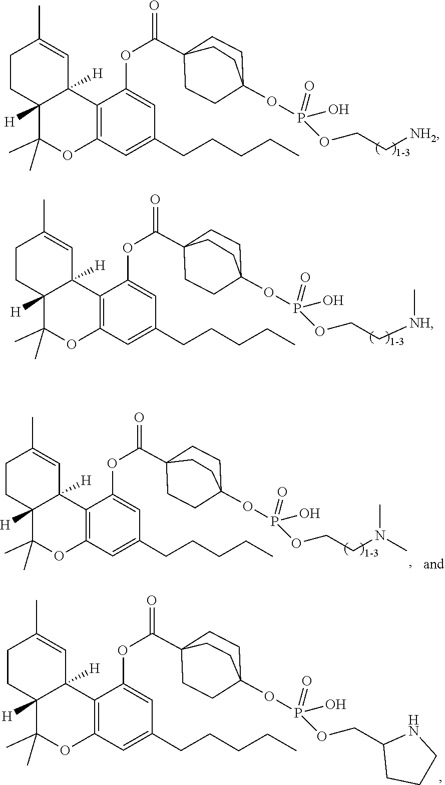

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

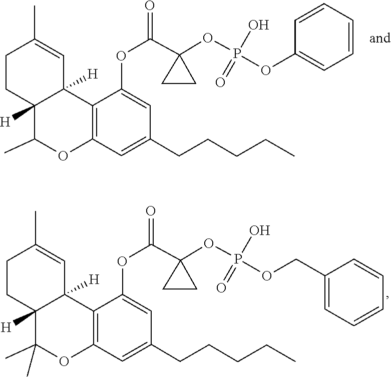

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

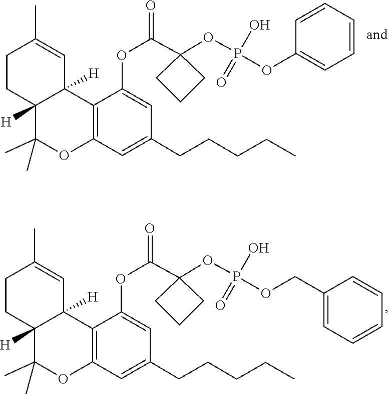

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

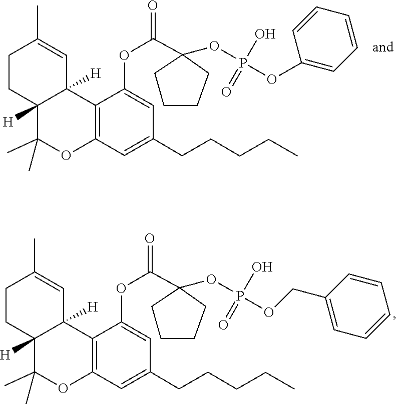

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

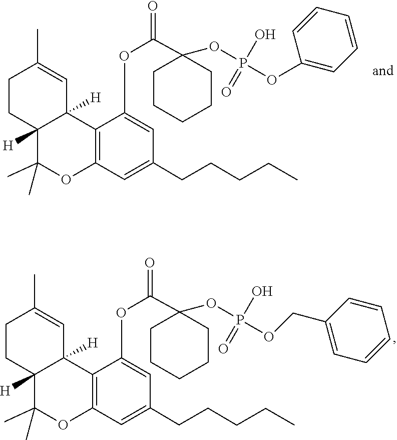

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

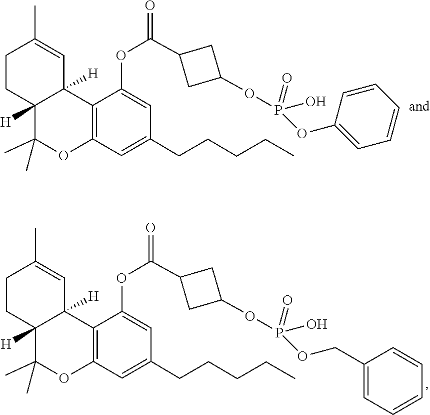

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

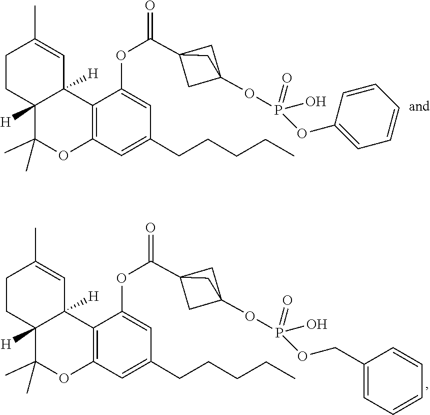

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

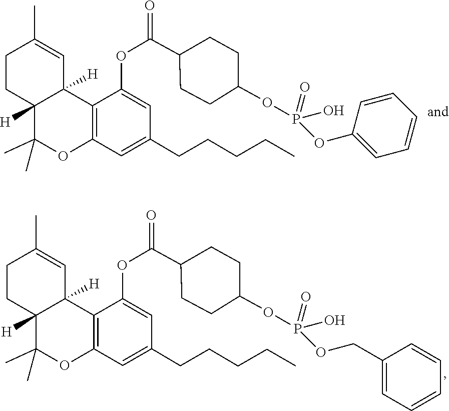

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

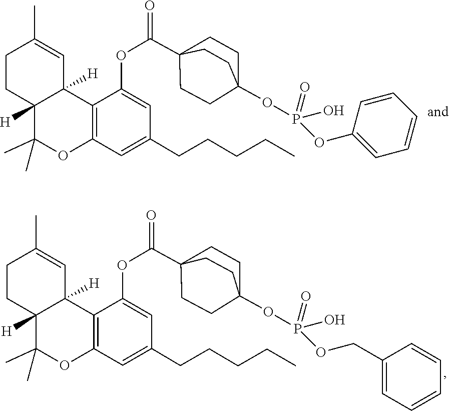

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

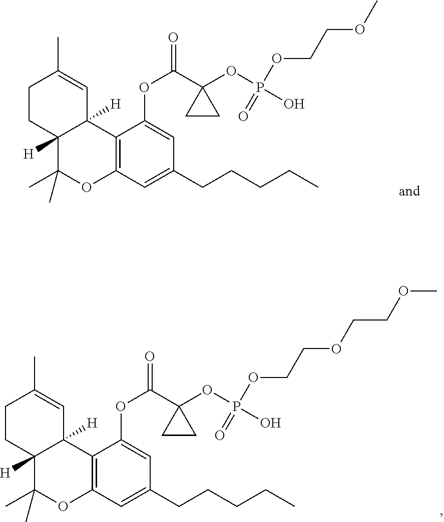 [SMILES: CCCCCc1cc(OC(=O)C2(OP(=O)(O)OCCOC)CC2)c2c(c1)OC(C)(C)[C@@H]1CCC(C)=C[C@@H]21 ; CCCCCc1cc(OC(=O)C2(OP(=O)(O)OCCOCCOC)CC2)c2c(c1)OC(C)(C)[C@@H]1CCC(C)=C[C@@H]21]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

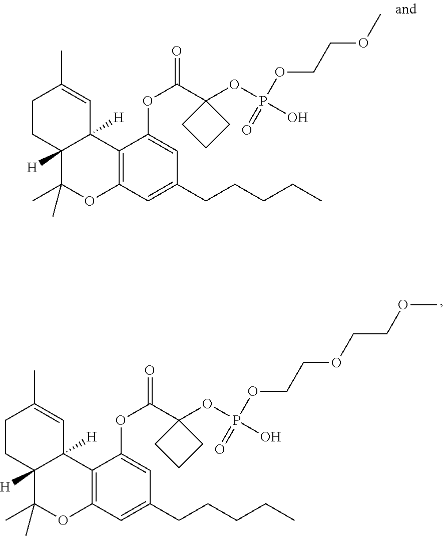

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

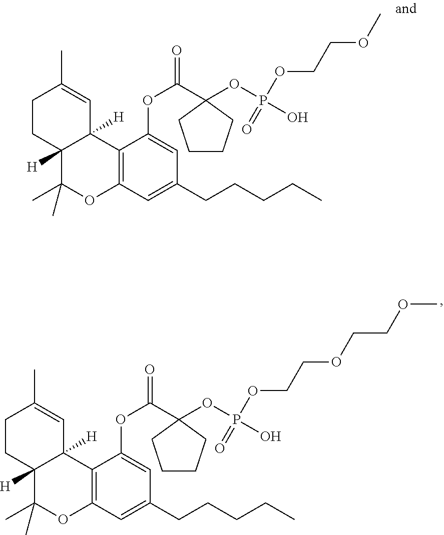

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

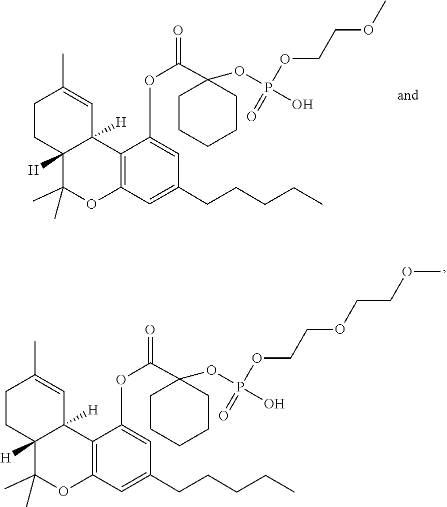

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

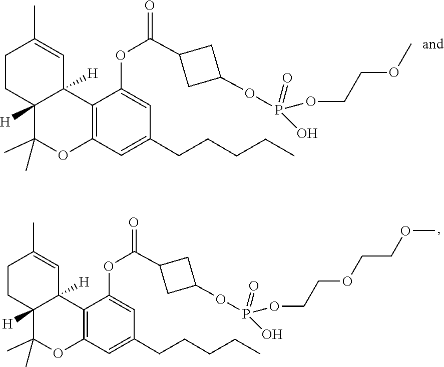

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

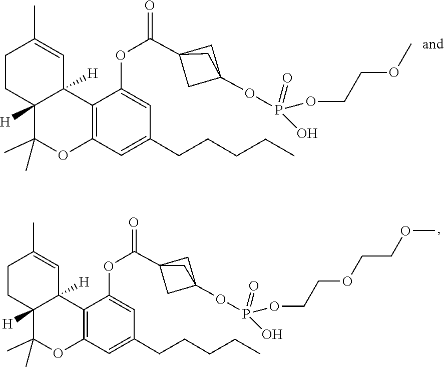

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

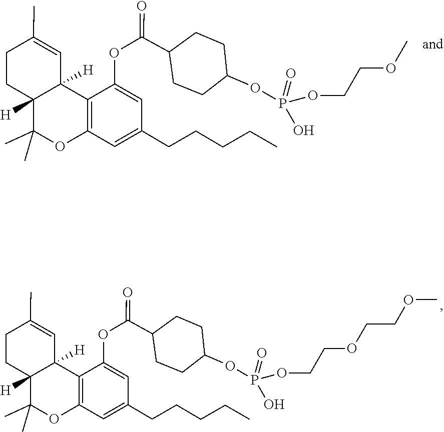

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

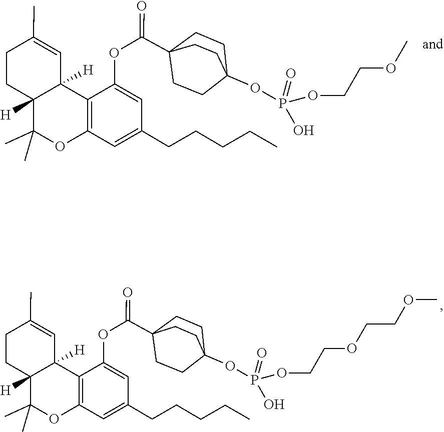

and pharmaceutically acceptable salts of any of the foregoing. In some embodiments, the pharmaceutically acceptable salts are ammonium salts.

In some embodiments, the compound of Formula (III)-1A is chosen from compounds having the following formula:

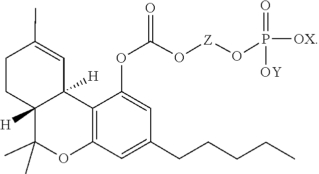

and pharmaceutically acceptable salts thereof. In some embodiments, the carbon in Z that is next to the carbonyloxy group

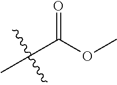 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

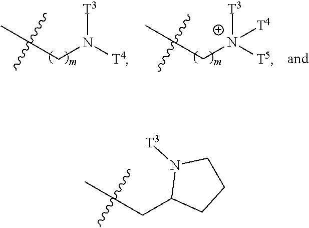

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

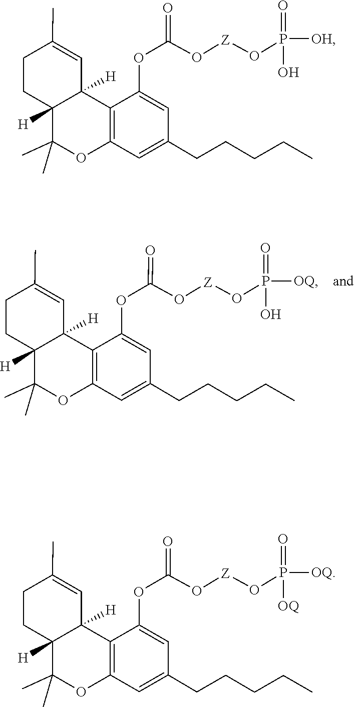

In some embodiments, the carbon in Z that is next to the carbonyloxy group

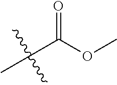 [SMILES: *C(=O)OC]

(i.e., the alpha carbon) has no hydrogen atoms. In some embodiments, the carbon in Z that is next to the carbonyloxy group (i.e., the alpha carbon) has one hydrogen atom. In some embodiments, the carbon in Z that is next to the carbonyloxy group (i.e., the alpha carbon) has two hydrogen atoms. In some embodiments, Z is substituted with the one or more groups as described above, wherein at least one of the foregoing substituents resides on the alpha carbon.

In some embodiments, the compound of Formula (III)-1A is chosen from compounds having the following formulae:

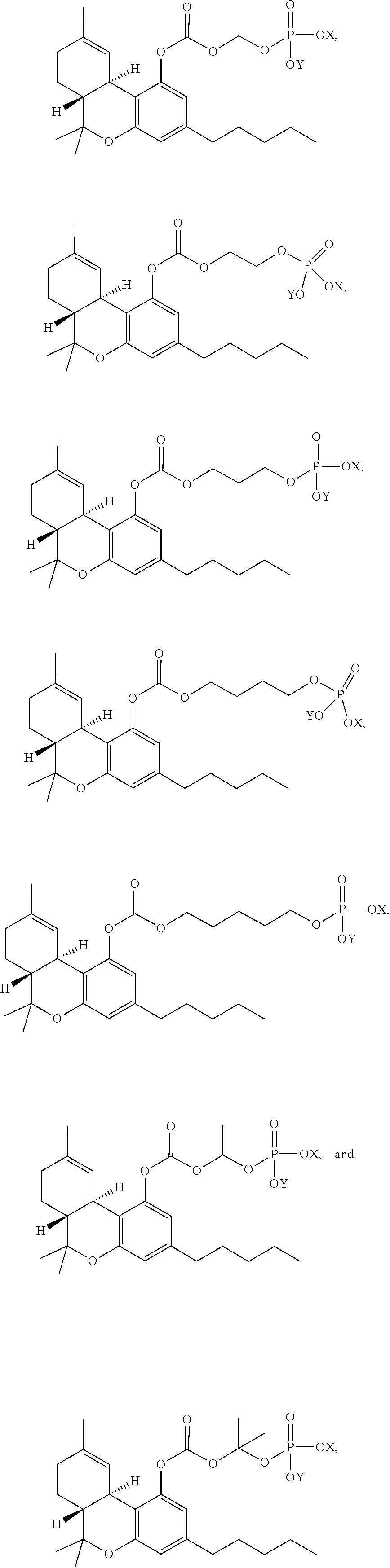

and pharmaceutically acceptable salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

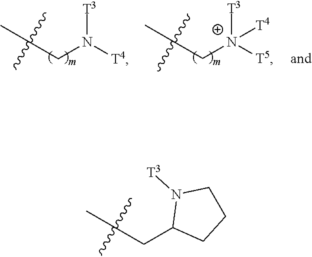

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

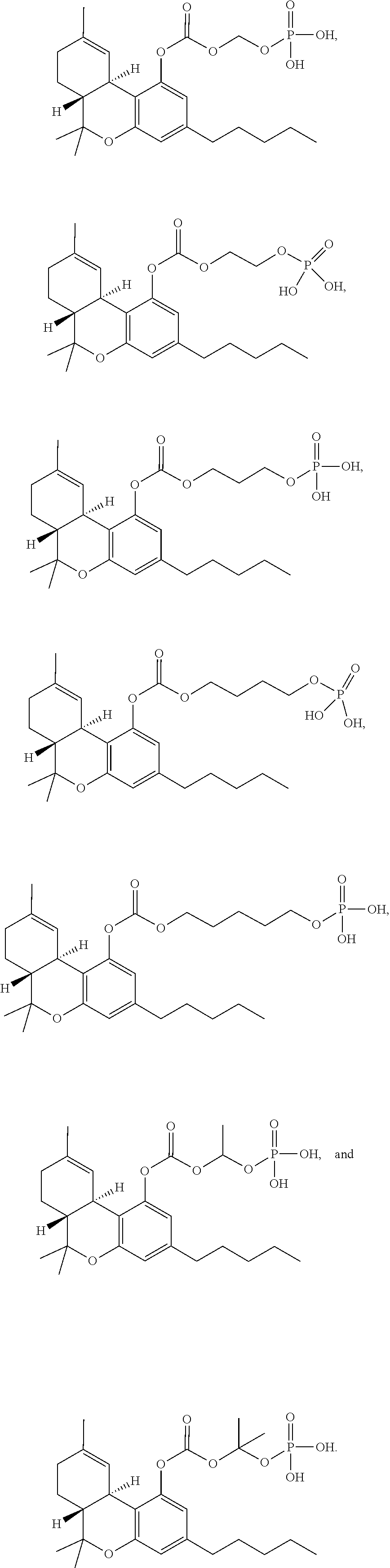

In some embodiments, the compound of Formula (III)-1A is chosen from compounds having the following formulae:

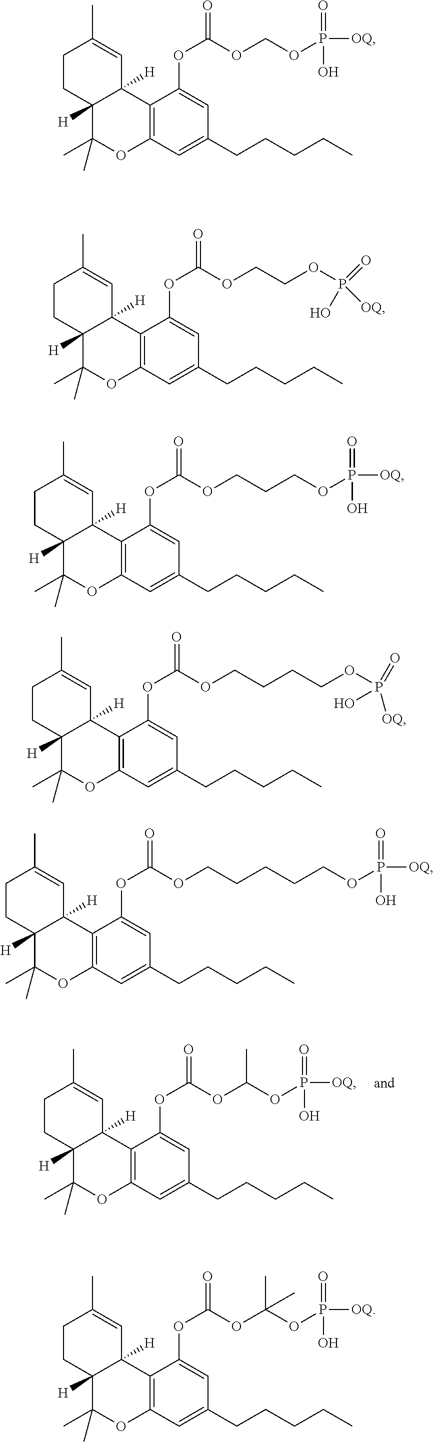

In some embodiments, the compound of Formula (III)-1A is chosen from compounds having the following formulae:

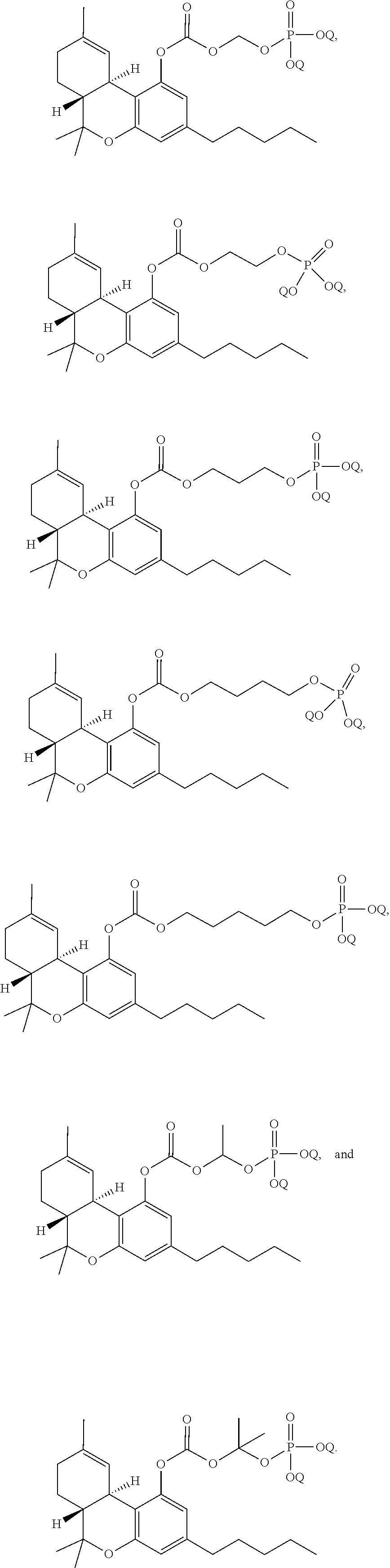

In some embodiments, the compound of Formula (III)-1A is chosen from compounds having the following formulae:

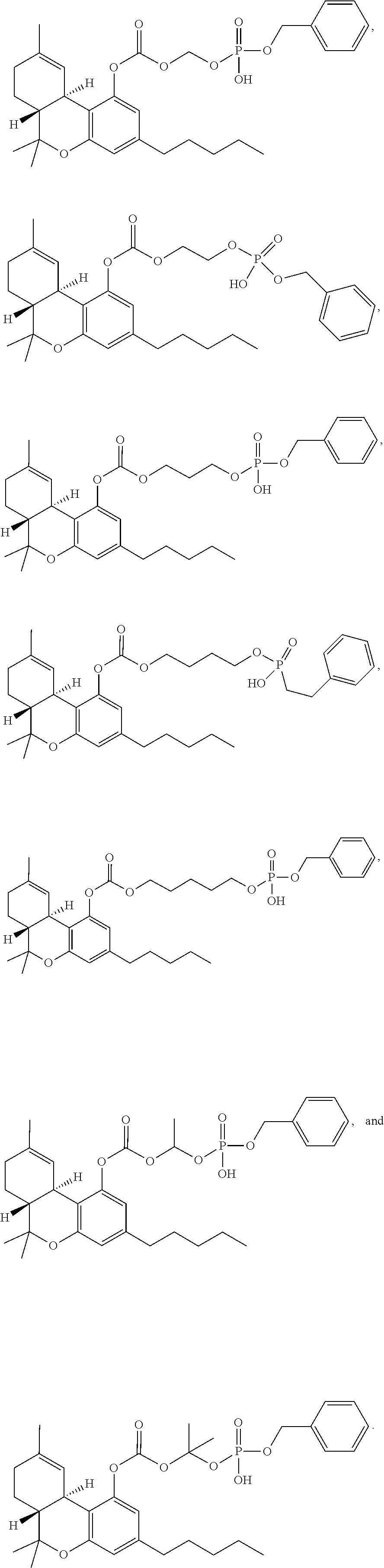

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

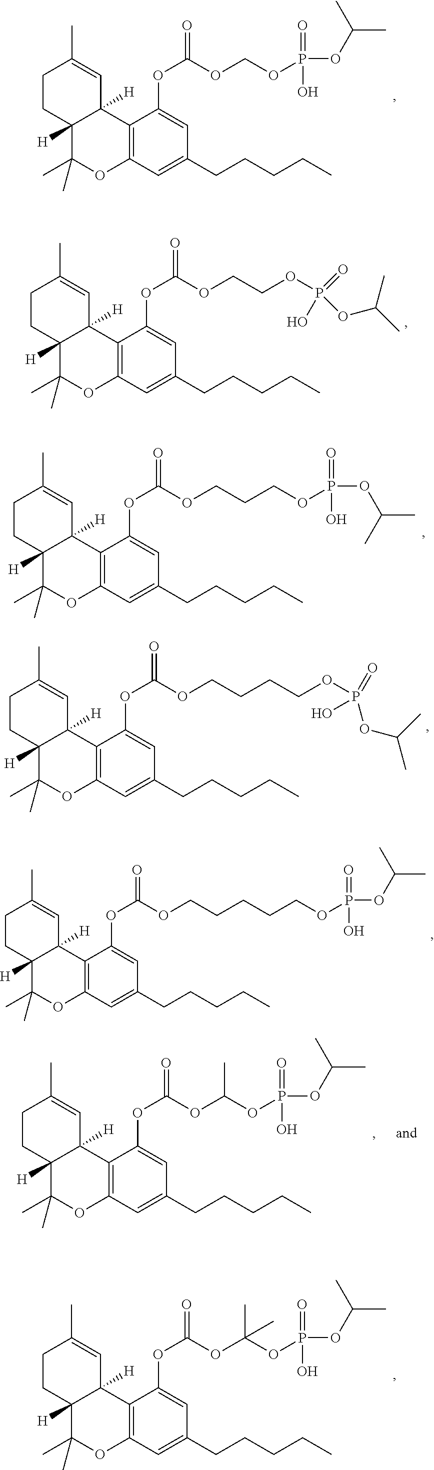

and pharmaceutically acceptable salts of any of the foregoing. In some embodiments, the pharmaceutically acceptable salts are ammonium salts.

In some embodiments, the compound of Formula (III)-1A is chosen from compounds having the following formula:

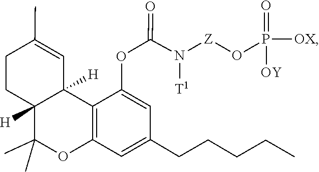

and pharmaceutically acceptable salts thereof. In some embodiments, the pharmaceutically acceptable salts are ammonium salts. In some embodiments, the carbon in Z that is next to the carbonylamino group

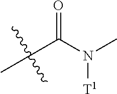

(i.e., the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, T¹ is H. In some embodiments, T¹ is methyl. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

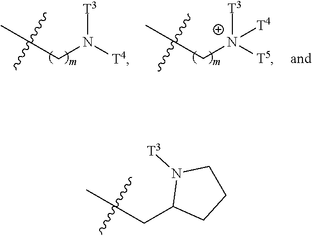

groups. In some embodiments, X is chosen from H and Q, and Y is chosen from phenyl, benzyl, and C₁₋₅ alkyl groups. In some embodiments, X is chosen from H and Q, and Y is chosen from phenyl, benzyl, ethyl, and isopropyl.

In some embodiments, the compound of Formula (III)-1A is chosen from compounds having the following formulae:

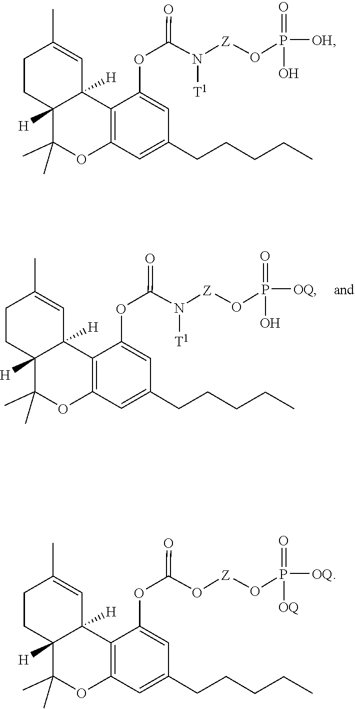

In some embodiments, the carbon in Z that is next to the carbonylamino group

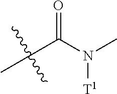

(i.e., the alpha carbon) has no hydrogen atoms. In some embodiments, the carbon in Z that is next to the carbonylamino group (i.e., the alpha carbon) has one hydrogen atom. In some embodiments, the carbon in Z that is next to the carbonylamino group (i.e., the alpha carbon) has two hydrogen atoms. In some embodiments, Z is substituted with the one or more groups as described above, wherein at least one of the foregoing substituents resides on the alpha carbon. In some embodiments, T¹ is H. In some embodiments, T¹ is methyl.

In some embodiments, the compound of Formula (III)-1A is chosen from compounds having the following formulae:

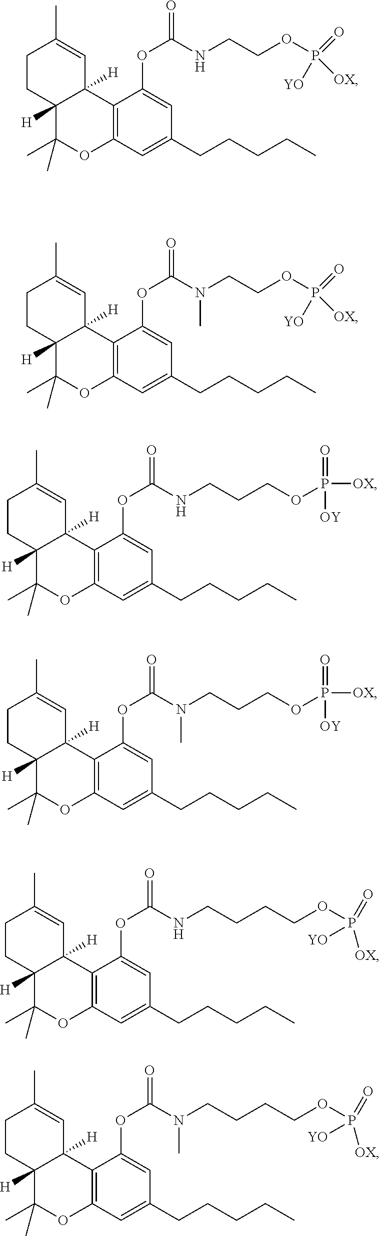

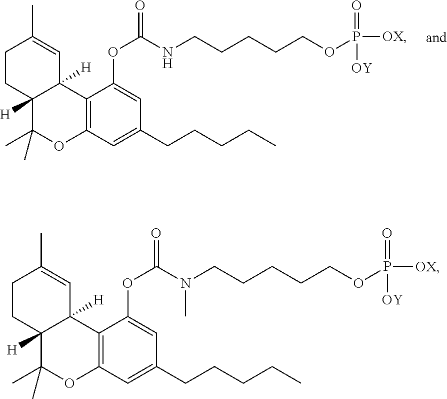

and pharmaceutical salts of any of the foregoing. In some embodiments, the pharmaceutically acceptable salts are ammonium salts. In some embodiments, X is H. In some embodiments, X is Q. In some embodiments, Y is H. In some embodiments, Y is Q. In some embodiments, X and Y are each H. In some embodiments, X and Y are each Q. In some embodiments, only one of X or Y is H. In some embodiments, only one of X or Y is Q. In some embodiments, X is H and Y is Q. In some embodiments, X is chosen from H and Q, and Y is chosen from C₁₋₁₈ alkyl, C₁₋₁₈ haloalkyl, C₆₋₁₈ aryl C₁₋₁₃ heteroaryl, C₇₋₁₉ arylalkyl, C₂₋₁₄ heteroarylalkyl, —(CH₂CH₂O)ₙCH₃,

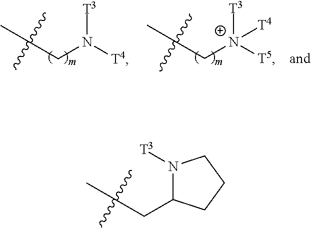

groups. In some embodiments, X is chosen from H and Q, and Y is chosen from phenyl, benzyl, and C₁₋₅ alkyl groups. In some embodiments, X is chosen from H and Q. and Y is chosen from phenyl, benzyl, ethyl, and isopropyl.

In some embodiments, the compound of Formula (III)-1A is chosen from compounds having the following formulae:

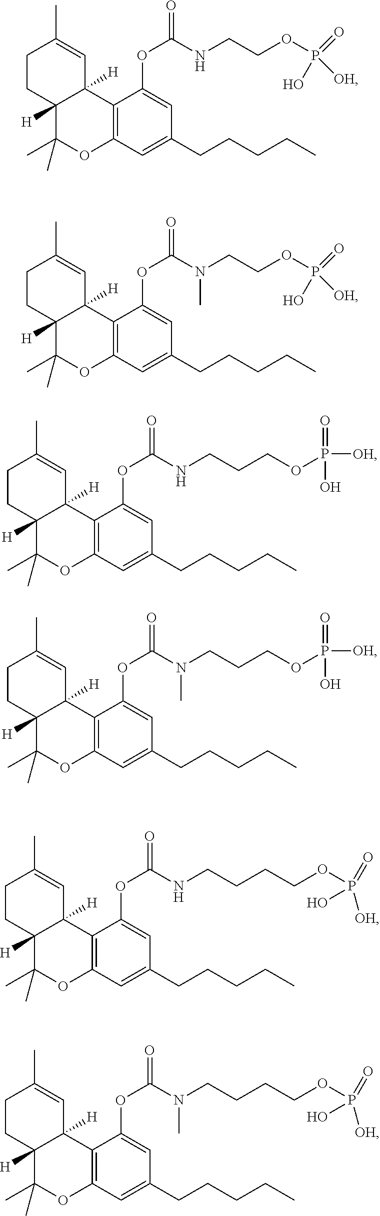

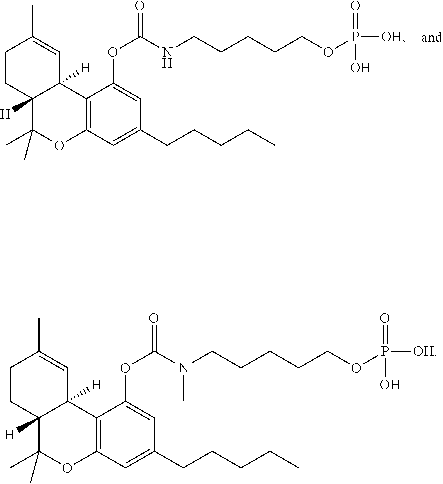 [SMILES: CCCCCc1cc(OC(=O)NCCCCCOP(=O)(O)O)c2c(c1)OC(C)(C)[C@@H]1CCC(C)=C[C@@H]21 ; CCCCCc1cc(OC(=O)N(C)CCCCCOP(=O)(O)O)c2c(c1)OC(C)(C)[C@@H]1CCC(C)=C[C@@H]21]

In some embodiments, the compound of Formula (III)-1A is chosen from compounds having the following formulae:

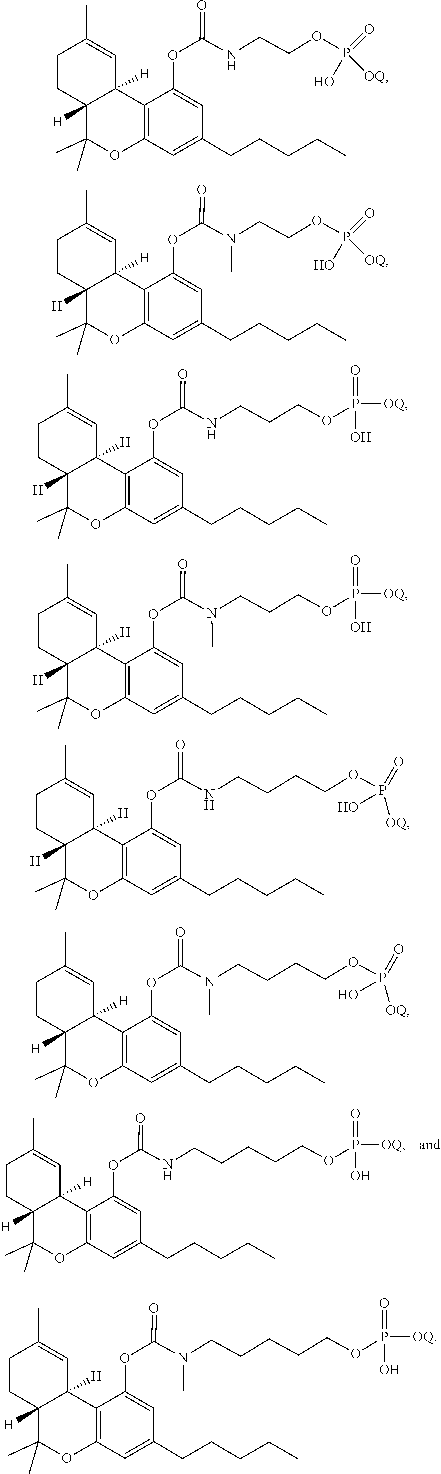

In some embodiments, the compound of Formula (III)-1A is chosen from compounds having the following formulae:

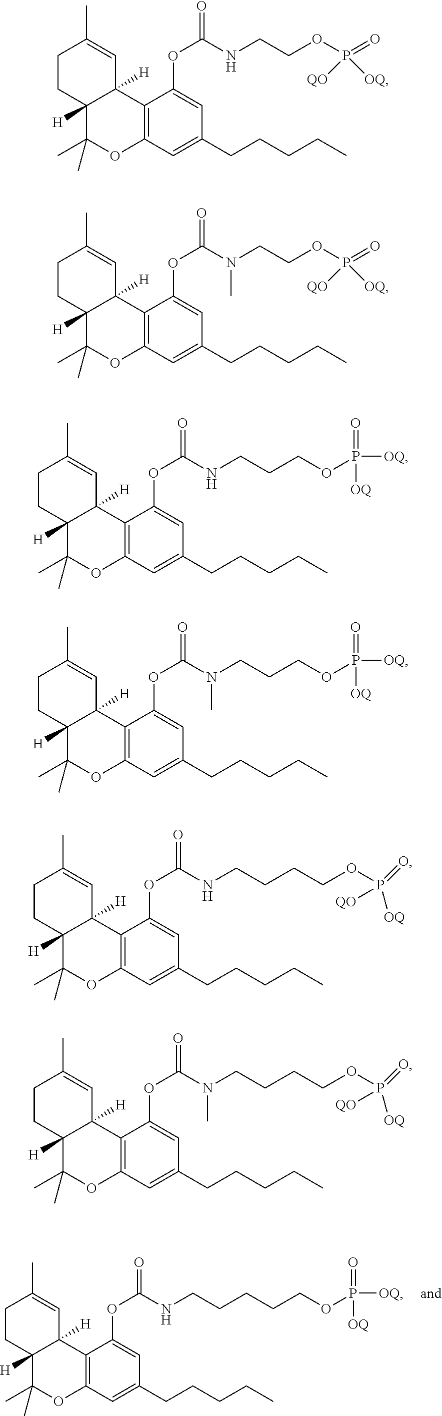

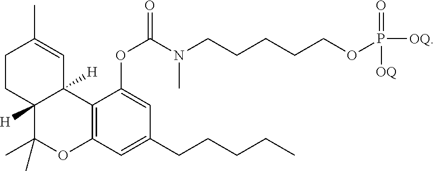

In some embodiments, the compound of Formula (III)-1A is chosen from compounds having the following formulae:

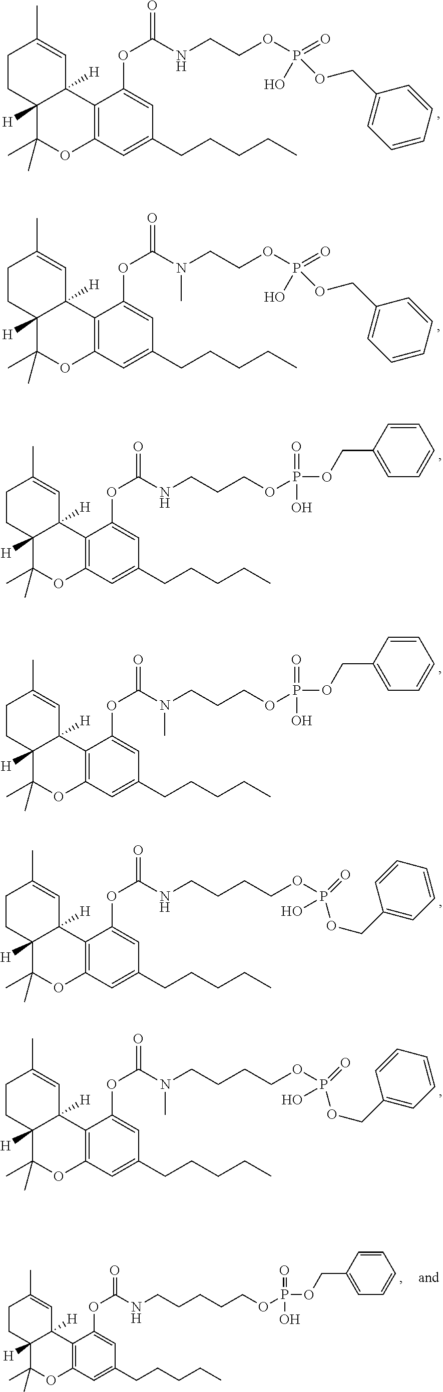

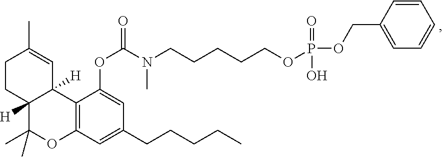 [SMILES: CCCCCc1cc(OC(=O)N(C)CCCCCOP(=O)(O)OCc2ccccc2)c2c(c1)OC(C)(C)[C@@H]1CCC(C)=C[C@@H]21]

and pharmaceutically acceptable salts of any of the foregoing. In some embodiments, the pharmaceutically acceptable salts are ammonium salts.

In some embodiments, the compound of Formula (III)-1A is chosen from compounds having the following formulae:

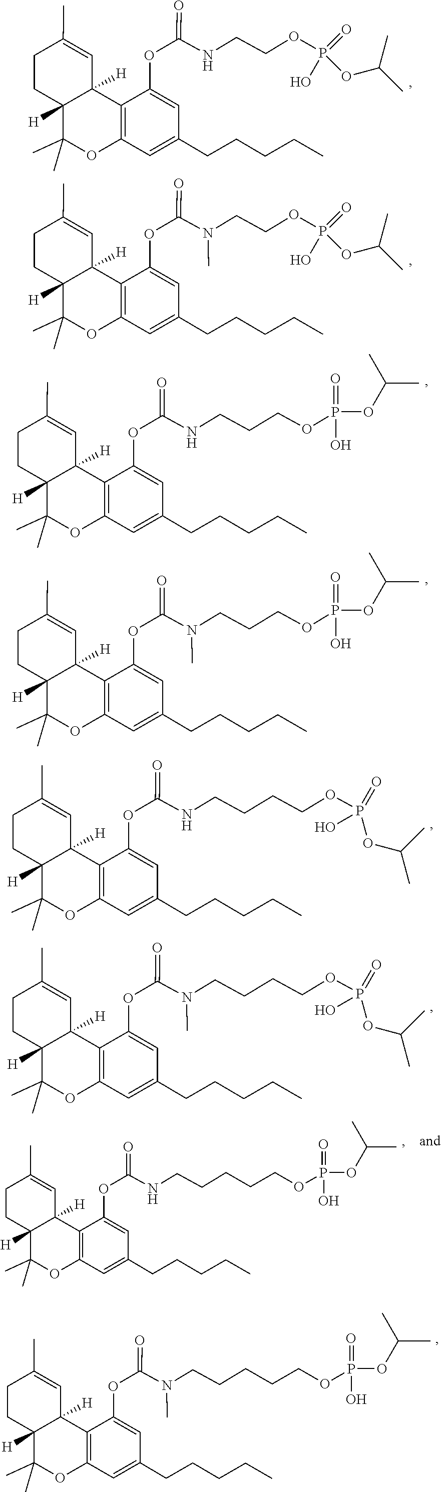

and pharmaceutically acceptable salts of any of the foregoing. In some embodiments, the pharmaceutically acceptable salts are ammonium salts.

In some embodiments, the compound of Formula (III)-1A is chosen from compounds having the following formula:

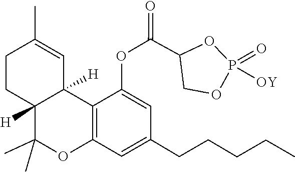

and pharmaceutically acceptable salts thereof. In some embodiments, the pharmaceutically acceptable salts are ammonium salts. In some embodiments, Y is H. In some embodiments, Y is Q. In some embodiments, Y is chosen from C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃,

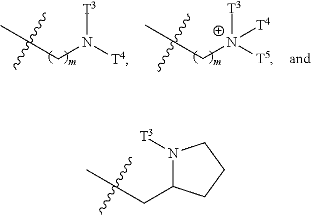

and groups. In some embodiments, Y is chosen from phenyl, benzyl, and C₁₋₈ alkyl groups. In some embodiments, Y is chosen from phenyl, benzyl, ethyl, and isopropyl.

In some embodiments, the compound of Formula (III)-1A has the following formula:

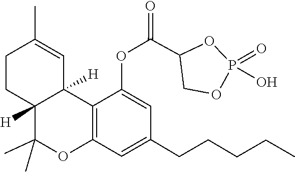 [SMILES: CCCCCc1cc(OC(=O)C2COP(=O)(O)O2)c2c(c1)OC(C)(C)[C@@H]1CCC(C)=C[C@@H]21]

and pharmaceutically acceptable salts thereof. In some embodiments, the pharmaceutically acceptable salts are ammonium salts.

In some embodiments, the compound of Formula (III)-1A has the following formula:

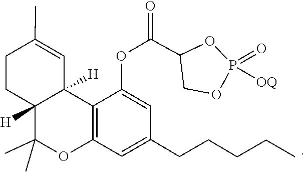

In some embodiments, the compound of Formula (III)-1A has the following formula:

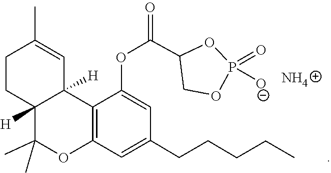

In some embodiments, the compound of Formula (III)-1A is chosen from compounds having the following formulae:

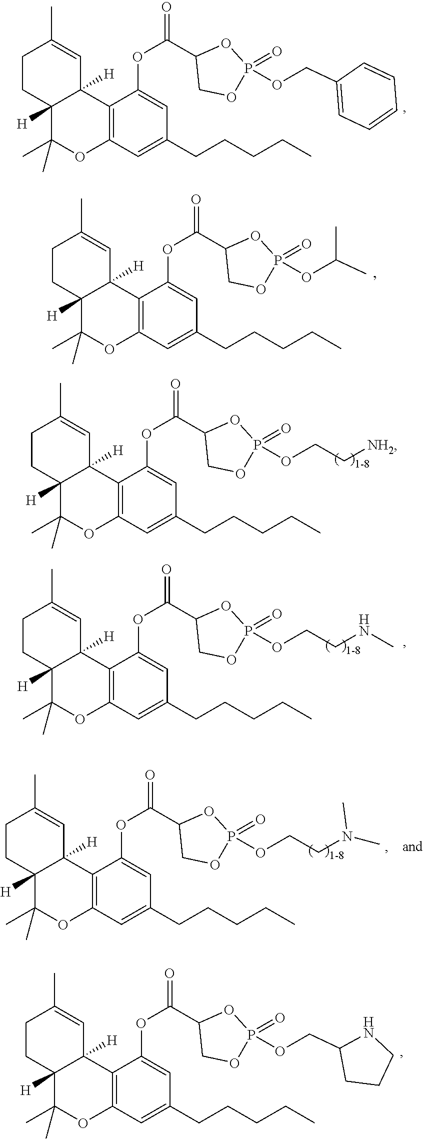

and pharmaceutically acceptable salts if any foregoing. In some embodiments, the pharmaceutically acceptable salts are ammonium salts.

### Formula (III)-1B

In some embodiments, Formula (III)-1B. R¹ is the same as described above. For example, exemplary embodiments of R are described in Formula (III)-1A.

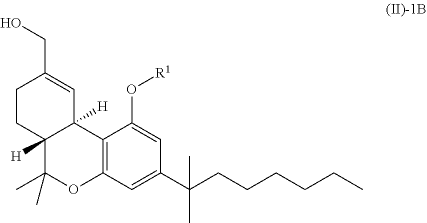

### Formula (III)-2A

In some embodiments, Formula (III)-2 is in the form of Formula (III)-2A. i.e., the compounds are prodrugs of (+)trans-Δ⁹-THC. R¹ is the same as described above. For example, exemplary embodiments of R¹ are described in Formula (III)-1A.

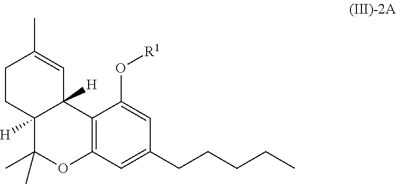

### Formula (III)-2B

In some embodiments, Formula (III)-2 is in the form of Formula (III)-2B. R¹ is the same as described above. For example, exemplary embodiments of R¹ are described in Formula (III)-1A.

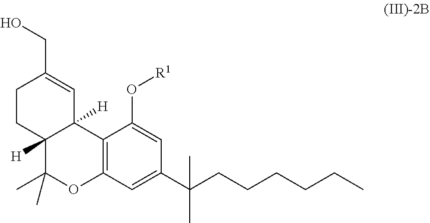

### 4. Formula (IV) and its Sub-Formulas

Optionally, the compounds have a structure of Formula (IV) or a pharmaceutically acceptable salt thereof.

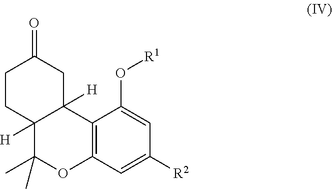

wherein R¹ and R² are the same as described above for Formula (I).

In some embodiments, the compounds are in a non-salt form. In some embodiments, the compounds are in a salt form. In some embodiments, the compounds are in an ammonium salt form.

In some embodiments, Formula (IV) is in the form of Formula (IV)-1.

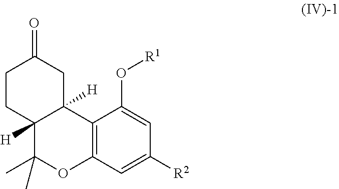

In some embodiments, Formula (IV) is in the form of Formula (IV)-2.

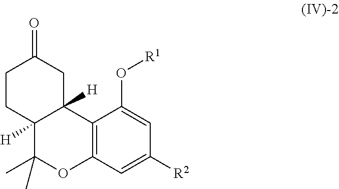

In some embodiments, R² is chosen from C₂₋₉ alkyl groups. In some embodiments, R² is chosen from n-propyl, n-pentyl, and 1,1-dimethylheptyl. In some embodiments, R² is n-propyl. In some embodiments, R² is n-pentyl. In some embodiments, R² is 1,1-dimethylheptyl.

### Formula (IV)-A

In some embodiments, Formula (IV) is in the form of Formula (IV)-A, i.e., the compounds are prodrugs of nabilone. R¹ is the same as described above. For example, exemplary embodiments of R¹ are described in Formula (II)-1A and Formula (III)-1A.

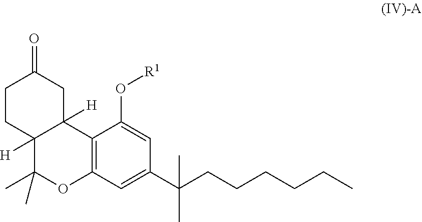

In some embodiments, Formula (IV)-A is in the form of Formula (IV)-1A, i.e., the compounds are prodrugs of (−)trans-nabilone. R¹ is the same as described above. For example, exemplary embodiments of R¹ are described in Formula (II)-1A and Formula (III)-1A.

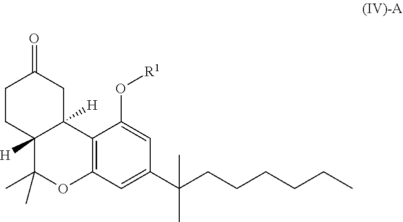

In some embodiments, Formula (IV)-A is in the form of Formula (IV)-2A, i.e., the compounds are prodrugs of (+)trans-nabilone. R¹ is the same as described above. For example, exemplary embodiments of R¹ are described in Formula (II)-1A and Formula (III)-1A.

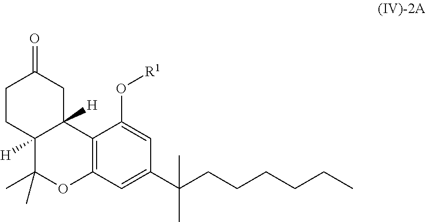

### C. Properties

In some embodiments, the compounds disclosed herein have a human liver microsomes stability, T_{1/2}, of no less than about 15 min. For example, the compounds disclosed herein have a human liver microsomes stability, T_{1/2}, of between about 15 min and about 2 hours. In some embodiments, the compounds disclosed herein have a human liver microsomes stability, T_{1/2}, of between about 15 and about 30 min. In some embodiments, the compounds disclosed herein have a human liver microsomes stability, T_{1/2}, of between about 30 and about 45 min. In some embodiments, the compounds disclosed herein have a human liver microsomes stability, T_{1/2}, of between about 45 min and about 1 hour. In some embodiments, the compounds disclosed herein have a human liver microsomes stability, T_{1/2}, of between about 1 and about 1.25 hours. In some embodiments, the compounds disclosed herein have a human liver microsomes stability, T_{1/2}, of between about 1.25 and about 1.5 hours. In some embodiments, the compounds disclosed herein have a human liver microsomes stability. T_{1/2}, of between about 1.5 and about 1.75 hours. In some embodiments, the compounds disclosed herein have a human liver microsomes stability, T_{1/2}, of between about 1.75 and about 2 hours. In some embodiments, the compounds disclosed herein have a human liver microsomes stability, T_{1/2}, of greater than about 2 hours.

In some embodiments, the compounds disclosed herein have a human liver microsomes stability, T_{1/2}, greater than the stability of their corresponding parent compounds, measured under the same experimental conditions. In some embodiments, the compounds disclosed herein have a human liver microsomes stability, T_{1/2}, of at least 1.5 times the stability of their corresponding parent compounds. In some embodiments, the compounds disclosed herein have a human liver microsomes stability. T_{1/2}, of at least 2 times the stability of their corresponding parent compounds. In some embodiments, the compounds disclosed herein have a human liver microsomes stability, T_{1/2}, of at least 2.5 times the stability of their corresponding parent compounds. In some embodiments, the compounds disclosed herein have a human liver microsomes stability, T_{1/2}, of at least 3 times the stability of their corresponding parent compounds. In some embodiments, the compounds disclosed herein have a human liver microsomes stability, T_{1/2}, of at least 3.5 times the stability of their corresponding parent compounds. In some embodiments, the compounds disclosed herein have a human liver microsomes stability, T_{1/2}, of at least 4 times the stability of their corresponding parent compounds. In some embodiments, the compounds disclosed herein have a human liver microsomes stability, T_{1/2}, of at least 4.5 times the stability of their corresponding parent compounds. In some embodiments, the compounds disclosed herein have a human liver microsomes stability. T_{1/2}, of at least 5 times the stability of their corresponding parent compounds.

In some embodiments, the compounds of Formula (II)-1A have a human liver microsomes stability. T_{1/2}, greater than the stability of (−)trans-Δ⁸-THC, measured under the same experimental conditions. In some embodiments, the compounds of Formula (II)-1A have a human liver microsomes stability, T_{1/2}, of at least 1.5 times the stability of (−)trans-Δ⁸-THC. In some embodiments, the compounds of Formula (II)-1A have a human liver microsomes stability. T_{1/2}, of at least 2 times the stability of (−)trans-Δ⁸-THC. In some embodiments, the compounds of Formula (II)-1A have a human liver microsomes stability, T_{1/2}, of at least 2.5 times the stability of (−)trans-Δ⁸-THC. In some embodiments, the compounds of Formula (II)-1A have a human liver microsomes stability, T_{1/2}, of at least 3 times the stability of (−)trans-Δ⁸-THC. In some embodiments, the compounds of Formula (II)-1A have a human liver microsomes stability, T_{1/2}, of at least 3.5 times the stability of (−)trans-Δ⁸-THC. In some embodiments, the compounds of Formula (II)-1A have a human liver microsomes stability, T_{1/2}, of at least 4 times the stability of (−)trans-Δ⁸-THC. In some embodiments, the compounds of Formula (II)-1A have a human liver microsomes stability, T_{1/2}, of at least 4.5 times the stability of (−)trans-Δ⁸-THC. In some embodiments, the compounds of Formula (II)-1A have a human liver microsomes stability, T_{1/2}, of at least 5 times the stability of (−)trans-Δ⁸-THC.

In some embodiments, the compounds of Formula (III)-1A have a human liver microsomes stability, T_{1/2}, greater than the stability of (−)trans-Δ⁹-THC, measured under the same experimental conditions. In some embodiments, the compounds of Formula (III)-1A have a human liver microsomes stability. T_{1/2}, of at least 1.5 times the stability of (−)trans-Δ⁹ -THC. In some embodiments, the compounds of Formula (III)-1A have a human liver microsomes stability. T_{1/2}, of at least 2 times the stability of (−)trans-Δ⁹-THC. In some embodiments, the compounds of Formula (III)-1A have a human liver microsomes stability, T_{1/2} of at least 2.5 times the stability of (−)trans-Δ⁹-THC. In some embodiments, the compounds of Formula (III)-1A have a human liver microsomes stability, T_{1/2}, of at least 3 times the stability of (−)trans-Δ⁹-THC. In some embodiments, the compounds of Formula (III)-1A have a human liver microsomes stability, T_{1/2}, of at least 3.5 times the stability of (−)trans-Δ⁹-THC. In some embodiments, the compounds of Formula (III)-1A have a human liver microsomes stability, T_{1/2}, of at least 4 times the stability of (−)trans-Δ⁹-THC. In some embodiments, the compounds of Formula (III)-1A have a human liver microsomes stability, T_{1/2}, of at least 4.5 times the stability of (−)trans-Δ⁹-THC. In some embodiments, the compounds of Formula (III)-1A have a human liver microsomes stability, T_{1/2}, of at least 5 times the stability of (−)trans-Δ⁹-THC.

As used herein, the “human liver microsomes stability, T_{1/2}” is determined according to the procedure described in Example 9.

In some embodiments, the compounds disclosed herein have a solubility in 1% DMSO of no less than about 50 μM at 25° C. For example, the compounds disclosed herein have a solubility in 1% DMSO of between about 50 and about 500 μM at 25° C. In some embodiments, the compounds disclosed herein have a solubility in 1% DMSO between about 50 and about 75 μM at 25° C. In some embodiments, the compounds disclosed herein have a solubility in 1% DMSO between about 75 and about 100 μM at 25° C. In some embodiments, the compounds disclosed herein have a solubility in 1% DMSO between about 100 and about 125 μM at 25° C. In some embodiments, the compounds disclosed herein have a solubility in 1% DMSO between about 125 and 150 μM at 25° C. In some embodiments, the compounds disclosed herein have a solubility in 1% DMSO of greater than about 150 μM at 25° C. In some embodiments, the compounds disclosed herein have a solubility in 1% DMSO of greater than about 200 μM at 25° C. In some embodiments, the compounds disclosed herein have a solubility in 1% DMSO of greater than about 300 μM at 25° C. In some embodiments, the compounds disclosed herein have a solubility in 1% DMSO of greater than about 400 μM at 25° C. In some embodiments, the compounds disclosed herein have a solubility in 1% DMSO of greater than about 500 μM at 25° C.

In some embodiments, the compounds disclosed herein have a solubility in 1% DMSO greater than their corresponding parent compounds, measured under the same experimental conditions. In some embodiments, the compounds disclosed herein have a solubility in 1% DMSO of at least 2 times the solubility of their corresponding parent compounds. In some embodiments, the compounds disclosed herein have a solubility in 1% DMSO of at least 3 times the solubility of their corresponding parent compounds. In some embodiments, the compounds disclosed herein have a solubility in 1% DMSO of at least 5 times the solubility of their corresponding parent compounds. In some embodiments, the compounds disclosed herein have a solubility in 1% DMSO of at least 10 times the solubility of their corresponding parent compounds. In some embodiments, the compounds disclosed herein have a solubility in 1% DMSO at least 20 times the solubility of their corresponding parent compounds. In some embodiments, the compounds disclosed herein have a solubility in 1% DMSO at least 30 times the solubility of their corresponding parent compounds.

In some embodiments, the compounds of Formula (II)-1A have a solubility in 1% DMSO greater than (−)trans-Δ⁸-THC, measured under the same experimental conditions. In some embodiments, the compounds of Formula (II)-1A have a solubility in 1% DMSO of at least 2 times the solubility of (−)trans-Δ⁸-THC. In some embodiments, the compounds of Formula (II)-1A have a solubility in 1% DMSO of at least 3 times the solubility of (−)trans-As-THC. In some embodiments, the compounds of Formula (II)-1A have a solubility in 1% DMSO of at least 5 times the solubility of (−)trans-Δ⁸-THC. In some embodiments, the compounds of Formula (II)-1A have a solubility in 1% DMSO of at least 10 times the solubility of (−)trans-Δ⁸-THC. In some embodiments, the compounds of Formula (II)-1A have a solubility in 1% DMSO at least 20 times the solubility of (−)trans-Δ⁸-THC. In some embodiments, the compounds of Formula (I)-1A have a solubility in 1% DMSO at least 30 times the solubility of (−)trans-Δ⁸-THC.

In some embodiments, the compounds of Formula (III)-1A have a solubility in 1% DMSO greater than (−)trans-Δ⁸-THC, measured under the same experimental conditions. In some embodiments, the compounds of Formula (III)-1A have a solubility in 1% DMSO of at least 2 times the solubility of (−)trans-Δ⁸-THC. In some embodiments, the compounds of Formula (III)-1A have a solubility in 1% DMSO of at least 3 times the solubility of (−)trans-Δ⁸-THC. In some embodiments, the compounds of Formula (III)-1A have a solubility in 1% DMSO of at least 5 times the solubility of (−)trans-Δ⁸-THC. In some embodiments, the compounds of Formula (III)-1A have a solubility in 1% DMSO of at least 10 times the solubility of (−)trans-Δ⁸-THC. In some embodiments, the compounds of Formula (III)-1A have a solubility in 1% DMSO at least 20 times the solubility of (−)trans-Δ⁸-THC. In some embodiments, the compounds of Formula (III)-1A have a solubility in 1% DMSO at least 30 times the solubility of (−)trans-Δ⁹-THC.

As used herein, the “solubility in 1% DMSO” is determined by performing nephelometry experiments according to the procedure described in Example 8.

In some embodiments, the compounds disclosed herein have a human plasma stability, T_{1/2}, of no less than about 15 min. For example, the compounds disclosed herein have a human plasma stability, T_{1/2}, of between about 15 min and about 15 hours. In some embodiments, the compounds disclosed herein have a human plasma stability, T_{1/2}, of between about 15 min and about 30 min. In some embodiments, the compounds disclosed herein have a human plasma stability, T_{1/2}, between about 30 min and about 45 min. In some embodiments, the compounds disclosed herein have a human plasma stability, T_{1/2}, between about 45 min and about 1 hour. In some embodiments, the compounds disclosed herein have a human plasma stability, T_{1/2}, between about 1 and about 2 hours. In some embodiments, the compounds disclosed herein have a human plasma stability, T_{1/2}, between about 2 and about 3 hours. In some embodiments, the compounds disclosed herein have a human plasma stability, T_{1/2}, between about 3 and about 5 hours, the compounds disclosed herein have a human plasma stability, T_{1/2}, between about 5 and about 10 hours, the compounds disclosed herein have a human plasma stability, T_{1/2}, of between about 10 and about 15 hours, the compounds disclosed herein have a human plasma stability, T_{1/2}, of greater than about 1 hour. In some embodiments, the compounds disclosed herein have a human plasma stability, T_{1/2}, of greater than about 2 hours. In some embodiments, the compounds disclosed herein have a human plasma stability, T_{1/2}, of greater than about 4 hours. In some embodiments, the compounds disclosed herein have a human plasma stability, T_{1/2}, of greater than about 10 hours. In some embodiments, the compounds disclosed herein have a human plasma stability, T_{1/2}, of greater than about 15 hours.

As used herein, the “human plasma stability, T_{1/2}” is determined according to the procedure described in Example 10.

### II. Mixture Compositions

Disclosed are compositions containing a compound disclosed herein. In some embodiments, the compound in the composition is in greater than 80%, 85%, 90%, or 95% enantiomeric or diastereomeric excess. In some embodiments, the compound in the composition is in greater than 95% enantiomeric or diastereomeric excess.

In some embodiments, the compositions contain a compound having a structure of Formula (I) or a pharmaceutically acceptable salt thereof, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula (I)-1 or a pharmaceutically acceptable salt thereof, wherein the compound is in greater than 80%, 85%, 90%, or 95% enantiomeric or diastereomeric excess with respect to the configuration depicted by Formula (I)-1. In some embodiments, the compound is in greater than 95% enantiomeric or diastereomeric excess with respect to the configuration depicted by Formula (I)-1.

In some embodiments, the compositions contain a compound having a structure of Formula (I)-2 or a pharmaceutically acceptable salt thereof with respect to the configuration depicted by Formula (I)-2, wherein the compound is in greater than 80%, 85%, 90%, or 95% enantiomeric or diastereomeric excess. In some embodiments, the compound is in greater than 95% enantiomeric or diastereomeric excess with respect to the configuration depicted by Formula (I)-2.

In some embodiments, the compositions contain a compound having a structure of Formula (II) or a pharmaceutically acceptable salt thereof, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula (II)-1 or a pharmaceutically acceptable salt thereof, wherein the compound is in greater than 80%, 85%, 90%, or 95% enantiomeric or diastereomeric excess with respect to the configuration depicted by Formula (II)-1. In some embodiments, the compound is in greater than 95% enantiomeric or diastereomeric excess with respect to the configuration depicted by Formula (II)-1.

In some embodiments, the compositions contain a compound having a structure of Formula (II)-1A or a pharmaceutically acceptable salt thereof, wherein the compound is in greater than 80%, 85%, 90%, or 95% enantiomeric or diastereomeric excess with respect to the configuration depicted by Formula (II)-1A. In some embodiments, the compound is in greater than 95% enantiomeric or diastereomeric excess with respect to the configuration depicted by Formula (II)-1A.

In some embodiments, the compositions contain a compound having a structure of Formula (II)-1B or a pharmaceutically acceptable salt thereof with respect to the configuration depicted by Formula (II)-1B, wherein the compound is in greater than 80%, 85%, 90%, or 95% enantiomeric or diastereomeric excess. In some embodiments, the compound is in greater than 95% enantiomeric or diastereomeric excess with respect to the configuration depicted by Formula (II)-1B.

In some embodiments, the compositions contain a compound having a structure of Formula (II)-2 or a pharmaceutically acceptable salt thereof, wherein the compound is in greater than 80%, 85%, 90%, or 95% enantiomeric or diastereomeric excess with respect to the configuration depicted by Formula (II)-2. In some embodiments, the compound is in greater than 95% enantiomeric or diastereomeric excess with respect to the configuration depicted by Formula (II)-2.

In some embodiments, the compositions contain a compound having a structure of Formula (II)-2A or a pharmaceutically acceptable salt thereof, wherein the compound is in greater than 80%, 85%, 90%, or 95% enantiomeric or diastereomeric excess with respect to the configuration depicted by Formula (II)-2A. In some embodiments, the compound is in greater than 95% enantiomeric or diastereomeric excess with respect to the configuration depicted by Formula (II)-2A.

In some embodiments, the compositions contain a compound having a structure of Formula (II)-2B or a pharmaceutically acceptable salt thereof, wherein the compound is in greater than 80%, 85%, 90%, or 95% enantiomeric or diastereomeric excess with respect to the configuration depicted by Formula (II)-2B. In some embodiments, the compound is in greater than 95% enantiomeric or diastereomeric excess with respect to the configuration depicted by Formula (II)-2B.

In some embodiments, the compositions contain a compound having a structure of Formula (III) or a pharmaceutically acceptable salt thereof, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula (III)-1 or a pharmaceutically acceptable salt thereof, wherein the compound is in greater than 80%, 85%, 90%, or 95% enantiomeric or diastereomeric excess with respect to the configuration depicted by Formula (III)-1. In some embodiments, the compound is in greater than 95% enantiomeric or diastereomeric excess with respect to the configuration depicted by Formula (III)-1.

In some embodiments, the compositions contain a compound having a structure of Formula (III)-1A or a pharmaceutically acceptable salt thereof, wherein the compound is in greater than 80%, 85%, 90%, or 95% enantiomeric or diastereomeric excess with respect to the configuration depicted by Formula (III)-1A. In some embodiments, the compound is in greater than 95% enantiomeric or diastereomeric excess with respect to the configuration depicted by Formula (III)-1A.

In some embodiments, the compositions contain a compound having a structure of Formula (III)-1B or a pharmaceutically acceptable salt thereof with respect to the configuration depicted by Formula (III)-1B, wherein the compound is in greater than 80%, 85%, 90%, or 95% enantiomeric or diastereomeric excess. In some embodiments, the compound is in greater than 95% enantiomeric or diastereomeric excess with respect to the configuration depicted by Formula (III)-1B.

In some embodiments, the compositions contain a compound having a structure of Formula (III)-2 or a pharmaceutically acceptable salt thereof, wherein the compound is in greater than 80%, 85%, 90%, or 95% enantiomeric or diastereomeric excess with respect to the configuration depicted by Formula (III)-2. In some embodiments, the compound is in greater than 95% enantiomeric or diastereomeric excess with respect to the configuration depicted by Formula (III)-2.

In some embodiments, the compositions contain a compound having a structure of Formula (III)-2A or a pharmaceutically acceptable salt thereof, wherein the compound is in greater than 80%, 85%, 90%, or 95% enantiomeric or diastereomeric excess with respect to the configuration depicted by Formula (III)-2A. In some embodiments, the compound is in greater than 95% enantiomeric or diastereomeric excess with respect to the configuration depicted by Formula (III)-2A.

In some embodiments, the compositions contain a compound having a structure of Formula (III)-2B or a pharmaceutically acceptable salt thereof, wherein the compound is in greater than 80%, 85%, 90%, or 95% enantiomeric or diastereomeric excess with respect to the configuration depicted by Formula (III)-2B. In some embodiments, the compound is in greater than 95% enantiomeric or diastereomeric excess with respect to the configuration depicted by Formula (III)-2B.

In some embodiments, the compositions contain a compound having a structure of Formula (IV) or a pharmaceutically acceptable salt thereof, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula (IV)-A or a pharmaceutically acceptable salt thereof, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula (IV)-1A or a pharmaceutically acceptable salt thereof, wherein the compound is in greater than 80%, 85%, 90%, or 95% enantiomeric or diastereomeric excess with respect to the configuration depicted by Formula (IV)-1A. In some embodiments, the compound is in greater than 95% enantiomeric or diastereomeric excess with respect to the configuration depicted by Formula (IV)-1A.

In some embodiments, the compositions contain a compound having a structure of Formula (IV)-2A or a pharmaceutically acceptable salt thereof, wherein the compound is in greater than 80%, 85%, 90%, or 95% enantiomeric or diastereomeric excess with respect to the configuration depicted by Formula (IV)-2A. In some embodiments, the compound is in greater than 95% enantiomeric or diastereomeric excess with respect to the configuration depicted by Formula (IV)-2A.

The disclosed compounds may be present in a mixture of a salt form and a non-salt form. In some embodiments, more than 50%, 60%, 70%, 80%, 90%, 95%, or 98% of the compound in the mixture may be in the non-salt form, calculated as the ratio of the weight of the non-salt form to the total weight of the mixture. In some embodiments, more than 90% of the compound in the mixture may be in the non-salt form. In some embodiments, more than 50%, 60%, 70%, 80%, 90%, 95%, or 98% of the compound in the mixture may be in the salt form, calculated as the ratio of the weight of the salt form to the total weight of the mixture. In some embodiments, more than 90% of the compound in the mixture may be in the salt form. In some embodiments, more than 50%, 60%, 70%, 80%, 90%, 95%, or 98% of the compound in the mixture may be in an ammonium salt form, calculated as the ratio of the weight of the ammonium salt form to the total weight of the mixture. In some embodiments, more than 90% of the compound in the mixture may be in the ammonium salt form.

### III. Pharmaceutical Compositions and Kits

The compounds of the present disclosure may be formulated as a pharmaceutical composition containing at least one compound of the present disclosure, optionally a pharmaceutically acceptable carrier, and optionally one other pharmaceutically active compound.

In some embodiments, pharmaceutical compositions for use in the present disclosure comprise an effective amount of at least one compound of Formulae (I), (II), (III), (IV), or their sub-formulae or a pharmaceutically acceptable salt thereof, optionally a pharmaceutically acceptable carrier, and optionally one other pharmaceutically active compound. The preparations may be prepared in a manner known per se, which usually involves mixing the at least one compound according to the disclosure with the pharmaceutically acceptable carrier and, if desired, in combination with the one or more other pharmaceutical active compounds, when necessary, under aseptic conditions. Reference is again made to standard handbooks, such as the latest edition of Remington's Pharmaceutical Sciences.

In some embodiments, the pharmaceutical compositions are in a form chosen from tablets, capsules, pills, gels, granules, aerosols, solutions (such as aqueous solutions, e.g., salines and buffered salines), suspensions, nanoparticle formulations (including liposomes), and emulsions. In some embodiments, the pharmaceutical compositions are in the form of an oral formulation. For example, the oral formulation may be in a solid form, such as tablets, capsules, pills, and granules. Alternatively, the oral formulation may be in a liquid form, such as solutions, suspensions, and emulsions.

### A. Physical Forms and Unit Dosages

Depending upon the manner of introduction, the compounds described herein may be formulated in a variety of ways. Pharmaceutical compositions containing one or more compounds can be prepared in various forms, such as granules, tablets, capsules, pills, suppositories, powders, controlled release formulations, solutions (such as aqueous solutions, e.g., salines and buffered salines), nanoparticle formulations (including liposomes), suspensions, emulsions, creams, gels, ointments, salves, lotions, aerosols, and the like.

In some embodiments, the pharmaceutical compositions are employed in solid dosage forms suitable for simple administration of precise dosages. For example, solid dosage forms for oral administration include, but are not limited to, tablets, pills, granules, soft or hard gelatin or non-gelatin capsules, and caplets. Optionally, the solid dosage form is a lyophilized powder that can be readily dissolved and converted to a liquid dosage form for oral, intravenous, or intramuscular administration. In some embodiments, the lyophilized powder is manufactured by dissolving the active ingredient (e.g., a compound of Formulae (I), (II), (III), (IV), or their sub-formulae or a pharmaceutically acceptable salt thereof) in an aqueous medium followed by lyophilization. In some embodiments, the aqueous medium is water, normal saline, or PBS.

In some embodiments, the pharmaceutical compositions are employed in lipid dosage forms, such as solutions, suspensions, emulsions, syrups, shakes, etc. Some liquid dosage forms, such as solutions, suspensions, emulsions, syrups, and shakes, are suitable for oral administration. Some liquid dosage forms, such as solutions and suspensions, are suitable for intravenous or intramuscular administration. In some embodiments, the pharmaceutical compositions are in the form of a sterile aqueous solution. In some embodiments, the sterile aqueous solution is a sterile normal saline solution. In some embodiments, the sterile aqueous solution is a sterile PBS solution. In some embodiments, the sterile aqueous solution is manufactured by dissolving a lyophilized powder containing the active ingredient (e.g., a compound of Formulae (I), (II), (III), (IV), or their sub-formulae or a pharmaceutically acceptable salt thereof) in an aqueous medium. For example, the sterile aqueous solution can be prepared by dissolving a lyophilized powder containing the active ingredient in a dose-appropriate volume of the aqueous medium. In some embodiments, the aqueous medium is sterile water, sterile normal saline, or sterile PBS.

In some embodiments, the at least one compound Formulae (I), (II), (III), (IV), or their sub-formulae or a pharmaceutically acceptable salt thereof is formulated for topical administration. Suitable topical dosage forms include lotions, creams, ointments, and gels. A “gel” is a semisolid system containing a dispersion of the active ingredient (e.g., a compound of Formulae (I), (II), (III), (IV), or their sub-formulae or a pharmaceutically acceptable salt thereof) in a liquid vehicle that is rendered semisolid by the action of a thickening agent or polymeric material dissolved or suspended in the liquid vehicle. The liquid vehicle may include a lipophilic component, an aqueous component, or both. Some emulsions may be gels or otherwise include a gel component. Some gels, however, are not emulsions because, for example, they do not contain a homogenized blend of immiscible components.

In some embodiments, the pharmaceutical composition contains at least one compound of at least one compound of Formulae (I), (II), (III), (IV), or their sub-formulae or a pharmaceutically acceptable salt thereof and a propellant. In some embodiments, the propellant is an aerosolizing propellant. In some embodiments, the aerosolizing propellant is chosen from compressed air, ethanol, nitrogen, carbon dioxide, nitrous oxide, hydrofluoroalkanes (HFAs), 1,1,1,2-tetrafluoroethane, 1,1,1,2,3,3,3-heptafluoropropane, and combinations thereof.

In some embodiments, the pharmaceutical compositions are in a unit dosage form and may be suitably packaged, for example in a box, blister, vial, bottle, sachet, ampoule or in any other suitable single-dose or multi-dose holder or container (which may be properly labelled), optionally with one or more leaflets containing product information and/or instructions for use. In some embodiments, such unit dosages comprise between 1 and 1000 mg, between 5 and 500 mg, between 5 and 300 mg, between 5 and 200 mg, or between 5 and 100 mg of at least one compound of Formulae (I), (II), (III), (IV), or their sub-formulae or a pharmaceutically acceptable salt thereof, e.g., about 10, about 25, about 50, about 100, about 200, about 300 or about 400 mg per unit dosage. For human patients (including both adult and pediatric patients), the dose of at least one compound of Formulae (I), (II), (III), (IV), or their sub-formulae or a pharmaceutically acceptable salt thereof may be lower than 25 mg/kg per day, lower than 20 mg/kg per day, lower than 15 mg/kg per day, lower than 12.5 mg/kg per day, lower than 10 mg/kg per day, lower than 5 mg/kg per day, or lower than 2.5 mg/kg per day. In some embodiments, the dose of at least one compound of Formulae (I), (II), (III), (IV), or their sub-formulae or a pharmaceutically acceptable salt thereof is lower than 5 mg/kg per day. In some embodiments, the dose of at least one compound of Formulae (I), (II). (III). (IV), or their sub-formulae or a pharmaceutically acceptable salt thereof is lower than 2.5 mg/kg per day.

### B. Pharmaceutically Acceptable Carrier

Pharmaceutical compositions containing at least one compound of Formulae (I), (II). (III), (IV), or their sub-formulae or a pharmaceutically acceptable salt thereof may be prepared using a pharmaceutically acceptable carrier composed of one or more materials that are considered safe and effective and may be administered to an individual without causing undesirable biological side effects or unwanted interactions. The pharmaceutically acceptable carrier may encapsulate, embed, entrap, dissolve, disperse, absorb, and/or bind to the at least one compound.

As used herein, “carrier” refers to components present in the pharmaceutical compositions other than the active ingredient or ingredients. Exemplary carriers of the pharmaceutical compositions may contain one or more of the following pharmaceutically acceptable excipients: diluents, binders, lubricants, disintegrators, fillers, pH modifying or modifying agents, preservatives, antioxidants, wetting or emulsifying agents, dyes, and solubility enhancers.

Suitable pharmaceutically acceptable excipients present in the drug-containing tablets, beads, granules, or particles include, but are not limited to, diluents, binders, lubricants, disintegrants, colorants, stabilizers, and surfactants.

Diluents, also referred to as “fillers,” may be desired to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminium silicate, and powdered sugar.

Binders may be used to impart cohesive qualities to a solid dosage formulation and thus ensure that a tablet, bead, or granule remains intact after the formation of the dosage formulation. Suitable binder materials include, but are not limited to, starch, pregelatinized starch, gelatin, sugars (such as sucrose, glucose, dextrose, lactose, and sorbitol), polyethylene glycol, waxes, natural and synthetic gums, acacia, tragacanth, sodium alginate, cellulose (such as hydroxypropylmethylcellulose, hydroxypropylcellulose, and ethylcellulose), VEEGUM, and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid, and polyvinylpyrrolidone).

Lubricants may be used to facilitate tablet manufacture. Examples of suitable lubricants include, but are not limited to, magnesium stearate, calcium stearate, stearic acid, glycerol behenate, polyethylene glycol, talc, and mineral oil.

Disintegrants may be used to facilitate dosage form disintegration or “breakup” after administration. Exemplary disintegrants include, but are not limited to, starch, sodium starch glycolate, sodium carboxymethyl starch, sodium carboxymethylcellulose, hydroxypropyl cellulose, pregelatinized starch, clays, cellulose, gums, and cross-linked polymers, such as cross-linked PVP (e.g., Polyplasdone XL).

Stabilizers may be used to inhibit or retard drug decomposition reactions which include, by way of example, oxidative reactions.

Surfactants may be anionic, cationic, amphoteric, or non-ionic surface-active agents. Suitable anionic surfactants include, but are not limited to, those containing carboxylate, sulfonate, and sulfate ions. Examples of anionic surfactants include sodium, potassium, ammonium of long chain alkyl sulfonates, alkylaryl sulfonates (such as sodium dodecylbenzene sulfonate), dialkyl sodium sulfosuccinates (such as sodium bis-(2-ethylthioxyl)-sulfosuccinate), and alkyl sulfates (such as sodium lauryl sulfate). Cationic surfactants include, but are not limited to, quaternary ammonium compounds, such as benzalkonium chloride, benzethonium chloride, cetrimonium bromide, stearyl dimethylbenzyl ammonium chloride, polyoxyethylene, and coconut amine. Examples of non-ionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether. PEG-1000 cetyl ether, polyoxyethylene tridecyl ether, polypropylene glycol butyl ether, Poloxamer 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

In some embodiments, the pharmaceutically acceptable carrier may contain one or more coating materials. Examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers and methacrylic resins that are commercially available under the trade name EUDRAGIT®, zein, shellac, and polysaccharides. Additionally, the coating materials may contain one or more excipients like plasticizers, pigments, colorants, glidants, stabilization agents, pore formers, and surfactants.

The concentration of the at least one compound of Formulae (I), (II), (III), (IV), or their sub-formulae or a pharmaceutically acceptable salt thereof in the pharmaceutical compositions may vary from about 0.5 to about 100% (weight percent). For oral use, the pharmaceutical compositions may contain from about 5 to about 100% by weight of the at least one compound of Formulae (I), (II), (III), (IV), or their sub-formulae or a pharmaceutically acceptable salt thereof. For other uses, the pharmaceutical compositions may contain from about 0.5 to about 50% by weight of the at least one compound of Formulae (I), (II), (III), (IV), or their sub-formulae or a pharmaceutically acceptable salt thereof.

### C. Controlled Release

The pharmaceutical compositions described herein can be formulated for modified or controlled release. Examples of controlled release dosage forms include extended release dosage forms, delayed release dosage forms, pulsatile release dosage forms, and combinations thereof.

Delayed release, extended release, and/or pulsatile release dosage forms may be prepared as described in standard references, such as “Pharmaceutical dosage form tablets” eds. Liberman et al., Marcel Dekker, Inc., 1989); “Remington—The science and practice of pharmacy”, 20th ed., Lippincott, Williams & Wilkins, 2000; and “Pharmaceutical dosage forms and drug delivery systems”, 6th Edition, Ansel et al., Williams & Wilkins, 1995). These references provide information on carriers, materials, equipment, and processes for preparing tablets and capsules and delayed release dosage forms of tablets, capsules, and granules.

Optionally, each dosage unit in the capsule may comprise a plurality of drug-containing beads, granules, or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that may or may not include one or more additional excipients or polymers. In contrast to drug-containing beads, granules and particles do not contain an inert support. Granules may comprise drug particles and require further processing. In some embodiments, particles are smaller than granules and are not further processed. Although beads, granules, and particles may be formulated to provide immediate release, beads and granules may be employed to provide delayed release.

### 1. Extended Release

The extended release formulations may be prepared as diffusion or osmotic systems, for example, as described in “Remington—The science and practice of pharmacy”, 20th ed., Lippincott. Williams & Wilkins, 2000. A diffusion system may consist of two types of devices, a reservoir and a matrix and is well known and described in the art. The matrix devices may be prepared by compressing the drug with a slowly dissolving polymer carrier into a tablet form. The three major types of materials used in the preparation of matrix devices are plastics, hydrophilic polymers, and fatty compounds. Plastics include, but are not limited to, methyl acrylate-methyl methacrylate, polyvinyl chloride, and polyethylene. Hydrophilic polymers include, but are not limited to, cellulosic polymers (such as methyl and ethyl cellulose, hydroxyalkylcelluloses (e.g., hydroxypropylcellulose, hydroxypropylmethylcellulose), and carboxymethylcellulose), CARBOPOL® 934, polyethylene oxides, and mixtures thereof. Fatty compounds include, but are not limited to, waxes (such as carnauba wax and glyceryl tristearate), wax-type substances including hydrogenated castor oil and hydrogenated vegetable oil, and mixtures thereof.

In some embodiments, the plastic is a pharmaceutically acceptable acrylic polymer. In some embodiments, the pharmaceutically acceptable acrylic polymer is chosen from acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acid), polymethacrylate, polyacrylamide, poly(methacrylic acid anhydride), and glycidyl methacrylate copolymers.

In some embodiments, the pharmaceutically acceptable acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some embodiments, the pharmaceutically acceptable acrylic polymer is an acrylic resin lacquer such as those commercially available under the tradename EUDRAGIT®. In some embodiments, the pharmaceutically acceptable acrylic polymer contains a mixture of two acrylic resin lacquers, EUDRAGIT® RL (such as EUDRAGIT® RL30D) and EUDRAGIT® RS (such as EUDRAGIT® RS30D). EUDRAGIT® RL30D and EUDRAGIT® RS30D are copolymers of acrylic and methacrylic acid esters with a low content of quaternary ammonium groups, the molar ratio of ammonium groups to the remaining neutral methacrylic esters being 1:20 in EUDRAGIT® RL30D and 1:40 in EUDRAGIT® RS30D. The code designations RL (high permeability) and RS (low permeability) refer to the permeability properties of these polymers. EUDRAGIT® RL/RS mixtures are insoluble in water and in digestive fluids. However, multi-particulate systems formed to include the same are swellable and permeable in aqueous solutions and digestive fluids. The EUDRAGIT® RL/RS mixtures may be prepared in any desired ratio in order to ultimately obtain a sustained-release formulation having a desirable release profile. Suitable sustained-release, multi-particulate systems may be obtained, for instance, from 90% EUDRAGIT® RL+10% EUDRAGIT® RS, to 50% EUDRAGIT® RL+50% EUDRAGIT® RS, and to 10% EUDRAGIT® RL+90% EUDRAGIT® RS. In some embodiments, the pharmaceutically acceptable acrylic polymer can also be or include other acrylic resin lacquers, such as EUDRAGIT® S-100, EUDRAGIT® L-100, and mixtures thereof.

Alternatively, the extended release formulations can be prepared using osmotic systems or by applying a semi-permeable coating to a solid dosage form. In the latter case, the desired release profile can be achieved by combining low permeable and high permeable coating materials in suitable proportions.

Matrices with different release mechanisms or profiles can be combined in a final dosage form containing single or multiple units. Examples of multiple units include, but are not limited to, multilayer tablets and capsules containing beads, granules, and/or particles of the active ingredient. An immediate release portion can be added to the extended release system by means of either applying an immediate release layer on top of the extended release core using a coating or compression process or in a multiple unit system such as a capsule containing both extended and immediate release beads.

Extended release tablets containing hydrophilic polymers can be prepared by techniques commonly known in the art, such as direct compression, wet granulation, or dry granulation processes. Their formulations may incorporate polymers, diluents, binders, and lubricants as well as the active pharmaceutical ingredient. The usual diluents include inert powdered substances, such as starches, powdered cellulose, crystalline and microcrystalline cellulose, sugars, such as fructose, mannitol and sucrose, grain flours, and similar edible powders. Diluents include, for example, various types of starch, lactose, mannitol, kaolin, calcium phosphate or sulfate, inorganic salts, such as sodium chloride, and powdered sugar. Powdered cellulose derivatives may also be useful. Tablet binders may include substances, such as starch, gelatin and sugars, such as lactose, fructose, and glucose. Natural and synthetic gums, including acacia, alginates, methylcellulose and polyvinylpyrrolidone can also be used. Polyethylene glycol, hydrophilic polymers, ethylcellulose, and waxes can also serve as binders. A lubricant may be used in a tablet formulation to prevent the tablet and punches from sticking in the die. The lubricant may be chosen from such slippery solids as talc, magnesium and calcium stearate, stearic acid, and hydrogenated vegetable oils.

Extended release tablets containing wax materials may be prepared using methods known in the art, such as a direct blend method, a congealing method, and an aqueous dispersion method. In the congealing method, the drug is mixed with a wax material and either spray-congealed or congealed and screened and processed.

### 2. Delayed Release

Delayed release formulations may be created by coating a solid dosage form with a polymer film, which is insoluble in the acidic environment of the stomach and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. In some embodiments, coating materials include bioerodible, gradually hydrolyzable, gradually water-soluble and/or enzymatically degradable polymers and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers, such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, formed from acrylic acid, methacrylic acid, methyl acrylate, ethyl acrylate, methyl methacrylate and/or ethyl methacrylate and other methacrylic resins that are commercially available under the tradename EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification) and EUDRAGIT® NE. RL and RS (water-insoluble polymers having different degrees of permeability and expandability); vinyl polymers and copolymers, such as polyvinyl pyrrolidone, vinyl acetate, vinylacetate phthalate, vinylacetate crotonic acid copolymer and ethylene-vinyl acetate copolymer; enzymatically degradable polymers, such as azo polymers, pectin, chitosan, amylose and guar gum; and zein and shellac. Combinations of different coating materials may also be used. Multi-layer coatings using different polymers may also be applied.

The coating weights for particular coating materials may be readily determined by those skilled in the art by evaluating individual release profiles for tablets, beads, and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer may be present to reduce the fragility of the coating. A plasticizer may represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent may be used to stabilize particles in the dispersion. Stabilizing agents may be nonionic emulsifiers, such as sorbitan esters, polysorbates, and polyvinylpyrrolidone. Glidants may reduce sticking effects during film formation and drying. Glidants may represent approximately 25 wt. % to 100 wt. % of the polymer weight in the coating solution. One effective glidant is talc. Other glidants, such as magnesium stearate and glycerol monostearates may also be used. Pigments, such as titanium dioxide may also be used. Small quantities of an anti-foaming agent, such as a silicone (e.g., simethicone), may also be added to the coating composition.

### 3. Pulsatile Release

Pulsatile release formulations can provide pulsatile delivery of the one or more compounds of the present disclosure. By “pulsatile” it is meant that a plurality of drug doses are released at spaced apart intervals of time. In some embodiments, upon ingestion of the dosage form, release of the initial dose is substantially immediate. i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In some embodiments, the pulsatile release profile is achieved with dosage forms that are closed and sealed capsules housing at least two drug-containing “dosage units” wherein each dosage unit within the capsule provides a different drug release profile. Control of the delayed release dosage unit(s) may be accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

### D. Co-Formulations

In some embodiments, the pharmaceutical compositions may include one other pharmaceutically active compound. In this context, the at least one compound of Formulae (I), (II), (III), (IV), or their sub-formulae or a pharmaceutically acceptable salt thereof may be formulated in the same dosage form or in separate dosage forms with the other pharmaceutically active compound. For example, the other pharmaceutically active compound may be formulated for immediate release, controlled release, or combinations thereof, either together with or separate from the at least one compound of Formulae (I), (II), (III), (IV), or their sub-formulae or a pharmaceutically acceptable salt thereof.

In some embodiments, the at least one compound of Formulae (I), (II), (III), (IV), or their sub-formulae or a pharmaceutically acceptable salt thereof and the other pharmaceutically active compound are formulated in the same dosage form, such as those described above. In some embodiments, the at least one compound of Formulae (I), (II), (III), (IV), or their sub-formulae or a pharmaceutically acceptable salt thereof and the other pharmaceutically active compound are formulated in the same oral formulation, such as those described above.

In some embodiments, the weight ratio of the at least one compound of Formulae (I), (II), (III), (IV), or their sub-formulae or a pharmaceutically acceptable salt thereof to the other pharmaceutically active compound in the pharmaceutical formulations may be between 1:10 and 10:1. In some embodiments, the weight ratio is between 1:5 and 5:1. In some embodiments, the weight ratio is between 1:4 and 4:1. In some embodiments, the weight ratio is between 1:3 and 3:1. In some embodiments, the weight ratio is between 1:2 and 2:1. In some embodiments, the weight ratio is between 1:1.5 and 1.5:1. In some embodiments, the weight ratio is between 1:1.2 and 1.2:1. In some embodiments, the weight ratio is about 1:1.

In some embodiments, the other pharmaceutically active compound is a cannabinoid or analogue thereof. In some embodiments, the other pharmaceutically active compound is CBD. In some embodiments, the other pharmaceutically active compound is cannabidivarin (CBDV).

In some embodiments, the other pharmaceutically active compound is a phosphate prodrug of cannabinoid, such as those described in the PCT Patent Application No. PCT/US2022/038876, the entirety of which is incorporated herein by reference.

In some embodiments, the other pharmaceutically active compound is a phosphate prodrug of CBD, such as those described in the PCT Patent Application No. PCT/US2022/038876. In some embodiments, the phosphate prodrug of CBD is chosen from the following compounds and their pharmaceutically acceptable salts thereof.

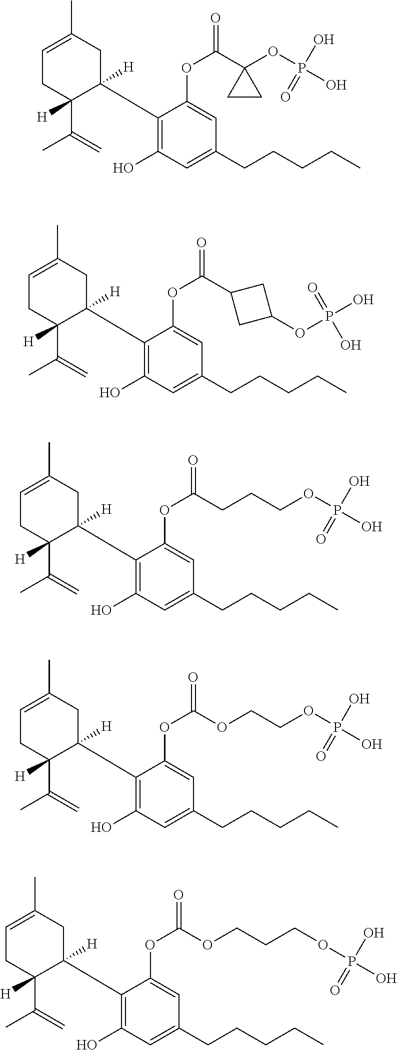 [SMILES: C=C(C)[C@@H]1CC=C(C)C[C@H]1c1c(O)cc(CCCCC)cc1OC(=O)C1(OP(=O)(O)O)CC1 ; C=C(C)[C@@H]1CC=C(C)C[C@H]1c1c(O)cc(CCCCC)cc1OC(=O)C1CC(OP(=O)(O)O)C1 ; C=C(C)[C@@H]1CC=C(C)C[C@H]1c1c(O)cc(CCCCC)cc1OC(=O)CCCOP(=O)(O)O ; C=C(C)[C@@H]1CC=C(C)C[C@H]1c1c(O)cc(CCCCC)cc1OC(=O)OCCOP(=O)(O)O ; C=C(C)[C@@H]1CC=C(C)C[C@H]1c1c(O)cc(CCCCC)cc1OC(=O)OCCCOP(=O)(O)O]

In some embodiments, the other pharmaceutically active compound is a phosphate prodrug of CBDV, such as those described in the PCT Patent Application No. PCT/US2022/038876. In some embodiments, the phosphate prodrug of CBDV is chosen from the following compounds and their pharmaceutically acceptable salts thereof.

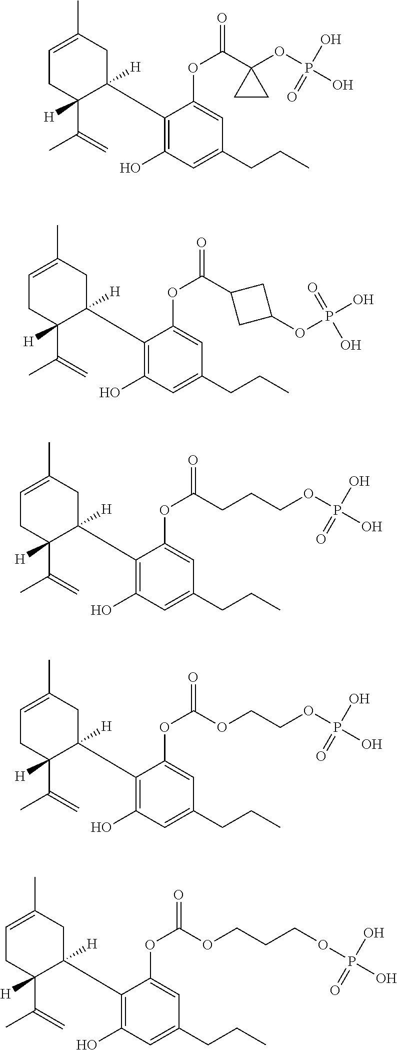 [SMILES: C=C(C)[C@@H]1CC=C(C)C[C@H]1c1c(O)cc(CCC)cc1OC(=O)C1(OP(=O)(O)O)CC1 ; C=C(C)[C@@H]1CC=C(C)C[C@H]1c1c(O)cc(CCC)cc1OC(=O)C1CC(OP(=O)(O)O)C1 ; C=C(C)[C@@H]1CC=C(C)C[C@H]1c1c(O)cc(CCC)cc1OC(=O)CCCOP(=O)(O)O ; C=C(C)[C@@H]1CC=C(C)C[C@H]1c1c(O)cc(CCC)cc1OC(=O)OCCOP(=O)(O)O ; C=C(C)[C@@H]1CC=C(C)C[C@H]1c1c(O)cc(CCC)cc1OC(=O)OCCCOP(=O)(O)O]

In some embodiments, the pharmaceutical compositions contain a phosphate prodrug of (−)trans-Δ⁹-THC or (−)trans-Δ⁸-THC or a pharmaceutically acceptable salt thereof, as described in this disclosure (e.g., Formula (III)-1A and Formula (II)-1A), and a phosphate prodrug of CBD or a pharmaceutically acceptable salt thereof, as described in the PCT Patent Application No. PCT/US2022/038876. In some embodiments, the phosphate prodrug of (−)trans-Δ⁹-THC or (−)trans-Δ⁸-THC or a pharmaceutically acceptable salt thereof and the phosphate prodrug of CBD or a pharmaceutically acceptable salt thereof have the same phosphate prodrug moiety, i.e., the R¹ group as defined herein and in the PCT Patent Application No. PCT/US2022/038876. In some embodiments, the weight ratio of the phosphate prodrug of (−)trans-Δ⁸-THC or (−)trans-Δ⁸-THC or a pharmaceutically acceptable salt thereof to the phosphate prodrug of CBD or a pharmaceutically acceptable salt thereof in the pharmaceutical formulations may be between 1:2 and 2:1. In some embodiments, the weight ratio is between 1:1.5 and 1.5:1. In some embodiments, the weight ratio is between 1:1.2 and 1.2:1. In some embodiments, the weight ratio is about 1:1. In some embodiments, the same prodrug moiety (i.e., the R¹ group) is chosen from

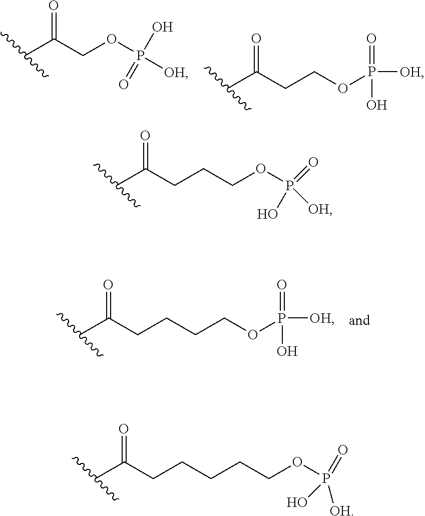 [SMILES: *C(=O)COP(=O)(O)O ; *C(=O)CCOP(=O)(O)O ; *C(=O)CCCOP(=O)(O)O ; *C(=O)CCCCOP(=O)(O)O ; *C(=O)CCCCCOP(=O)(O)O]

In some embodiments, the same prodrug moiety (i.e., the R¹ group) is chosen

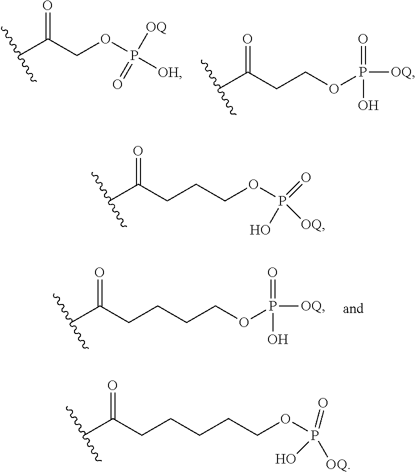

In some embodiments, the same prodrug moiety (i.e., the R¹ group) is chosen from

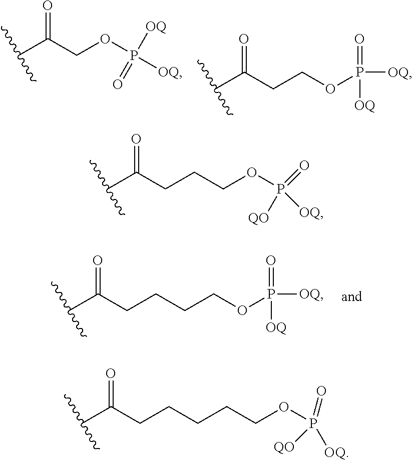

In some embodiments, the same prodrug moiety (i.e., the R¹ group) is chosen from chosen from

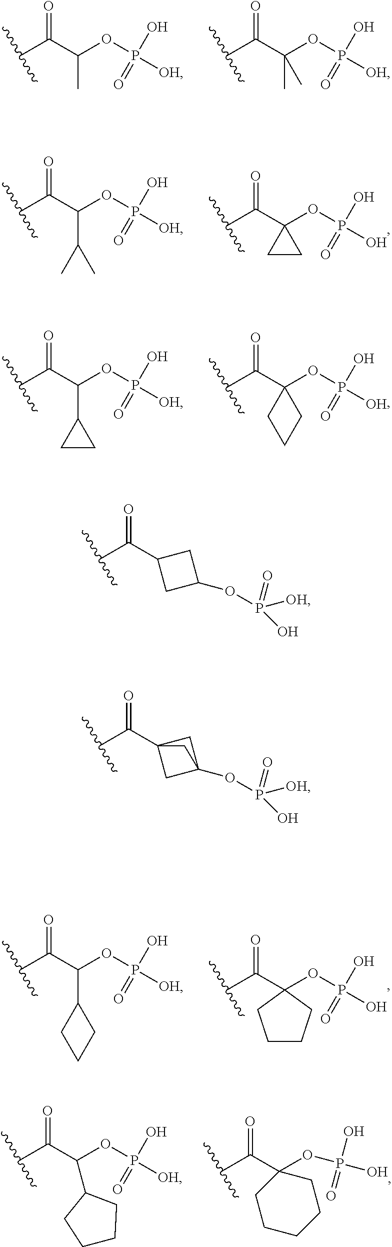

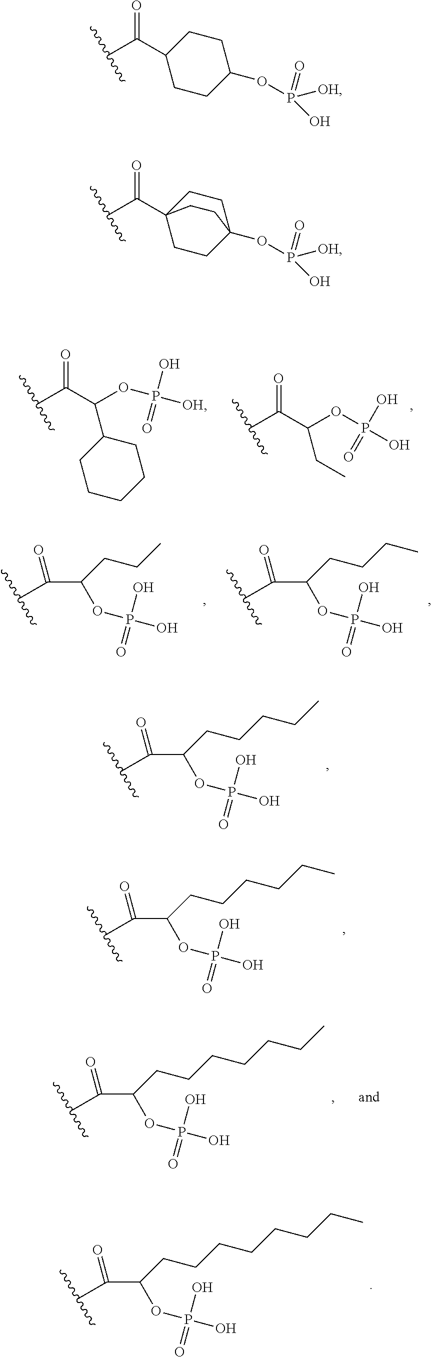

In some embodiments, the same prodrug moiety (i.e., the R¹ group) is chosen from chosen from

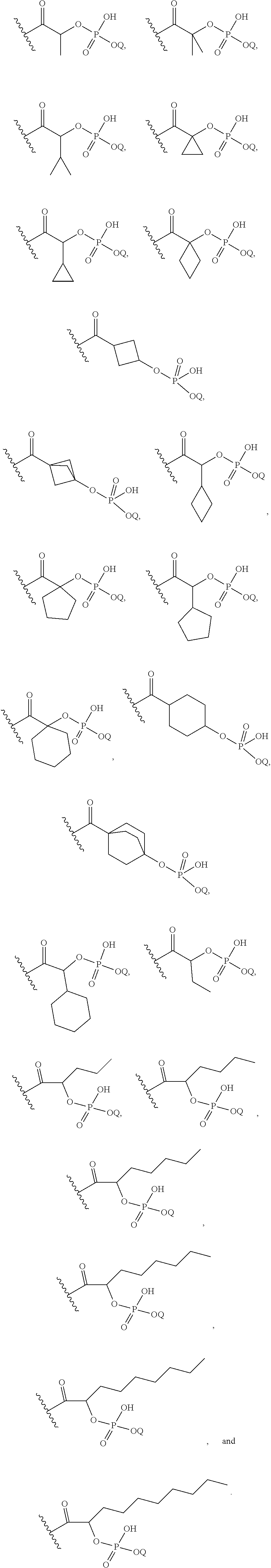

In some embodiments, the same prodrug moiety (i.e., the R¹ group) is chosen from chosen from

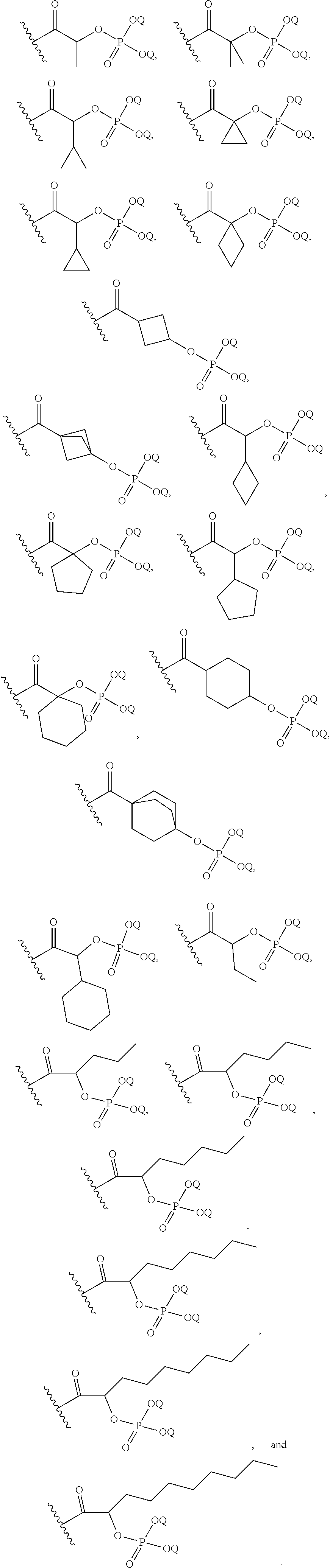

In some embodiments, the same prodrug moiety (i.e., the R¹ group) is chosen from chosen from

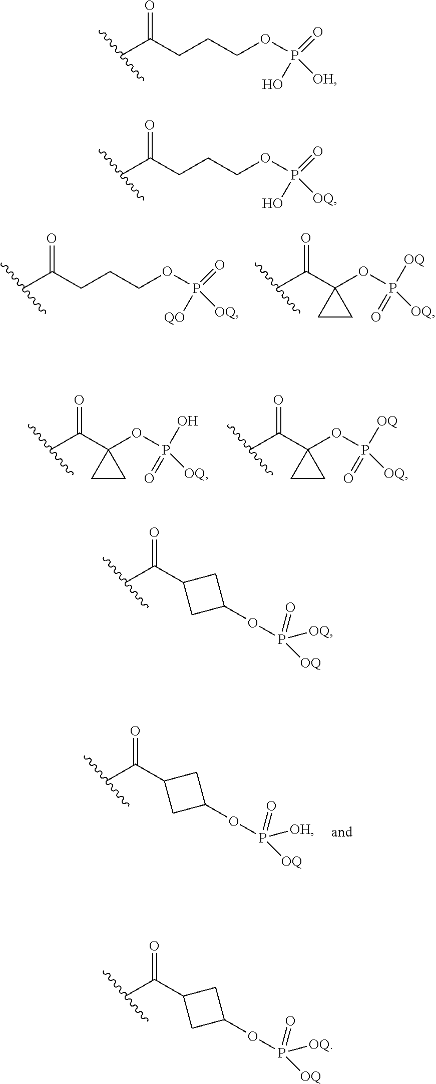

In some embodiments, the same prodrug moiety (i.e., the R¹ group) is chosen from chosen from

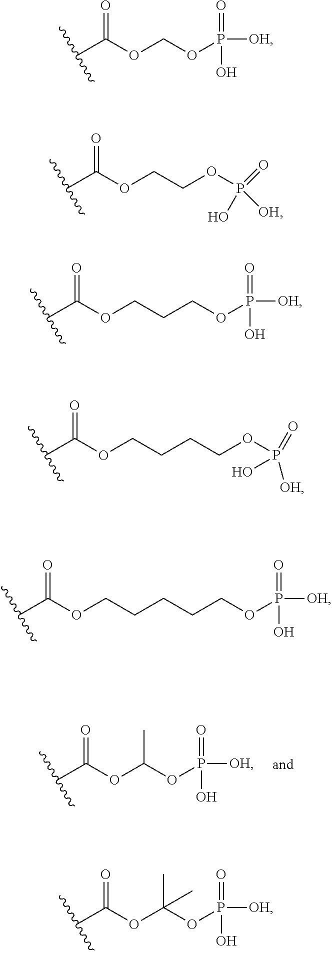

In some embodiments, the same prodrug moiety (i.e., the R¹ group) is chosen from chosen from

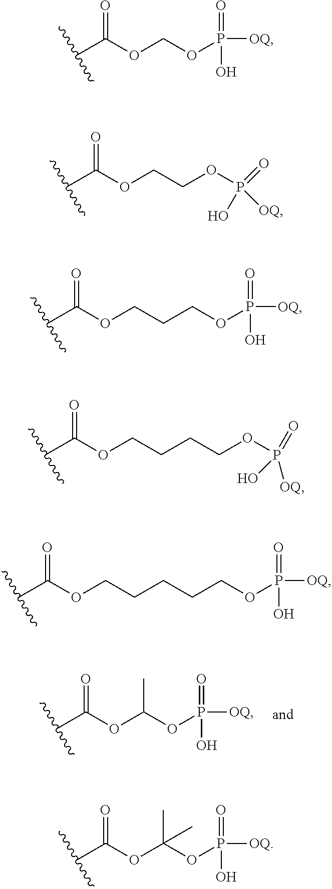

In some embodiments, the same prodrug moiety (i.e., the R¹ group) is chosen from chosen from

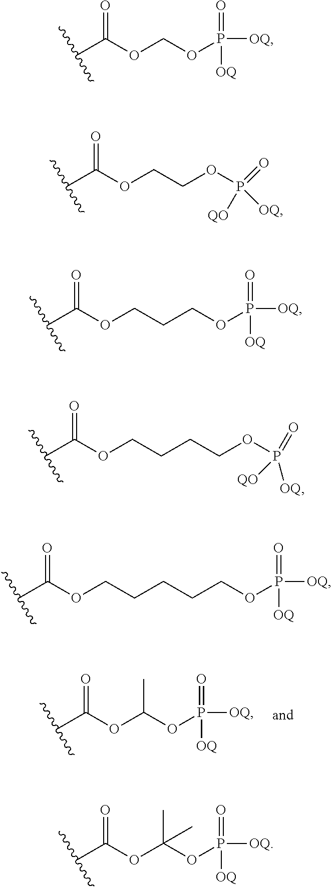

In some embodiments, the same prodrug moiety (i.e., the R¹ group) is chosen from chosen from

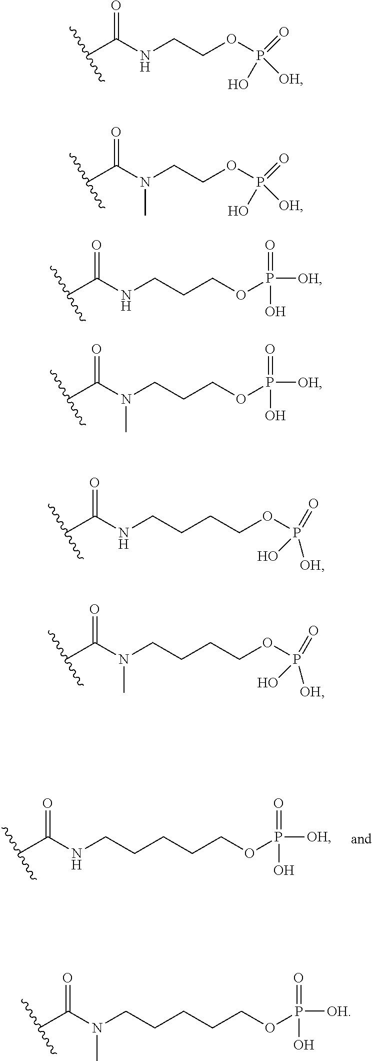

In some embodiments, the same prodrug moiety (i.e., the R¹ group) is chosen from chosen from

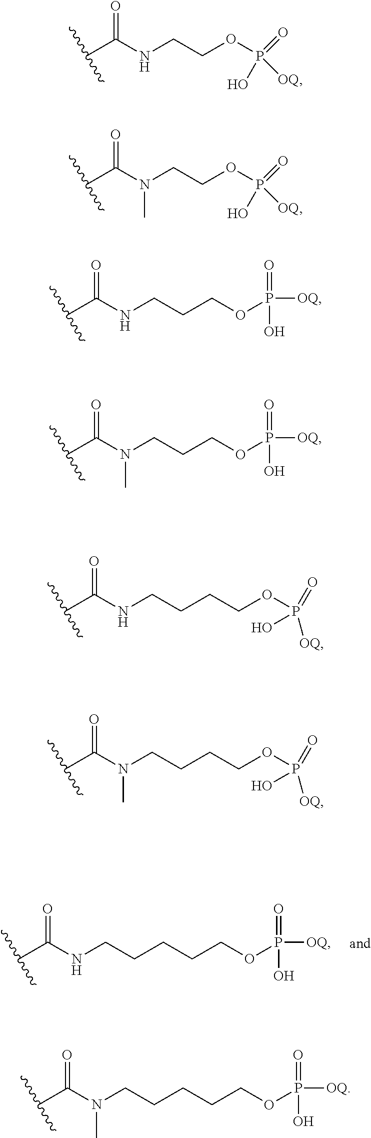

In some embodiments, the same prodrug moiety (i.e., the R¹ group) is chosen from chosen from

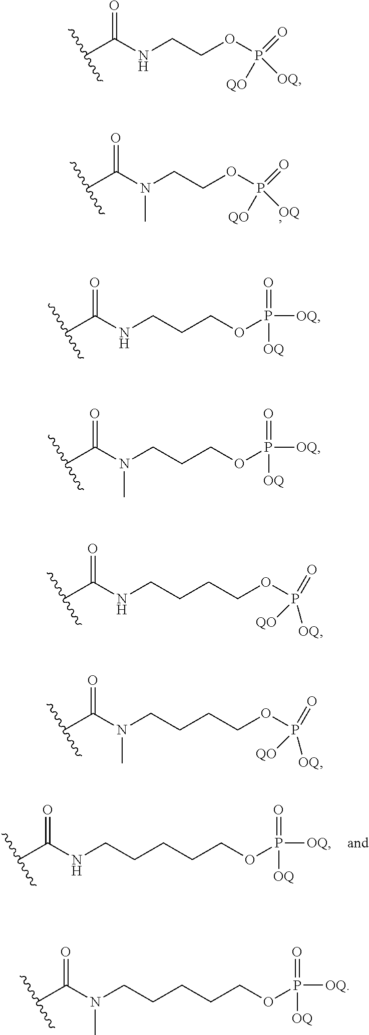

In some embodiments, the same prodrug moiety (i.e., the R¹ group) is

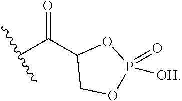 [SMILES: *C(=O)C1COP(=O)(O)O1]

In some embodiments, the same prodrug moiety (i.e., the R¹ group) is

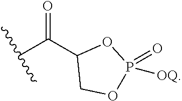

In some embodiments, the same prodrug moiety (i.e., the R¹ group) is

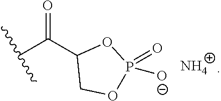 [SMILES: *C(=O)C1COP(=O)([O-])O1 ; [NH4+]]

In some embodiments, the pharmaceutical compositions contain a phosphate prodrug of (−)trans-Δ⁹-THC or (−)trans-Δ⁸-THC or a pharmaceutically acceptable salt thereof, as described in this disclosure (e.g., Formula (III)-1A and Formula (II)-1A), and a phosphate prodrug of CBDV or a pharmaceutically acceptable salt thereof, as described in the PCT Patent Application No. PCT/US2022/038876. In some embodiments, the phosphate prodrug of (−)trans-Δ⁹-THC or (−)trans-Δ⁸-THC or a pharmaceutically acceptable salt thereof and the phosphate prodrug of CBDV or a pharmaceutically acceptable salt thereof have the same phosphate prodrug moiety, i.e., the R¹ group as defined herein and in the PCT Patent Application No. PCT/US2022/038876. In some embodiments, the weight ratio of the phosphate prodrug of (−)trans-Δ⁹-THC or (−)trans-Δ⁸-THC or a pharmaceutically acceptable salt thereof to the phosphate prodrug of CBDV or a pharmaceutically acceptable salt thereof in the pharmaceutical formulations may be between 1:2 and 2:1. In some embodiments, the weight ratio is between 1:1.5 and 1.5:1. In some embodiments, the weight ratio is between 1:1.2 and 1.2:1. In some embodiments, the weight ratio is about 1:1. In some embodiments, the same prodrug moiety (i.e., the R¹ group) is chosen from

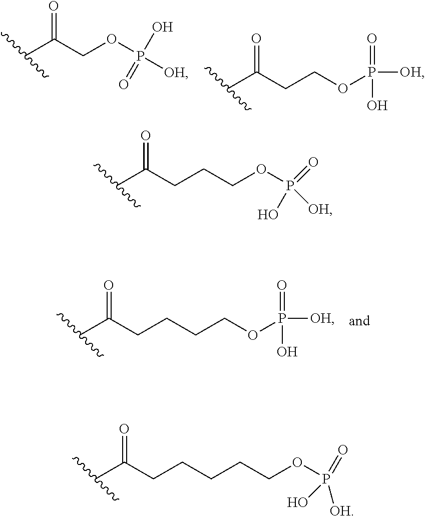 [SMILES: *C(=O)COP(=O)(O)O ; *C(=O)CCOP(=O)(O)O ; *C(=O)CCCOP(=O)(O)O ; *C(=O)CCCCOP(=O)(O)O ; *C(=O)CCCCCOP(=O)(O)O]

In some embodiments, the same prodrug moiety (i.e., the R¹ group) is chosen from

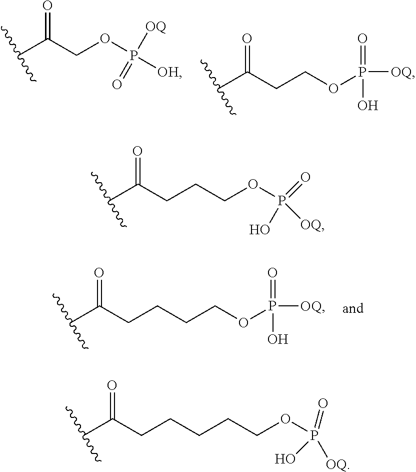

In some embodiments, the same prodrug moiety (i.e., the R¹ group) is chosen from

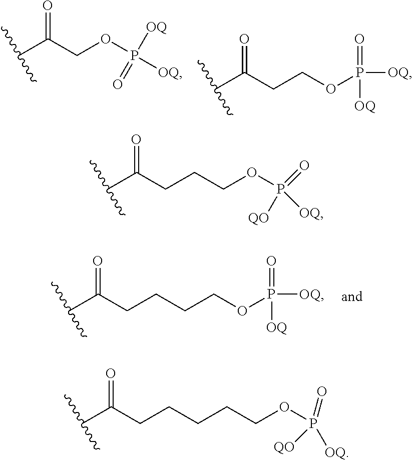

In some embodiments, the same prodrug moiety (i.e., the R¹ group) is chosen from chosen from

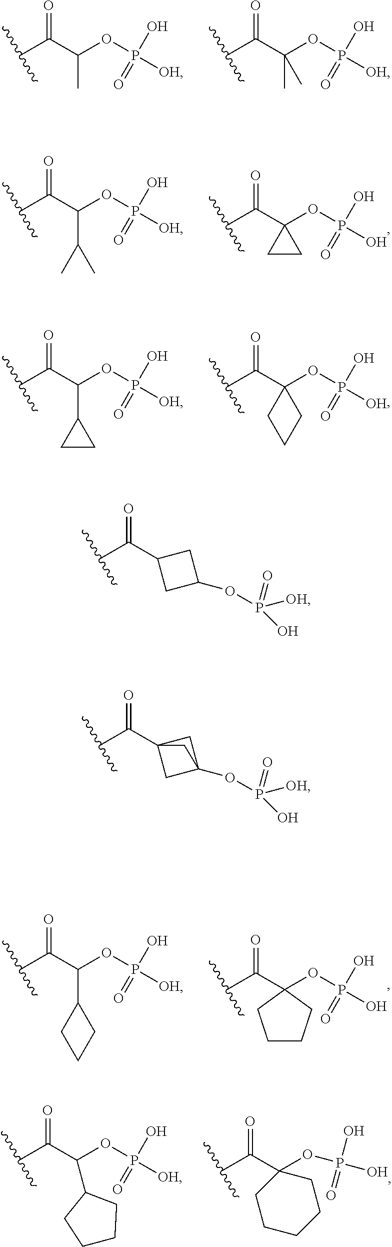

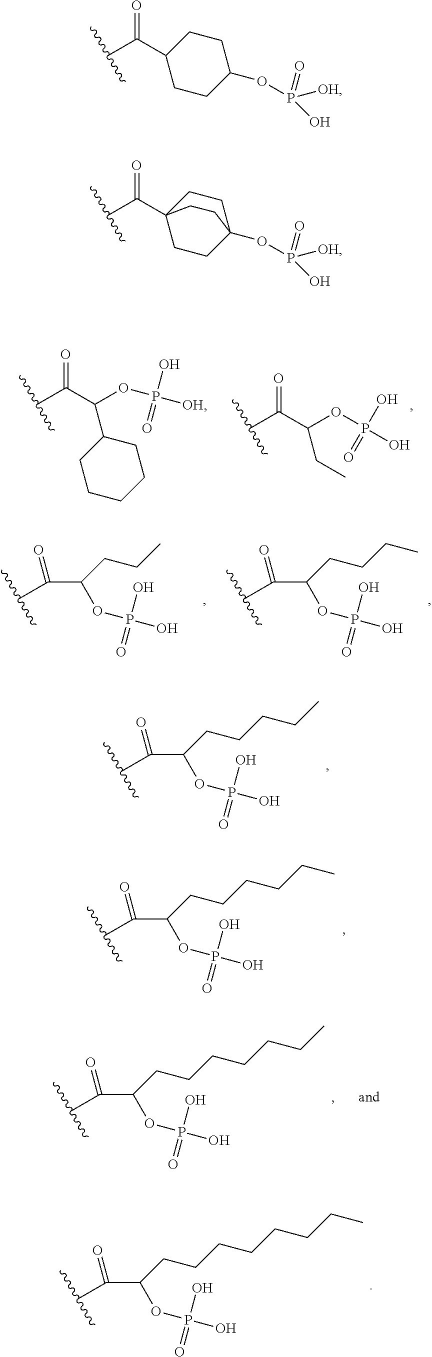

In some embodiments, the same prodrug moiety (i.e., the R¹ group) is chosen from chosen from

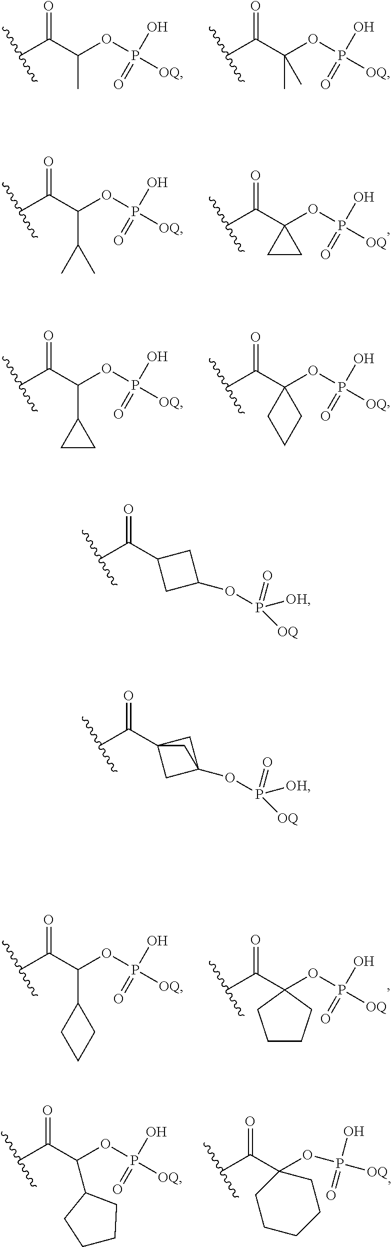

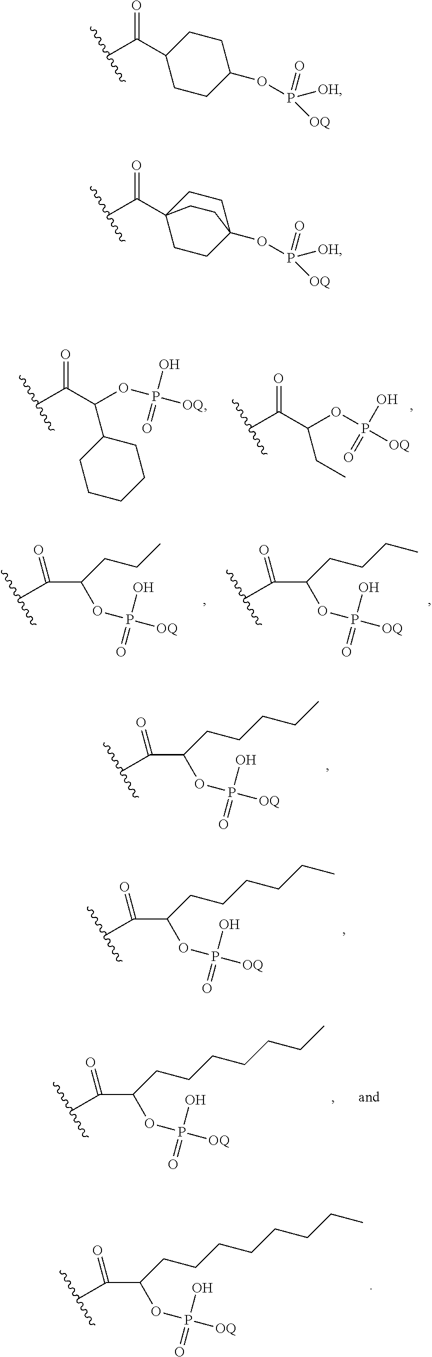

In some embodiments, the same prodrug moiety (i.e., the R¹ group) is chosen from chosen from

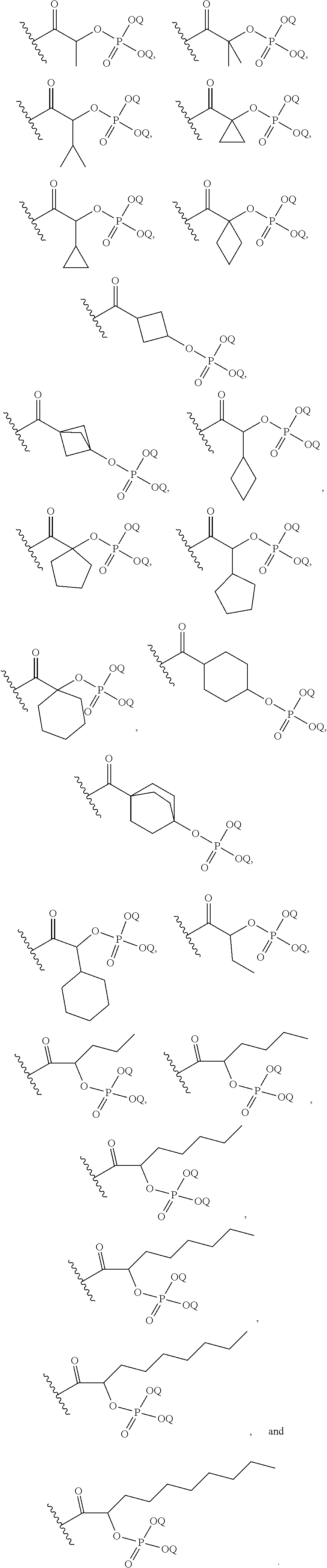

In some embodiments, the same prodrug moiety (i.e., the R¹ group) is chosen from chosen from

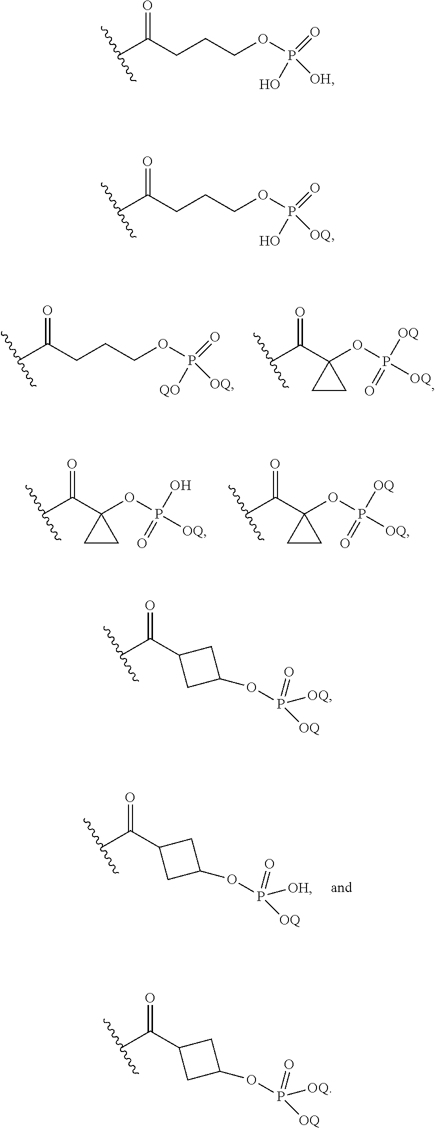

In some embodiments, the same prodrug moiety (i.e., the R¹ group) is chosen from chosen from

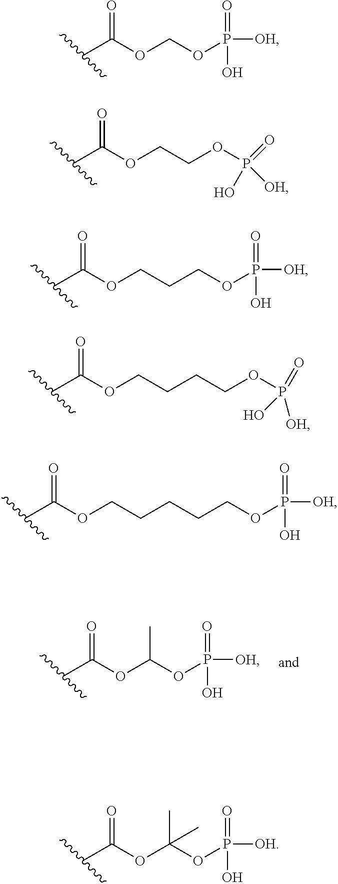

In some embodiments, the same prodrug moiety (i.e., the R¹ group) is chosen from chosen from

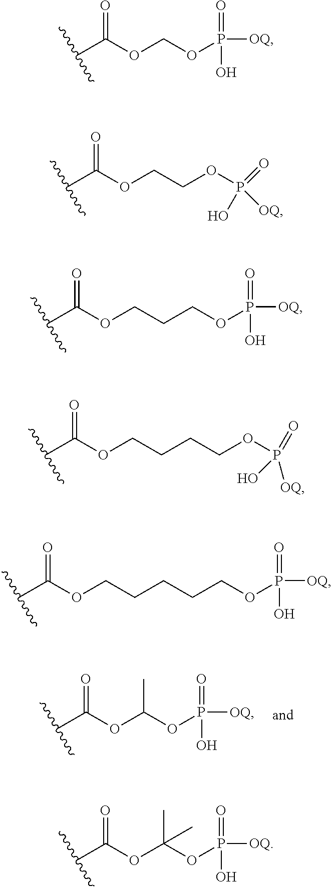

In some embodiments, the same prodrug moiety (i.e., the R¹ group) is chosen from chosen from

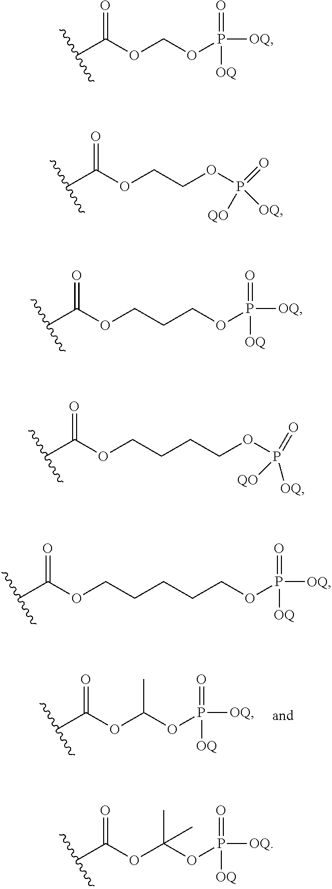

In some embodiments, the same prodrug moiety (i.e., the R¹ group) is chosen from chosen from

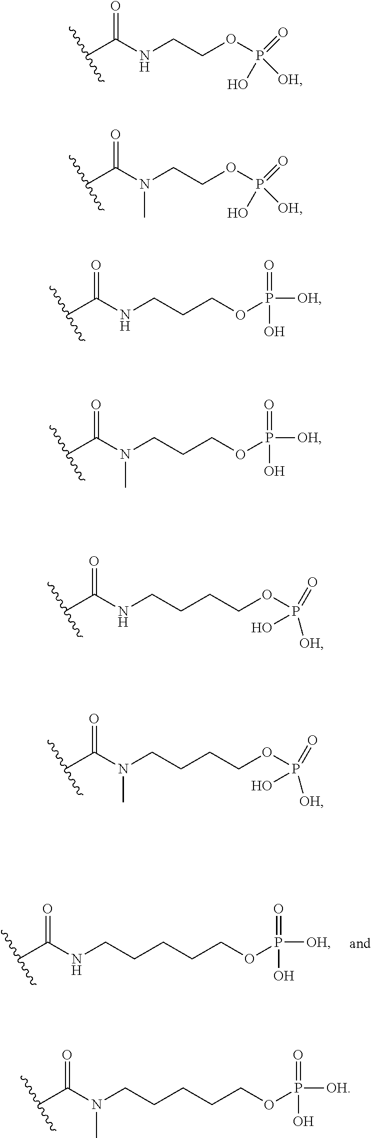

In some embodiments, the same prodrug moiety (i.e., the R¹ group) is chosen from chosen from

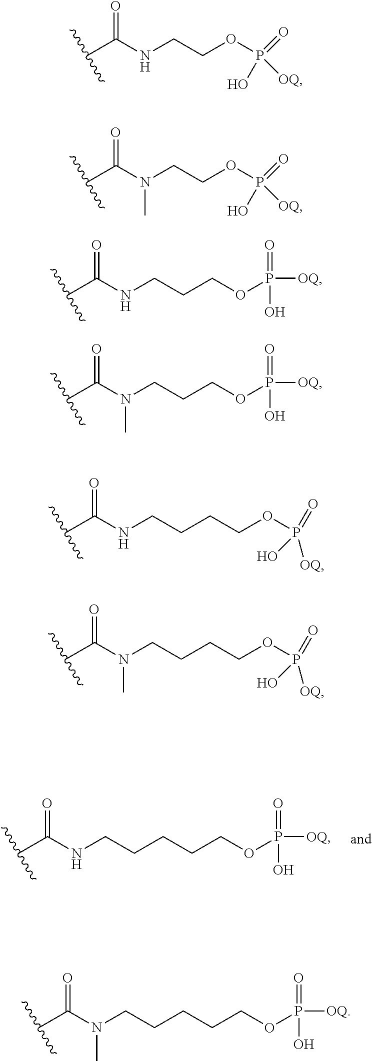

In some embodiments, the same prodrug moiety (i.e., the R¹ group) is chosen from chosen from

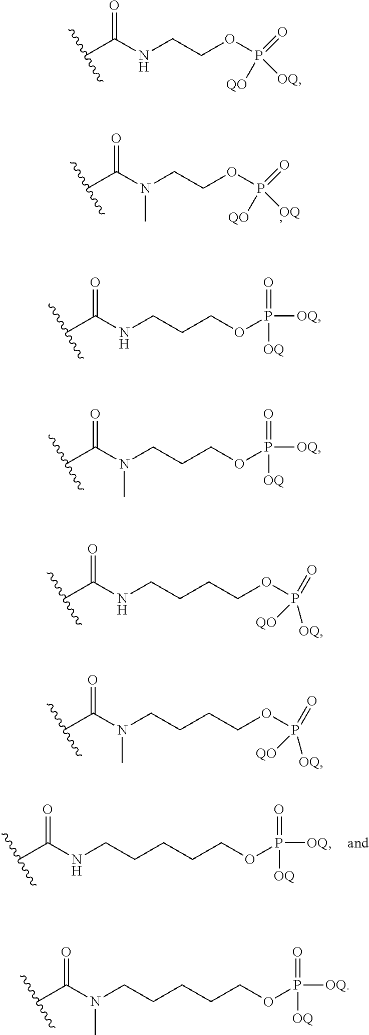

In some embodiments, the same prodrug moiety (i.e., the R¹ group) is

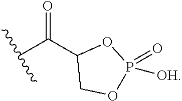 [SMILES: *C(=O)C1COP(=O)(O)O1]

In some embodiments, the same prodrug moiety (i.e., the R¹ group) is

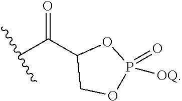

In some embodiments, the same prodrug moiety (i.e., the R¹ group) is

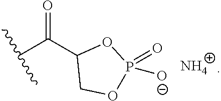 [SMILES: *C(=O)C1COP(=O)([O-])O1 ; [NH4+]]

In some embodiments, the other active compound is chosen from analgesics, antinociceptive agents, anti-inflammatory agents, antipyretics, antidepressants, antiepileptics, antihistamines, antimigraine agents, antimuscarinics, anxiolytics, sedatives, hypnotics, antipsychotics, bronchodilators, anti-asthma agents, cardiovascular agents (such as antihypertensive agents and antiarrhythmic agents), corticosteroids, dopaminergics, electrolytes, mood stabilizers (such as those for treating bipolar disorders), muscle relaxants, nutritional agents, vitamins, parasympathomimetics, stimulants, anorectics, and anti-narcoleptics. Specific examples include, but are not limited to, acetazolamide, aceclofenac, acetaminophen, atomoxetine, almotriptan, alprazolam, amantadine, amcinonide, aminocyclopropane, amitriptyline, amlodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clobazam, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproex, dizatriptan, dolasetron, donepezil, dothiepin, doxepin, duloxetine, ergotamine, escitalopram, estazolam, ethosuximide, etodolac, felbamate, femoxetine, fenamates, fenoprofen, fentanyl, fludiazepam, fluoxetine, fluphenazine, flurazepam, flurbiprofen, flutazolam, fluvoxamine, frovatriptan, gabapentin, gabitril, galantamine, gepirone, Ginkgo biloba, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lacosamide, lamotrigine (an anticonvulsant and mood stabilizer), lesopitron, levodopa, levetiracetam, lipase, lithium (a mood stabilizer), lofepramine, lorazepam, loxapine, maprotiline, mazindol, mefenamic acid, melatonin, melitracen, memantine, meperidine, meprobamate, mesalamine, methsuximide, metapramine, metaxalone, methadone, methadone, methamphetamine, methocarbamol, methyldopa, methylphenidate, methylsalicylate, methysergide, metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nitrazepam, nomifensine, nortriptyline, olanzapine, olsalazine, ondansetron, opipramol, orphenadrine, oxaflozane, oxaprozin, oxazepam, oxitriptan, oxycodone, oxymorphone, pancrelipase, parecoxib, paroxetine, pemoline, pentazocine, pepsin, perphenazine, phenobarbital, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, primidone, propranolol, propizepine, propoxyphene, protriptyline, quazepam, quinupramine, reboxetine, reserpine, risperidone, ritanserin, rivastigmine, rizatriptan, rofecoxib, ropinirole, rotigotine, rufinamide, salsalate, sertraline, sibutramine, sildenafil, stiripentol, sulfasalazine, sulindac, sumatriptan, tacrine, temazepam, tetrabenazine, thiazides, thioridazine, thiothixene, tiapride, taziprinone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, vigabatrin, viloxazine, vitamin E, zimeldine, ziprasidone, zolmitriptan, zolpidem, zonisamide, zopiclone, and combinations thereof.

### E. Kits

Kits containing one or more unit doses of at least one compound of the present disclosure or a pharmaceutical composition comprising the same are provided. In some embodiments, the one or more unit doses are for oral administration. Such kits may include a container comprising the one or more unit doses, an informational package insert describing the use and attendant benefits of the therapeutic in treating the pathological condition of interest, and/or optionally an appliance or device for delivery of the at least one compound of the present disclosure or the pharmaceutical composition comprising the same. In some embodiments, the container is a bottle or vial.

In some embodiments, the disclosure contemplates a pressurized or unpressurized container containing at least one compound of the present disclosure or a pharmaceutical composition comprising the same. In some embodiments, the container is a manual pump spray, inhaler, meter-dosed inhaler, dry powder inhaler, nebulizer, vibrating mesh nebulizer, jet nebulizer, or ultrasonic wave nebulizer.

### IV. Methods of Use

In some embodiments, a method for treating and/or preventing at least one disease, disorder, and/or condition where treatment with a cannabinoid may be useful is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing at least one disease, disorder, and/or condition where treatment with (−)trans-Δ⁸-THC may be useful is disclosed, the method comprising administering to a subject in need thereof an effective amount of at least one compound of Formula (II)-1A or a pharmaceutically acceptable salt thereof or a pharmaceutical composition comprising the same.

In some embodiments, a method for treating and/or preventing at least one disease, disorder, and/or condition where treatment with (−)trans-Δ⁹-THC may be useful is disclosed, the method comprising administering to a subject in need thereof an effective amount of at least one compound of Formula (III)-1A or a pharmaceutically acceptable salt thereof or a pharmaceutical composition comprising the same.

In some embodiments, a method for treating and/or preventing at least one disease, disorder, and/or condition where treatment with HU-210 may be useful is disclosed, the method comprising administering to a subject in need thereof an effective amount of at least one compound of Formula (II)-1B or a pharmaceutically acceptable salt thereof or a pharmaceutical composition comprising the same.

In some embodiments, a method for treating and/or preventing at least one disease, disorder, and/or condition where treatment with dexanabinol may be useful is disclosed, the method comprising administering to a subject in need thereof an effective amount of at least one compound of Formula (II)-2B or a pharmaceutically acceptable salt thereof or a pharmaceutical composition comprising the same.

In some embodiments, a method for treating and/or preventing at least one disease, disorder, and/or condition where treatment with nabilone may be useful is disclosed, the method comprising administering to a subject in need thereof an effective amount of at least one compound of Formula (IV)-A or a pharmaceutically acceptable salt thereof or a pharmaceutical composition comprising the same.

In some embodiments, a method for treating and/or preventing at least one disease, disorder, and/or condition where treatment with an agonist of cannabinoid 1 receptors (CB₁Rs) and/or cannabinoid 2 receptors (CB₂Rs) may be useful is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing at least one disease, disorder, and/or condition where treatment with an anxiolytic agent, analgesic agent, antiemetic agent, mood-stabilizing agent, antipsychotic agent, muscle relaxation agent, immunosuppressant, anti-inflammatory agent, anti-allergic agent, antioxidant agent, anticancer agent, neuroprotective agent, anti-convulsant agent, antineoplastic agent, appetite stimulant, intraocular pressure regulator, and/or bronchodilator may be useful is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments of the methods described herein, the subject is a human. In some embodiments of the methods described herein, the subject is a human under the age of 18. In some embodiments of the methods described herein, the subject is a non-human animal. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, the subject is a domestic pet, such as a cat or dog.

The compounds and compositions of the present disclosure may be administered by a variety of routes including the oral, ocular, rectal, transdermal, subcutaneous, intravenous, intramuscular, or intranasal routes. In some embodiments, the at least one compound of Formulae (I), (II), (III), (IV), or their sub-formulae or a pharmaceutically acceptable salt thereof or a pharmaceutical composition comprising the same is administered orally. In some embodiments, at least one compound of Formulae (I), (II), (III), (IV), or their sub-formulae or a pharmaceutically acceptable salt thereof or a pharmaceutical composition comprising the same is administered intramuscularly. In some embodiments, at least one compound of Formulae (I), (II), (III), (IV), or their sub-formulae or a pharmaceutically acceptable salt thereof or a pharmaceutical composition comprising the same is administered intravenously. In some embodiments, the at least one compound of Formulae (I), (II), (III), (IV), or their sub-formulae or a pharmaceutically acceptable salt thereof or a pharmaceutical composition comprising the same is administered by inhalation through the lungs. In some embodiments, the at least one compound of Formulae (I), (II), (III), (IV), or their sub-formulae or a pharmaceutically acceptable salt thereof or a pharmaceutical composition comprising the same is administered topically. In some embodiments, at least one compound of Formulae (I), (II), (III), (IV), or their sub-formulae or a pharmaceutically acceptable salt thereof or a pharmaceutical composition comprising the same is administered intranasally. In some embodiments, at least one compound of Formulae (I), (II), (III), (IV), or their sub-formulae or a pharmaceutically acceptable salt thereof or a pharmaceutical composition comprising the same is administered oromucosally.

The terms “treat” and “treatment” include medical management of a disease, disorder, and/or condition of a subject as would be understood by a person of ordinary skill in the art (see, e.g., Stedman's Medical Dictionary). In general, an appropriate dose and treatment regimen provide at least one of the compounds of the present disclosure in an amount sufficient to provide therapeutic benefit. Therapeutic benefit includes, for example, an improved clinical outcome, wherein the object is to slow or lessen an undesired physiological change or disorder, or to slow or lessen the expansion or severity of such disorder. As discussed herein, improved clinical outcomes from treating a subject include, but are not limited to, abatement, lessening, or alleviation of symptoms that result from or are associated with the disease, condition, and/or disorder to be treated; decreased occurrence of symptoms; improved quality of life; diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

The terms “prevent” and “preventing” include the reducing or decreasing the likelihood of occurrence, recurrence, spread, or onset in a statistically or clinically significant manner. It is not intended that the present disclosure be limited to complete prevention. In some embodiments, the onset is delayed, or the severity of the disease is reduced.

The effectiveness of the compounds and pharmaceutical compositions of the present disclosure in treating and/or preventing diseases, disorders, and/or conditions can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

### A. Exemplary Uses and Indications

In some embodiments, a method for treating and/or preventing at least one disease, disorder, and/or condition where treatment with an anti-inflammatory, anti-oxidative, and/or neuroprotective agent may be useful is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing at least one psychiatric disease, disorder, and/or condition is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the at least one psychiatric disease, disorder, and/or condition is chosen from depression, anxiety disorder, dementia, bipolar disorder, schizophrenia, addiction, and nausea. In some embodiments, the at least one psychiatric disease, disorder, and/or condition is anxiety disorder. In some embodiments, the at least one psychiatric disease, disorder, and/or condition is nausea. In some embodiments, the at least one psychiatric disease, disorder, and/or condition is addiction, such as alcohol use disorder and opioid addiction. In some embodiments, the at least one psychiatric disease, disorder, and/or condition is depression.

In some embodiments, a method for treating and/or preventing pain is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the pain is neuropathic pain. In some embodiments, the pain is chronic pain. In some embodiments, the pain is peri-operative pain or pain associated with surgical procedures.

In some embodiments, the neuropathic pain is chosen from peripheral diabetic neuropathy, postherpetic neuralgia, complex regional pain syndromes, peripheral neuropathies, rheumatoid arthritis, chemotherapy-induced neuropathic pain, cancer neuropathic pain, neuropathic low back pain, HIV neuropathic pain, trigeminal neuralgia and/or central post-stroke pain.

In some embodiments, the neuropathic pain results from peripheral or central nervous system pathologic events.

In some embodiments, the neuropathic pain that results from trauma, ischemia; cancer (such as the neuropathic pain caused by sensory fibers at the site of the tumor); infections or from ongoing metabolic or toxic diseases, infections or endocrinologic disorders, including, but not limited to, diabetes mellitus, diabetic neuropathy, amyloidosis, amyloid polyneuropathy (primary and familial), neuropathies with monoclonal proteins, vasculitic neuropathy, HIV infection: neuropathy associated with Guillain-Barre syndrome: neuropathy associated with Fabry's disease; trigeminal and other CNS neuralgias; inflammatory conditions or autoimmune disorders, including, but not limited to, demyelinating inflammatory disorders, rheumatoid arthritis, inflammatory bowel disease, and systemic lupus erythematosus; and cryptogenic causes, including, but not limited to idiopathic distal small-fibre neuropathy. Other causes of neuropathic pain that can be treated according to the methods described herein include, but are not limited to, exposure to toxins or drugs (such as arsenic, thallium, alcohol, vincristine, cisplatin, and dideoxynucleosides), dietary or absorption abnormalities, and immunoglobulinemias. Neuropathic pain can also result from compression of nerve fibres, such as radiculopathies and carpal tunnel syndrome.

In some embodiments, a method for treating and/or preventing epilepsy or similar seizure disorders is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. Patients can include those with epilepsy that are inadequately controlled by existing medications, individuals with developmental epileptic encephalopathy, or individuals with rare diseases or genetic conditions that produce epilepsy, seizures, spasms, abnormally hypersynchronous brain activity, or other conditions associated with enhanced neuronal synchrony. In some embodiments, the patients may be paediatric patients with epilepsy.

In some embodiments, a method for treating and/or preventing at least one neurological disease, disorder, and/or condition is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the at least one neurological disease, disorder, and/or condition is chosen from major mental disorders, conditions that involve basal ganglia or altered dopamine, movement disorders, substance abuse/addiction or predisposition to substance abuse/addiction, pain disorders, developmental delay or situations with impaired learning, intellectual disability, memory, and/or cognition, multiple sclerosis, Tourette Syndrome, and circuit disorder.

In some embodiments, the at least one neurological disease, disorder, and/or condition is chosen from depression, autism, postpartum depression, attention-deficit disorder, schizophrenia, anxiety, various psychoses, and epilepsies.

In some embodiments, the at least one neurological disease, disorder, and/or condition is multiple sclerosis.

In some embodiments, the at least one neurological disease, disorder, and/or condition is Tourette Syndrome.

In some embodiments, the at least one neurological disease, disorder, and/or condition is chosen from dystonia and related motor disorders, Parkinson's disease, and L-DOPA-induced dyskinesias or dyskinesias that result from medication.

In some embodiments, the at least one neurological disease, disorder, and/or condition is chosen from dystonia and related motor disorders.

In some embodiments, the at least one neurological disease, disorder, and/or condition is chosen from L-DOPA-induced dyskinesias or dyskinesias that result from medication.

In some embodiments, the at least one neurological disease, disorder, and/or condition is chosen from Alzheimer's disease, Parkinson's disease, Lewy body dementia, and frontal lobe dementia, and motor retraining after acute injury, spasticity, and spasticity due to brain or spinal cord injury.

In some embodiments, the at least one neurological disease, disorder, and/or condition is circuit disorder.

In some embodiments, the at least one neurological disease, disorder, and/or condition is chosen from neurodegenerative diseases. Exemplary neurodegenerative diseases include, but are not limited to, Parkinson's disease, Alzheimer's disease, Huntington's disease, multiple sclerosis, and amyotrophic lateral sclerosis.

In some embodiments, a method for reducing inflammation is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating at least one inflammatory condition and/or autoimmune disorder is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, at least one inflammatory condition and/or autoimmune disorder is chosen from demyelinating inflammatory disorders, rheumatoid arthritis, inflammatory bowel disease, and systemic lupus erythematosus.

In some embodiments, at least one inflammatory condition and/or autoimmune disorder is rheumatoid arthritis.

In some embodiments, a method for treating and/or preventing at least one disease, disorder, and/or condition associated with multiple sclerosis, Tourette Syndrome, schizophrenia, Parkinson's disease, depression, anxiety, neuropsychiatric or mood disorders, motor dysfunction, spasticity, movement disorders, neuropathic pain, amyotrophic lateral sclerosis, epilepsy or other neurologic events, neurocognitive disorders, tardive dyskinesia, motor disorders, and/or mood disorders is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. An exemplary movement disorder is Huntington's disease (Akinyemi et al., J Pharm Pharm Sci, 2020, 23).

In some embodiments, a method for treating at least one symptom associated with epilepsies and/or similar seizure disorders is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating at least one symptom associated with Lennox-Gastaut syndrome, Dravet syndrome, developmental epileptic encephalopathy, and/or tuberous sclerosis complex is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing the severity and/or intensity of seizures associated with epilepsies and/or rare genetic disorders/diseases (e.g., those that can cause occasional seizures or abnormal electroencephalography) is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing the frequency of seizures associated with epilepsies and/or rare genetic disorders/diseases (e.g., those that can cause occasional seizures or abnormal electroencephalography) is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating multiple sclerosis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating at least one symptom associated with multiple sclerosis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing the severity and/or intensity of at least one symptom associated with multiple sclerosis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing the frequency of at least one symptom associated with multiple sclerosis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the at least one symptom of multiple sclerosis is chosen from neuropathic pain, spasticity, and overactive bladder.

In some embodiments, a method for treating Parkinson's disease is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The efficacy of cannabinoids in treating Parkinson's disease is documented in the art (for example, Luigia Cristino et al., Nat Rev Neurol, 2020, 16(1), 9-29).

In some embodiments, a method for treating at least one symptom of Parkinson's disease is disclosed, the method comprising administering to a subject in need thereof an effective amount of Formulae (I), (II), (III), (IV), or their sub-formulae or a pharmaceutically acceptable salt thereof or a pharmaceutical composition comprising the same.

In some embodiments, the at least one symptom of Parkinson's disease is chosen from spasticity, rigidity, dystonia, and movement disorders.

In some embodiments, a method for treating Alzheimer's disease is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The efficacy of cannabinoids in treating Alzheimer's disease is documented in the art (for example, Luigia Cristino et al., Nat Rev Neurol. 2020, 16(1), 9-29).

In some embodiments, a method for treating at least one symptom of Alzheimer's disease is disclosed, the method comprising administering to a subject in need thereof an effective amount of Formulae (I), (II), (III), (IV), or their sub-formulae or a pharmaceutically acceptable salt thereof or a pharmaceutical composition comprising the same.

In some embodiments, the at least one symptom of Alzheimer's disease is chosen from cognitive impairment, memory loss, and mood changes.

In some embodiments, a method for treating Huntington's disease is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The efficacy of cannabinoids in treating Huntington's disease is documented in the art (for example, Luigia Cristino et al., Nat Rev Neurol, 2020, 16(1), 9-29).

In some embodiments, a method for treating at least one symptom of Huntington's disease is disclosed, the method comprising administering to a subject in need thereof an effective amount of Formulae (I), (II), (III), (IV), or their sub-formulae or a pharmaceutically acceptable salt thereof or a pharmaceutical composition comprising the same.

In some embodiments, the at least one symptom of Huntington's disease is chosen from difficulty concentrating, memory lapses, depression, stumbling and clumsiness, and mood swings.

In some embodiments, a method for treating amyotrophic lateral sclerosis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The efficacy of cannabinoids in treating amyotrophic lateral sclerosis is documented in the art (for example, Luigia Cristino et al., Nat Rev Neurol, 2020, 16(1), 9-29).

In some embodiments, a method for treating at least one symptom of amyotrophic lateral sclerosis is disclosed, the method comprising administering to a subject in need thereof an effective amount of Formulae (I), (II), (III), (IV), or their sub-formulae or a pharmaceutically acceptable salt thereof or a pharmaceutical composition comprising the same.

In some embodiments, the at least one symptom of amyotrophic lateral sclerosis is chosen from muscle twitches, muscle cramps, spasticity, muscle weakness, slurred and nasal speech, and difficulty chewing or swallowing.

In some embodiments, a method for treating rheumatoid arthritis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating at least one symptom associated with rheumatoid arthritis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing the severity and/or intensity of at least one symptom associated with rheumatoid arthritis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing the frequency of at least one symptom associated with rheumatoid arthritis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the at least one symptom associated with rheumatoid arthritis is neuropathic pain.

In some embodiments, a method for treating and/or preventing nausea is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing emesis is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating headache or migraine is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The efficacy of cannabis in treating headache and migraine is documented in the art (for example, Poudel et al., Cureus, 2021, 13(8), e17407).

In some embodiments, a method for treating fiber myalgia is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating anxiety disorder is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating Tourette Syndrome is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating dystonia is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating addiction is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the addition is alcohol use disorder. In some embodiments, the addiction is opioid addiction, including opioid abuse and opioid overdose.

In some embodiments, a method for treating glaucoma is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating anorexia is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing body weight is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing ischemic injury, stroke, or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the ischemic injury is caused by coronary artery bypass graft (CABG) or subarachnoid hemorrhage (SAH).

In some embodiments, a method for treating and/or preventing stroke or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing ischemic injury, stroke, or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered under emergency care for an ischemic injury or stroke. In some embodiments, the ischemic injury is caused by CABG or SAH.

In some embodiments, a method for treating and/or preventing stroke or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered under emergency care for a stroke.

In some embodiments, a method for treating and/or preventing ischemic injury, stroke, or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered under maintenance treatment of ischemic injury or stroke. In some embodiments, the ischemic injury is caused by CABG or SAH.

In some embodiments, a method for treating and/or preventing stroke or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered under maintenance treatment of stroke.

In some embodiments, a method for treating and/or preventing ischemic injury, stroke or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered for rehabilitation of ischemic injury or stroke. In some embodiments, the ischemic injury is caused by CABG or SAH.

In some embodiments, a method for treating and/or preventing stroke or stroke associated damages is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered for rehabilitation of stroke.

In some embodiments, a method for treating traumatic brain injuries (TBIs) is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The efficacy of cannabis in treating TBIs is documented in the art (for example, Grenier et al., Can J Neurol Sci, 2020, 47, 11-17).

In some embodiments, a method for treating TBIs is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered under emergency care for TBIs.

In some embodiments, a method for treating TBIs is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered under maintenance treatment of TBIs.

In some embodiments, a method for treating TBIs is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same, wherein the at least one compound of Formulae (I), (II), (III), (IV), or their sub-formulae or a pharmaceutically acceptable salt thereof or a pharmaceutical composition comprising the same is administered for rehabilitation of TBIs.

In some embodiments, a method for treating sleep disorders is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The use of cannabinoids in treating sleep disorders is documented in the art (for example, Suraev et al., Sleep Med Rev, 2020, 53, 101339).

In some embodiments, a method for treating high blood pressure or hypertension is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. The use of cannabis in reducing blood pressure is documented in the art (for example, Abuhasira et al., Eur J Intern Med. 2021, 86, 79-85).

In some embodiments, a method for treating and/or preventing at least one disease, disorder, and/or condition chosen from chronic nerve injury, chronic pain syndromes, seizures, spreading depression, restless leg syndrome, hypoxic-ischemic encephalopathy, spinal cord injury, status epilepticus, concussion, migraine, hyperventilation, and/or retinopathiesis disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating and/or preventing ischemia following transient or permanent vessel occlusion is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for reducing at least one symptom of neuropathic pain, stroke, epilepsy, and/or other neurologic events or neurodegeneration is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating a subject being treated for cancer is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the cancer is glioblastoma. The efficacy of cannabinoids in treating glioblastoma is documented in the art (for example, Luigia Cristino et al., Nat Rev Neurol. 2020, 16(1), 9-29).

In some embodiments, a method for reducing pain and/or nausea in a subject being treated for cancer is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the cancer is glioblastoma.

In some embodiments, a method for increasing appetite in a subject being treated for cancer is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the cancer is glioblastoma.

In some embodiments, a method for increasing cancer cell death in a subject being treated for cancer is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the cancer is glioblastoma.

In some embodiments, a method for decreasing tumour growth in a subject being treated for cancer is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the cancer is glioblastoma.

In some embodiments, a method for inhibiting metastasis of cancer is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the cancer is glioblastoma.

In some embodiments, a method for treating long COVID is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, a method for treating at least one symptom associated with long COVID is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the at least one symptom associated with long COVID is chosen from brain fog, headache, sleep disorders, lightheadedness, pins-and-needles feelings, depression, anxiety, stomach pain, joint and muscle pain, fatigue, and post-exertional malaise.

In some embodiments, a method for hypoxia is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same. In some embodiments, the hypoxia is induced by respiratory insufficiency, prolonged use of ventilator, or both. In some embodiments, the respiratory insufficiency, prolonged use of ventilator, or both is associated with COVID-19, including hospitalization caused by COVID-19.

In some embodiments, a method for treating at least one symptom associated with hypoxia is disclosed, the method comprising administering to a subject in need thereof an effective amount of at least one compound of Formulae (I), (II), (III), (IV), or their sub-formulae or a pharmaceutically acceptable salt thereof or a pharmaceutical composition comprising the same.

In some embodiments, the at least one symptom associated with hypoxia is psychosis.

### B. Co-Administration and Combination Therapies

In some embodiments, the administration of at least one compound of Formulae (I), (II), (III), (IV), or their sub-formulae or a pharmaceutically acceptable salt thereof may be in conjunction with one or more other therapies. Optionally, the at least one compound of Formulae (I), (II), (III), (IV), or their sub-formulae or a pharmaceutically acceptable salt thereof may be administered in combination with one other pharmaceutically active compound. The administration of the at least one compound of Formulae (I), (II), (III), (IV), or their sub-formulae or a pharmaceutically acceptable salt may occur before, after, or concurrently with the administration of the other pharmaceutically active compound. When the co-administration is concurrent, the combination therapy may be administered from a single pharmaceutical composition or from separate pharmaceutical compositions.

In some embodiments, the weight ratio of the at least one compound of Formulae (I), (II), (III), (IV), or their sub-formulae or pharmaceutically acceptable salts thereof to the other pharmaceutically active compound in the combination therapy may be between 1:10 and 10:1. In some embodiments, the weight ratio is between 1:5 and 5:1. In some embodiments, the weight ratio is between 1:4 and 4:1. In some embodiments, the weight ratio is between 1:3 and 3:1. In some embodiments, the weight ratio is between 1:2 and 2:1. In some embodiments, the weight ratio is between 1:1.5 and 1.5:1. In some embodiments, the weight ratio is between 1:1.2 and 1.2:1. In some embodiments, the weight ratio is about 1:1.

In some embodiments, the other pharmaceutically active compound is a cannabinoid or analogue thereof. In some embodiments, the other pharmaceutically active compound is CBD. In some embodiments, the other pharmaceutically active compound is cannabidivarin (CBDV).

In some embodiments, the other pharmaceutically active compound is a phosphate prodrug of cannabinoid or a pharmaceutically acceptable salt thereof, such as those described in the PCT Patent Application No. PCT/US2022/038876, the entirety of which is incorporated herein by reference.

In some embodiments, the other pharmaceutically active compound is a phosphate prodrug of CBD or a pharmaceutically acceptable salt thereof, such as those described in the PCT Patent Application No. PCT/US2022/038876. In some embodiments, the phosphate prodrug of CBD is chosen from the following compounds and their pharmaceutically acceptable salts thereof.

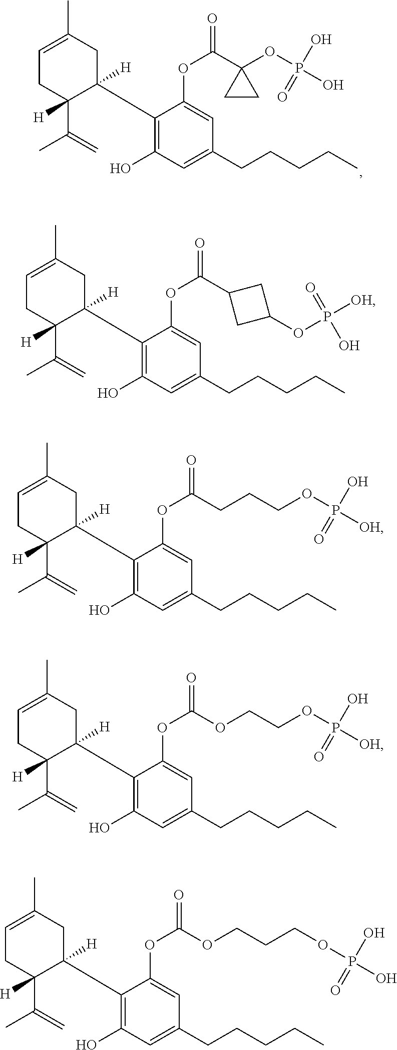 [SMILES: C=C(C)[C@@H]1CC=C(C)C[C@H]1c1c(O)cc(CCCCC)cc1OC(=O)C1(OP(=O)(O)O)CC1 ; C=C(C)[C@@H]1CC=C(C)C[C@H]1c1c(O)cc(CCCCC)cc1OC(=O)C1CC(OP(=O)(O)O)C1 ; C=C(C)[C@@H]1CC=C(C)C[C@H]1c1c(O)cc(CCCCC)cc1OC(=O)CCCOP(=O)(O)O ; C=C(C)[C@@H]1CC=C(C)C[C@H]1c1c(O)cc(CCCCC)cc1OC(=O)OCCOP(=O)(O)O ; C=C(C)[C@@H]1CC=C(C)C[C@H]1c1c(O)cc(CCCCC)cc1OC(=O)OCCCOP(=O)(O)O]

In some embodiments, the other pharmaceutically active compound is a phosphate prodrug of CBDV or a pharmaceutically acceptable salt thereof, such as those described in the PCT Patent Application No. PCT/US2022/038876. In some embodiments, the phosphate prodrug of CBDV is chosen from the following compounds and their pharmaceutically acceptable salts thereof.

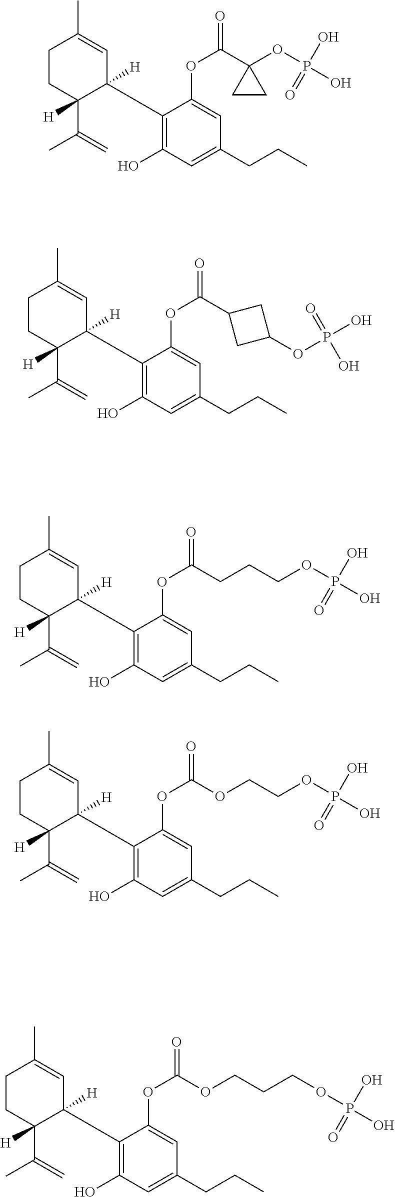 [SMILES: C=C(C)[C@@H]1CCC(C)=C[C@H]1c1c(O)cc(CCC)cc1OC(=O)C1(OP(=O)(O)O)CC1 ; C=C(C)[C@@H]1CCC(C)=C[C@H]1c1c(O)cc(CCC)cc1OC(=O)C1CC(OP(=O)(O)O)C1 ; C=C(C)[C@@H]1CCC(C)=C[C@H]1c1c(O)cc(CCC)cc1OC(=O)CCCOP(=O)(O)O ; C=C(C)[C@@H]1CCC(C)=C[C@H]1c1c(O)cc(CCC)cc1OC(=O)OCCOP(=O)(O)O ; C=C(C)[C@@H]1CCC(C)=C[C@H]1c1c(O)cc(CCC)cc1OC(=O)OCCCOP(=O)(O)O]

In some embodiments, the combination therapy includes co-administration a phosphate prodrug of (−)trans-Δ⁹-THC or (−)trans-Δ⁸-THC or a pharmaceutically acceptable salt thereof, as described in this disclosure (e.g., Formula (III)-1A and Formula (II)-1A), and a phosphate prodrug of CBD or a pharmaceutically acceptable salt thereof, as described in the PCT Patent Application No. PCT/US2022/038876. In some embodiments, the phosphate prodrug of (−)trans-Δ⁹-THC or (−)trans-Δ⁸-THC or a pharmaceutically acceptable salt thereof and the phosphate prodrug of CBD or a pharmaceutically acceptable salt thereof have the same phosphate prodrug moiety, i.e., the R¹ group as defined herein and in the PCT Patent Application No. PCT/US2022/038876. In some embodiments, the administration of the phosphate prodrug of (−)trans-Δ⁹-THC or (−)trans-Δ⁸-THC or a pharmaceutically acceptable salt thereof may occur before, after, or concurrently with the administration of the phosphate prodrug of CBD or a pharmaceutically acceptable salt thereof. In some embodiments, the weight ratio of the phosphate prodrug of (−)trans-Δ⁹-THC or (−)trans-Δ⁸-THC or a pharmaceutically acceptable salt thereof to the phosphate prodrug of CBD or a pharmaceutically acceptable salt thereof in the combination therapy may be between 1:2 and 2:1. In some embodiments, the weight ratio is between 1:1.5 and 1.5:1. In some embodiments, the weight ratio is between 1:1.2 and 1.2:1. In some embodiments, the weight ratio is about 1:1. In some embodiments, the combination therapy may be administered from a single pharmaceutical composition containing both the phosphate prodrug of (−)trans-Δ⁹-THC or (−)trans-Δ⁸-THC or a pharmaceutically acceptable salt thereof and the phosphate prodrug of CBD or a pharmaceutically acceptable salt thereof. In some embodiments, the same prodrug moiety (i.e., the R¹ group) is chosen from

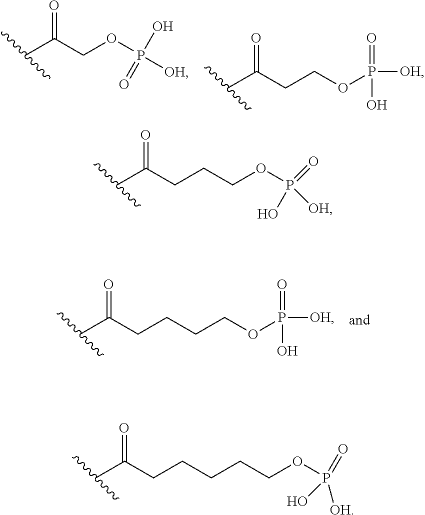 [SMILES: *C(=O)COP(=O)(O)O ; *C(=O)CCOP(=O)(O)O ; *C(=O)CCCOP(=O)(O)O ; *C(=O)CCCCOP(=O)(O)O ; *C(=O)CCCCCOP(=O)(O)O]

In some embodiments, the same prodrug moiety (i.e., the R¹ group) is chosen from

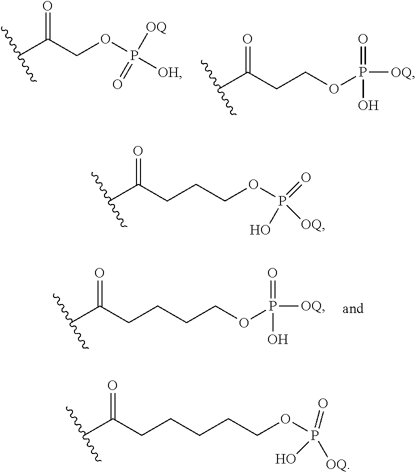

In some embodiments, the same prodrug moiety (i.e., the R¹ group) is chosen from

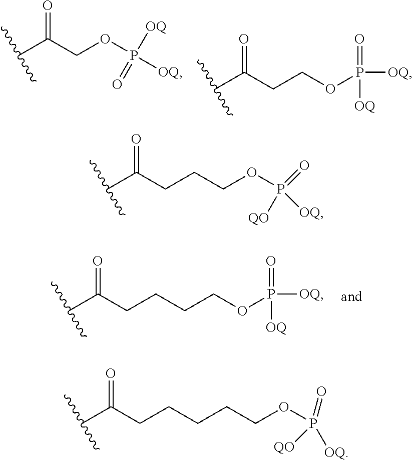

In some embodiments, the same prodrug moiety (i.e., the R¹ group) is chosen from chosen from

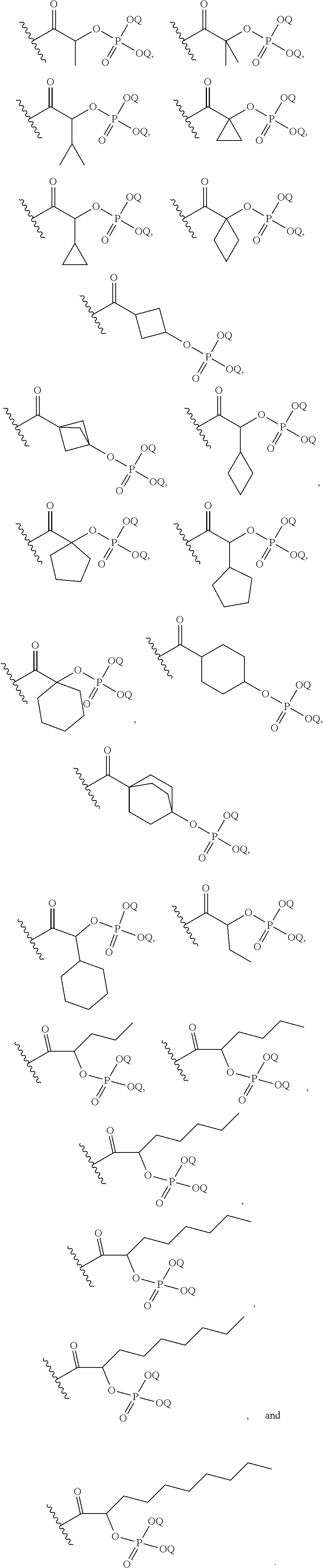

In some embodiments, the same prodrug moiety (i.e., the R¹ group) is chosen from chosen from

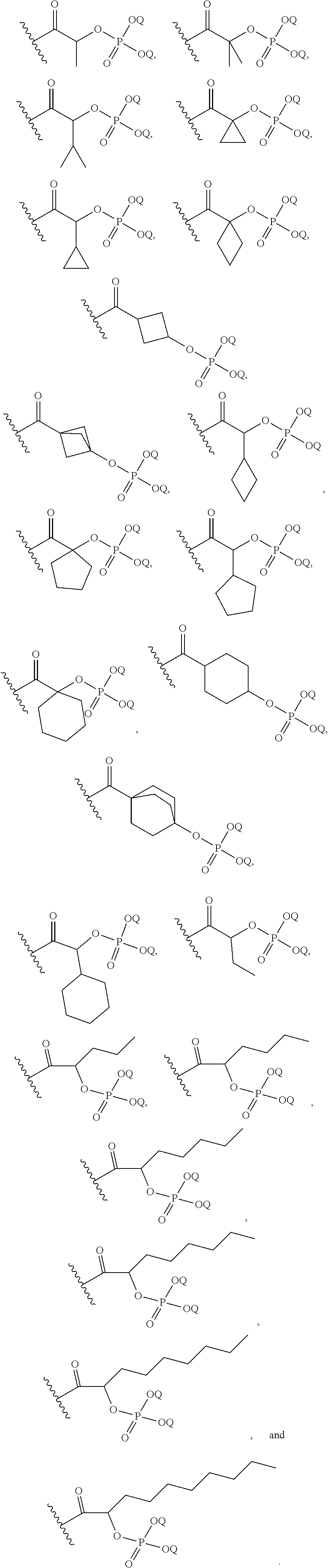

In some embodiments, the same prodrug moiety (i.e., the R¹ group) is chosen from chosen from

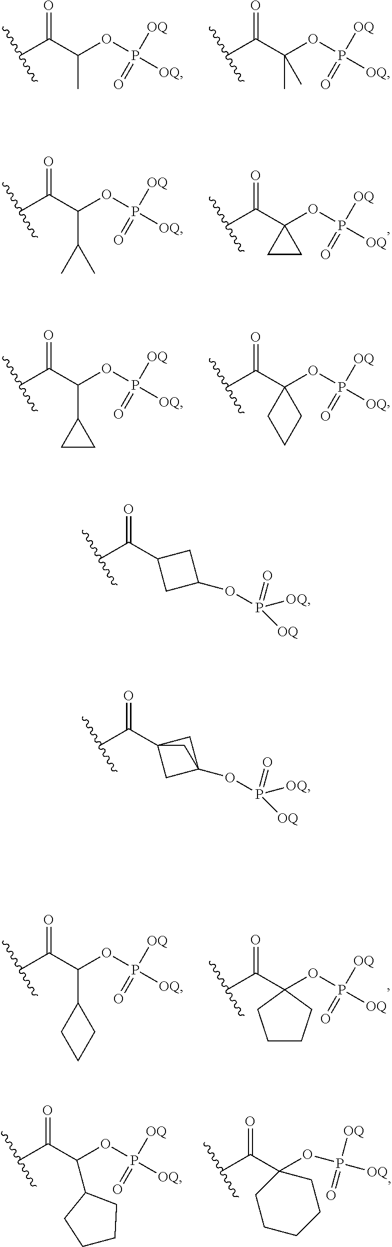

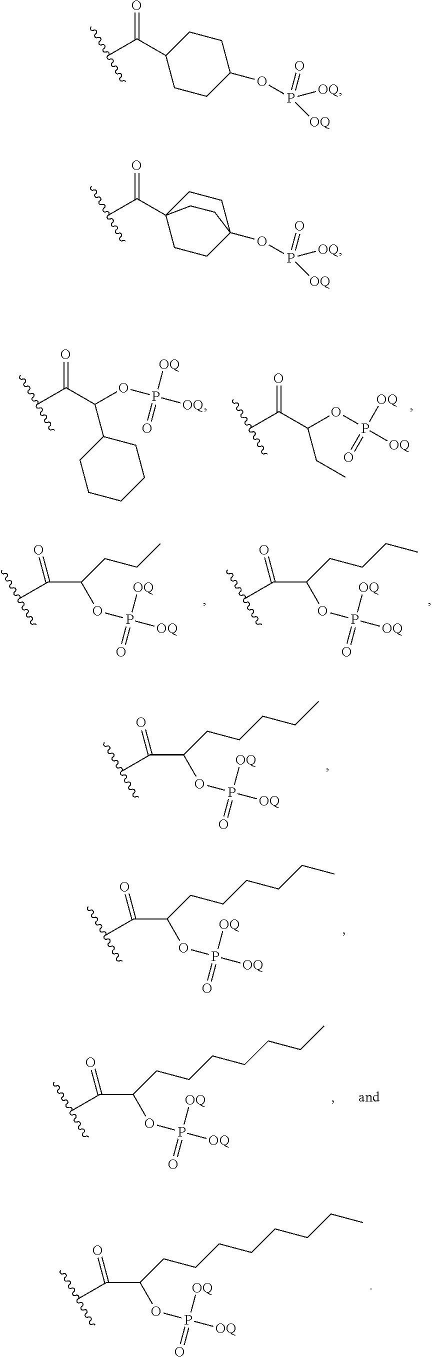

In some embodiments, the same prodrug moiety (i.e., the R¹ group) is chosen from chosen from

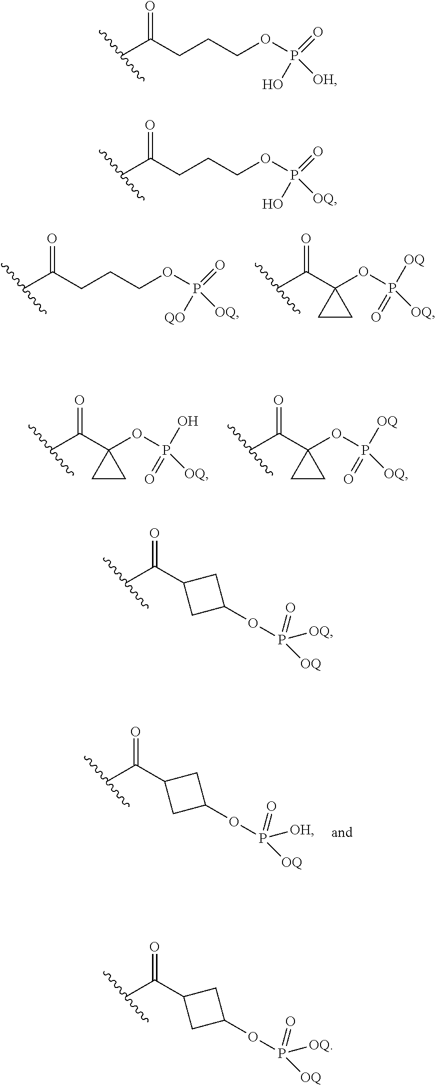

In some embodiments, the same prodrug moiety (i.e., the R¹ group) is chosen from chosen from

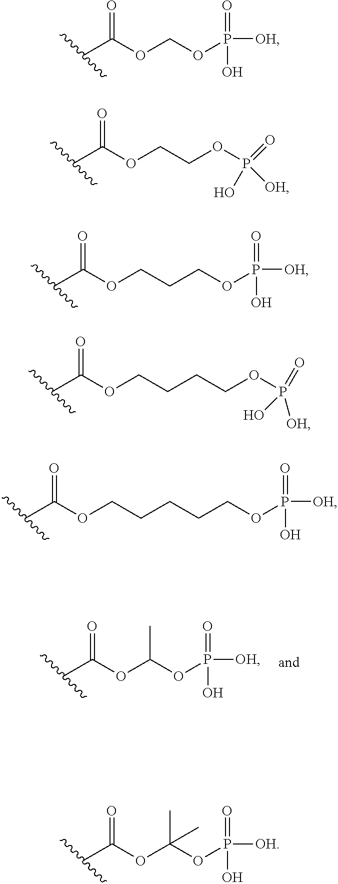

In some embodiments, the same prodrug moiety (i.e., the R¹ group) is chosen from chosen from

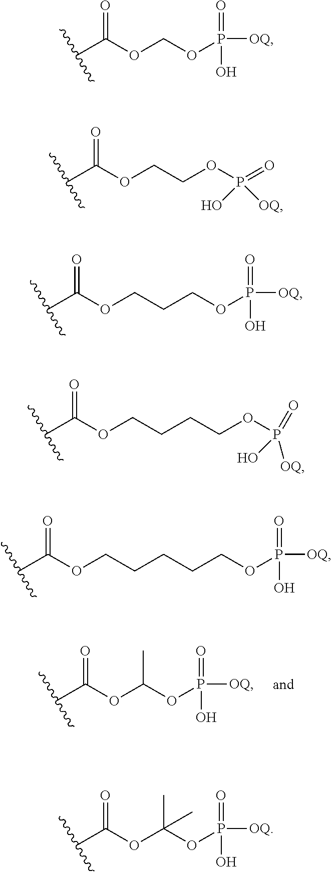

In some embodiments, the same prodrug moiety (i.e., the R¹ group) is chosen from chosen from

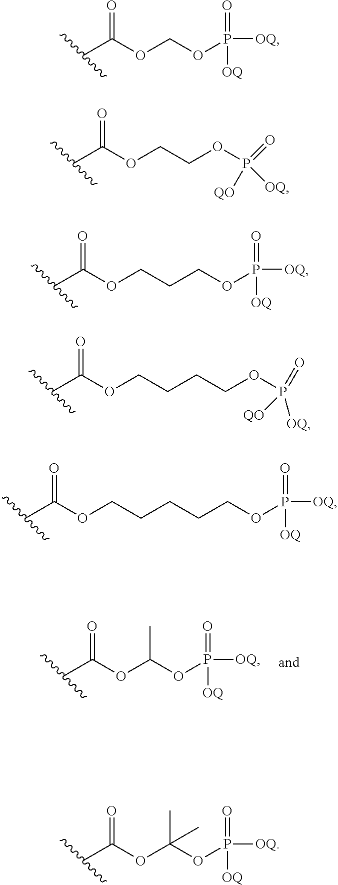

In some embodiments, the same prodrug moiety (i.e., the R¹ group) is chosen from chosen from

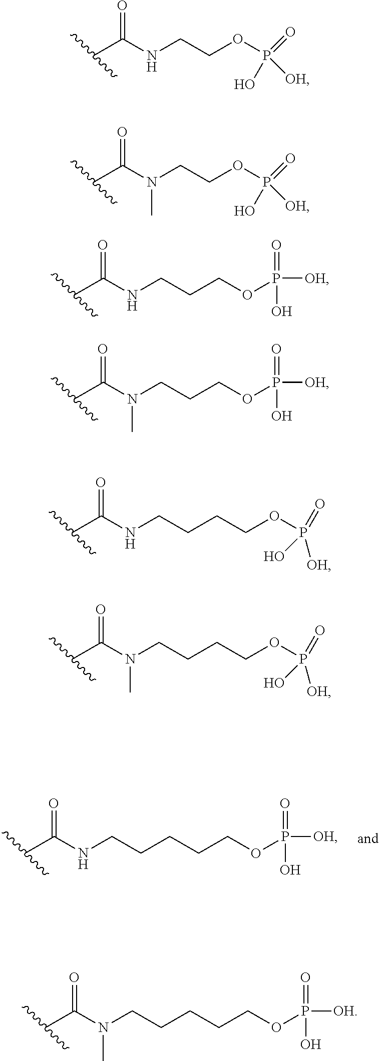

In some embodiments, the same prodrug moiety (i.e., the R¹ group) is chosen from chosen from

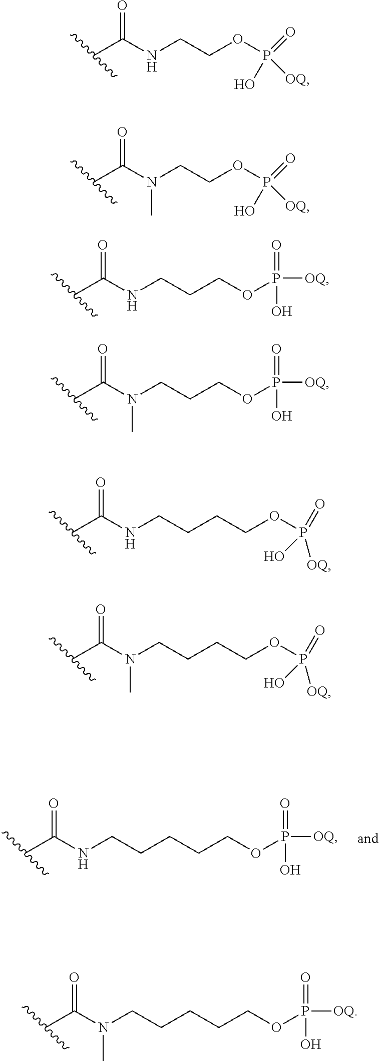

In some embodiments the same prodrug moiety (i.e., the R¹ group) is chosen from chosen from

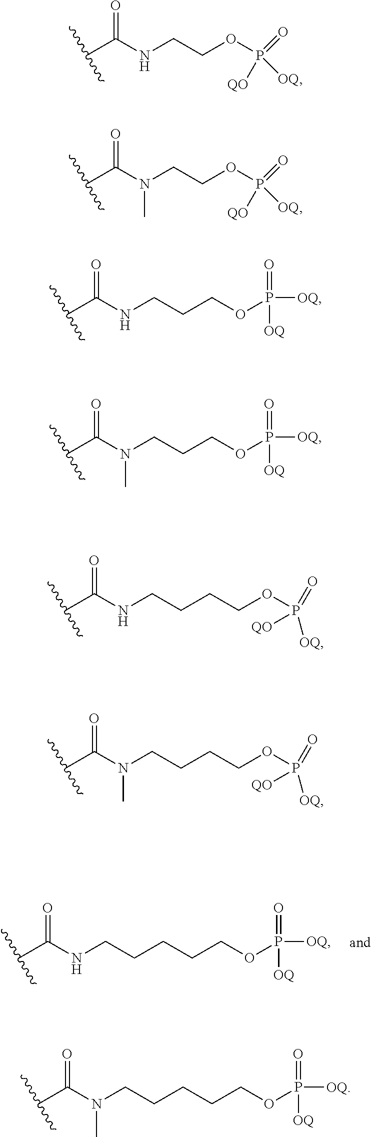

In some embodiments, the same prodrug moiety (i.e., the R¹ group) is

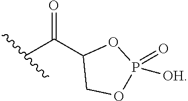 [SMILES: *C(=O)C1COP(=O)(O)O1]

In some embodiments, the same prodrug moiety (i.e., the R¹ group) is

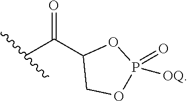

In some embodiments, the same prodrug moiety (i.e., the R¹ group) is

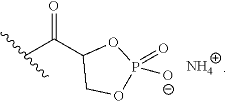 [SMILES: *C(=O)C1COP(=O)([O-])O1 ; [NH4+]]

In some embodiments, the combination therapy includes co-administration a phosphate prodrug of (−)trans-Δ⁹-THC or (−)trans-Δ⁸-THC or a pharmaceutically acceptable salt thereof, as described in this disclosure (e.g., Formula (III)-1A and Formula (II)-1A), and a phosphate prodrug of CBDV or a pharmaceutically acceptable salt thereof, as described in the PCT Patent Application No. PCT/US2022/038876. In some embodiments, the phosphate prodrug of (−)trans-Δ⁹-THC or (−)trans-Δ⁸-THC or a pharmaceutically acceptable salt thereof and the phosphate prodrug of CBDV or a pharmaceutically acceptable salt thereof have the same phosphate prodrug moiety, i.e., the R¹ group as defined herein and in the PCT Patent Application No. PCT/US2022/038876. In some embodiments, the administration of the phosphate prodrug of (−)trans-Δ⁹-THC or (−)trans-Δ⁸-THC or a pharmaceutically acceptable salt thereof may occur before, after, or concurrently with the administration of the phosphate prodrug of CBDV or a pharmaceutically acceptable salt thereof. In some embodiments, the weight ratio of the phosphate prodrug of (−)trans-Δ⁹-THC or (−)trans-Δ⁸-THC or a pharmaceutically acceptable salt thereof to the phosphate prodrug of CBDV or a pharmaceutically acceptable salt thereof in the combination therapy may be between 1:2 and 2:1. In some embodiments, the weight ratio is between 1:1.5 and 1.5:1. In some embodiments, the weight ratio is between 1:1.2 and 1.2:1. In some embodiments, the weight ratio is about 1:1. In some embodiments, the combination therapy may be administered from a single pharmaceutical composition containing both the phosphate prodrug of (−)trans-Δ⁹ -THC or (−)trans-Δ⁸-THC or a pharmaceutically acceptable salt thereof and the phosphate prodrug of CBDV or a pharmaceutically acceptable salt thereof. In some embodiments, the same prodrug moiety (i.e., the R¹ group) is chosen from

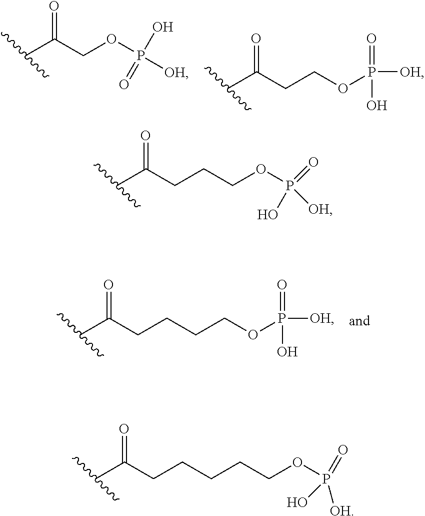 [SMILES: *C(=O)COP(=O)(O)O ; *C(=O)CCOP(=O)(O)O ; *C(=O)CCCOP(=O)(O)O ; *C(=O)CCCCOP(=O)(O)O ; *C(=O)CCCCCOP(=O)(O)O]

In some embodiments, the same prodrug moiety (i.e., the R¹ group) is chosen from

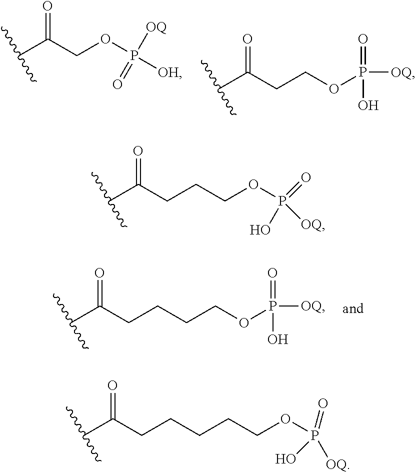

In some embodiments, the same prodrug moiety (i.e., the R¹ group) is chosen from

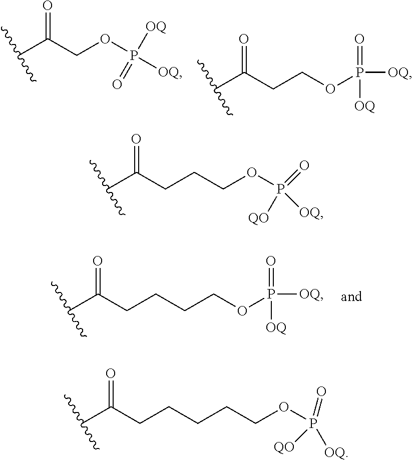

In some embodiments, the same prodrug moiety (i.e., the R¹ group) is chosen from chosen from

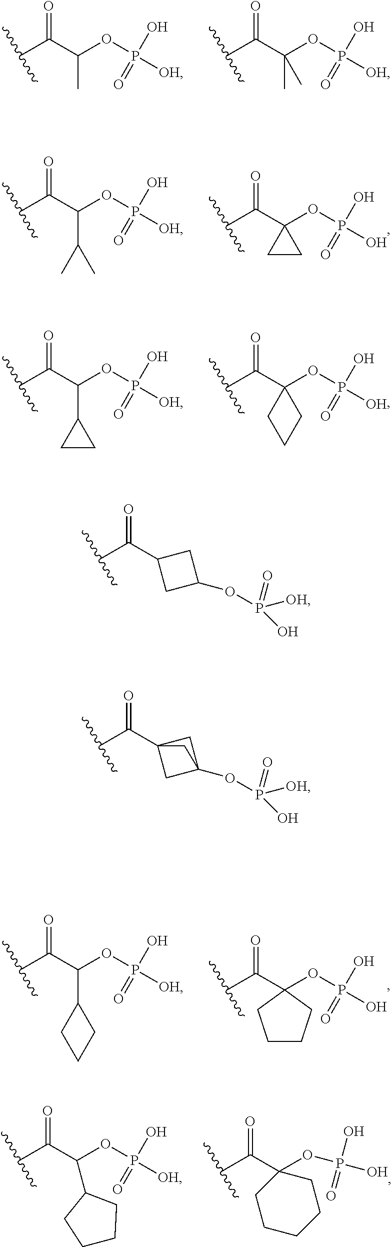

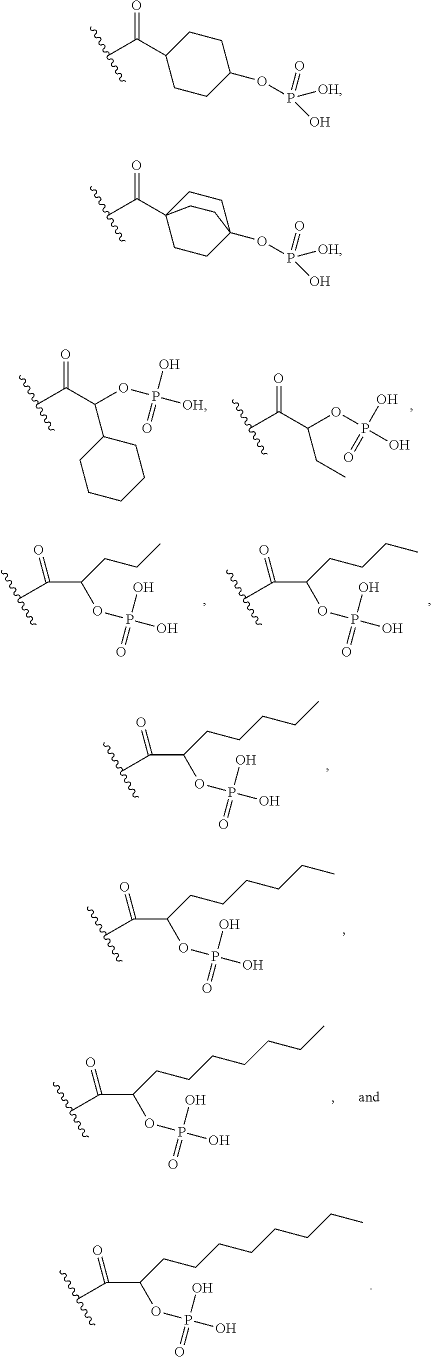

In some embodiments, the same prodrug moiety (i.e., the R¹ group) is chosen from chosen from

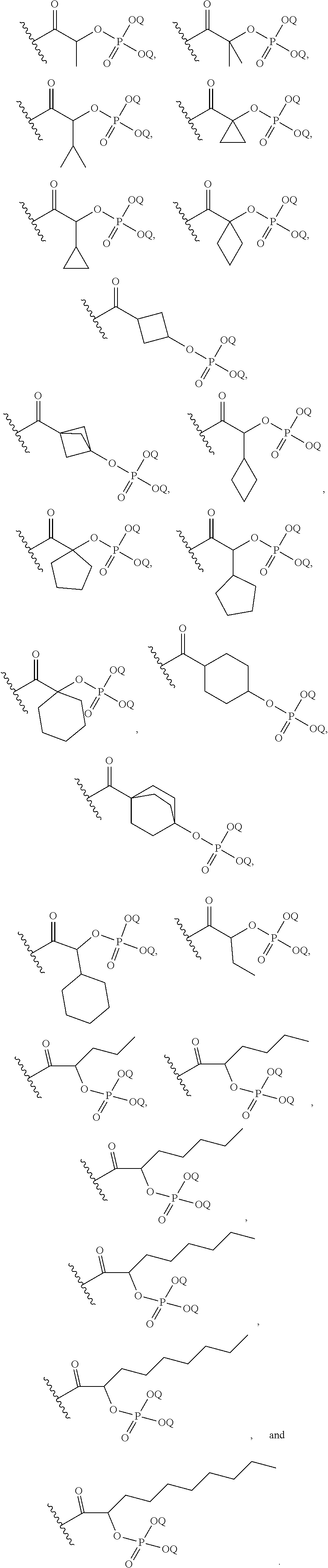

In some embodiments, the same prodrug moiety (i.e., the R¹ group) is chosen from chosen from

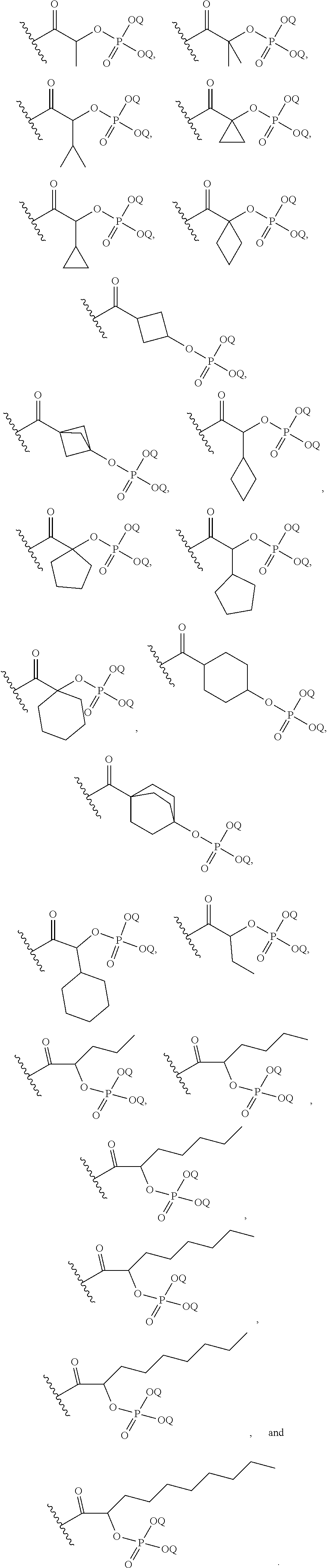

In some embodiments, the same prodrug moeity (i.e., the R¹ group) is chosen from chosen from

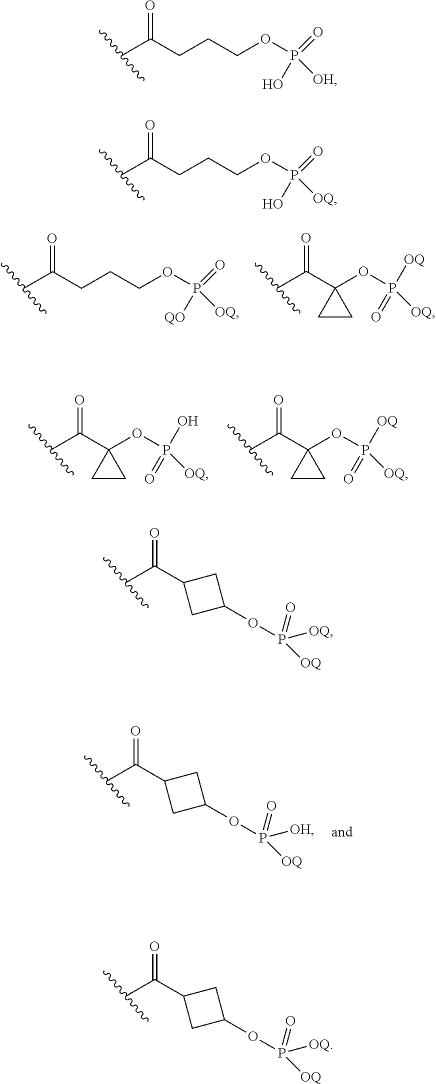

In some embodiments, the same prodrug moiety (i.e., the R¹ group) is chosen from chosen from

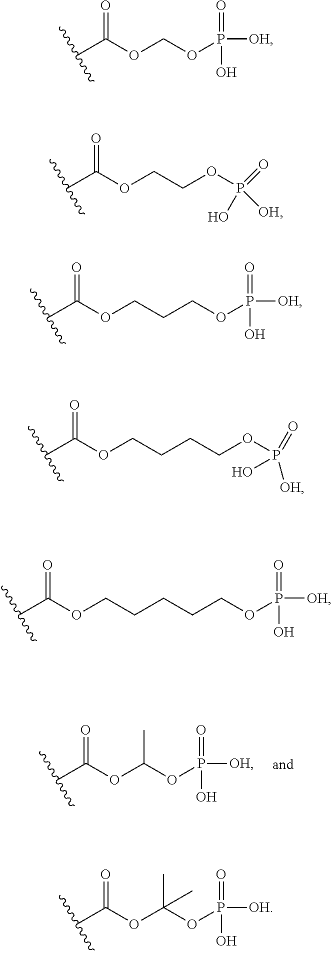

In some embodiments, the same prodrug moiety (i.e., the R¹ group) is chosen from chosen from

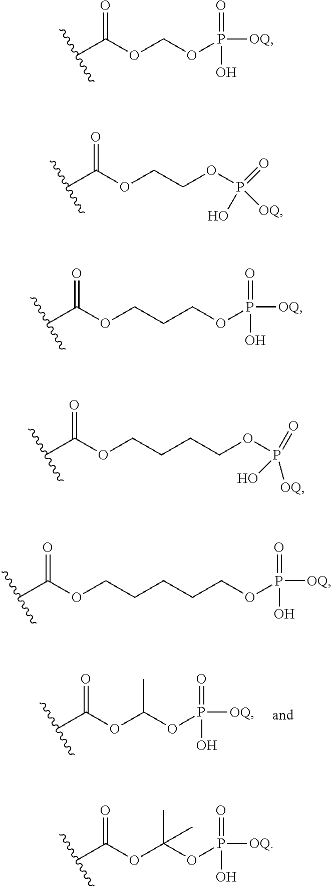

In some embodiments, the same prodrug moiety (i.e., the R¹ group) is chosen from chosen from

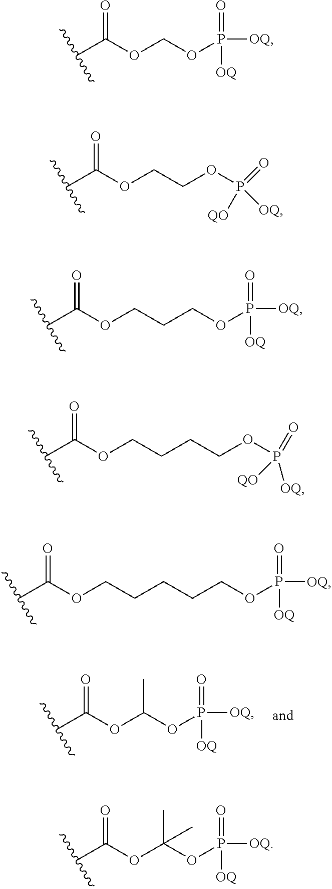

In some embodiments, the same prodrug moiety (i.e., the R¹ group is chosen from chosen from

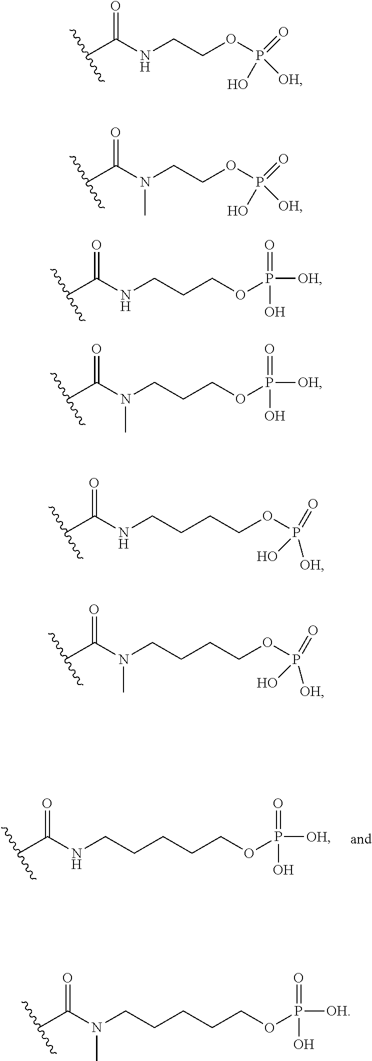

In some embodiments, the same prodrug moiety (i.e., the R¹ group) is chosen from chosen from

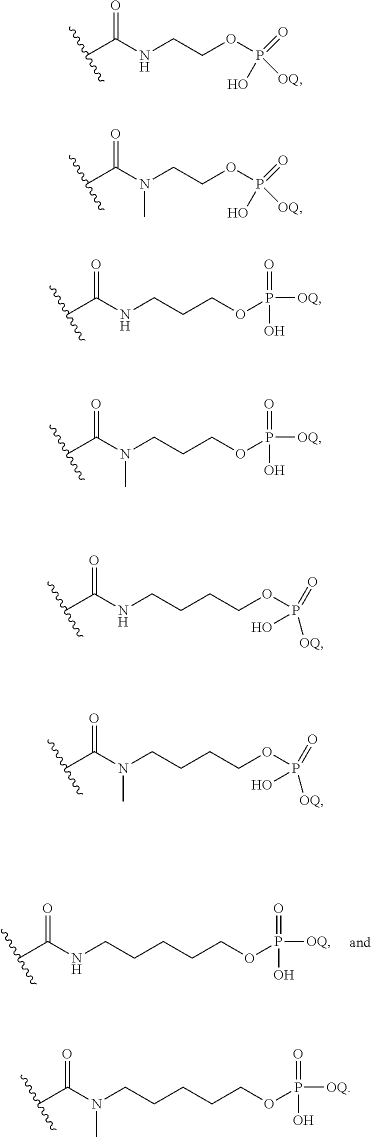

In some embodiments, the same prodrug moiety (i.e., the R¹ group) is chosen from chosen from

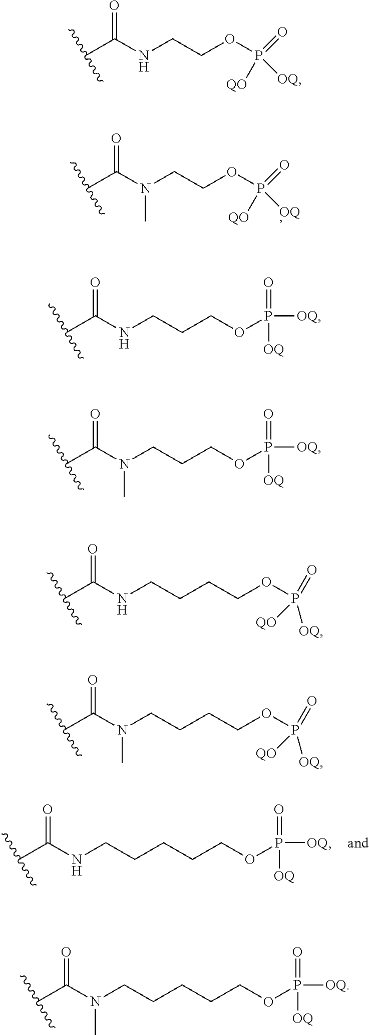

In some embodiments, the same prodrug moiety (i.e., the R¹ group) is

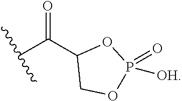 [SMILES: *C(=O)C1COP(=O)(O)O1]

In some embodiments, the same prodrug moiety (i.e., the R¹ group) is

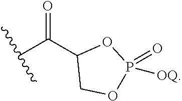

In some embodiments, the same prodrug moiety (i.e., the R¹ group) is

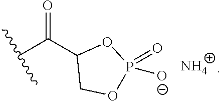 [SMILES: *C(=O)C1COP(=O)([O-])O1 ; [NH4+]]

In some embodiments, the other active compound is chosen from analgesics, antinociceptive agents, anti-inflammatory agents, antipyretics, antidepressants, antiepileptics, antihistamines, antimigraine agents, antimuscarinics, anxiolytics, sedatives, hypnotics, antipsychotics, bronchodilators, anti-asthma agents, cardiovascular agents (such as antihypertensive agents and antiarrhythmic agents), corticosteroids, dopaminergics, electrolytes, mood stabilizers (such as those for treating bipolar disorders), muscle relaxants, nutritional agents, vitamins, parasympathomimetics, stimulants, anorectics, and anti-narcoleptics. Specific examples include, but are not limited to, acetazolamide, aceclofenac, acetaminophen, atomoxetine, almotriptan, alprazolam, amantadine, amcinonide, aminocyclopropane, amitriptyline, amlodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clobazam, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproex, dizatriptan, dolasetron, donepezil, dothiepin, doxepin, duloxetine, ergotamine, escitalopram, estazolam, ethosuximide, etodolac, felbamate, femoxetine, fenamates, fenoprofen, fentanyl, fludiazepam, fluoxetine, fluphenazine, flurazepam, flurbiprofen, flutazolam, fluvoxamine, frovatriptan, gabapentin, gabitril, galantamine, gepirone, Ginkgo biloba, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lacosamide, lamotrigine (an anticonvulsant and mood stabilizer), lesopitron, levodopa, levetiracetam, lipase, lithium (a mood stabilizer), lofepramine, lorazepam, loxapine, maprotiline, mazindol, mefenamic acid, melatonin, melitracen, memantine, meperidine, meprobamate, mesalamine, methsuximide, metapramine, metaxalone, methadone, methadone, methamphetamine, methocarbamol, methyldopa, methylphenidate, methylsalicylate, methysergide, metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nitrazepam, nomifensine, nortriptyline, olanzapine, olsalazine, ondansetron, opipramol, orphenadrine, oxaflozane, oxaprozin, oxazepam, oxitriptan, oxycodone, oxymorphone, pancrelipase, parecoxib, paroxetine, pemoline, pentazocine, pepsin, perphenazine, phenobarbital, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, primidone, propranolol, propizepine, propoxyphene, protriptyline, quazepam, quinupramine, reboxetine, reserpine, risperidone, ritanserin, rivastigmine, rizatriptan, rofecoxib, ropinirole, rotigotine, rufinamide, salsalate, sertraline, sibutramine, sildenafil, stiripentol, sulfasalazine, sulindac, sumatriptan, tacrine, temazepam, tetrabenazine, thiazides, thioridazine, thiothixene, tiapride, taziprinone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, vigabatrin, viloxazine, vitamin E, zimeldine, ziprasidone, zolmitriptan, zolpidem, zonisamide, zopiclone, and combinations thereof.

### EXAMPLES

Compounds of Formulae (I), (II), (III), (IV), and their sub-formulae may be prepared as shown in, for example, in Schemes 1 to 7. It is understood that one of ordinary skill in the art may be able to make these compounds by similar methods or by combining other methods known to one of ordinary skill in the art. It is also understood that one of ordinary skill in the art would be able to make other compounds of Formulae (I), (II), (III), (IV), and their sub-formulae not specifically illustrated herein by using appropriate starting components and modifying the parameters of the synthesis as needed. In general, starting components may be obtained from sources such as Sigma-Aldrich, Alfa Aesar, Maybridge. Matrix Scientific, TCI, Fluorochem USA, etc. and/or synthesized according to sources known to those of ordinary skill in the art (for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to —C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T.W. and P.G.M. Wutz, Protective Groups in Organic Synthesis (1999), 3rd Ed., Wiley. As one of skill in the art would appreciate, the protecting group may also be a polymer resin such as a Wang resin, Rink resin or 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972: T. L. Gilchrist. “Heterocyclic Chemistry,” 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin, G., “Organic Synthesis: Concepts, Methods, Starting Materials,” Second, Revised and Enlarged Edition, John Wiley & Sons ISBN: 3-527-29074-5, 1994; Hoffman, R. V., “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J., “Advanced Organic Chemistry: Reactions, Mechanisms, and Structure,” 4th Edition (1992) John Wiley & Sons, ISBN: 0-471-60180-2; Otera, J. (editor), “Modem Carbonyl Chemistry” (2000) Wiley-VCH, ISBN: 3-527-29871-1; Patai. S., “Patai's 1992 Guide to the Chemistry of Functional Groups” (1992) Interscience ISBN: 0-471-93022-9; Quin, L. D., et al., “A Guide to Organophosphorus Chemistry” (2000) Wiley-Interscience, ISBN: 0-471-31824-8; Solomons, T.W.G., “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopaedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The present disclosure will now be described in more detail with reference to the following non-limiting examples. It should be noted that the particular assays used in the examples section are designed to provide an indication of activity.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

The examples below described studies to generate phosphate-based prodrugs of Δ⁸-THC with increased aqueous solubility, enhanced cellular absorption, improved metabolic stability, and/or more favorable tissue distribution. Phosphate-based prodrugs of Δ⁹-THC, Δ⁸-tetrahydrocannabivarin, Δ⁹-tetrahydrocannabivarin, and nabilone can be generated using similar methods. Phosphate-based prodrugs of other cannabinoids, including HU-210 and dexanabinol, can be generated using similar methods as well, optionally with additional procedures for protection and deprotection of reactive group(s) such as hydroxyl.

### General Chemical Synthesis and Characterization

Automated flash column chromatography was performed using a Teledyne ISCO CombiFlash Companion system with silica gel-packed columns (SiliCycle Inc. or REDISEP® Rf). Analytical thin-layer chromatography (TLC, commercially available from Sigma-Aldrich) was carried out on aluminum-supported silica gel plates (thickness: 200 μm) or glass-supported (thickness: 240 μm) silica gel plates with fluorescent indicator (F-254). Visualization of compounds on TLC plates was accomplished with UV light (254 nm) and/or with phosphomolybdic acid (PMA), potassium permanganate (KMnO₄) or ceric ammonium molybdate (CAM) stains. NMR spectra (¹H, ³¹P) were obtained using either a Varian INOVA 600 MHz spectrometer, a Varian INOVA 500 MHz spectrometer, a Varian INOVA 400 MHz spectrometer, a Varian VNMR 400 MHz spectrometer, a Bruker AVIIIHD 600 MHz spectrometer, or a Mercury 300 MHz spectrometer. NMR samples were prepared in deuterated dimethylsulfoxide (DMSO-d₆) or deuterated methanol (CD₃OD) using the residual solvent peak (DMSO-d₆: ¹H=2.54 ppm. CD₃OD: ¹H=3.31 ppm) as an internal reference. Alternatively, the residual dimethylsulfoxide or methanol peak in ¹H NMR was used as an absolute reference for ³¹P NMR. In some cases, phosphoric acid (³¹P=40.48 ppm) was used as an external reference for ³¹P NMR. MestReNova software was used to process all NMR spectra. NMR data are reported to include chemical shifts (δ) reported in ppm, multiplicities indicated as s (singlet), d (doublet), t (triplet), q (quartet), m (multiplet), br (broad), or app (apparent), coupling constants (J) reported in Hz, and integration normalized to 1 atom (H, P). Final compound purity was assessed using ¹H NMR and LC-MS.

### General Bioanalytical Experimental, Instrumentation, and Materials

All solvents for the LC-MS/MS analysis are UHPLC grade. Methanol and water were purchased from Thermo Fischer, and acetonitrile was obtained from Sigma-Aldrich. The HPLC grade formic acid was obtained from Thermo Fischer. The LC-MS vials with embedded inserts and screw caps were obtained from VWR.

An Agilent 1260 Infinity II HPLC system which includes a micro vacuum degasser, quaternary pump, high-performance autosampler with thermostat, and a thermostatted column compartment coupled to an Agilent 6460C Triple Quadrupole Mass Spectrometer was utilised. The mass spectrometer operated in the ESI mode with Agilent's Jet Stream Technology. The Agilent MassHunter Workstation (version B.02.00) was used for data acquisition, and the MassHunter Quantitative analysis software (version B.01.04) was used for data analysis. We employed the Agilent MassHunter Optimizer software (B.02.00) to optimize two important MS parameters: the fragmentor voltage and collision energies. The optimizer also provided the most abundant MRM transitions used in this study. Individual methods were developed for each compound in the presence of an internal standard (ISTD) d₅₋₇-ethoxy coumarin in a positive mode. All compounds were analyzed using multiple reaction monitoring (MRM) with quantifying and qualifying ions for increased reliability. Reverse-phase HPLC separation for each compound was achieved on an Agilent Infinity Poroshell 120 EC C18 or C8 column or Eclipse XDB C18 column (2.1×50 mm. 2.7 microns). Mobile phases consisted of water (0.1% formic acid) and ACN (0.1% formic acid) at a 0.5 mL/min flow rate. The column temperature was maintained at 40° C. for most of the samples unless otherwise noted. Other MS conditions were as follows: dwell time 100 ms; gas flow 10 L/min; nebulizer pressure 45 psi; delta EMV 200 V.

### Example 1. Synthesis of Alpha-Substituted Ester Linked Mono-Ester Phosphate Prodrugs of Δ⁸-THC (Compounds 6a-f)

### A. Synthetic Procedure

Compounds with a mono-ester phosphate prodrug moiety attached by an alpha substituted ester-linker to (−)trans-Δ⁸-THC were designed and synthesized using the procedures described below. The synthesis of the mono-ester phosphate prodrugs 6a-f is shown in Schemes 1A and 1B.

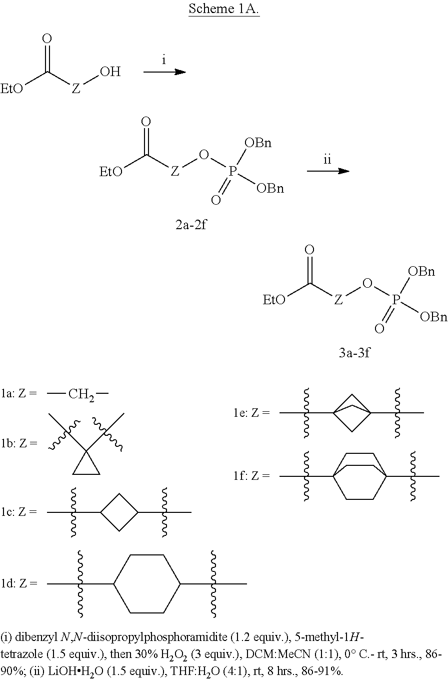

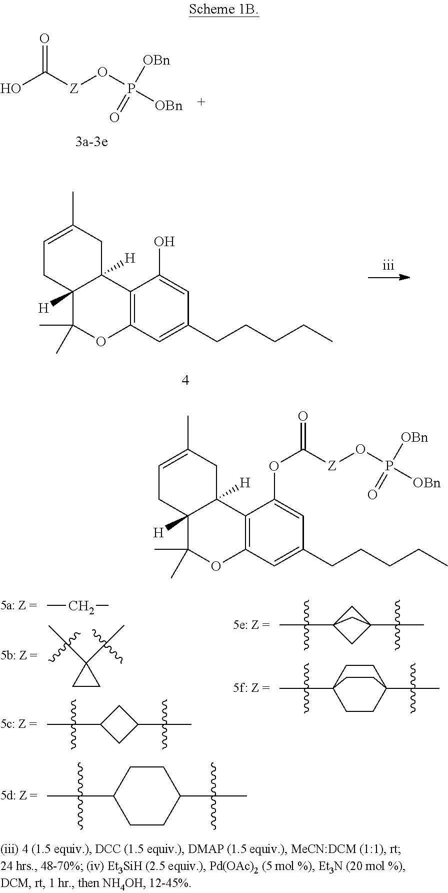

The carboxylic acid derivatives 3a-f were accessed via reaction of the substituted ester analogues 1a-f with dibenzyl N,N-diisopropylphosphoramidite in the presence of 5-methyl-1H-tetrazole, followed by oxidation to the phosphate via hydrogen peroxide to afford the dibenzyl phosphate analogues 2a-f. These ester derivatives 2a-f were then hydrolyzed using lithium hydroxide monohydrate to afford the carboxylic acid derivatives 3a-f. Commercially available (−)trans-Δ⁸-THC 4 isolate then underwent Steglich esterification with the previously synthesized carboxylic acids 3a-f Following aqueous workup and column chromatography, the penultimate dibenzyl phosphate compounds 5a-f were furnished in moderate yield. Lastly, compounds 5a-f underwent palladium acetate catalyzed and triethylsilane facilitated debenzylation of the phosphate moiety. Compounds were subjected to NH₄OH prior to purification to afford the desired prodrugs 6a-f as bis-ammonium salts.

### B. Chemical Characterization

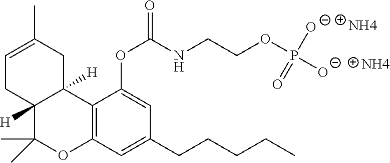 [SMILES: CCCCCc1cc(OC(=O)NCCOP(=O)([O-])[O-])c2c(c1)OC(C)(C)[C@@H]1CC=C(C)C[C@@H]21 ; [NH4+] ; [NH4+]]

Compound 6a: (6aR,10aR)-6,6,9-trimethyl-3-pentyl-6a,7,10,10a-tetrahydro-6H-benzo[c]chromen-1-yl 2-(phosphonooxy)acetate di-ammonium salt.

¹H NMR (600 MHz, DMSO) δ 6.48 (d, J=1.7 Hz, 1H), 6.42 (d, J=1.7 Hz, 1H), 5.39 (br s, 1H), 4.56-4.42 (m, 2H), 2.71-2.64 (m, 1H), 2.44-2.40 (m, 2H), 2.11-2.04 (m, 1H), 1.83-1.68 (m, 3H), 1.65 (s, 3H), 1.63-1.56 (m, 1H), 1.53-1.44 (m, 2H), 1.31 (s, 3H), 1.27-1.21 (m, 4H), 1.00 (s, 3H), 0.85 (t, J=7.2, 1.6 Hz, 3H). ³¹P NMR (243 MHz, DMSO) δ −0.98 (t).

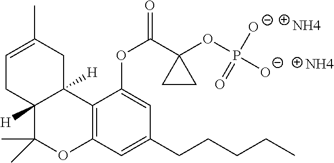 [SMILES: CCCCCc1cc(OC(=O)C2(OP(=O)([O-])[O-])CC2)c2c(c1)OC(C)(C)[C@@H]1CC=C(C)C[C@@H]21 ; [NH4+] ; [NH4+]]

Compound 6b: (6aR,10aR)-6,6,9-trimethyl-3-pentyl-6a,7,10,10a-tetrahydro-6H-benzo[c]chromen-1-yl 1-(phosphonooxy)cyclopropane-1-carboxylate di-ammonium salt.

¹H NMR (400 MHz, DMSO-d₆) δ 6.47 (d, J=1.8 Hz, 1H), 6.46 (d, J=1.8 Hz, 1H), 5.43-5.36 (m, 1H), 2.77 (dd, J=16.7, 4.5 Hz, 1H), 2.49-2.41 (m, 3H), 2.08 (app d, J=16.3 Hz, 1H), 1.83-1.68 (m, 2H), 1.65 (s, 3H), 1.64-1.45 (m, 5H), 1.33-1.22 (m, 7H), 1.21-1.10 (m, 2H), 1.01 (s, 3H), 0.85 (t, J=6.9 Hz, 3H). ³¹P NMR (162 MHz, DMSO-d₆) δ −2.23.

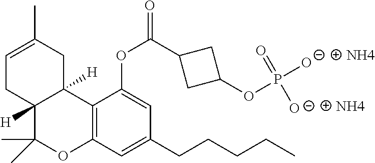 [SMILES: CCCCCc1cc(OC(=O)C2CC(OP(=O)([O-])[O-])C2)c2c(c1)OC(C)(C)[C@@H]1CC=C(C)C[C@@H]21 ; [NH4+] ; [NH4+]]

Compound 6c: (6aR,10aR)-6,6,9-trimethyl-3-pentyl-6a,7,10,10a-tetrahydro-6H-benzo[c]chromen-1-yl 3-(phosphonooxy)cyclobutane-1-carboxylate di-ammonium salt.

¹H NMR (600 MHz, DMSO) δ 6.48 (d, J=1.7 Hz, 1H), 6.38 (d, J=1.7 Hz, 1H), 5.41 (br s, 1H), 4.52-4.43 (m, 1H), 2.92 (tt, J=10.0, 7.8 Hz, 1H), 2.67-2.53 (m, 3H), 2.47-2.38 (m, 3H), 2.25 (td, J=10.5, 8.1 Hz, 1H), 2.16 (td, J=10.4, 8.1 Hz, 1H), 2.10-2.04 (m, 1H), 1.84-1.68 (m, 2H), 1.64 (s, 3H), 1.63-1.59 (m, 1H), 1.54-1.45 (m, 2H), 1.30 (s, 3H), 1.26-1.20 (m, 4H), 1.00 (s, 3H), 0.85 (t, J=7.1 Hz, 3H). ³¹P NMR (243 MHz, DMSO) δ −1.64 (d).

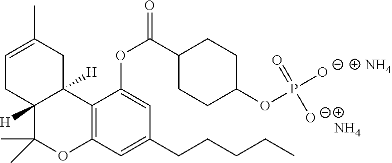 [SMILES: CCCCCc1cc(OC(=O)C2CCC(OP(=O)([O-])[O-])CC2)c2c(c1)OC(C)(C)[C@@H]1CC=C(C)C[C@@H]21 ; [NH4+] ; [NH4+]]

Compound 6d: (6aR,10aR)-6,6,9-trimethyl-3-pentyl-6a,7,10,10a-tetrahydro-6H-benzo[c]chromen-1-yl 4-(phosphonooxy)cyclohexane-1-carboxylate di-ammonium salt.

¹H NMR (600 MHz, DMSO) δ 6.48 (d, J=1.7 Hz, 1H), 6.36 (d, J=1.7 Hz, 1H), 5.41 (br s, 1H), 4.28-4.22 (m, 1H), 2.71-2.65 (m, 1H), 2.63-2.56 (m, 1H), 2.47-2.38 (m, 3H), 2.12-2.05 (m, 1H), 1.91-1.76 (m, 5H), 1.76-1.67 (m, 3H), 1.67-1.60 (m, 1H), 1.64 (s, 3H), 1.59-1.54 (m, 1H), 1.54-1.47 (m, 3H), 1.31 (s, 3H), 1.28-1.21 (m, 4H), 1.01 (s, 3H), 0.85 (t, J=7.1 Hz, 3H). ³¹P NMR (243 MHz, DMSO) δ −0.44 (d).

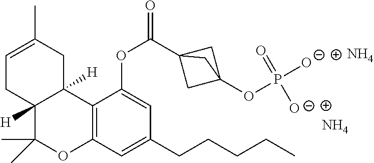 [SMILES: CCCCCc1cc(OC(=O)C23CC(OP(=O)([O-])[O-])(C2)C3)c2c(c1)OC(C)(C)[C@@H]1CC=C(C)C[C@@H]21 ; [NH4+] ; [NH4+]]

Compound 6e: (6aR,10aR)-6,6,9-trimethyl-3-pentyl-6a,7,10,10a-tetrahydro-6H-benzo[c]chromen-1-yl 3-(phosphonooxy)bicyclo[1.1.1]pentane-1-carboxylate phosphate di-ammonium salt.

¹H NMR (600 MHz, DMSO-d₆) δ 6.48 (d, J=1.7 Hz, 1H), 6.38 (d, J=1.7 Hz, 1H), 5.45-5.39 (m, 1H), 2.70-2.60 (m, 1H), 2.47-2.36 (m, 3H), 2.31 (s, 6H), 2.08 (d, J=16.9 Hz, 1H), 1.84-1.68 (m, 2H), 1.66 (s, 3H), 1.64-1.57 (m, 1H), 1.56-1.44 (m, 2H), 1.32-1.20 (m, 7H), 0.99 (s, 3H), 0.85 (t, J=7.0 Hz, 3H). ³¹P NMR (243 MHz, DMSO-d₆) δ −4.42.

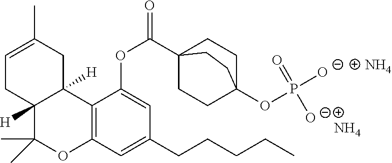 [SMILES: CCCCCc1cc(OC(=O)C23CCC(OP(=O)([O-])[O-])(CC2)CC3)c2c(c1)OC(C)(C)[C@@H]1CC=C(C)C[C@@H]21 ; [NH4+] ; [NH4+]]

Compound 6f: (6aR,10aR)-6,6,9-trimethyl-3-pentyl-6a,7,10,10a-tetrahydro-6H-benzo[c]chromen-1-yl 4-(phosphonooxy)bicyclo[2.2.2]octane-1-carboxylate di-ammonium salt.

¹H NMR (400 MHz, DMSO-d₆) δ 6.45 (s, 1H), 6.26 (s, 1H), 5.39 (s, 1H), 2.62 (d, J=15.0 Hz, 1H), 2.47-2.27 (m, 3H), 2.13-1.87 (m, 12H), 1.82-1.66 (m, 2H), 1.64 (s, 3H), 1.51-1.43 (m, 2H), 1.35-1.14 (m, 7H), 0.96 (s, 3H), 0.84 (t, J=6.9 Hz, 3H). ³¹P NMR (162 MHz, DMSO-d₆) δ −5.31.

### Example 2. Synthesis of Carbonate Linked Mono-Ester Phosphate Prodrugs of Δ⁸-THC (Compounds 11a-b)

A. Synthetic procedure

Compounds with a mono-ester phosphate prodrug moiety attached by a carbonate linker to (−)trans-Δ⁸-THC were designed and synthesized using the procedures described below. The synthesis of these carbonate linked mono-ester phosphate prodrugs 11a-b is shown in Scheme 2.

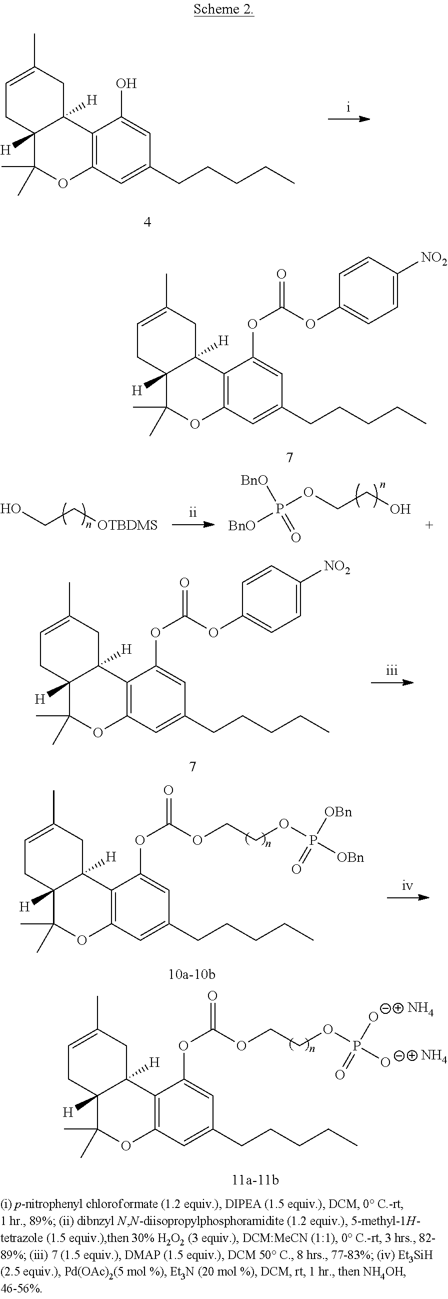

For the synthesis of longer chain carbonate linked phosphate prodrugs, acylation of the parent (−)trans-Δ⁸-THC 4 isolate with p-nitrophenyl chloroformate afforded the reactive electrophile 7. This compound was purified by directly loading onto silica after removal of the solvent in vacuo and carrying out column chromatography. The commercially available TBDMS protected alcohols 8a-b were reacted with dibenzyl N,N-diisopropylphosphoramidite and subsequently oxidized with hydrogen peroxide to furnish the dibenzyl phosphate intermediates 9a-b. These compounds were then reacted with 7 and 4-dimethylaminopyridine to yield the penultimate intermediates 10a-b. Finally, debenzylation utilizing triethylsilane and palladium acetate, and treatment with NH₄OH, afforded the desired carbonate linked phosphate prodrugs 11a-b as the di-ammonium salts.

### B. Chemical Characterization

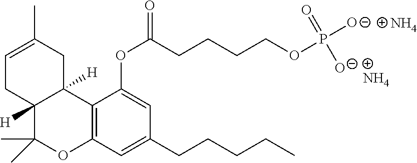 [SMILES: CCCCCc1cc(OC(=O)CCCCOP(=O)([O-])[O-])c2c(c1)OC(C)(C)[C@@H]1CC=C(C)C[C@@H]21 ; [NH4+] ; [NH4+]]

Compound 11a: 3-(phosphonooxy)propyl ((6aR,10aR)-6,6,9-trimethyl-3-pentyl-6a,7,10,10a-tetrahydro-6H-benzo[c]chromen-1-yl) carbonate di-ammonium salt.

¹H NMR (500 MHz, CDCl₃) δ 7.47 (s, 2H), 6.57 (d, J=1.4 Hz, 1H), 6.50 (s, 1H), 5.41 (s, 1H), 4.36 (s, 2H), 4.16 (s, 2H), 2.73 (d, J=17.0 Hz, 1H), 2.64 (td, J=10.8, 4.9 Hz, 1H), 2.54-2.44 (m, 2H), 2.10 (s, 3H), 1.91 (d, J=14.6 Hz, 1H), 1.84-1.70 (m, 2H), 1.66 (s, 3H), 1.57 (p, J=7.6 Hz, 2H), 1.37 (s, 3H), 1.34-1.24 (m, 4H), 1.08 (s, 3H), 0.87 (t, J=6.9 Hz, 3H). ³¹P NMR (162 MHz, CDCl₃) δ 0.74.

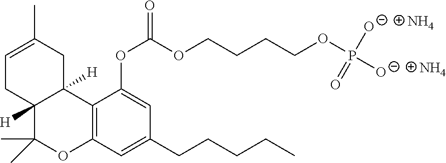 [SMILES: CCCCCc1cc(OC(=O)OCCCCOP(=O)([O-])[O-])c2c(c1)OC(C)(C)[C@@H]1CC=C(C)C[C@@H]21 ; [NH4+] ; [NH4+]]

Compound 11b: 4-(phosphonooxy)butyl ((6aR,10aR)-6,6,9-trimethyl-3-pentyl-6a,7,10,10a-tetrahydro-6H-benzo[c]chromen-1-yl) carbonate di-ammonium salt.

¹H NMR (500 MHz, CDCl₃) δ 7.05 (s, 2H), 6.57 (d, J=1.6 Hz, 1H), 6.49 (d, J=1.6 Hz, 1H), 5.42 (d, J=4.0 Hz, 1H), 4.28 (t, J=5.9 Hz, 2H), 4.11 (s, 2H), 2.75 (dd, J=17.1, 4.5 Hz, 1H), 2.64 (td, J=10.9, 4.8 Hz, 1H), 2.50 (dt, J=8.5, 3.8 Hz, 2H), 2.16-2.07 (m, 1H), 1.94-1.71 (m, 7H), 1.67 (s, 3H), 1.58 (p, J=7.6 Hz, 2H), 1.37 (s, 3H), 1.35-1.24 (m, 5H), 1.09 (s, 3H), 0.90-0.84 (m, 3H).; ³¹P NMR (162 MHz, CDCl₃) δ 1.31.

### Example 3. Synthesis of Long Chain Ester Linked Mono-Ester Phosphate Prodrugs of Δ⁸-THC (Compounds 16a-b)

### A. Synthetic Procedure

Compounds with a mono-ester phosphate prodrug moiety attached by a long chain ester linker to (−)trans-Δ⁸-THC were designed and synthesized using the procedures described below. The synthesis of the long chain ester linked mono-ester phosphate prodrugs 16a-b is shown in Scheme 3.

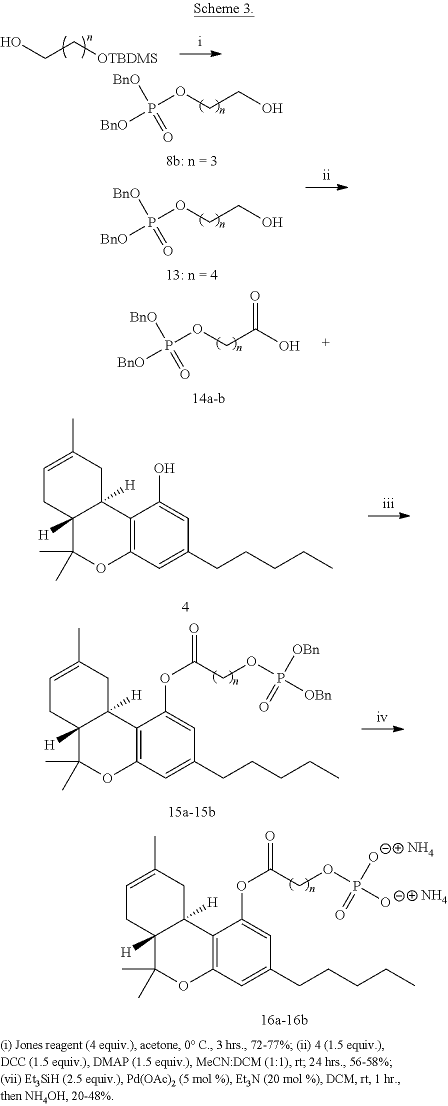

For the longer chain ester linked phosphate (−)trans-Δ⁸-THC prodrugs, commercially available compound 13 was synthesized from 12 via reaction with dibenzyl N,N-diisopropylphosphoramidite and subsequent oxidation with hydrogen peroxide. The previously synthesized analogue 8b together with compound 13 then underwent Jones oxidation to afford the carboxylic acids 14a-b. Subsequent Steglich esterification afforded the dibenzyl phosphate intermediate 15a-b. Lastly, debenzylation via triethylsilane and palladium acetate furnished the desired prodrugs 16a-b as the di-ammonium salt following treatment with NH₄OH.

### B. Chemical Characterization

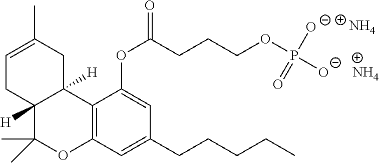 [SMILES: CCCCCc1cc(OC(=O)CCCOP(=O)([O-])[O-])c2c(c1)OC(C)(C)[C@@H]1CC=C(C)C[C@@H]21 ; [NH4+] ; [NH4+]]

Compound 16a: (6aR,10aR)-6,6,9-trimethyl-3-pentyl-6a,7,10,10a-tetrahydro-6H-benzo[c]chromen-1-yl 4-(phosphonooxy)butanoate di-ammonium salt.

¹H NMR (600 MHz, DMSO) δ 6.47 (d, J=1.7 Hz, 1H), 6.42 (d, J=1.8 Hz, 1H), 5.40 (br s, 1H), 3.80-3.72 (m, 2H), 2.68-2.56 (m, 3H), 2.49-2.40 (m, 3H), 2.12-2.04 (m, 1H), 1.90-1.82 (m, 2H), 1.82-1.70 (m, 2H), 1.66-1.58 (m, 1H), 1.65 (s, 3H), 1.54-1.46 (m, 2H), 1.31 (s, 3H), 1.27-1.20 (m, 4H), 1.00 (s, 3H), 0.85 (t, J=7.1 Hz, 3H). MP NMR (243 MHz, DMSO) δ −0.09 (t).

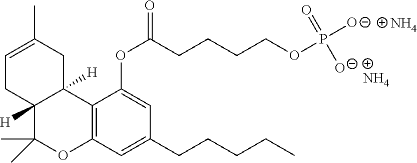 [SMILES: CCCCCc1cc(OC(=O)CCCCOP(=O)([O-])[O-])c2c(c1)OC(C)(C)[C@@H]1CC=C(C)C[C@@H]21 ; [NH4+] ; [NH4+]]

Compound 16b: (6aR,10aR)-6,6,9-trimethyl-3-pentyl-6a,7,10,10a-tetrahydro-6H-benzo[c]chromen-1-yl 5-(phosphonooxy)pentanoate di-ammonium salt.

¹H NMR (400 MHz, DMSO) δ 6.47 (d, J=1.7 Hz, 1H), 6.38 (d, J=1.7 Hz, 1H), 5.43-5.37 (m, 1H), 3.73 (app q, J=6.3 Hz, 2H), 2.69-2.53 (m, 3H), 2.48-2.37 (m, 3H), 2.07 (app d, J=16.2 Hz, 1H), 1.82-1.43 (m, 12H), 1.32-1.18 (m, 7H), 1.00 (s, 3H), 0.84 (t, J=6.9 Hz, 3H). ³¹P NMR (162 MHz, DMSO) δ −1.48.

### Example 4. Synthesis of Carbamate Linked Mono-ESTER Phosphate Prodrugs of Δ⁸-THC (Compounds 21a-b)

### A. Synthetic Procedure

Compounds with a mono-ester phosphate prodrug moiety attached by a carbamate linker to (−)trans-Δ⁸-THC were designed and synthesized using the procedures described below. The synthesis of the carbamate linked mono-ester phosphate prodrugs 21a-b is shown in Scheme 4.

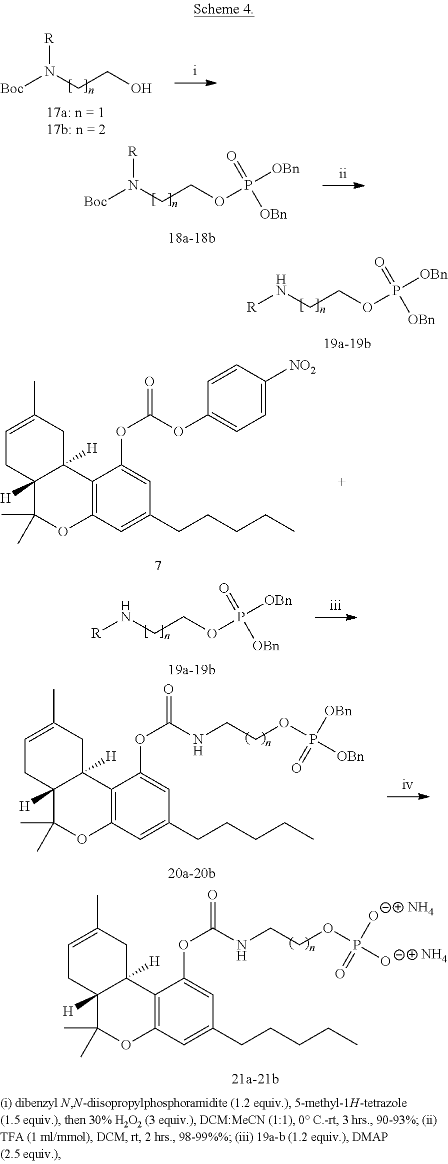

The dibenzyl phosphate moiety was introduced to commercially available N-boc-amino alcohols 17a-b through reaction with dibenzyl N,N-diisopropylphosphoramidite and subsequent oxidation with hydrogen peroxide, affording the intermediates 18a-b in good yield. Boc deprotection was then accomplished by stirring 18a-b in a solution of trifluoroacetic acid in DCM resulted in formation of amines 19a-b, which was dried under vacuum and used without further purification. The previously synthesized activated carbonate 7 was reacted with amines 19a-b in DCM with DMAP serving as base to afford the dibenzyl phosphate intermediates 20a-b. Lastly, these compounds were submitted to debenzylation conditions with triethylsilane and palladium acetate, yielding the desired carbamate linked phosphate prodrugs 21a-b as the di-ammonium salt following treatment with NH₄OH.

### B. Chemical Characterization

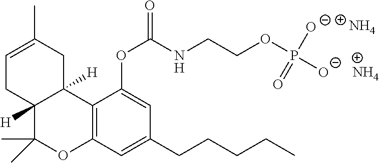 [SMILES: CCCCCc1cc(OC(=O)NCCOP(=O)([O-])[O-])c2c(c1)OC(C)(C)[C@@H]1CC=C(C)C[C@@H]21 ; [NH4+] ; [NH4+]]

Compound 21a: (6aR,10aR)-6,6,9-trimethyl-3-pentyl-6a,7,10,10a-tetrahydro-6H-benzo[c]chromen-1-yl (2-(phosphonooxy)ethyl)carbamate di-ammonium salt.

¹H NMR (500 MHz, CDCl₃) δ 6.52 (s, 1H), 6.43 (s, 1H), 4.23-4.00 (m, 2H), 2.65 (d, J=11.2 Hz, 2H), 2.50-2.38 (m, 2H), 1.93-1.67 (m, 3H), 1.63 (s, 3H), 1.59-1.50 (m, 2H), 1.34 (s, 3H), 1.28 (d, J=3.3 Hz, 4H), 1.06 (s, 3H), 0.86 (t, J=6.9 Hz, 3H). ³¹P NMR (162 MHz, CDCl₃) δ 0.17.

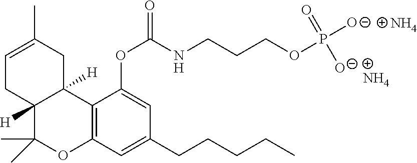 [SMILES: CCCCCc1cc(OC(=O)NCCCOP(=O)([O-])[O-])c2c(c1)OC(C)(C)[C@@H]1CC=C(C)C[C@@H]21 ; [NH4+] ; [NH4+]]

Compound 21b: (6aR,10aR)-6,6,9-trimethyl-3-pentyl-6a,7,10,10a-tetrahydro-6H-benzo[c]chromen-1-yl (3-(phosphonooxy)propyl)carbamate di-ammonium salt.

¹H NMR (500 MHz, CDCl₃) δ 6.53 (d, J=15.7 Hz, 1H), 6.41 (s, 1H), 5.39 (s, 1H), 4.35-3.87 (m, 2H), 3.70-3.12 (m, 2H), 2.64 (d, J=8.6 Hz, 2H), 2.46 (s, 2H), 1.99-1.67 (m, 5H), 1.59-1.51 (m, 2H), 1.35 (d, J=4.9 Hz, 3H), 1.30-1.24 (m, 4H), 1.07 (s, 3H), 0.86 (t, J=6.7 Hz, 3H). ³¹P NMR (162 MHz, CDCl₃) δ 0.50.

### Example 5. Synthesis of Ester Linked Di-Ester Phosphate Prodrugs of Δ⁸-THC (Prophetic Compounds 22 and 28a-k)

### A. Synthetic Procedure

Compounds with a di-ester phosphate prodrug moiety attached by a cyclopropyl-glycolic ester linker to (−)trans-Δ⁸-THC are designed and can be synthesized using the procedures described below and in the PCT Patent Application No. PCT/US2022/038876. A prophetic synthetic route of the di-ester phosphate prodrugs 22 and 28a-k is shown in Scheme 5.

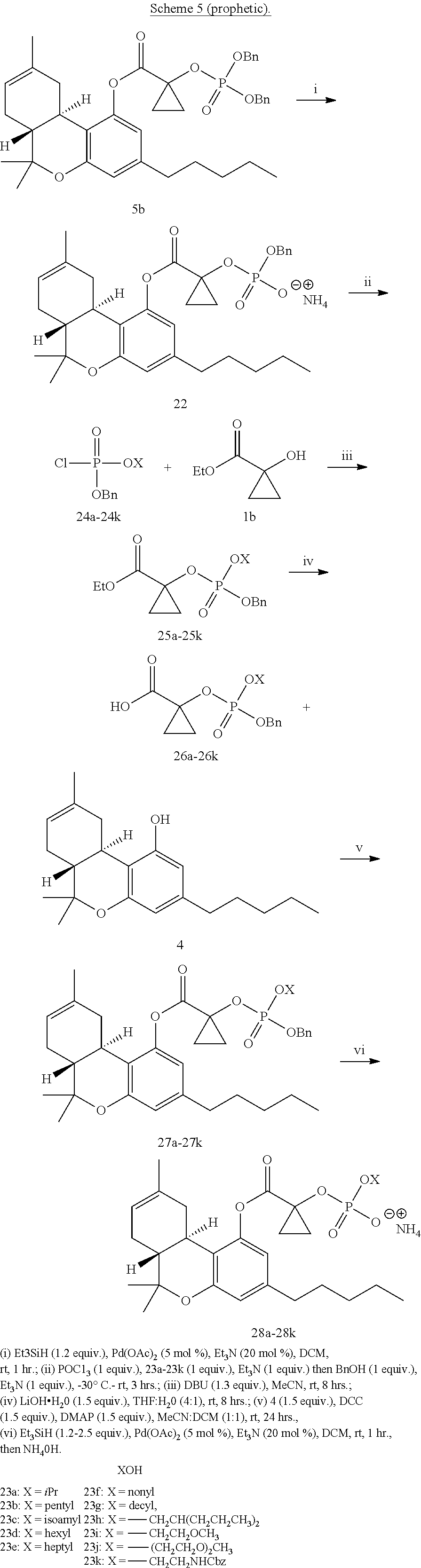

Firstly, for efficient mono-debenzylation of (−)trans-Δ⁸-THC prodrugs, the stoichiometric amount of triethylsilane added in the palladium acetate catalyzed benzyl phosphate deprotection of 5b should be reduced to 1.2 equivalents. This can facilitate mono-debenzylation of 5b and can afford the mono-benzyl di-ester phosphate prodrug 22, which will be furnished as the ammonium salt following treatment with NH₄OH.

To access the remaining di-ester phosphate prodrugs 28a-k, the corresponding substituted phosphorochloridates can first be synthesized. A cooled solution of phosphoryl chloride can be sequentially treated by dropwise addition of one equivalent of triethylamine and the respective alcohol (9a-k), followed by one equivalent of triethylamine and benzyl alcohol. Following aqueous workup with 1 M citric acid, the phosphorochloridates 10a-k can be isolated by column chromatography. Acylation of 1b with 24a-k can be carried out utilizing DBU as base to afford 25a-k, which can be followed by subsequent ester hydrolysis using lithium hydroxide monohydrate in a THF/H₂O mixture to afford the carboxylic acid derivatives 26a-k. As previously described, Steglich esterification between 4 and 26a-k can afford the respectively substituted phosphate compounds 27a-k. Debenzylation using 1.2 equivalents of triethylsilane and palladium acetate can afford the desired di-ester phosphate prodrugs 28a-k as ammonium salts following treatment with NH₄OH.

### Example 6. Synthesis of Ester Linked Cyclical-Diester Phosphate Prodrug of Δ⁸-THC (Prophetic Compound 33)

### A. Synthetic Procedure

Compounds with a cyclical di-ester phosphate prodrug moiety attached by an ester linker to (−)trans-Δ⁸-THC are designed and can be synthesized using the procedures described below. The prophetic synthesis of the ester linked cyclical di-ester phosphate prodrug 33 is shown in Scheme 6.

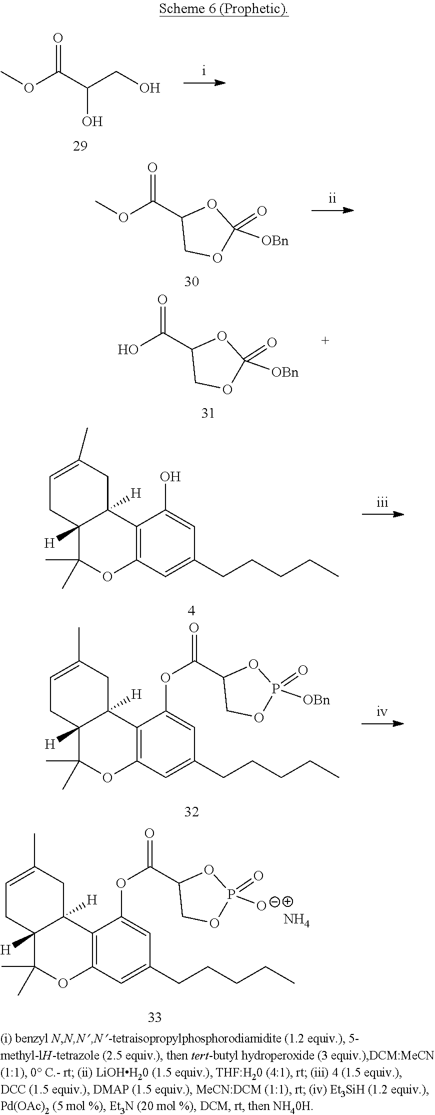

For the prophetic synthesis of the cyclical di-ester phosphate prodrugs, the steps will be like that undertaken for the previously synthesized prodrugs. Reaction of commercially available 29 with benzyl N,N,N′,N′-tetraisopropylphosphorodiamidite using 5 -methyl-1H-tetrazole, followed by oxidation to the desired phosphate can yield intermediate 30. This will be followed by lithium hydroxide mediated hydrolysis, and Steglich esterification with 4 to then yield the mono-benzyl protected cyclical phosphate intermediate 32. Lastly, debenzylation via triethylsilane and palladium acetate and treatment with ammonium hydroxide will afford the desired prodrug 33 as the ammonium salt.

### Example 7. Synthesis of Methylene Carbonate Linked Mono-Ester Phosphate Prodrug of Δ⁸-THC (Prophetic Compound 43)

### A. Synthetic Procedure:

Compounds with a mono-ester phosphate prodrug moiety attached by carbonate linker to (−)trans-Δ⁸-THC are designed and can be synthesized using the procedures described below. The prophetic synthesis of the methylene carbonate linked mono-ester phosphate prodrug 43 is shown in Scheme 7.

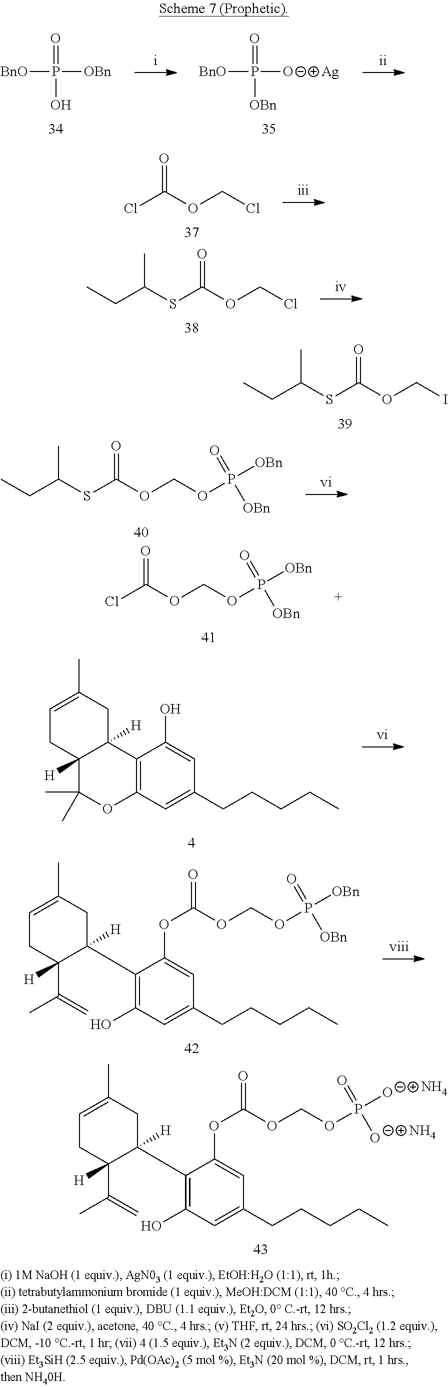

To access intermediate 40, the two component fragments 36 and 39 will first have to be synthesized. The stepwise conversion of dibenzyl phosphate 15 to its silver salt using silver nitrate, followed by treatment with tetrabutylammonium bromide can afford the tetrabutylammonium salt 36. For fragment 39, acylation between 2-butanethiol and chloromethyl chloroformate yielded intermediate 38, which can undergo a Finkelstein reaction to furnish 39. Fragments 36 and 39 can then reacted in THF for 24 hr to afford intermediate 40. Conversion to the acid chloride 41 will facilitated by sulfuryl chloride, which will be immediately followed by acylation with (−)trans-Δ⁸-THC 4, to afford the dibenzyl phosphate compound 42. Lastly, palladium acetate and triethylsilane mediated debenzylation and treatment with NH₄OH will afford the target methylene carbonate linked phosphate prodrug 43 as the di-ammonium salt.

### Example 8. Nephelometry

### A. General Experimental

Nephelometry experiments were performed using untreated CORNING® COSTAR® 96-well black polystyrene plates with clear flat bottoms. Sample stock solutions and serial dilutions were prepared with DRISOLV® DMSO purchased from MilliporeSigma. All 100-fold dilutions and replicate experiments were prepared using GIBCO® Dulbecco's phosphate-buffered saline (DPBS, no calcium, no magnesium) with apH range of 7.0-7.3 as aqueous media. Incubation of the 96-well plates was achieved with a Benchmark Incu-Shaker™ Mini shaking incubator. Nephelometry data was obtained using a NEPHELOSTAR® microplate reader and processed with MARS data analysis software from BMG LabTech.

### B. Procedure for Nephelometry Experiments

Tested compounds were dissolved in 100% DMSO to make stock solutions of specified concentrations, ranging from 10 mM minimum up to 50 mM maximum. The sample then underwent serial dilution in a 96-well plate (CORNING® COSTAR®). Well A1 of the plate contained 100% DMSO. Wells A2-A12 possessed the test compound in DMSO with concentration factors as follows: X mM for A2, (0.8)X mM for A3, (0.6)X mM for A4, (0.4)X mM for A5, (0.2)X mM for A6, (0.1)X mM for A7, (0.05)X mM for A8, (0.025)X mM for A9, (0.0125)X mM for A10. (0.00625)X mM for A11, and (0.003125)X mM for A12. Using a 12-channel multichannel pipette, 2.5 μL of sample from row A was transferred to each well in row B through row H of the plate. Next, 30 μL of PBS (pH=7.0-7.3) was added to row B through row H, providing each well with 32.5 μL. The plate was then incubated for 30 sec with shaking. Finally. 217.5 μL of PBS buffer was added to row B through row H, and the entire plate was incubated with shaking at 25° C. for 90 min. The final volume of DMSO in actual experiments with PBS buffer was 1% throughout the plate. After 90 min. the 96-well plate was analyzed with a NEPHELOSTAR® instrument and the data was processed with MARS data analysis software. The solubility data determined by the nephelometry experiments are summarized in Table 1.

### Example 9. Liver Microsome Stability Assay

### A. General Experimental

Human and mouse liver microsomes were purchased from Xenotech at 20 mg/mL. NADPH (Sigma-Aldrich) 10 mM stocks were prepared in deionized water.

### B. Procedure for Liver Microsome Stability Assay

Test compounds and positive controls were dissolved in 100% DMSO to make 10 mM stock solutions. Verapamil (Sigma-Aldrich) and diphenhydramine (Sigma-Aldrich) were used as positive controls for both human liver microsomes (HLM) and mouse liver microsomes (MLM). The 10 mM stock solutions of test and control compounds were further diluted in potassium phosphate buffer (100 mM, pH 7.4) to 500 μM to ensure that the organic solvent content was <0.2%. The liver microsome (HLM or MLM) assay was prepared in a 1.5 mL Eppendorf tube with a final volume of 1100 μL for duplicate runs. Each reaction contained phosphate buffer (928.4 μL), liver microsomes (55 μL), and test compound (6.6 μL of 500 μM), resulting in a final concentration of 3 μM for the test compound. The reaction was initiated with 110 μL of 10 mM NADPH. Aliquots (100 μL) were removed in duplicate at 0, 5, 10, 15, and 30 min and quenched in 100 mL of 100% cold methanol which contained internal standard (ISTD: d₅-7-ethoxy coumarin 2 μM). The aliquots were centrifuged at 12,000g for 5 min, and the supernatant was transferred and placed in an LC-MS vial. Each time point was assessed by LC-MS/MS, and the area under the m/z curve (AUC), based on the MRM transition, was integrated w.r.t the ISTD. Positive controls were conducted at a final volume of 550 μL to enable a single run for each time point. Negative controls in the absence of NADPH were performed with the test and control compounds, respectively, at a final volume of 150 μL and measured at the most prolonged time point. Control compounds were processed and analyzed like test compounds. Each time point was run in triplicates on LC-MS/MS followed by in-between blank washes to avoid the carryover and to equilibrate the column. Half-life (T_{1/2}) was calculated using the data obtained from LC-MS/MS and by plotting ln (% remaining of the parent prodrug) versus time and performing linear regression to determine slope. Slope=−k and T_{1/2=0.693}/k for first-order kinetics. The results of the HLM stability assays are summarized in Table 1.

### Example 10. Plasma Stability Assay

### A. General Experimental

Procaine and Procainamide were purchased from Sigma-Aldrich. Human plasma was obtained from BioIVT (Cat. No. HUMANPLLHP2N) and 1× Dulbecco's PBS (pH 7.4, Gibco) was obtained from Thermos Fischer. Stock solutions were prepared in 100% DMSO, and suitable dilutions of target analytes were prepared in methanol for method development.

### B. Procedure for Plasma Stability Assay

Test compounds were dissolved in DMSO to make a stock solution of 10 mM and then diluted to 500 μM in buffer (or 70% MeOH). Human plasma was thawed at ambient temperature and is aliquoted (994.0 μL) to a 1.5 mL Eppendorf tube in duplicates (A and B) for each compound. Plasma was incubated at 37° C. for 10 minutes in an incubator shaker at 150 rpm; the reaction was initiated by the addition of the test compound (6.0 μL) and vortex mixing. The total reaction volume was 1000 μL where the final organic solvent concentrations were 0.6% MeOH and 0.03% DMSO. The spiked plasma samples were incubated at 37° C. for 4 hrs. The reactions were terminated at time points 0, 15, 30, 60, and 120 min by taking a 100 μL aliquot from the test incubation mixture and immediately quenching it in ice-cold mathanol containing internal standard (150 μL with 2 μM ISTD), followed by a quick vortex mixing. In addition, matrix blank was prepared by adding methanol solutions containing ISTD to plasma samples without any of the analytes or control compounds. Also, an additional tube was made to measure compound degradation in PBS buffer. The samples were centrifuged at 15,000 rpm for 25 min at 4° C. The supernatant was transferred to an LC-MS vial for analysis by LC/MS-MS. Each time point was run in duplicates followed by in-between blank washes to avoid the carryover and to equilibrate the column. Half-life (T_{1/2}) was calculated using the data obtained from LC-MS/MS and by plotting ln (% remaining of the parent prodrug) versus time and performing linear regression to determine slope. Slope=−k and T_{1/2}=0.693/k for first-order kinetics. Procaine (poor plasma stability) was used as a positive control at a final concentration of 3 μM. The positive control was run in parallel to test the systems for competency.

An exemplary experimental setup is described below. Conical polypropylene microcentrifuge tubes (1.5 mL) were labelled in duplicate for 0, 15, 30, 60, and 120 min for each compound being tested.
Example: No. of tubes prepared per Test Compound (TC):
Test compound: 994 μL human plasma+6.0 μL TC (Vial A)
   994 μL human plasma+6.0 μL TC (Vial B)
Positive control: 596 μL human plasma+3.6 μL (procaine)
Matrix blank: 500 μL PBS buffer+100 μL human plasma
Negative control: 142 μL PBS buffer+0.9 μL TC1
Quenching mixture: 150 μL methanol with ISID (2 μM)
Final volume after quenching: 250 μL (100 μL from the incubation mixture and 150 μL from the quenching mixture; ISTD conc. 1.2 μM)

The results of the human plasma stability assays are summarized in Table 1.

### Example 11. Bioanalytical Results

The following table details the bioanalytical results of exemplary compounds of the present disclosure.

TABLE 1
Bioanalytical data of phosphate prodrugs of Δ⁸-THC.

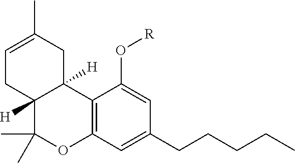

| | | Solubility | | T½ in | |
|---|---|---|---|---|---|
| | | in 1% | T½ in | human | Δ⁸-THC |
| | | DMSO | HLM | plasma | detected |
| Cpd | R | (μM) | (min) | (h) | in plasma |
| Δ⁸-THC | H | 15.93 ± 0.8 | 5.51 | 24 ± 6 | — |
| 5a | 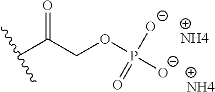 [SMILES: *C(=O)COP(=O)([O-])[O-] ; [NH4+] ; [NH4+]] | >500 | 76 | — | — |
| 5b | 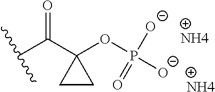 [SMILES: *C(=O)C1(OP(=O)([O-])[O-])CC1 ; [NH4+] ; [NH4+]] | >500 | 173 | >2 | Yes |
| 5c | 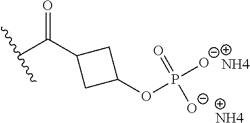 [SMILES: *C(=O)C1CC(OP(=O)([O-])[O-])C1 ; [NH4+] ; [NH4+]] | >500 | 165 | — | — |
| 5d | 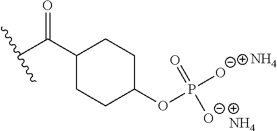 [SMILES: *C(=O)C1CCC(OP(=O)([O-])[O-])CC1 ; [NH4+] ; [NH4+]] | >500 | 203 | — | — |
| 5e | 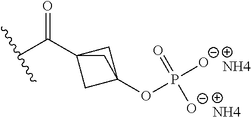 [SMILES: *C(=O)C12CC(OP(=O)([O-])[O-])(C1)C2 ; [NH4+] ; [NH4+]] | >500 | 154 | — | — |
| 5f | 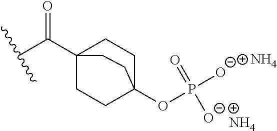 [SMILES: *C(=O)C12CCC(OP(=O)([O-])[O-])(CC1)CC2 ; [NH4+] ; [NH4+]] | >500 | 315 | — | — |
| 11a | 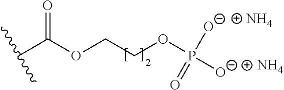 | >500 | 18.8 | — | — |
| 11b | 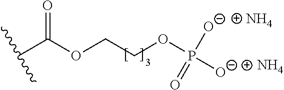 | >500 | 17.2 | >2 | Yes |
| 16a | 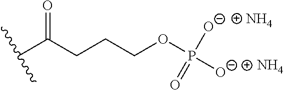 [SMILES: *C(=O)CCCOP(=O)([O-])[O-] ; [NH4+] ; [NH4+]] | >500 | 51.3 | >2 | Yes |
| 16b | 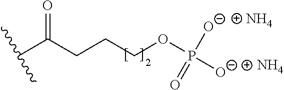 | >500 | 40.1 | — | — |
| 21a | 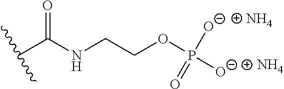 [SMILES: *C(=O)NCCOP(=O)([O-])[O-] ; [NH4+] ; [NH4+]] | >500 | 5.6 | — | — |
| 21b | 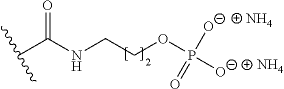 | >500 | 12.5 | >2 | Yes |

What is claimed is:

1. A compound of Formula (I): 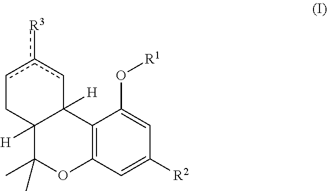
and pharmaceutically acceptable salts thereof, wherein
R¹ is chosen from 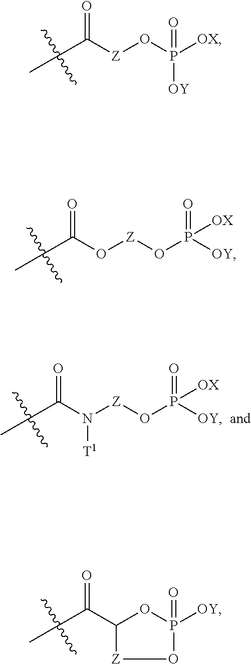
wherein
Z is chosen from divalent C₁₋₁₈ alkyl and divalent C₁₋₁₈ haloalkyl groups, wherein the divalent C₁₋₁₈ alkyl and divalent C₁₋₁₈ haloalkyl groups are optionally substituted with one or more groups independently chosen from C₆₋₁₈ aryl, C₁₋₁₃ heteroaryl, C₂₋₁₂ heterocyclyl, —OC₁₋₁₈ alkyl, —SC₁₋₁₈ alkyl, —N(T²)C₁₋₁₈ alkyl, and —N(T²)AA groups, wherein T² is chosen from H and C₁₋₈ alkyl groups and AA is chosen from amino acid residues and is attached to the nitrogen via its C-terminus carbonyl group,
X and Y, which may be identical or different, are independently chosen from H, Q, C₁₋₁₈ alkyl, C₁₋₁₈ haloalkyl, C₆₋₁₈ aryl, C₁₋₁₃ heteroaryl, C₇₋₁₉ arylalkyl, C₂₋₁₄ heteroarylalkyl, —(CH₂CH₂O)ₙCH₃, 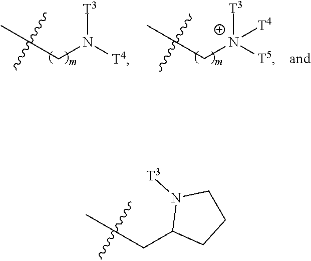
groups, wherein
each T³, T⁴, and T⁵, which may be the same or different, are independently chosen from H and C₁₋₈ alkyl groups,
each Q is independently chosen from pharmaceutically acceptable cations,
each n is independently chosen from integers ranging from 1 to 12, and
each m is independently chosen from integers ranging from 1 to 8, and
T¹ is chosen from H, C₁₋₁₈ alkyl, and C₁₋₁₈ haloalkyl groups;
R² is chosen from C₁₋₁₀ alkyl groups and C₁₋₁₀ haloalkyl groups; and
R³ is chosen from —CH₃, —CD₃, —CH₂OH, —CH₂OR^{a}, and ═O, wherein R^{a} is chosen from C₁₋₈ alkyl groups and C₁₋₈ haloalkyl groups.

2. The compound according to claim 1, wherein R¹ is chosen from 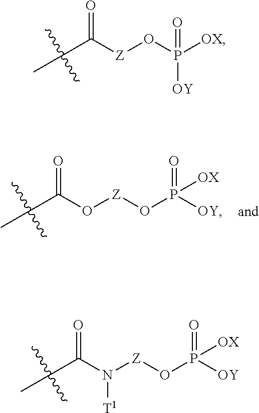
groups.

3. The compound according to claim 1, wherein R¹ is chosen from 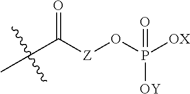
groups.

4. The compound according to claim 1, wherein R¹ is chosen from 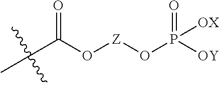
groups.

5. The compound according to claim 1, wherein R¹ is chosen from 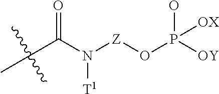
groups.

6. The compound according to claim 1, wherein R¹ is chosen from 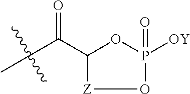
groups.

7. The compound according to claim 3, wherein the carbon in Z that is next to the carbonyl group has no hydrogen atoms.

8. The compound according to claim 3, wherein the carbon in Z that is next to the carbonyl group has one hydrogen atom.

9. The compound according to claim 3, wherein the carbon in Z that is next to the carbonyl group is substituted.

10. The compound according to claim 1, wherein Z is chosen from divalent C₁₋₁₈ alkyl groups.

11. The compound according to claim 10, wherein Z is chosen from —CH₂—, —(CH₂)₂—, —(CH₂)₃—, —(CH₂)₄—, —(CH₂)₅—, —(CH₂)₆—, —(CH₂)₇—, —(CH₂)₈—, —(CH₂)₉—,

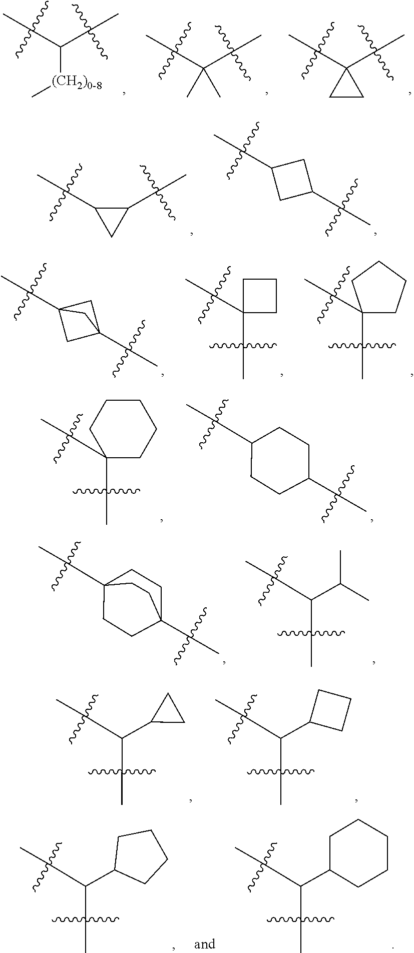

12-14. (canceled)

15. The compound according to claim 11, wherein Z is

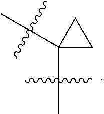 [SMILES: *C1(*)CC1]

16. The compound according to claim 11, wherein Z is

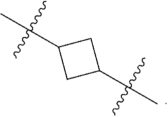 [SMILES: *C1CC(*)C1]

17-98. (canceled)

99. The compound according to claim 1, wherein the 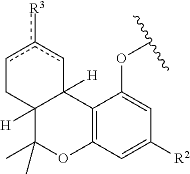
moiety is chosen from

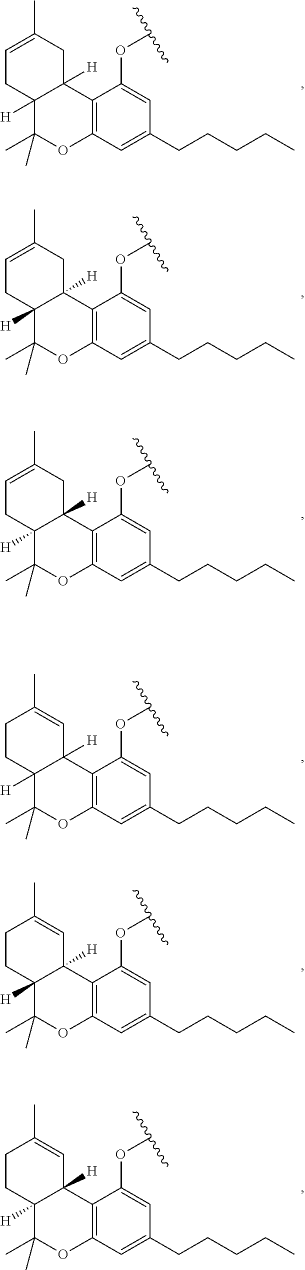

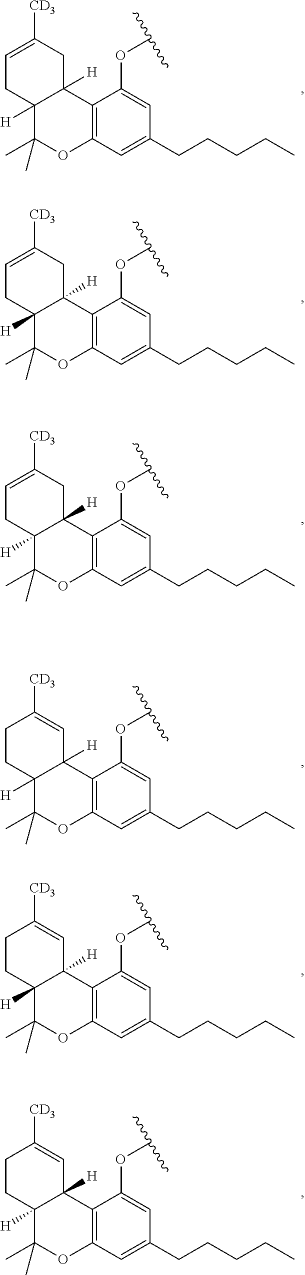

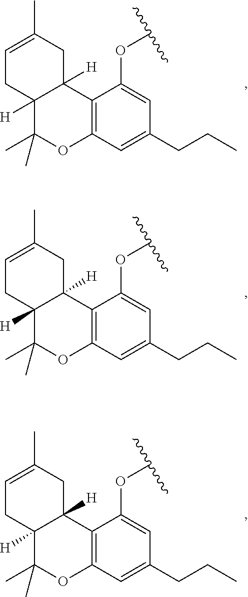

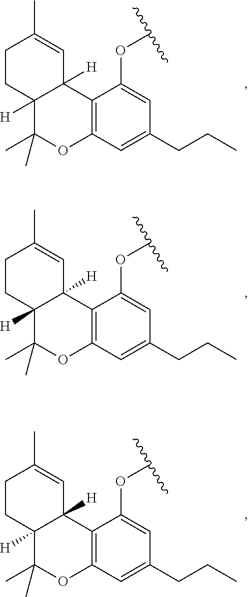

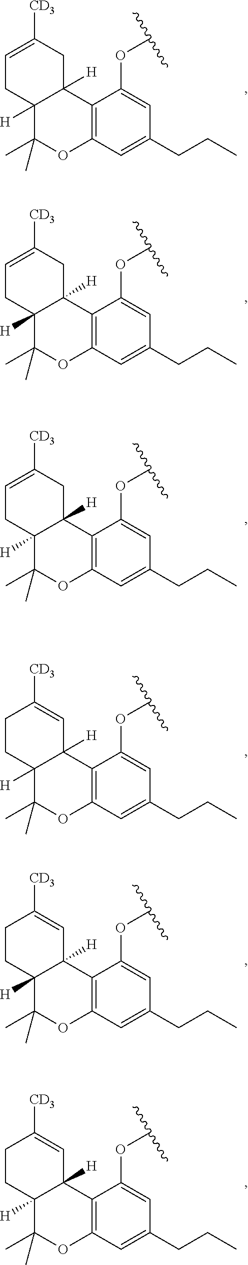

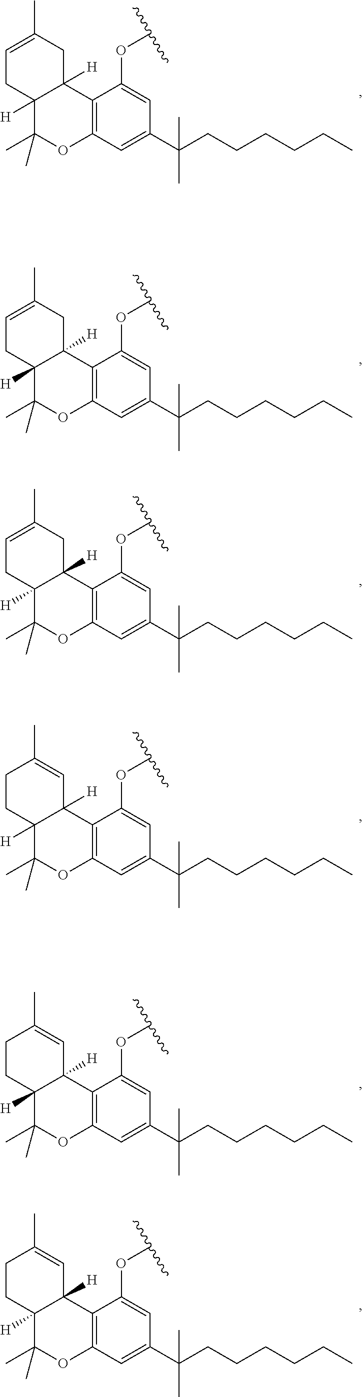 [SMILES: *Oc1cc(C(C)(C)CCCCCC)cc2c1C1CC(C)=CCC1C(C)(C)O2 ; *Oc1cc(C(C)(C)CCCCCC)cc2c1[C@@H]1CC(C)=CC[C@H]1C(C)(C)O2 ; *Oc1cc(C(C)(C)CCCCCC)cc2c1[C@H]1CC(C)=CC[C@@H]1C(C)(C)O2 ; *Oc1cc(C(C)(C)CCCCCC)cc2c1C1C=C(C)CCC1C(C)(C)O2 ; *Oc1cc(C(C)(C)CCCCCC)cc2c1[C@@H]1C=C(C)CC[C@H]1C(C)(C)O2 ; *Oc1cc(C(C)(C)CCCCCC)cc2c1[C@H]1C=C(C)CC[C@@H]1C(C)(C)O2]

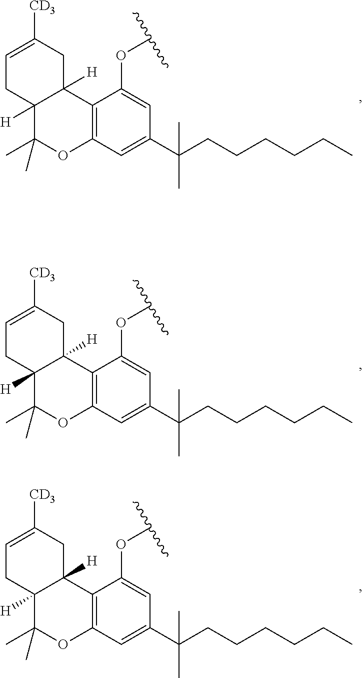 [SMILES: *Oc1cc(C(C)(C)CCCCCC)cc2c1C1CC(C([2H])([2H])[2H])=CCC1C(C)(C)O2 ; *Oc1cc(C(C)(C)CCCCCC)cc2c1[C@@H]1CC(C([2H])([2H])[2H])=CC[C@H]1C(C)(C)O2 ; *Oc1cc(C(C)(C)CCCCCC)cc2c1[C@H]1CC(C([2H])([2H])[2H])=CC[C@@H]1C(C)(C)O2]

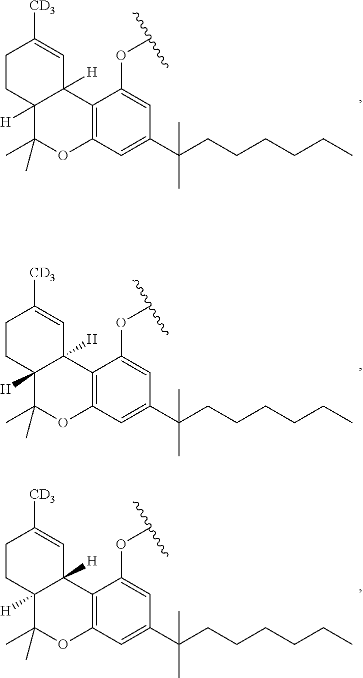 [SMILES: *Oc1cc(C(C)(C)CCCCCC)cc2c1C1C=C(C([2H])([2H])[2H])CCC1C(C)(C)O2 ; *Oc1cc(C(C)(C)CCCCCC)cc2c1[C@@H]1C=C(C([2H])([2H])[2H])CC[C@H]1C(C)(C)O2 ; *Oc1cc(C(C)(C)CCCCCC)cc2c1[C@H]1C=C(C([2H])([2H])[2H])CC[C@@H]1C(C)(C)O2]

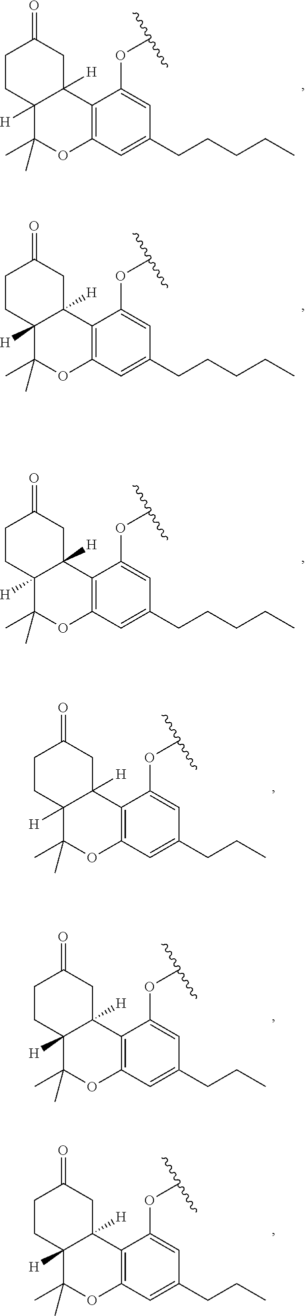 [SMILES: *Oc1cc(CCCCC)cc2c1C1CC(=O)CCC1C(C)(C)O2 ; *Oc1cc(CCCCC)cc2c1[C@@H]1CC(=O)CC[C@H]1C(C)(C)O2 ; *Oc1cc(CCCCC)cc2c1[C@H]1CC(=O)CC[C@@H]1C(C)(C)O2 ; *Oc1cc(CCC)cc2c1C1CC(=O)CCC1C(C)(C)O2 ; *Oc1cc(CCC)cc2c1[C@@H]1CC(=O)CC[C@H]1C(C)(C)O2 ; *Oc1cc(CCC)cc2c1[C@@H]1CC(=O)CC[C@H]1C(C)(C)O2]

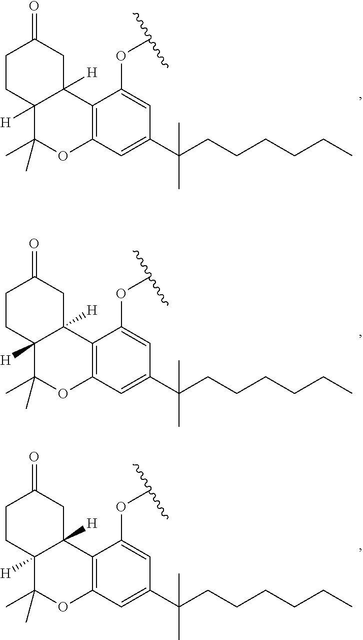 [SMILES: *Oc1cc(C(C)(C)CCCCCC)cc2c1C1CC(=O)CCC1C(C)(C)O2 ; *Oc1cc(C(C)(C)CCCCCC)cc2c1[C@@H]1CC(=O)CC[C@H]1C(C)(C)O2 ; *Oc1cc(C(C)(C)CCCCCC)cc2c1[C@H]1CC(=O)CC[C@@H]1C(C)(C)O2]

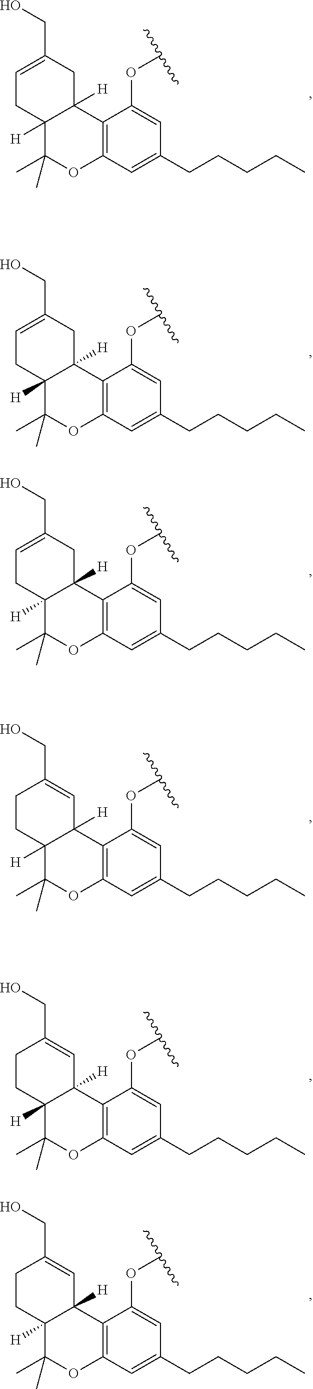

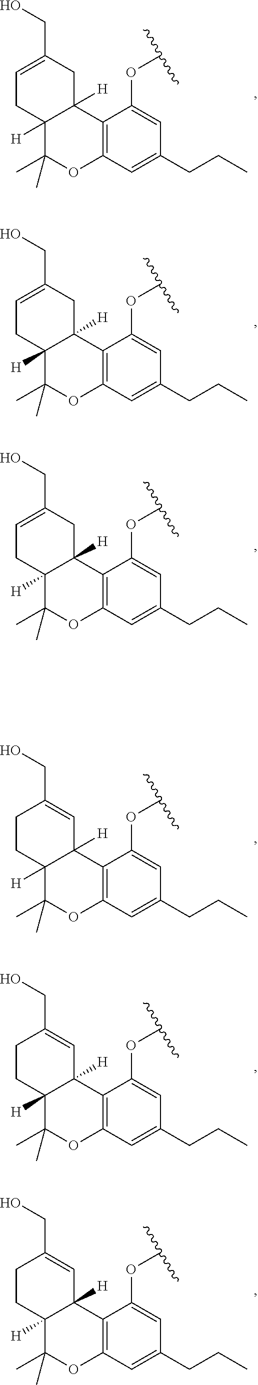

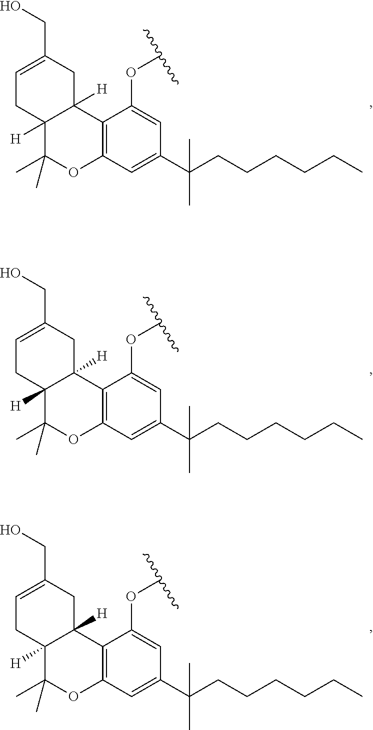 [SMILES: *Oc1cc(C(C)(C)CCCCCC)cc2c1C1CC(CO)=CCC1C(C)(C)O2 ; *Oc1cc(C(C)(C)CCCCCC)cc2c1[C@@H]1CC(CO)=CC[C@H]1C(C)(C)O2 ; *Oc1cc(C(C)(C)CCCCCC)cc2c1[C@H]1CC(CO)=CC[C@@H]1C(C)(C)O2]

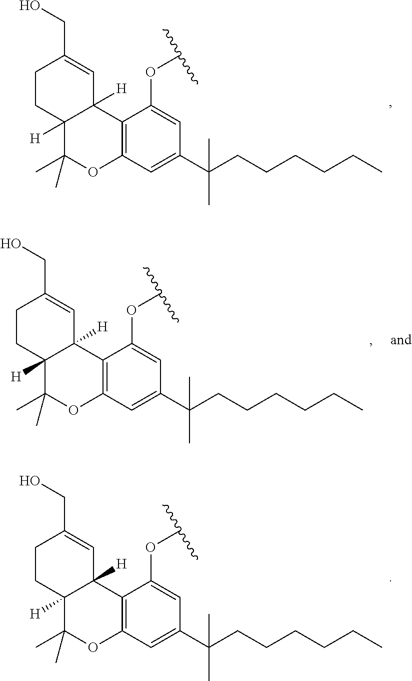 [SMILES: *Oc1cc(C(C)(C)CCCCCC)cc2c1C1C=C(CO)CCC1C(C)(C)O2 ; *Oc1cc(C(C)(C)CCCCCC)cc2c1[C@@H]1C=C(CO)CC[C@H]1C(C)(C)O2 ; *Oc1cc(C(C)(C)CCCCCC)cc2c1[C@H]1C=C(CO)CC[C@@H]1C(C)(C)O2]

100. A composition comprising the compound of claim 1 and a pharmaceutically acceptable carrier.

101-103. (canceled)

104. A method for treatment and/or prevention of at least one disease, disorder, and/or condition where treatment with an anxiolytic agent, analgesic agent, antiemetic agent, mood-stabilizing agent, antipsychotic agent, muscle relaxation agent, immunosuppressant, anti-inflammatory agent, anti-allergic agent, antioxidant agent, anticancer agent, neuroprotective agent, anti-convulsant agent, antineoplastic agent, appetite stimulant, intraocular pressure regulator, and/or bronchodilator is useful, the method comprising administering to a subject in need thereof an effective amount of the compound or claim 1.

105. A method for treatment and/or prevention of at least one psychiatric disease, disorder, and/or condition, the method comprising administering to a subject in need thereof an effective amount of the compound of claim 1.

106-121. (canceled)

122. A compound of claim 1 selected from:

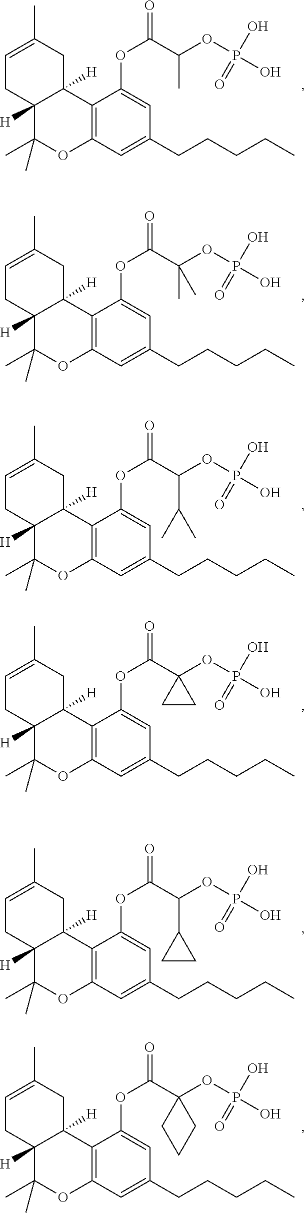

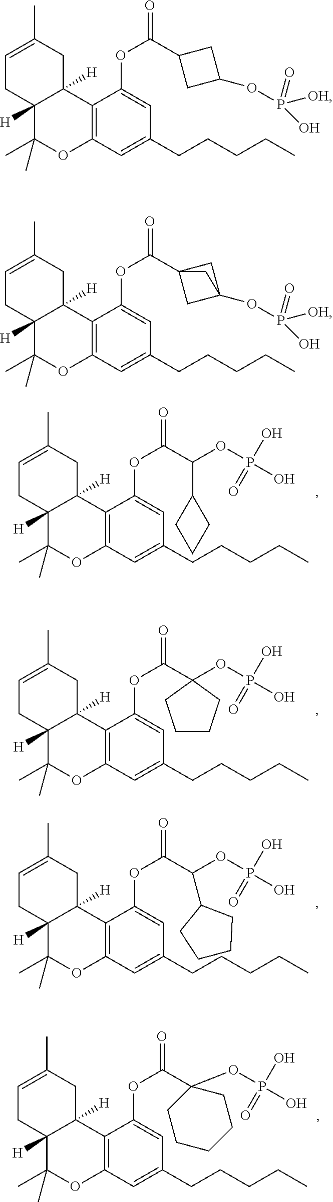

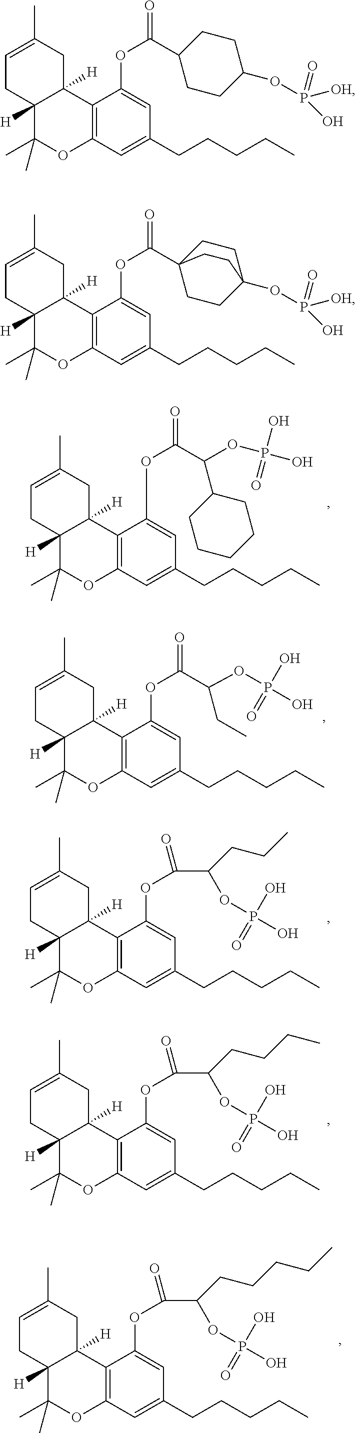

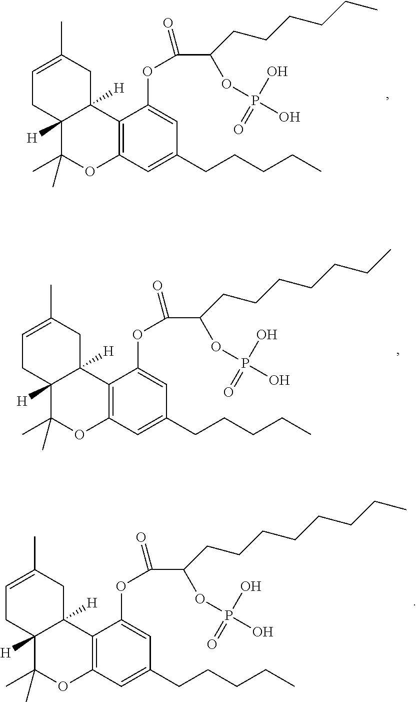 [SMILES: CCCCCCC(OP(=O)(O)O)C(=O)Oc1cc(CCCCC)cc2c1[C@@H]1CC(C)=CC[C@H]1C(C)(C)O2 ; CCCCCCCC(OP(=O)(O)O)C(=O)Oc1cc(CCCCC)cc2c1[C@@H]1CC(C)=CC[C@H]1C(C)(C)O2 ; CCCCCCCCC(OP(=O)(O)O)C(=O)Oc1cc(CCCCC)cc2c1[C@@H]1CC(C)=CC[C@H]1C(C)(C)O2]

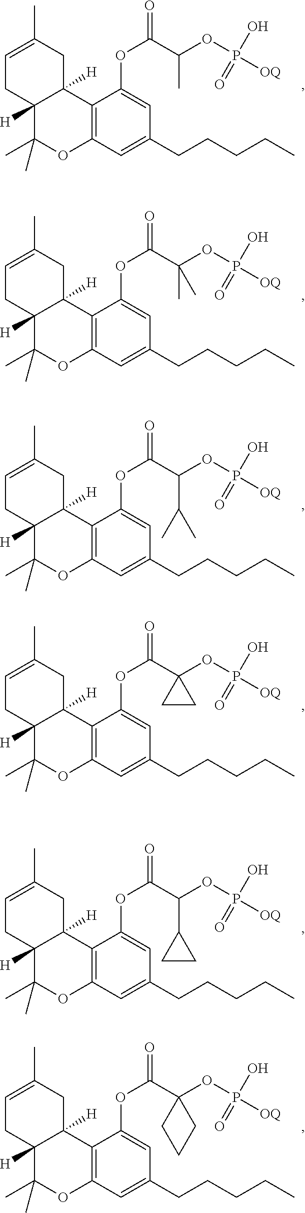

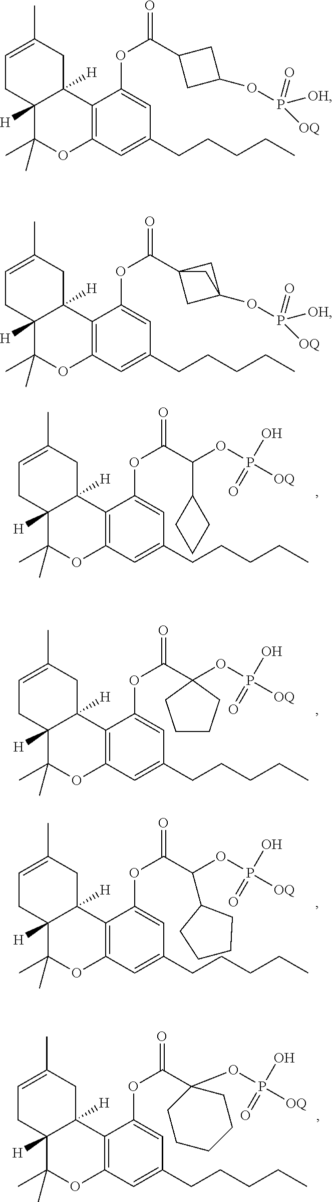

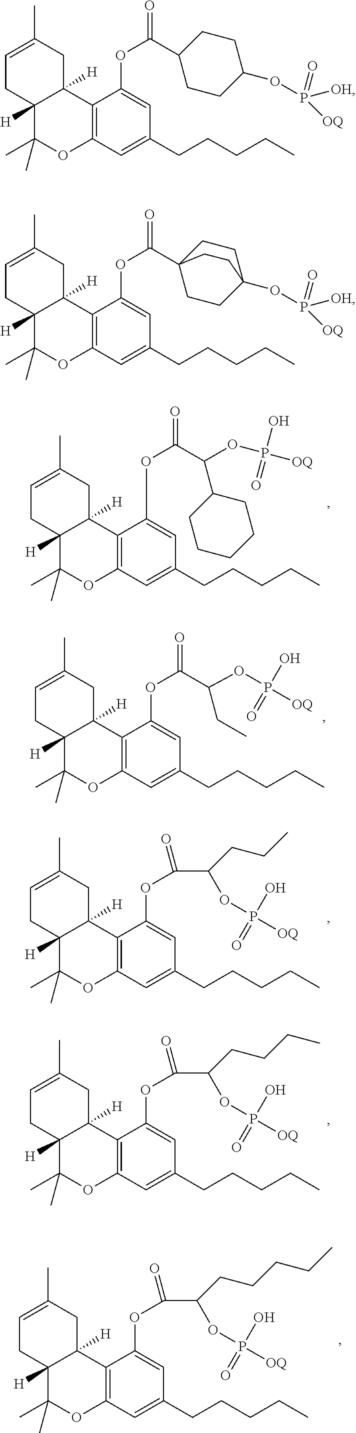

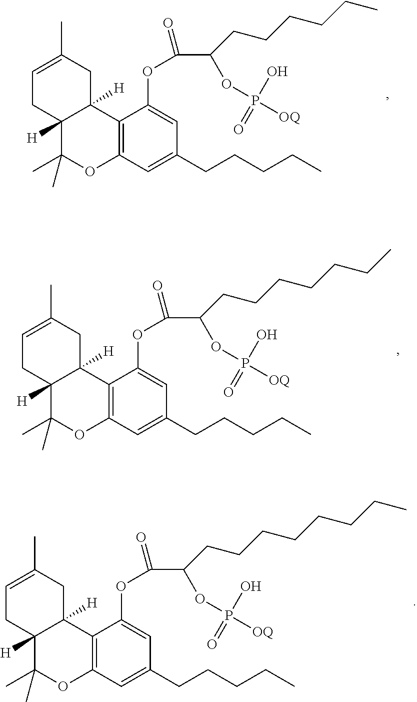

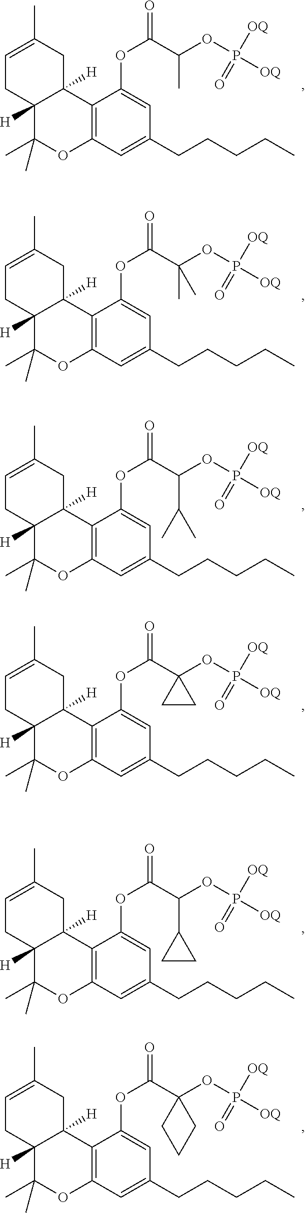

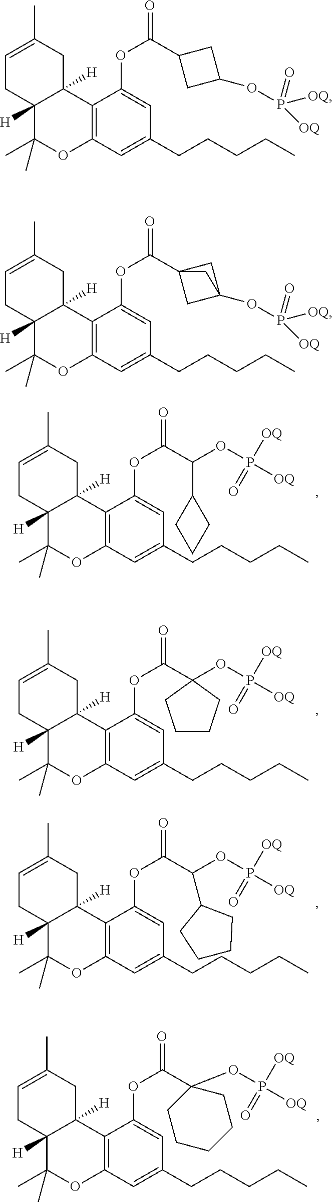

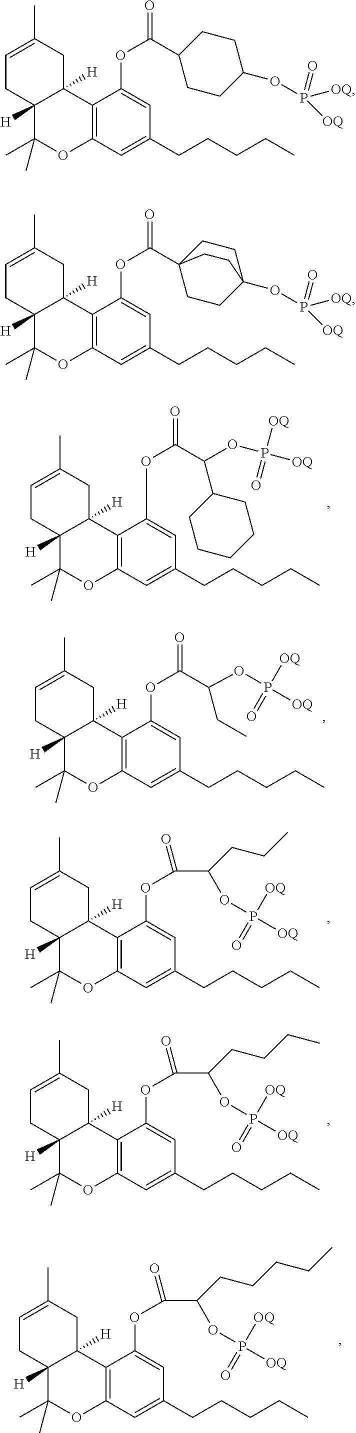

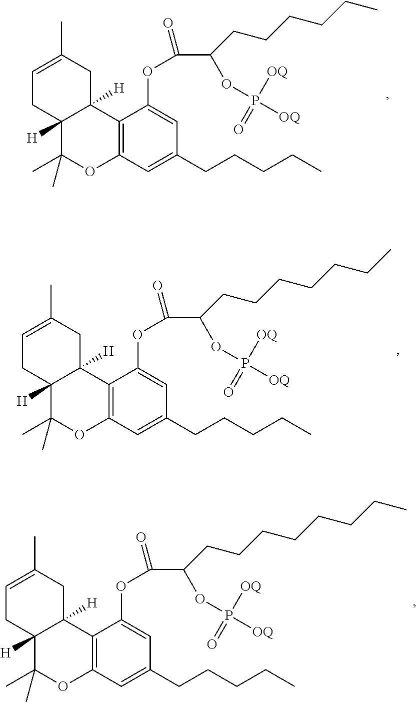

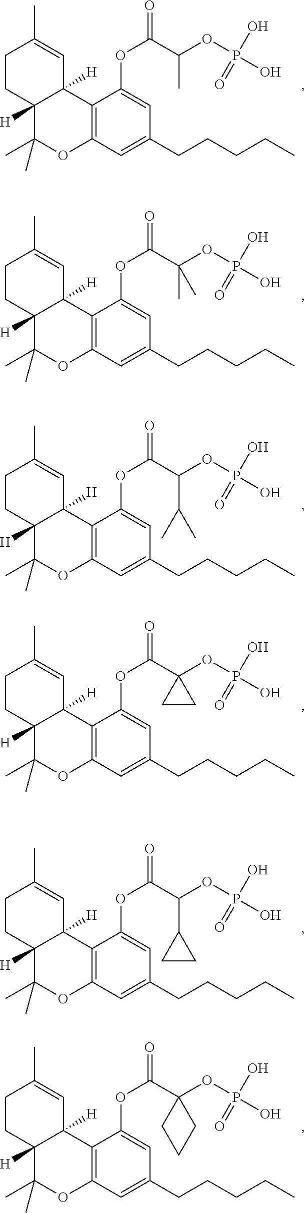

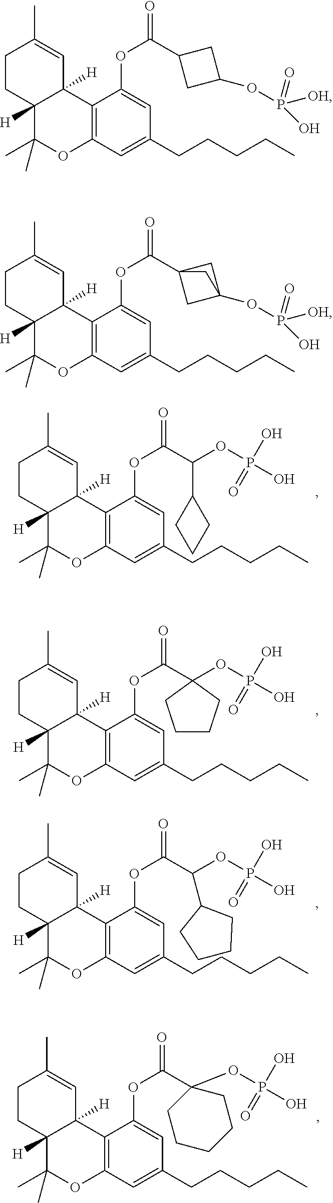

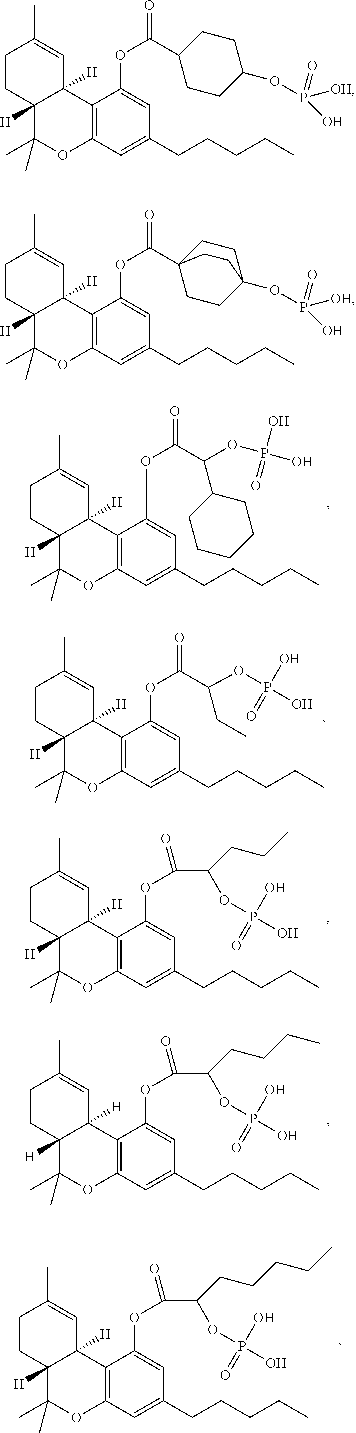

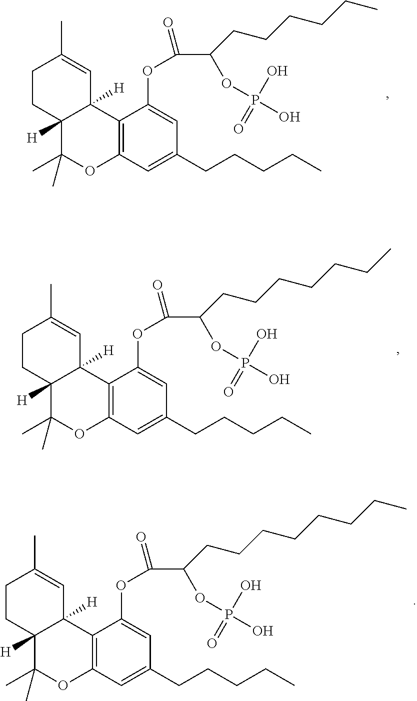 [SMILES: CCCCCCC(OP(=O)(O)O)C(=O)Oc1cc(CCCCC)cc2c1[C@@H]1C=C(C)CC[C@H]1C(C)(C)O2 ; CCCCCCCC(OP(=O)(O)O)C(=O)Oc1cc(CCCCC)cc2c1[C@@H]1C=C(C)CC[C@H]1C(C)(C)O2 ; CCCCCCCCC(OP(=O)(O)O)C(=O)Oc1cc(CCCCC)cc2c1[C@@H]1C=C(C)CC[C@H]1C(C)(C)O2]

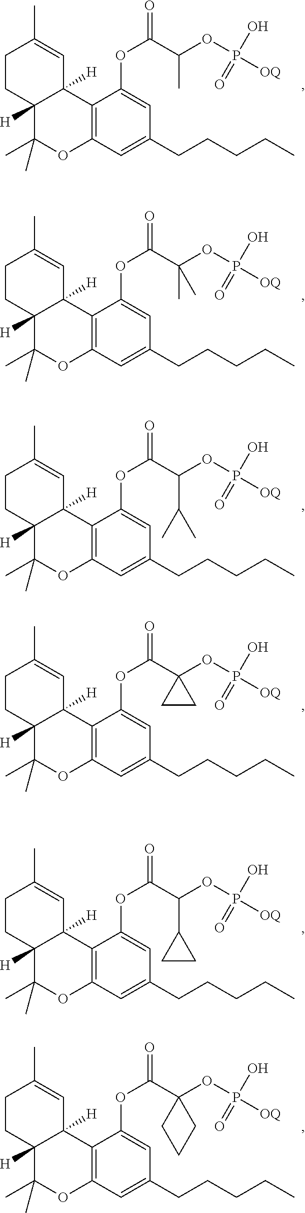

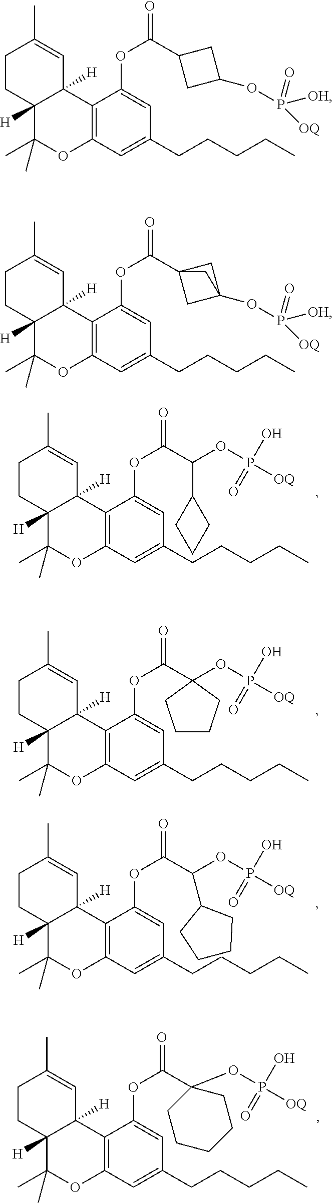

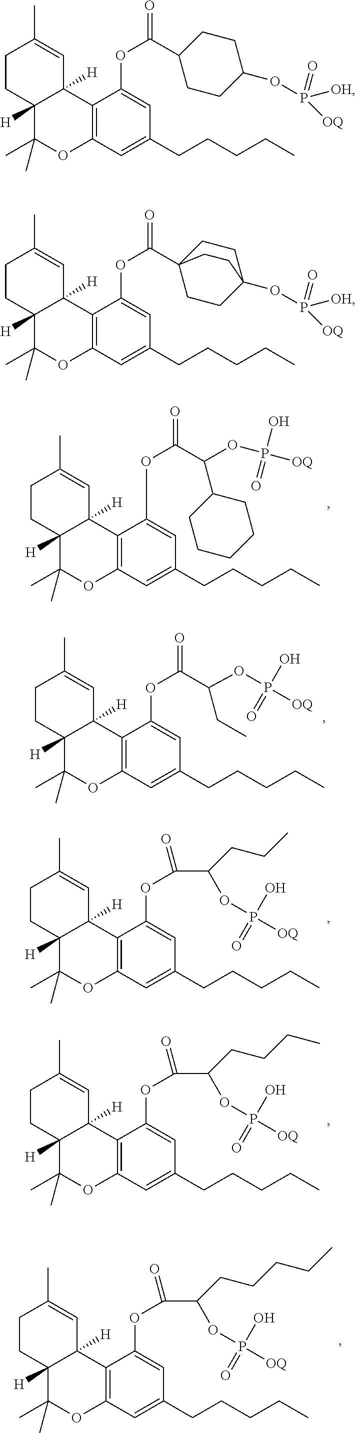

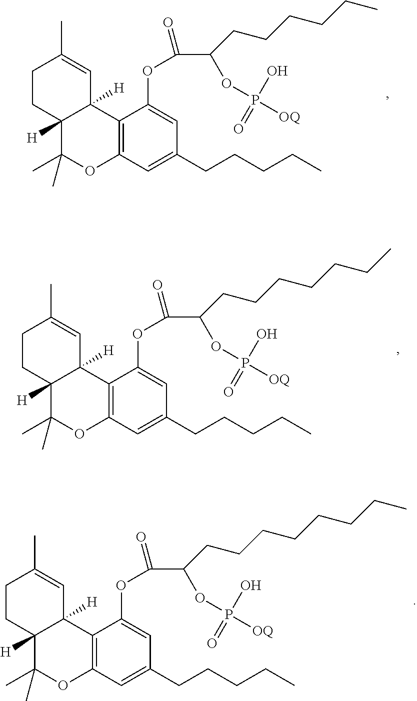

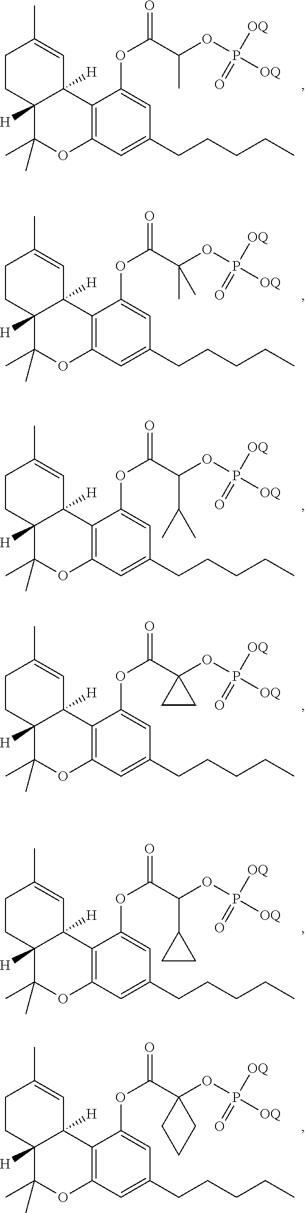

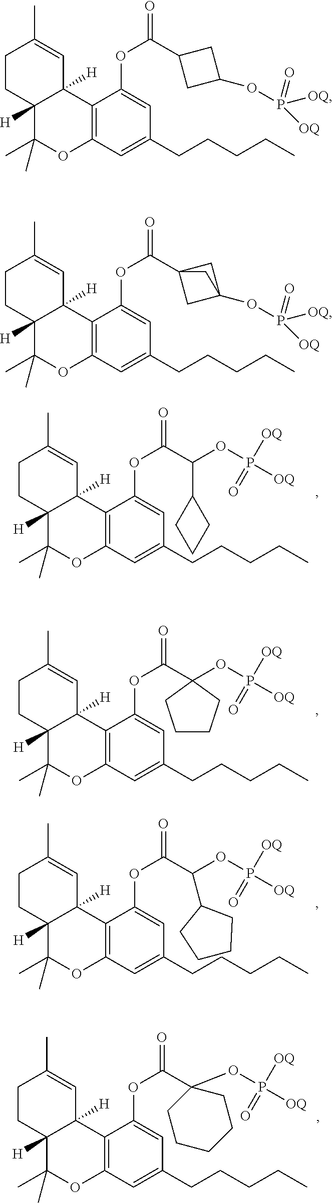

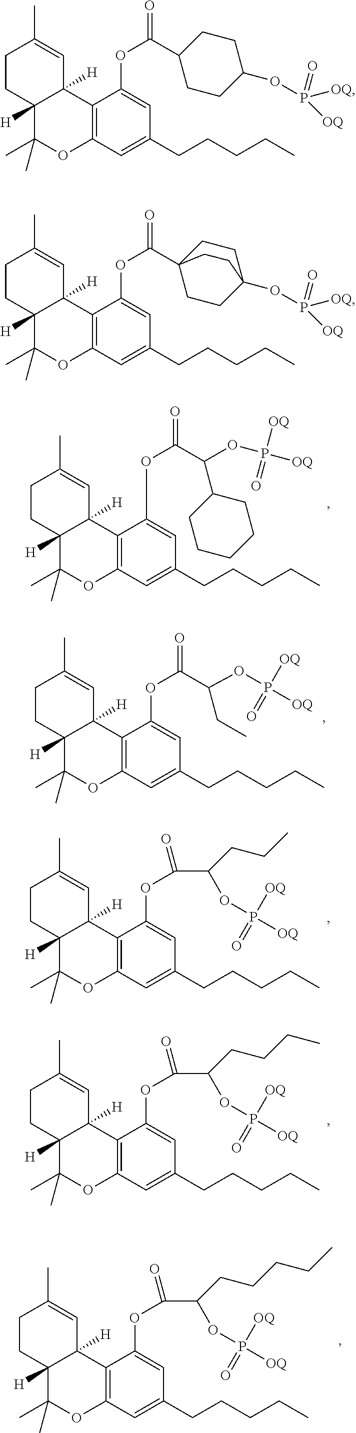

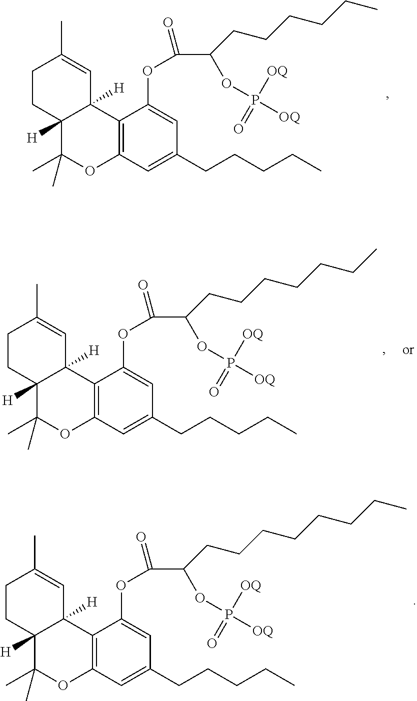

123. The compound of claim 1, which is (6aR,10aR)-6,6,9-trimethyl-3-pentyl-6a,7,10,10a-tetrahydro-6H-benzo[c]chromen-1-yl 1-(phosphonooxy)cyclopropane-1-carboxylate or salt thereof.

124. The compound of claim 1, which is (6aR,10aR)-6,6,9-trimethyl-3-pentyl-6a,7,10,10a-tetrahydro-6H-benzo[c]chromen-1-yl 3-(phosphonooxy)cyclobutane-1-carboxylate or salt thereof.
